(19) United States
(12) Patent Application Publication
(10) Pub. No.: US 2021/0213066 A1
(43) Pub. Date: Jul. 15, 2021

(54) IMPROVED CELL THERAPY COMPOSITIONS FOR HEMATOPOIETIC STEM CELL TRANSPLANT PATIENTS

(71) Applicant: CHILDREN'S NATIONAL MEDICAL CENTER, Washington, DC (US)

(72) Inventors: Catherine Mary Bollard, Bethesda, MD (US); Conrad Russell Y. Cruz, Bethesda, MD (US); Patrick Hanley, Washington, DC (US)

(73) Assignee: CHILDREN'S NATIONAL MEDICAL CENTER, Washington, DC (US)

(21) Appl. No.: 17/056,749

(22) PCT Filed: May 20, 2019
(86) PCT No.: PCT/US19/33186

§ 371 Date: Nov. 18, 2020

Related U.S. Application Data
(60) Provisional application No. 62/673,756, filed on May 18, 2018.

(51) Int. Cl. A61K 35/28; A61K 39/00; A61K 39/12; C12N 5/0783; C12N 5/0775

(52) U.S. Cl. CPC A61K 35/28; A61K 39/001149; A61K 39/12; C12N 5/0638; C12N 5/0663; A61K 39/001186; A61K 39/001153; A61K 39/00116; A61K 39/001184; A61K 39/001189; A61K 39/001188; A61K 39/00115

(57) ABSTRACT

The present disclosure provides for isolated and processed cell therapeutic compositions and Methods of using those compositions for the treatment of a patient undergoing a hematopoietic stem cell transplant (HSCT). In some embodiments, the disclosure provides for methods of making these cells by exposing the isolated T cell populations to one or more tumor antigens.

### RELATED APPLICATIONS

This application claims the benefit of provisional U.S. Application No. 62/673,756, filed, May 18, 2018, the entirety of which is hereby incorporated by reference for all purposes.

### FIELD OF THE INVENTION

The present invention provides isolated and processed cell therapeutic compositions and methods of using them for the treatment of a patient undergoing a hematopoietic stem cell transplant (HSCT) during treatment for a disorder such as a malignancy, primary immune deficiency, genetic disorder, metabolic disorder or a form of abnormal cellular proliferation such as an autoimmune disease. In certain aspects, the invention can be used for the combined prevention and/or treatment of cancer recurrence, viral infection, and graft versus host disease (GVHD). The isolated cell compositions provided herein include multiple cell subpopulations, wherein each specific cell subpopulation is directed to the prevention of, or treatment of, a particular comorbidity common with HSCT. The present invention also extends to methods of manufacturing such cell therapeutic compositions and the generation of a bank of multiple antigen-specific T-cell and mesenchymal stem cell compositions from healthy donors to provide an improved personalized cell therapy.

### BACKGROUND OF THE INVENTION

Hematopoietic stem cell transplantation (HSCT) involves the intravenous infusion of autologous or allogeneic stem cells collected from bone marrow, peripheral blood, or umbilical cord blood to reestablish hematopoietic function in patients whose bone marrow or immune system is damaged or defective. This procedure is often performed as part of therapy to eliminate a bone marrow infiltrative process, such as leukemia, or to correct congenital immunodeficiency disorders. In addition, HSCT is used to allow patients with cancer to receive higher doses of chemotherapy than bone marrow can usually tolerate. Bone marrow function is then salvaged by replacing the marrow with previously harvested stem cells. Examples of emerging indications for HSCT include replacement of marrow progenitors for the purpose of making normal red cells (e.g., in hemoglobinopathies), making corrective enzymes (e.g., in storage disorders), and mediating tissue repair (e.g., in epidermolysis bullosa). More than 50,000 first HSCTs—53% autologous and 47% allogeneic—are performed every year worldwide, according to the Worldwide Network of Blood and Marrow Transplantation. The number continues to increase by 10-20% annually.

The preparative or conditioning regimen is a critical element in hematopoietic stem cell transplantation (HSCT). The purpose of the preparative regimen is to provide immunosuppression sufficient to prevent rejection of the transplanted graft and to eradicate the disease for which the transplantation is being performed. These goals have traditionally been achieved by delivering maximally tolerated doses of multiple chemotherapeutic agents with nonoverlapping toxicities (with or without radiation). Infusion of hematopoietic cells circumvents the problem of prolonged myelosuppression from chemotherapy, permitting escalation to considerably higher dose levels. However, marrow recovery still takes weeks and requires sophisticated supportive care until the effects of chemotherapy have lessened. Unfortunately, significant morbidity and mortality is associated with the underlying disease as well as complications due to the treatment itself. The three major causes of mortality after HSCT are relapse of the underlying malignancy, infection, and graft versus host disease.

Allogeneic hematopoietic cell transplantation (alloHSCT) is a potentially curative treatment option for patients with acute myeloid leukemia (AML); however, relapse accounts for approximately 40% of alloHSCT treatment failures. Among relapsed patients the 2-year post-relapse survival rate is reported at less than 20% (Devillier et al., Blood (2012) 119(6): 1228-1234). Unfortunately, sustainable remissions are rare in patients with post-transplant AML relapse, especially for those relapsing soon after alloHSCT (Arellano et al., Biol. of Blood and Marrow Trans. (2007) 1:116-123). Commonly used treatment options for relapsed patients include intensive chemotherapy with or without donor lymphocyte infusion (DLI), second alloHSCT, withdrawal of immunosuppression, or supportive care (Schmid et al., Jour Clin Onc (2007) 25(31) 4938-4945).

Viral infections remain a leading cause of morbidity and mortality after allogeneic hematopoietic stem cell transplantation (HSCT) (Moss et al., Nat Rev Immunol (2005); 5(1):9-20). Infections caused 8-16% of deaths in post-HCT recipients in 2008-09 (Pasquini M C. Current Uses and Outcomes of Hematopoietic Stem Cell Transplantation: CIBMTR Summary Slides. Available at www.cibmtr.org 2011). Viral infections play a major role in the post-transplant recipients (Wingard J R. Leuk Lymphoma (1993); 11 (Suppl 2): 115-25) and constitute up to 43% of all infections (George et al., Bone Marrow Transplant (2004); 33: 311-5). The use of prophylactic pharmacotherapy is effective in reducing the risk for some viral infections, but therapeutic options for breakthrough infections are complicated by toxicities, and for many viral infections there are limited/no effective prophylactic or therapeutic pharmacotherapies (Tomblyn et al., Biol Blood Marrow Transplant (2010); 16(2):294). T-cell reconstitution is a key requirement for effective antiviral control following HSCT, and factors that influence the speed of T-cell recovery also impact the risk of viral infection in this period (Leen et al., Immunol Rev (2014); 258(1):12-29.

Acute and chronic graft-versus-host disease (GVHD) are multisystem disorders that are common complications of allogeneic hematopoietic cell transplant (HCT). GVHD occurs when immune cells transplanted from a non-identical donor (the graft) recognize the transplant recipient (the host) as foreign, thereby initiating an immune reaction that causes disease in the transplant recipient. Acute GVHD is a significant cause of medical problems and death following an allogeneic stem cell transplantation. The frequency of acute GVHD varies significantly among populations, making it impossible to specify how common it is. Somewhere between 30 and 70 percent of transplant recipients develop acute GVHD, depending on donor type, transplant technique, and other features. Acute GVHD primarily affects the skin, the liver and the gastrointestinal tract (stomach, intestines and colon). Chronic GVHD is a syndrome that may involve a single organ or several organs. It is one of the leading causes of medical problems and death after allogeneic stem cell transplantation. Approximately 30-70 percent of patients receiving an allogeneic stem cell transplantation develop chronic GVHD. Since it is a chronic condition, it can last for years or even a lifetime. Chronic GVHD symptoms range from mild to life-threatening.

Intravenously administered glucocorticoids, such as prednisone, are the standard of care in acute GvHD (Goker et al., Experimental Hematology (2001) 29 (3): 259-77) and chronic GVHD (Menillo et al., Bone Marrow Transplantation (2001) 28 (8):807-8). The use of these glucocorticoids is designed to suppress the T-cell-mediated response by the host immune system; however, in high doses, this immune-suppression raises the risk of infections and cancer relapse.

Although significant improvement and advances in HSCT have occurred in the 50 years of treatment there remains a significant clinical need to reduce the significant morbidity and mortality associated with HSCT and improve the treatment and overall survival of patients who get HSCTs.

### SUMMARY OF THE INVENTION

The present invention provides isolated processed cell therapeutic compositions and methods of using such cell therapeutic compositions for the treatment of a patient with a disorder that is given a hematopoietic stem cell transplant (HSCT). The HSCT can be administered to a patient in conjunction with strong treatment for a tumor, including a hematopoietic or solid cancer, or for treatment of another type of disorder such as a primary immune deficiency, a genetic disorder, or abnormal cellular proliferation such as an autoimmune disorder including multiple sclerosis, lupus, or other disorder serious enough to require treatment in conjunction with an HSCT.

One aspect is for a cell composition comprising: (i) one or more primed and expanded T-cell subpopulations having specificity for one or more tumor associated antigens; (ii) one or more primed and expanded T-cell subpopulations having specificity for one or more viral associated antigens; and (iii) one or more mesenchymal stem cell (MSC) subpopulations. In some aspects, the one or more T-cell subpopulations of (i) have specificity for a tumor associated antigen expressed by a tumor of the patient.

In some aspects, the one or more tumor associated antigens are selected from the group consisting of WT1, PRAME, Survivin, NY-ESO-1, MAGE-A3, MAGE-A4, Pr3, Cyclin A1, SSX2, Neutrophil Elastase (NE), and combination thereof. In some aspects, the one or more tumor associated antigens are PRAME, Survivin, and WT1.

In some aspects, the one or more virus associated antigens are selected from the group consisting of immediate-early protein 1 (IE-1), immediate-early protein 2 (IE-2), 65 kDa phosphoprotein (pp65), EBNA-leader protein (EBNA-LP), EBNA1, EBNA2, EBNA3a, EBNA3b, EBNA3c, latent membrane protein 1 (LMP1), latent membrane protein 2 (LMP2); envelope glycoprotein GP350/GP340, BARF1 mRNA export factor EB2 (BMLF1), DNA polymerase processivity factor (BMRF1), trans-activator protein (BZLF1), hexon protein of Human adenovirus 3 (HAdV-3), penton protein of Human adenovirus 5 (HAdV-5), capsid protein VP-1, capsid protein VP-2, large T antigen, small T antigen, U14, U54, U90, fusion glycoprotein (F), major surface glycoprotein G, small hydrophobic protein (SH), nucleocapsid (N) protein, matrix protein (MP) 1, matrix protein (MP) 2, nucleocapsid protein (NP) 1, neuroaminidase, hemagglutinin (HA), protein E4, protein E5, protein E6, protein E7, late major capsid protein (L) 1, replication protein E1, replication protein E2, envelope glycoprotein gp160 (Env), Gag polyprotein, Nef protein, Pol polyprotein, and a combination thereof. In such aspects, the one or more virus associated antigens can be selected from the group consisting of IE-1, pp65, EBNA1, LMP1, LMP2, Hexon, Penton, LT, VP-1, MP1, NP1, N, F, U14, U90, and a combination thereof.

In some aspects, the one or more virus associated antigens comprise: (a) a viral associated antigen selected from the group consisting of IE-1, pp65, and a combination thereof; (b) a viral associated antigen selected from the group consisting of EBNA1, LMP1, LMP2, BARF1, BZLF1, and a combination thereof; (c) a viral associated antigen selected from the group consisting of Hexon, Penton, and a combination thereof; (d) a viral associated antigen selected from the group consisting of LT, VP-1, and a combination thereof; (e) a viral associated antigen selected from the group consisting of MP1, NP1, and a combination thereof; (f) a viral associated antigen selected from the group consisting of N, F, and a combination thereof; and (g) a viral associated antigen selected from the group consisting of U14, U90, and a combination thereof.

In some aspects, the MSC subpopulation is from bone marrow or cord blood.

In some aspects, the MSC subpopulation comprises greater than 95% of cells having a positive antigen expression pattern CD29, CD105, CD73, and CD90, and less than 2% of cells having an antigen expression pattern CD45, CD34, CD3, CD14, CD19, and HLA-DR.

In some aspects, the T-cell subpopulations of (i) are from an allogeneic donor. In some aspects, the T-cell subpopulations of (i) are from cord blood. In some aspects, the T-cell subpopulations of (i) are primed ex vivo.

In some aspects, the T-cell subpopulations of (ii) are from an allogeneic donor. In some aspects, the T-cell subpopulations (ii) are from cord blood. In some aspects, the T-cell subpopulations of (ii) are primed ex vivo.

Another aspect is for a method of treating a malignancy or tumor in a subject in need thereof, comprising administering an effective amount of the cell composition to the subject. In some aspects, the malignancy is a hematological malignancy. In such aspects, the hematological malignancy can be selected from the group consisting of leukemia, lymphoma, and multiple myeloma.

In some aspects, the tumor is a solid tumor. In such aspects, the solid tumor can be selected from the group consisting of a neuroblastoma, glioma, soft tissue cancer, germ cell cancer, breast cancer, Ewing's sarcoma, lung cancer, ovarian cancer, renal cell carcinoma, colon cancer, and melanoma.

In some aspects, the subject is receiving or has received an hematopoietic stem cell transplantation (HSCT).

A further aspect is for a cell composition comprising: (i) one or more primed and expanded T-cell subpopulations having specificity for one or more viral associated antigens; and (ii) one or more mesenchymal stem cell (MSC) subpopulations. In some aspects, the one or more virus associated antigens are selected from the group consisting of immediate-early protein 1 (IE-1), immediate-early protein 2 (IE-2), 65 kDa phosphoprotein (pp65), EBNA-leader protein (EBNA-LP), EBNA1, EBNA2, EBNA3a, EBNA3b, EBNA3c, latent membrane protein 1 (LMP1), latent membrane protein 2 (LMP2); envelope glycoprotein GP350/GP340, BARF1 mRNA export factor EB2 (BMLF1), DNA polymerase processivity factor (BMRF1), trans-activator protein (BZLF1), hexon protein of Human adenovirus 3 (HAdV-3), penton protein of Human adenovirus 5 (HAdV-5), capsid protein VP-1, capsid protein VP-2, large T antigen, small T antigen, U14, U54, U90, fusion glycoprotein (F), major surface glycoprotein G, small hydrophobic protein (SH), nucleocapsid (N) protein, matrix protein (MP) 1, matrix protein (MP) 2, nucleocapsid protein (NP) 1, neuroaminidase, hemagglutinin (HA), protein E4, protein E5, protein E6, protein E7, late major capsid protein (L) 1, replication protein E1, replication protein E2, envelope glycoprotein gp160 (Env), Gag polyprotein, Nef protein, Pol polyprotein, and a combination thereof. In such aspects, the one or more virus associated antigens can be selected from the group consisting of IE-1, pp65, EBNA1, LMP1, LMP2, Hexon, Penton, LT, VP-1, MP1, NP1, N, F, U14, U90, and a combination thereof.

In some aspects, the one or more virus associated antigens comprise: (a) a viral associated antigen selected from the group consisting of IE-1, pp65, and a combination thereof; (b) a viral associated antigen selected from the group consisting of EBNA1, LMP1, LMP2, BARF1, BZLF1, and a combination thereof (c) a viral associated antigen selected from the group consisting of Hexon, Penton, and a combination thereof; (d) a viral associated antigen selected from the group consisting of LT, VP-1, and a combination thereof (e) a viral associated antigen selected from the group consisting of MP1, NP1, and a combination thereof (f) a viral associated antigen selected from the group consisting of N, F, and a combination thereof; and (g) a viral associated antigen selected from the group consisting of U14, U90, and a combination thereof.

In some aspects, the MSC subpopulation is from bone marrow or cord blood.

In some aspects, the MSC subpopulation comprises greater than 95% of cells having a positive antigen expression pattern CD29, CD105, CD73, and CD90, and less than 2% of cells having an antigen expression pattern CD45, CD34, CD3, CD14, CD19, and HLA-DR.

In some aspects, the T-cell subpopulations are from an allogeneic donor.

In some aspects, the T-cell subpopulations are from cord blood.

In some aspects, the T-cell subpopulations are primed and expanded ex vivo.

An additional aspect is for a method of treating a non-malignant indication in a subject, comprising administering an effective amount of the cell composition to the subject. In some aspects, the non-malignant indications is an autoimmune disease, a metabolic disorder, or a primary immune deficiency disorder. In such aspects, the autoimmune disease can be multiple sclerosis, myasthenia gravis, Crohn's disease, or lupus; the metabolic disorder can be Mucopolysaccaridosis, Krabbe Disease, or Gaucher Disease; and the primary immune deficiency disorder can be Wiskott-Aldrich Syndrome or Severe combined immunodeficiency (SCID).

In some aspects, the subject is receiving or has received an hematopoietic stem cell transplantation (HSCT).

Another aspect is for a method of treating a malignancy or tumor in a subject, comprising:

(i) determining a human leukocyte antigen (HLA) subtype of the subject;

(ii) diagnosing a malignancy or tumor type of the subject;

(iii) identifying two or more tumor associated antigens associated with the tumor type for targeting with a tumor associated antigen (TAA)-specific T-cell subpopulation;

(iv) selecting at least one banked T-cell subpopulation for each targeted TAA, wherein the T-cell subpopulation selected has at least one shared allele or allele combination with the targeted TAA;

(v) identifying one or more viral associated antigens for targeting with viral associated antigen (VAA)-specific T-cell subpopulations;

(vi) selecting at least one banked T-cell subpopulation for each targeted VAA, wherein the T-cell subpopulation selected has at least one shared allele or allele combination with the targeted VAA;

(vii) selecting at least one banked mesenchymal stem cell (MSC) population;

(viii) combining each selected banked T-cell subpopulation and MSC population to create a cell composition; and

(ix) administering an effective amount of the cell composition to the subject.

A further aspect is for a method of selecting a therapy for a subject in need thereof, comprising:

(i) determining a human leukocyte antigen (HLA) subtype of the subject;

(ii) determining a tumor associated antigen (TAA) expression profile of the malignancy or tumor;

(iii) identifying two or more tumor associated antigens expressed by the tumor for targeting with TAA-specific T-cell subpopulations;

(iv) selecting one banked T-cell subpopulation for each targeted TAA, wherein the T-cell subpopulation selected has at least one shared allele or allele combination with the targeted TAA;

(v) identifying one or more viral associated antigens for targeting with viral associated antigen (VAA)-specific T-cell subpopulations;

(vi) selecting at least one banked T-cell subpopulation for each targeted VAA, wherein the T-cell subpopulation selected has at least one shared allele or allele combination with the targeted VAA; and

(vii) selecting at least one banked mesenchymal stem cell (MSC) population.

An additional aspect is for a method of treating a non-malignant indication in a subject, comprising:

(i) determining a human leukocyte antigen (HLA) subtype of the subject;

(ii) identifying one or more viral associated antigens for targeting with viral associated antigen (VAA)-specific T-cell subpopulations;

(iii) selecting at least one banked T-cell subpopulation for each targeted VAA, wherein the T-cell subpopulation selected has at least one shared allele or allele combination with the targeted VAA;

(iv) selecting at least one banked mesenchymal stem cell (MSC) population;

(v) combining each selected banked T-cell subpopulation and MSC population to create a T-cell/mesenchymal stem cell composition; and

(vi) administering an effective amount of the T-cell/mesenchymal stem cell composition to the subject.

In some aspects of the aforementioned methods, the subject is receiving or has received an hematopoietic stem cell transplantation (HSCT).

A further aspect is for a bank of T-cell subpopulations and mesenchymal stem cells (MSC) subpopulations comprising: (i) one or more primed and expanded T-cell subpopulations having specificity for one or more tumor associated antigens; (ii) one or more primed and expanded T-cell subpopulations having specificity for one or more viral associated antigens; and (iii) one or more mesenchymal stem cell (MSC) subpopulations.

In some aspects, the T-cell subpopulations of (i) are from an allogeneic donor.

In some aspects, the T-cell subpopulations of (ii) are from an allogeneic donor.

In some aspects, the T-cell subpopulations of (i) are primed and expanded ex vivo.

In some aspects, the T-cell subpopulations of (ii) are primed and expanded ex vivo.

Isolated and Processed Cell Therapies for Treatment of HSCT Patients with Malignancies

In some embodiments, the inventive isolated processed cell therapeutic compositions are used for the combined prevention and/or treatment of cancer recurrence, viral infection, and graft versus host disease (GVHD). The isolated cell compositions provided herein for this aspect include multiple cell subpopulations, wherein each specific cell subpopulation is directed to the prevention of, or treatment of, a particular comorbidity common with HSCT in conjunction with cancer therapy. In particular, the isolated cell subpopulations provided herein include i) one or more of a first T-cell subpopulation specific for one or more tumor associated antigens (TAAs) for the prevention and/or targeting of residual or relapsed cancer cells; ii) one or more of a second T-cell subpopulation specific for one or more viral-associated antigens (VAAs) for the targeting and/or prevention of one or more viral infections such as, but not limited to, cytomegalovirus (CMV), Epstein Barr Virus (EBV), adenovirus (AdV), human herpesvirus (HHV), BK virus (BKV), and human parainfluenza virus (HPIV), adeno-associated virus (AAV), human papillomavirus (HPV), and respiratory syncytial virus (RSV), among others; and iii) a mesenchymal stem cell (MSC) subpopulation for the prevention and/or treatment of GVHD. By providing a single dosage form comprising multiple targeted and specific cell subpopulations, a patient receiving HSCT can be treated for the common adverse events associated with HSCT with a single product. The resulting cell therapeutic composition is known as a “TVM” composition.

The TVM composition for treatment of cancer-related HSCT is comprised of three separate cellular subpopulations each directed to prevent and/or treat a common adverse event associated with HSCT. The TVM composition in this embodiment is administered to a patient that has undergone a HSCT for the purposes of treating an underlying hematological malignancy or solid tumor. As such, the TVM composition includes one or more T-cell subpopulations directed to one or more tumor-associated antigens (TAAs) associated with the underlying hematological malignancy or solid tumor of the patient. These TAA-specific T-cell subpopulations are primed to one or more TAAs and expanded ex vivo. The TAA-specific T-cell subpopulation may be activated by the use of pooled, multi-TAA overlapping peptide libraries, wherein the multi-TAA overlapping peptide library includes two or more tumor antigen peptide libraries. In an alternative embodiment, the T-cell subpopulation for inclusion in the TVM composition is comprised of a combined set of TAA-specific T-cell subpopulations, wherein each T-cell subpopulation is directed to a single TAA. For example, the TAA T-cell subpopulations are each exposed to single TAA overlapping peptide libraries or one or more peptides from a single TAA, including and perhaps substantially comprised of selected peptide epitope(s) of the TAA. In some embodiments, the mastermix of peptides includes both an overlapping peptide library and specifically selected HLA-restricted peptides generated by determining the HLA profile of the donor source. The TAA T-cell subpopulation of the TVM composition may include more than one, for example two, three, four, or five T-cell subpopulations, wherein each T-cell subpopulation is specific for a single TAA; for example, the separate T-cell subpopulations that comprise the TVM composition are each primed with a single tumor antigen, for example each T-cell subpopulation is capable of recognizing one TAA.

In some embodiments, the TAA T-cell subpopulation is primed with a single TAA peptide mix, wherein the peptide mix comprises antigenic epitopes derived from a TAA based on one or more of the donor's HLA phenotypes, for example, the peptides are restricted through one or more of the cell donor's HLA alleles such as, but not limited to, HLA-A, HLA-B, and HLA-DR. By including specifically selected donor HLA-restricted peptides from a single TAA in the peptide mix for priming and expanding each TAA T-cell subpopulation, a TAA T-cell subpopulation can be generated that provides greater TAA targeted activity through one or more donor HLA alleles, improving potential efficacy of the T-cell subpopulation for patients that share at least one HLA allele with the donor. In addition, by generating a TAA T-cell subpopulation with TAA targeted activity through more than one donor HLA allele, a single donor TAA T-cell subpopulation may be included in the TVM composition for multiple recipients with different HLA profiles by matching one or more donor HLA alleles showing TAA-activity. In some embodiments, the TAA peptides used to prime and expand a TAA T-cell subpopulation are generated based on a cell donor's HLA profile, wherein the peptides are HLA-restricted epitopes specific to at least one or more of a donor's HLA-A alleles, HLA-B alleles, or HLA-DR alleles, or a combination thereof. In some embodiments, the HLA-A alleles are selected from a group comprising HLA-A*01, HLA-A*02:01, HLA-A*03, HLA-A*11:01, HLA-A*24:02, HLA-A*26, and HLA-A*68:01. In some embodiments, the HLA-B alleles are selected from a group comprising HLA-B*07:02, HLA-B*08, HLA-B*15:01 (B62), HLA-B*18, HLA-B*27:05, HLA-B*35:01, and HLA-B*58:02. In some embodiments, the HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, and HLA-DRB1*1501 (DR2b). In some embodiments, the mastermix of peptides includes both an overlapping peptide library and specifically selected HLA-restricted peptides generated by determining the HLA profile of the donor source.

The particular T-cell subpopulations that are included in the TVM composition target TAAs that are representative of, or associated with, the TAA expression profile of the patient's underlying malignancy. In some embodiments, the TAA-targeting T-cell subpopulation in the TVM composition correlates with the tumor-associated antigen expression profile of the malignancy in the patient receiving the treatment. In an alternative embodiment, the TAA targeting T-cell subpopulations that are included in the TVM composition target TAAs that are typically associated with the patient's malignancy. For example, the TAAs targeted may be one or more TAAs that are generally or commonly expressed in the particular hematological malignancy or solid tumor of the patient.

The generation of the TAA T-cell subpopulation can be accomplished through the ex vivo priming and activation of the T-cell subpopulation to one or more peptides from a single TAA, or in an alternative, one or more peptides from multiple TAAs. If more than one peptide from a single, targeted tumor antigen is used, the peptide segments can be generated by making overlapping peptide fragments of the tumor antigen, as provided for example, in commercially available overlapping peptide libraries, or can be selected to be limited to, or enriched with, certain antigenic epitopes of the targeted TAA, for example, a single, or multiple specific epitopes of the TAA. In some embodiments, the T-cell subpopulation is primed with a single TAA peptide mix, wherein the peptide mix includes a overlapping peptide library that has been further enriched with one or more specific known or identified epitopes expressed by the patient's malignancy. In some embodiments, the T-cell subpopulation is primed with a multi-TAA peptide mix, wherein the peptide mix includes a overlapping peptide library that has been further enriched with one or more specific known or identified tumor antigenic epitopes expressed by the patient's malignancy. In some embodiments, the peptide segments are the same length. In some embodiments, the peptide segments are of varying lengths. In other embodiments, the peptide segments substantially only include known tumor antigenic epitopes. In some embodiments, the T-cell subpopulation is primed and activated with one or more epitopes expressed by the patient's malignancy. In some embodiments, the tumor antigen is a neoantigen. In some embodiments, the neoantigen is a mutated form of an endogenous protein derived through a single point mutation, a deletion, an insertion, a frameshift mutation, a fusion, mis-spliced peptide, or intron translation.

In some embodiments, a T-cell subpopulation used in the TVM composition is capable of recognizing one epitope, two epitopes, three epitopes, or more than three epitopes of a single TAA. In some embodiments, the TVM composition includes more than one T-cell subpopulation targeting the same TAA, wherein each T-cell subpopulation is capable of recognizing discrete and separate epitopes within the same TAA.

The TAA T-cell subpopulations of the TVM composition are generated to be specific to one or more TAAs. TAAs for targeting by the TAA T-cell subpopulations may include any TAA expressed by the malignancy, for example, an oncofetal, an oncoviral, overexpressed/accumulated, cancer-testis, lineage-restricted, mutated, post-translationally altered, or idiotypic antigen. Although they are preferentially expressed by cancer cells, TAAs are oftentimes found in normal tissues. However, their expression differs from that of normal tissues by their degree of expression in the malignancy, alterations in their protein structure in comparison with their normal counterparts or by their aberrant subcellular localization within malignant cells. Non-limiting examples of TAAs, in certain embodiments, for targeting may be selected from one or more peptide segment(s), overlapping peptide libraries, or selected epitope(s) of Carcinoembryonic antigen (CEA), immature laminin receptor, and tumor-associated glycoprotein (TAG) 72, latent membrane protein (LMP) 1 and 2, BING-4, calcium-activated chloride channel (CLCA) 2, Cyclin B1, 9D7, epithelial cell adhesion molecule (Ep-Cam), EphA3, Her2/neu, telomerase, mesothelin, stomach cancer-associated protein tyrosine phosphatase 1 (SAP-1), survivin, b melanoma antigen (BAGE) family, cancer-associated gene (CAGE) family, G antigen (GAGE) family, melanoma antigen (MAGE) family, sarcoma antigen (SAGE) family, X antigen (XAGE) family, CT9, CT10, NY-ESO-1, L antigen (LAGE) 1, Melanoma antigen preferentially expressed in tumors (PRAME), synovial sarcoma X (SSX) 2, melanoma antigen recognized by T cells-1/2 (Melan-A/MART-1/2), Gp100/pmel17, tyrosinase, tyrosine-related protein (TRP) 1 and 2, P.polypeptide, melanocortin 1 receptor (MC1R), prostate-specific antigen, β-catenin, breast cancer antigen (BRCA) 1/2, cyclin-dependent kinase (CDK) 4, chronic myelogenous leukemia antigen (CML) 66, fibronectin, MART-2, p53, Ras, TGF-βRII, mucin (MUC) 1, immunoglobulin (Ig) and T cell receptor (TCR).

In some embodiments, the TVM composition includes one or more T-cell subpopulations targeting WT1, PRAME, Survivin, NY-ESO-1, MAGE-A3, MAGE-A4, Pr3, Cyclin A1, SSX2, Neutrophil Elastase (NE), or a combination thereof. In some embodiments, the TVM composition includes one or more T-cell subpopulations targeting WT1, PRAME, and Survivin. In some embodiments, the targeted antigens do not include MAGE-A3.

Importantly, TAA T-cell subpopulations can be optimized for personal efficacy in the patient by testing each T-cell subpopulation for activity against and responsiveness to the patient's underlying malignant cells. Therefore, in some embodiments, the invention includes priming and activating TAA T-cell subpopulations for inclusion in a TVM composition which have been primed and activated with specific TAAs based on malignancy-type of the patient. In some embodiments, epitopes expressed by a patient's malignancy are first identified and T-cell subpopulations primed to those epitopes are included in the TVM composition. In an alternative embodiment, specific epitopes expressed by a patient's malignancy are first identified and included in a overlapping peptide library used to prime and activate a T-cell subpopulation. By using or including specifically expressed patient tumor associated epitopes in a peptide mix to prime and activate specific T-cell subpopulations, the peptide mix for the specific TAA can be optimized, and the ability of the T-cell subpopulation to recognize the TAA confirmed ex vivo. In some embodiments, the generated T-cell subpopulation can be tested for activity against the patient's malignant cells ex vivo to confirm a robust response. This can be repeated for some or all of the remaining TAA T-cell subpopulations comprising the TVM composition until it is confirmed that one, some or all of the TAA T-cell subpopulations are primed and activated against the targeted TAAs of the patient.

In some embodiments, a sample of the patient's malignant cells is taken by biopsy, blood sample or other isolation and is used to derive a profile of antigenic proteins expressed in the malignancy, and the TAA T-cell subpopulations of the TVM composition target one or more of the expressed tumorigenic antigens. In another embodiment, an epitope profile of expressed antigenic proteins is identified, and the TAA T-cell subpopulations of the TVM composition target one or more of the identified epitopes. It is preferred to select antigenic proteins that are not overexpressed self-proteins which have not been mutated, rearranged or otherwise altered over the normal sequence and conformation, as these typically do not evoke a strong response in vivo.

Patients undergoing HSCT generally undergo a myelo-ablative preparative regimen—with or without radiation—in order to eliminate the hematological malignancy or tumor. In doing so, the patient's endogenous immune system is largely, if not entirely, eliminated. While the patient's recipient HSCT will naturally include immune effector cells directed to a number of viruses, that is, the donor is likely to be seropositive for certain viruses, a common side effect following HSCT is susceptibility to a large number of viruses. The TVM composition provided herein includes one or more T-cell subpopulations directed to one or more viral-associated antigens (VAAs) targeting common viruses that HSCT recipients are susceptible to. These VAA-specific T-cell subpopulations are primed to one or more VAAs and expanded ex vivo. The VAA-specific T-cell subpopulation may be derived by the use of pooled, multi-VAA overlapping peptide libraries, wherein the multi-VAA overlapping peptide library includes two or more viral antigen peptide libraries. In an alternative embodiment, the T-cell subpopulation for inclusion in the TVM composition is comprised of a combined set of VAA-specific T-cell subpopulations, wherein each T-cell subpopulation used for combining is directed to a single virus, for example, the VAA T-cell subpopulations are each exposed to single viral associated antigen overlapping peptide libraries or one or more peptides from a single viral associated antigen, including and perhaps substantially comprised of selected peptide epitope(s) of the viral associated antigen. The VAA T-cell subpopulation of the TVM composition may include more than one, for example two, three, four, five, or six T-cell subpopulations, wherein each T-cell subpopulation is specific for a single virus; for example, the separate T-cell subpopulations that comprise the TVM composition are each primed to one or more viral antigens from a single virus, for example each T-cell subpopulation is capable of recognizing one virus.

The generation of the VAA T-cell subpopulation can be accomplished through the ex vivo priming and activation of the T-cell subpopulation to one or more peptides from a single VAA, or in an alternative, one or more peptides from multiple VAAs. If more than one peptide from a single, targeted viral antigen is used, the peptide segments can be generated by making overlapping peptide fragments of the viral antigen, as provided for example, in commercially available overlapping peptide libraries, or can be selected to be limited to, or enriched with, certain antigenic epitopes of the targeted virus, for example, a single, or multiple specific epitopes of the virus. In some embodiments, the mastermix of peptides includes both an overlapping peptide library and specifically selected HLA-restricted peptides generated by determining the HLA profile of the donor source. In some embodiments, the peptide segments are the same length. In some embodiments, the peptide segments are of varying lengths. In other embodiments, the peptide segments substantially only include known viral antigenic epitopes. In some embodiments, the VAA T-cell subpopulation is primed and activated with one or more epitopes from a virus that the donor of the HSCT is seronegative for. In some embodiments, the VAA T-cell subpopulation is primed and activated with one or more epitopes from a virus that the patient was seropositive for before receiving the HSCT.

In some embodiments, a VAA T-cell subpopulation used in the TVM composition is capable of recognizing one epitope, two epitopes, three epitopes, or more than three epitopes of a single VAA. In some embodiments, the TVM composition includes more than one T-cell subpopulation targeting the same VAA, wherein each T-cell subpopulation is capable of recognizing discrete and separate epitopes within the same VAA.

In some embodiments, the VAA T-cell subpopulation is primed with a single VAA peptide mix, wherein the peptide mix comprises antigenic epitopes derived from a VAA based on one or more of the donor's HLA phenotypes, for example, the peptides are restricted through one or more of the cell donor's HLA alleles such as, but not limited to, HLA-A, HLA-B, and HLA-DR. By including specifically selected donor HLA-restricted peptides from a single VAA in the peptide mix for priming and expanding each T-cell subpopulation, a T-cell subpopulation can be generated that provides greater VAA targeted activity through one or more donor HLA alleles, improving potential efficacy of the T-cell subpopulation for patients that share at least one HLA allele with the donor. In addition, by generating a T-cell subpopulation with VAA targeted activity through more than one donor HLA allele, a single donor T-cell subpopulation may be included in a TVM composition for multiple recipients with different HLA profiles by matching one or more donor HLA alleles showing VAA-activity. In some embodiments, the TAA peptides used to prime and expand a T-cell subpopulation are generated based on a cell donor's HLA profile, wherein the peptides are HLA-restricted epitopes specific to at least one or more of a donor's HLA-A alleles, HLA-B alleles, or HLA-DR alleles, or a combination thereof. In some embodiments, the HLA-A alleles are selected from a group comprising HLA-A*01, HLA-A*02:01, HLA-A*03, HLA-A*11:01, HLA-A*24:02, HLA-A*26, and HLA-A*68:01. In some embodiments, the HLA-B alleles are selected from a group comprising HLA-B*07:02, HLA-B*08, HLA-B*15:01 (B62), HLA-B*18, HLA-B*27:05, HLA-B*35:01, and HLA-B*58:02. In some embodiments, the HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, and HLA-DRB1*1501 (DR2b). In some embodiments, the mastermix of peptides includes both an overlapping peptide library and specifically selected HLA-restricted peptides generated by determining the HLA profile of the donor source.

The VAA T-cell subpopulations of the TVM composition are generated to be specific to one or more VAAs. Each virus has its own VAAs. Non-limiting examples of VAAs, in certain embodiments, for targeting may be selected from one or more peptide segment(s), overlapping peptide libraries, or selected epitope(s) of immediate-early protein 1 (IE-1), immediate-early protein 2 (IE-2), 65 kDa phosphoprotein (pp65), Epstein-Barr Nuclear Antigen (EBNA) family, which includes EBNA-leader protein (EBNA-LP), EBNA1, EBNA2, EBNA3a, EBNA3b, EBNA3c; latent membrane protein (LMP) family, which includes LMP1 and LMP2; envelope glycoprotein GP350/GP340; secreted protein BARF1; mRNA export factor EB2 (BMLF1); DNA polymerase processivity factor (BMRF1) and trans-activator protein (BZLF1), the hexon protein of Human adenovirus 3 (HAdV-3), the penton protein of Human adenovirus 5 (HAdV-5), capsid protein VP-1, capsid protein VP-2, large T antigen, small T antigen, U14, U54, U90, fusion glycoprotein (F), major surface glycoprotein G, small hydrophobic protein (SH), nucleocapsid (N) protein, matrix protein (MP) 1, matrix protein (MP) 2, nucleocapsid protein (NP) 1, neuroaminidase, hemagglutinin (HA), protein E4, protein E5, protein E6, protein E7, late major capsid protein (L) 1, replication protein E1, replication protein E2, envelope glycoprotein gp160 (Env), Gag polyprotein, Nef protein, and Pol polyprotein.

In some embodiments, the TVM composition includes one or more T-cell subpopulations specific to the viral-associated antigens IE-1, pp65, EBNA1, LMP1, LMP2, Hexon, Penton, LT, VP-1, MP1, NP1, N, F, U14 and U90, or a combination thereof. In some embodiments, the TVM compositions includes one or more T-cell subpopulations specific to at least one of the viral-associated antigens of CMV selected from IE-1 and pp65; at least one of the viral-associated antigens of EBV selected from EBNA1, LMP1, LMP2, BARF1 and BZLF1; at least one of the viral-associated antigens of AdV selected from Hexon and Penton; at least one of the viral-associated antigens of BK virus selected from LT and VP-1; at least one of the viral-associated antigens of parainfluenza selected from MP1 and NP1; at least one of the viral-associated antigens of RSV selected from N and F; and at least one of the viral-associated antigens from HHV6 selected from U14 and U90.

In certain nonlimiting embodiments, each TAA and VAA T-cell subpopulation is prepared by pulsing antigen presenting cells (APCs) or artificial antigen presenting cells (aAPCs) with a single peptide or epitope, several peptides or epitopes, or even with peptide libraries of one or more targeted antigens, that for example, include peptides that are about 7, 8, 9, 10, 11, 12, 13, 14, 15 16 or more amino acids long and overlapping one another by 5, 6, 7, 8, 9, 10, 11, 12 or 13 amino acids, in certain aspects. Examples include overlapping peptide libraries from JPT Technologies or Miltenyi. In particular embodiments, the peptides are 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and there is overlap of 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length.

Graft versus host disease is a difficult and potentially lethal complication of HSCT. It occurs with minor human leukocyte antigen (HLA) mismatch and is normally treated with corticosteroid and other immunosuppressive therapy. When it is refractory to steroid therapy, mortality approaches 80%. Graft-versus-host-disease is characterized by selective damage to the recipient patient's liver, skin (rash), mucosa, and the gastrointestinal tract induced by the donor's immune effector cells contained in the HSCT, and long term GVHD (chronic GVHD) may result in damage to the patient recipient's connective tissue and exocrine glands. The acute or fulminant form of the disease (aGVHD) is normally observed within the first 100 days post-transplant and is a major challenge to transplants owing to associated morbidity and mortality. The chronic form of graft-versus-host-disease (cGVHD) normally occurs after 100 days. The appearance of moderate to severe cases of cGVHD adversely influences long-term survival.

In order to treat and/or prevent GVHD, the TVM composition includes a mesenchymal stem cell subpopulation. Mesenchymal stem cells (MSCs) are multipotent stromal cells that can differentiate into a variety of cell types, including osteoblasts (bone cells), chondrocytes (cartilage cells), myocytes (muscle cells) and adipocytes (fat cells which give rise to marrow adipose tissue). The MSC subpopulation can be derived from bone marrow or cord blood. As multipotent stem cells, MSCs can differentiate into cells derived from the mesoderm germ layer, namely chondroblasts, adipocytes, and osteocytes. MSCs can be expanded in culture and possess complex and diverse immunomodulatory activity. Moreover, human MSCs carry low levels of class 1 and no class 2 HLA antigens, making them immunoprivileged and able to be used without HLA matching. In some embodiments, the MSC subpopulation contains greater than 95% of cells having the positive antigen expression pattern CD29, CD105, CD73, and CD90, and less than 2% of cells having the antigen expression pattern CD45, CD34, CD3, CD14, CD19, and HLA-DR.

The TAA T-cell, VAA T-cell, and MSC subpopulations can be generated from the same donor as used in the HSCT. In some embodiments, the TAA T-cell and VAA T-cell subpopulations for inclusion in the TVM composition are autologously derived. In some embodiments, the TAA T-cell and VAA T-cell subpopulations for inclusion in the TVM composition are derived from an allogeneic donor, for example, from the peripheral blood, apheresis product or bone marrow from a naïve, healthy donor. In some embodiments, the TAA-specific T-cell subpopulations for inclusion in the TVM composition are derived from cord blood. When derived from an allogeneic donor, the TAA T-cell subpopulation starting material will generally be naïve to the targeted TAA, while the VAA T-cell subpopulation may include one or more T-cell subpopulations that are initially naïve to the targeted viruses.

The TVM composition can be administered to a patient at the time of HSCT to treat a hematological malignancy or solid tumor. Alternatively, the TVM composition can be administered to a patient who has already received an HSCT to treat a hematological malignancy or solid tumor. The hematological malignancy may be a leukemia, lymphoma, or myeloma, including but not limited to acute myeloid leukemia (AML), acute lymphoblastic leukemia (ALL), chronic lymphoblastic leukemia (CLL), or multiple myeloma. The solid tumor may be neuroblastoma, glioma, soft tissue cancer, germ cell cancer, breast cancer, Ewing's sarcoma, lung cancer, ovarian cancer, renal cell carcinoma, colon cancer, melanoma and other solid tumors. In some embodiments, the hematological malignancy is a relapsed or refractory leukemia, lymphoma, or myeloma. In some embodiments, the solid tumor is a relapsed or refractory solid tumor.

Isolated and Processed T-Cell Therapies for Treatment of HSCT Patients with Disorders Other than Malignancies

The present invention also includes a method and composition to treat patients undergoing HSCT for a disorder other than a malignancy. In this alternative embodiment, the isolated cell subpopulations include i) one or more T-cell subpopulations specific for one or more viral-associated antigens (VAAs) for the targeting and/or prevention of one or more viral infections such as, but not limited to, cytomegalovirus (CMV), Epstein Barr Virus (EBV), adenovirus (AdV), human herpesvirus (HHV), BK virus (BKV), and human parainfluenza virus (HPIV), adeno-associated virus (AAV), human papillomavirus (HPV), and respiratory syncytial virus (RSV), among others; and ii) a mesenchymal stem cell (MSC) subpopulation for the prevention and/or treatment of GVHD. By providing a single dosage form comprising multiple targeted and specific cell subpopulations, a patient receiving HSCT can be treated for the common adverse events associated with HSCT with a single product. The resulting cell therapeutic composition is known as a “VM” composition.

The VM composition can be administered to a patient at the time of HSCT during treatment of a non-malignant disorder. Alternatively, the VM composition can be administered to a patient who has already received an HSCT to treat a non-malignant disorder. In these indications the patients are at risk for the HSCT complications including viral infections and GVHD. In some embodiments, the VM composition is used after an allogeneic HSCT as a treatment for a non-malignant indication. Non-malignant indications where allogeneic HSCT is currently employed include, but are limited to, autoimmune diseases, metabolic disorders and primary immune deficiency disorders. The autoimmune diseases could include, but are not limited to, multiple sclerosis, myasthenia gravis, Crohn's disease and lupus. The metabolic disorders could include, but are not limited to, Mucopolysaccharidosis, Krabbe Disease, and Gaucher Disease. The primary immune deficiency disorders could include, but are not limited to, Wiskott-Aldrich Syndrome and Severe combined immunodeficiency (SCID).

### Cell Banks for Patients Undergoing HSCT Therapies

In one aspect, the invention further includes a bank, and methods of manufacturing a bank, of individual T-cell subpopulations with an associated phenotypic characteristic database, which can be used in either TVM or VM therapy in conjunction with HSCT.

For TVM compositions, the bank includes individual TAA T-cell subpopulations which have been primed and activated to one or more TAAs, individual VAA T-cell subpopulations, which have been primed and activated to one or more viruses, and expanded MSC subpopulations. The cell subpopulations are derived from allogeneic donor sources, for example, the peripheral blood, apheresis product or bone marrow from a naïve, healthy donor and/or cord blood sample. The T-cell subpopulations are HLA-typed and the donor source recorded. In some embodiments, the donor source is the original HSCT donor for the patient. The T-cell subpopulations' antigenic recognition response is verified and characterized, for example, via ELISPOT IFN-γ assay, TNF-α assay, or other suitable activity indicator, to quantify the activity of the T-cell population against the specific, targeted TAA and VAA. Furthermore, the T-cell subpopulations' antigenic recognition response is further characterized through its corresponding HLA-allele, for example through an HLA restriction assay. The T-cell subpopulations and MSCs can be cryopreserved and stored. In some embodiments, the T-cell subpopulations and MSCs are stored by the donor source. In some embodiments, the T-cell subpopulations are stored by TAA and VAA specificity, respectively. In some embodiments, the T-cell subpopulations are stored by human leukocyte antigen (HLA) subtype and restrictions.

By characterizing each T-cell subpopulations' reactivity and corresponding HLA-allele, the T-cell subpopulations included in the TVM composition can be optimized for each patient based on specific T-cell subpopulation reactivity and HLA matching, providing a highly personalized therapy. Accordingly, if a patient has a malignancy that expresses one epitope of a TAA but not another, or if one epitope of a TAA invokes a greater T-cell response, that T-cell subpopulation can be taken from the bank and used in the TVM composition. Similarly, if a patient has a particular virus or is susceptible to a particular virus, that VAA T-cell subpopulation can be taken from the bank and used in the TVM composition. In this way, the T-cell therapy can be tailored to evoke a maximal response against the patient's tumor or viral complications.

This invention thus acknowledges and accounts for the fact that T-cells from various donors may have variable activity against the same tumor- or viral-associated antigen, or even the same epitope, generating T-cell responses with varying efficiency. This fact is taken into account when producing the comprehensive bank of a wide variety of allogeneic activated T-cells for personalized T-cell therapeutic composition of the invention. Derived T-cell subpopulations having shared HLA-alleles that exhibit strong activity to the targeted tumor- or viral-associated antigen can be selected from the bank for inclusion in the TVM composition. In some embodiments, one or more of the T-cell subpopulations for consideration for inclusion in the TVM composition are tested against malignant cells from the patient prior to administration in vivo by exposing the malignant cells in vitro to the one or more T-cell subpopulations and determining the T-cell subpopulation's ability to lyse the malignant cell. In this way, the probability of the TVM composition inducing a therapeutic response to a relapse or providing an effective prophylactic effect against a relapse upon administration to the patient is greatly enhanced.

For VM therapy, a cellular composition is provided as described above or generally herein where the TAA is excluded and T-cells that have been primed against one or more selected viral antigens are combined with mesenchymal cells.

In some embodiments, instead of using a banked T cell subpopulation or MSC population, a newly produced T cell subpopulation or MSC population, that has yet to be banked, can be used.

In some aspects, a portion of the newly produced T cell subpopulation, or MSC population, can be used to treat a patient and another portion can be banked for future use.

### Summary of Embodiments for TVM Composition, Use and Manufacture

In one aspect, provided herein is a method of treating a patient with a malignancy or tumor receiving HSCT comprising:
i) determining the HLA subtype of the patient;
ii) diagnosing the malignancy or tumor type of the patient;
iii) identifying two or more tumor associated antigens associated with the tumor type for targeting with TAA-specific T-cell subpopulations;
iv) selecting at least one banked T-cell subpopulation having the good activity against each targeted TAA through one or more HLA-alleles shared between the patient and the TAA T-cell subpopulations, wherein each T-cell subpopulation is specific for one or multiple tumor associated antigens, wherein each of the T-cell subpopulations are primed and expanded ex vivo;
v) identifying one or more viral associated antigens for targeting with VAA-specific T-cell subpopulations,
vi) selecting at least one banked T-cell subpopulation having the highest activity against one or more targeted VAA through one or more HLA-alleles shared between the patient and the VAA T-cell subpopulations, wherein each T-cell subpopulation is specific for multiple virus associated antigens, wherein each of the T-cell subpopulations are primed and expanded ex vivo;
vii) selecting at least one banked mesenchymal stem cell population
viii) combining each selected banked T-cell subpopulation and MSC population to create a TVM composition; and,
ix) administering an effective amount of the TVM composition to the patient; and,
x) repeating the administration of the TVM composition as necessary
   In some embodiments, the TVM composition is administered concomitantly with the HSCT. In some embodiments, the TVM composition is administered following HSCT.

In one aspect, provided herein is a method of treating a patient with a malignancy or tumor receiving HSCT comprising:
i) determining the HLA subtype of the patient;
ii) determining the TAA expression profile of the patient's malignancy or tumor;
iii) identifying two or more tumor associated antigens associated with the tumor type for targeting with TAA-specific T-cell subpopulations;
iv) selecting at least one banked T-cell subpopulation having the highest activity against each targeted TAA through one or more HLA-alleles shared between the patient and the TAA T-cell subpopulations, wherein each T-cell subpopulation is specific for one or multiple tumor associated antigens, wherein each of the T-cell subpopulations are primed and expanded ex vivo;
v) identifying one or more viral associated antigens for targeting with VAA-specific T-cell subpopulations;
vi) selecting at least one banked T-cell subpopulation having good activity against each targeted VAA through one or more HLA-alleles shared between the patient and the VAA T-cell subpopulations, wherein each T-cell subpopulation is specific for multiple virus associated antigens, wherein each of the T-cell subpopulations are primed and expanded ex vivo;
vii) selecting at least one banked mesenchymal stem cell population;
viii) combining each selected banked T-cell subpopulation and MSC population to create a TVM composition;
ix) administering an effective amount of the TVM composition to the patient; and,
x) repeating the administration of the TVM composition as necessary
   In some embodiments, the shared HLA alleles are selected from one or more of HLA-A, HLA-B, or HLA-DR. In some embodiments, the TVM composition is administered concomitantly with the HSCT. In some embodiments, the TVM composition is administered following HSCT.

In some embodiments, the TAA-specific T-cell subpopulation used in the TVM composition is selected based on the TAA expression profile of the patient. In some embodiments, the TAAs to target by the T-cell subpopulations used to create the TVM composition are selected by the healthcare practitioner based on the type of tumor that is diagnosed. In some embodiments, the multi-VAA-specific T-cell subpopulation used in the TVM composition is selected to provide coverage against viruses selected from the group comprising cytomegalovirus, Epstein-Barr virus, Adenovirus, Human Herpes Virus 6, BK polyoma virus and parainfluenza.

In a typical embodiment, a patient, such as a human, is infused or injected with an effective dose of a TVM composition ranging from 1×10⁶ to 1×10⁸ cells/m² of a TAA T-cell subpopulation, 1×10⁶ to 1×10⁸ cells/m² of a multi-VAA T-cell subpopulation, and 1−5×10⁶/kg of a MSC subpopulation. Alternatively, the cell subpopulations of a TVM composition are not combined into a single dosage form, but rather each cell subpopulation is administered separately. The patient may receive a second or additional infusion or injection about 1 or more weeks later if recommended by the health care practitioner and may receive additional doses subsequent thereto as useful and recommended.

The T-cells can be primed and activated using a number of known procedures. In one non-limiting aspect, the present invention includes a process for generating a T-cell subpopulation specific to either multiple TAA or multiple VAA to form TVM therapeutic compositions that includes but is not limited to:
i) identifying eligible donors who are negative to the patient's disease, and preferably healthy, and wherein the donor can be cord blood or PBMCs;
ii) collecting the mononuclear cells from the negative donor and optionally removing any effector or other memory T-cells optionally based on CD45RA⁻, CD45R⁺, CCR7⁻, CD62L⁻, CCR7⁺, and/or CD62L⁺ markers;
iii) separating the mononuclear cells into two components;
iv) separating the cells in the first component into nonadherent T-cells and precursors and adherent dendritic cells and precursors, using any method known in the art, for example exposure to a solid medium, separation magnetically, use of antibodies, etc., and if not done already, optionally removing any effector or other memory T-cells optionally based on CD45RA−, CD45RO+, CCR7, CD62L−, or CCR7+, CD62L+ markers;
v) differentiating monocytes and precursors to dendritic cells with IL-4 and GM-CSF, followed by treatment with maturing cytokines such as LPS, TNFα, IL-1β, IL-4, IL-6 and GM-CSF and then pulsing with one or more peptide(s) and/or epitope(s) from multiple selected TAAs or VAAs; and then irradiating to form dendritic antigen presenting cells (APCs);
vi) treating the nonadherent T-cells and precursors with cytokines IL-7 and IL-15 to polarize to Th1 cells (and in some embodiments, without the use of IL-12);
vii) mixing the dendritic antigen presenting cells from (v) with the non-adherent T-cells and T-cell precursor cells from (vi) in the presence of cytokines IL-6, optionally in a ratio of between 5:1 and 20:1 of (vi) to (v) to produce a T-cell subpopulation specific for a single TAA or VAA;
viii) treating the second component of mononuclear cells with a mitogen such as PHA, a T-blast, B-blast, lymphoblastic cell or CD3/CD28 Blast optionally in the presence of IL-2 to produce activated T-cells; and then irradiating the cells to inhibit growth;
ix) pulsing the PHA blasts in (viii) with selected antigenic peptide(s) and/or epitope(s) from the selected tumor-associated antigens and irradiating to inhibit growth;
x) mixing the antigen specific T-cells from (vii) with the activated T-cell subpopulation from (ix) optionally in the presence of K562 accessory cells (preferably HLA-negative, K562 cells expressing CD80, CD83, CD86 and/or 4-IBBL) or LPS, and optionally IL-15 and/or IL-2;
xi) recovering the produced multi-antigen-specific T-cell subpopulation;
xii) optionally characterizing the resulting T-cell subpopulation for banking; and,
xiii) optionally cryopreserving and storing in the bank until use.

In the above process, unless specific steps are taken to remove cell components of the donor blood starting material, for example, removal based on cell surface markers, etc., the final T-cell subpopulation will normally also include a range of cell types, such as Natural Killer T-cells, γδ T-cells, CD4+ T-cells, CD8+ (cytotoxic) T-cells, and Natural Killer T-cells, among others, and may have naïve, and effector memory or central memory cells. The ratios of these cell types in the TVM composition will vary according to the donor's blood and processing conditions.

In another aspect, the present invention includes a method of manufacturing a T-cell subpopulation of the present invention comprising (i) collecting a mononuclear cell product from a healthy donor; (ii) determining the HLA subtype of the mononuclear cell product; (iii) separating the monocytes and the lymphocytes of the mononuclear cell product; (iv) generating and maturing dendritic cells (DCs) from the monocyte fraction; (v) pulsing the DCs with one or more peptides and/or epitopes from multiple TAAs or VAAs; (vi) carrying out a CD45RA+ selection to isolate naïve lymphocytes from the lymphocyte fraction; (vii) stimulating the naïve lymphocytes with the peptide-pulsed DCs in the presence of a cytokine cocktail; (viii) repeating the T cell stimulation with fresh peptide-pulsed DCs or other peptide-pulsed antigen presenting cells in the presence of a cytokine cocktail; and (ix) harvesting the T-cell subpopulation, (x) characterizing the T-cell subpopulation as described herein; and (xi) banking the T-cell subpopulation for future use in a TVM composition.

In another aspect, the present invention includes a method of isolating and expanding a homogeneous mesenchymal stem cell population of the present invention comprising i) collecting bone marrow from a donor; ii) priming and coating the cell expansion set in the bioreactor; iii) loading bone marrow into the bioreactor; iv) feeding the MSCs; v) harvesting MSCs; vi) performing additional passages; and vii) cryopreservation.

In a further aspect, the present invention includes a bank of isolated T-cell and mesenchymal stem cell subpopulations. The T-cell and mesenchymal stem cell subpopulations are characterized, the characterization is recorded in a database for future use, and the T-cell subpopulations cryopreserved. The T-cell subpopulation has been characterized by, for example, HLA-phenotype, its specificity to its specific TAA or VAA, the epitope or epitopes each T-cell subpopulation is specific to, which MHC Class I and Class II the T-cell subpopulation is restricted to, antigenic activity through the T-cell's corresponding HLA-allele, and immune effector subtype concentration. The mesenchymal stem cell subpopulation has been characterized by, for example, donor source.

In some aspects, as described above, the T-cell subpopulation and/or MSC population can be a newly produced T cell subpopulation and/or MSC population, that has yet to be banked,

### Summary of Embodiments for VM Composition, Use and Manufacture

In another aspect, the invention is a composition and method of treating a patient undergoing a HSCT due to a disorder other than a malignancy that includes:
i) determining the HLA subtype of the patient;
ii) identifying one or more viral associated antigens for targeting with one or more VAA-specific T-cell subpopulations;
iii) selecting at least one banked T-cell subpopulation having the highest activity against each targeted VAA through one or more HLA-alleles shared between the patient and the VAA T-cell subpopulations, wherein each T-cell subpopulation is specific for multiple virus associated antigens, wherein each of the T-cell subpopulations are primed and expanded ex vivo;
iv) selecting at least one banked mesenchymal stem cell population;
v) combining each selected banked T-cell subpopulation and MSC population to create a VM composition;
vi) administering an effective amount of the VM composition to the patient; and,
vii) repeating the administration of the VM composition as necessary.
   In some embodiments, the shared HLA alleles are selected from one or more of HLA-A, HLA-B, or HLA-DR. In some embodiments, the VM composition is administered concomitantly with the HSCT. In some embodiments, the VM composition is administered following HSCT.

In a typical embodiment, a patient, such as a human, is infused or injected with an effective dose of a VM composition ranging from 1×10⁶ to 1×10⁸ cells/m² of a multi-VAA T-cell subpopulation, and 1-5×10⁶/kg of a MSC population. Alternatively, the cell subpopulations of the VM composition are not combined into a single dosage form, but rather each cell population is administered separately. The patient may receive a second or additional infusion or injection up to 1, 2, 3 or more weeks later if recommended by the health care practitioner and may receive additional doses subsequent thereto as useful and recommended.

The viral T-cells for the VM composition can be primed and activated using a number of known procedures, including but limited to the below process:
i) identifying eligible donors who are negative to the patient's disease, and preferably healthy, and wherein the donor can be cord blood or PBMCs;
ii) collecting the mononuclear cells from the negative donor and optionally removing any effector or other memory T-cells optionally based on CD45RA⁻, CD45RO⁺, CCR7⁻, CD62L⁻, CCR7⁺, and/or CD62L⁺ markers;
iii) separating the mononuclear cells into two components;
iv) separating the cells in the first component into nonadherent T-cells and precursors and adherent dendritic cells and precursors, using any method known in the art, for example exposure to a solid medium, separation magnetically, use of antibodies, etc., and if not done already, optionally removing any effector or other memory T-cells optionally based on CD45RA−, CD45RO+, CCR7, CD62L−, or CCR7+, CD62L+ markers;
v) differentiating monocytes and precursors to dendritic cells with IL-4 and GM-CSF, followed by treatment with maturing cytokines such as LPS, TNFα, IL-1β, IL-4, IL-6 and GM-CSF and then pulsing with one or more peptide(s) and/or epitope(s) from single or multiple selected VAAs; and then irradiating to form dendritic antigen presenting cells (APCs);
vi) treating the nonadherent T-cells and precursors with cytokines IL-7 and IL-15 to polarize to Th1 cells (and in some embodiments, without the use of IL-12);
vii) mixing the dendritic antigen presenting cells from (v) with the non-adherent T-cells and T-cell precursor cells from (vi) in the presence of cytokines IL-6, optionally in a ratio of between 5:1 and 20:1 of (vi) to (v) to produce a T-cell subpopulation specific for a single TAA or VAA;
viii) treating the second component of mononuclear cells with a mitogen such as PHA, a T-blast, B-blast, lymphoblastic cell or CD3/CD28 Blast optionally in the presence of IL-2 to produce activated T-cells; and then irradiating the cells to inhibit growth;
ix) pulsing the PHA blasts in (viii) with selected antigenic peptide(s) and/or epitope(s) from the selected tumor-associated antigens and irradiating to inhibit growth;
x) mixing the antigen specific T-cells from (vii) with the activated T-cell subpopulation from (ix) optionally in the presence of K562 accessory cells (preferably HLA-negative, K562 cells expressing CD80, CD83, CD86 and/or 4-IBBL) or LPS, and optionally IL-15 and/or IL-2;
xi) recovering the produced multi-antigen-specific T-cell subpopulation;
xii) optionally characterizing the resulting T-cell subpopulation for banking; and,
xiii) optionally cryopreserving and storing in the bank until use.

In the above process, unless specific steps are taken to remove cell components of the donor blood starting material, for example, removal based on cell surface markers, etc., the final T-cell subpopulation will normally also include a range of cell types, such as Natural Killer T-cells, γδ T-cells, CD4+ T-cells, CD8+ (cytotoxic) T-cells, and Natural Killer T-cells, among others, and may have naïve, and effector memory or central memory cells. The ratios of these cell types in the TVM composition will vary according to the donor's blood and processing conditions.

In another aspect, the present invention includes a method of manufacturing a T-cell subpopulation of the present invention comprising (i) collecting a mononuclear cell product from a healthy donor; (ii) determining the HLA subtype of the mononuclear cell product; (iii) separating the monocytes and the lymphocytes of the mononuclear cell product; (iv) generating and maturing dendritic cells (DCs) from the monocyte fraction; (v) pulsing the DCs with one or more peptides and/or epitopes from multiple VAAs; (vi) carrying out a CD45RA+ selection to isolate naïve lymphocytes from the lymphocyte fraction; (vii) stimulating the naïve lymphocytes with the peptide-pulsed DCs in the presence of a cytokine cocktail; (viii) repeating the T cell stimulation with fresh peptide-pulsed DCs or other peptide-pulsed antigen presenting cells in the presence of a cytokine cocktail; and (ix) harvesting the T-cell subpopulation, (x) characterizing the T-cell subpopulation as described herein; and (xi) banking the T-cell subpopulation for future use in a TVM composition.

In another aspect, the present invention includes a method of manufacturing a T-cell subpopulation of the present invention comprising (i) collecting a mononuclear cell product from a healthy donor; (ii) determining the HLA subtype of the mononuclear cell product; (iii) separating the monocytes and the lymphocytes of the mononuclear cell product; (iv) generating and maturing dendritic cells (DCs) from the monocyte fraction; (v) pulsing the DCs with one or more peptides and/or epitopes from multiple VAAs; (vi) carrying out a CD45RA+ selection to isolate naïve T cells from the lymphocyte fraction; (vii) stimulating the naïve T cells with the peptide-pulsed DCs in the presence of a cytokine cocktail; (viii) repeating the T cell stimulation with fresh peptide-pulsed DCs or other peptide-pulsed antigen presenting cells in the presence of a cytokine cocktail, creating a primed T cell subpopulation; and (ix) harvesting the primed T cell subpopulation, (x) characterizing the primed T cell subpopulation as described herein; and (xi) banking the primed T cell subpopulation for future use in a TVM composition.

In another aspect, the present invention includes a method of isolating and expanding a homogeneous mesenchymal stem cell population of the present invention comprising i) collecting bone marrow from a donor; ii) priming and coating the cell expansion set in the bioreactor; iii) loading bone marrow into the bioreactor; iv) feeding the MSCs; v) harvesting MSCs; vi) performing additional passages; and vii) cryopreservation.

In a further aspect, the present invention includes a bank of isolated T-cell and mesenchymal stem cell subpopulations. The T-cell and mesenchymal stem cell subpopulations are characterized, the characterization is recorded in a database for future use, and the T-cell subpopulations cryopreserved. The T-cell subpopulation has been characterized by, for example, HLA-phenotype, its specificity to its specific TAA or VAA, the epitope or epitopes each T-cell subpopulation is specific to, which MHC Class I and Class II the T-cell subpopulation is restricted to, antigenic activity through the T-cell's corresponding HLA-allele, and immune effector subtype concentration. The mesenchymal stem cell subpopulation has been characterized by, for example, donor source.

### DETAILED DESCRIPTION OF THE INVENTION

Complications of hematopoietic stem cell transplant (HSCT) can be reduced by administering to a patient in need thereof an effective amount of a cell therapy composition that includes in the same dosage form a multiplicity of T-cell and mesenchymal stem cell subpopulations as further described herein. In some embodiments, for the treatment of patients with a malignancy, the composition (“TVM”) and method comprises one or more T-cell subpopulations specific for multiple tumor-associated antigens (TAAs), one or more T-cell subpopulations specific for one or more virus-associated antigens (VAAs), and a mesenchymal stem cell population, wherein the TAA T-cell subpopulations that comprise the TVM composition for administration are chosen specifically based on the TAA expression profile of the patient's tumor. In another embodiment, for the treatment of patients undergoing HSCT for a disorder other than a malignancy, the composition (“VM”) and method comprises one or more T-cell subpopulations specific for one or more virus-associated antigens (VAAs), and a mesenchymal stem cell population.

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “allogeneic” as used herein refers to medical therapy in which the donor and recipient are different individuals of the same species.

The term “antigen” as used herein refers to molecules, such as polypeptides, peptides, or glyco- or lipo-peptides that are recognized by the immune system, such as by the cellular or humoral arms of the human immune system. The term “antigen” includes antigenic determinants, including but not limited to peptides with lengths of 6, 7, 8, 9, 10, 11, 12, 13, 14, 15, 16, 17, 18, 19, 20, 21, 22 or more amino acid residues that bind to MHC molecules, form parts of MHC Class I or II complexes, or that are recognized when complexed with such molecules.

The term “antigen presenting cell (APC)” as used herein refers to a class of cells capable of presenting one or more antigens in the form of peptide-MHC complex recognizable by specific effector cells of the immune system, and thereby inducing an effective cellular immune response against the antigen or antigens being presented. Examples of professional APCs are dendritic cells and macrophages, though any cell expressing MHC Class I or II molecules can potentially present peptide antigen.

The term “autologous” as used herein refers to medical therapy in which the donor and recipient are the same person.

The term “cord blood” as used herein has its normal meaning in the art and refers to blood that remains in the placenta and umbilical cord after birth and contains hematopoietic stem cells. Cord blood may be fresh, cryopreserved, or obtained from a cord blood bank.

The term “cytokine” as used herein has its normal meaning in the art. Nonlimiting examples of cytokines used in the invention include IL-2, IL-6, IL-7, IL-12, IL-15, and IL-27.

The term “cytotoxic T-cell” or “cytotoxic T lymphocyte” as used herein is a type of immune cell that bears a CD8+ antigen and that can kill certain cells, including foreign cells, tumor cells, and cells infected with a virus. Cytotoxic T cells can be separated from other blood cells, grown ex vivo, and then given to a patient to kill tumor or viral cells. A cytotoxic T cell is a type of white blood cell and a type of lymphocyte.

The term “dendritic cell” or “DC” as used herein describes a diverse population of morphologically similar cell types found in a variety of lymphoid and non-lymphoid tissues, see Steinman, Ann. Rev. Immunol. 9:271-296 (1991).

The term “effector cell” as used herein describes a cell that can bind to or otherwise recognize an antigen and mediate an immune response. Tumor, virus, or other antigen-specific T-cells and NKT-cells are examples of effector cells.

The term “endogenous” as used herein refers to any material from or produced inside an organism, cell, tissue or system.

The term “epitope” or “antigenic determinant” as used herein refers to the part of an antigen that is recognized by the immune system, specifically by antibodies, B cells, or T cells.

The term “exogenous” as used herein refers to any material introduced from or produced outside an organism, cell, tissue or system.

The term “HLA” as used herein refers to human leukocyte antigen. There are 7,196 HLA alleles. These are divided into 6 HLA class I and 6 HLA class II alleles for each individual (on two chromosomes). The HLA system or complex is a gene complex encoding the major histocompatibility complex (MHC) proteins in humans. HLAs corresponding to MHC Class I (A, B, or C) present peptides from within the cell and activate CD8-positive (i.e., cytotoxic) T-cells. HLAs corresponding to MHC Class II (DP, DM, DOA, DOB, DQ and DR) stimulate the multiplication of CD4-positive T-cells) which stimulate antibody-producing B-cells.

The term “isolated” as used herein means separated from components in which a material is ordinarily associated with, for example, an isolated cord blood mononuclear cell can be separated from red blood cells, plasma, and other components of cord blood.

The terms “mesenchymal stem cell” and “mesenchymal stromal cell” as used herein are used interchangeably and are defined as a plastic-adherent cell population that can be directed to differentiate in vitro into cells of osteogenic, chondrogenic, adipogenic, myogenic, and other lineages. As part of their stem cell nature, MSCs proliferate and give rise to daughter cells that have the same pattern of gene expression and phenotype and, therefore, maintain the ‘sternness’ of the original cells.

A “naive” T-cell or other immune effector cell as used herein is one that has not been exposed to or primed by an antigen or to an antigen-presenting cell presenting a peptide antigen capable of activating that cell.

The term “passaging” as used herein is a technique that enables cells to be kept alive and growing under cultured conditions for extended periods of time. Passaging involves transferring some or all cells from a previous culture to fresh growth medium. Cells are generally passaged when they reach confluence.

A “peptide library” or “overlapping peptide library” as used herein within the meaning of the application is a complex mixture of peptides which in the aggregate covers the partial or complete sequence of a protein antigen. Successive peptides within the mixture overlap each other, for example, a peptide library may be constituted of peptides 15 amino acids in length which overlapping adjacent peptides in the library by 11 amino acid residues and which span the entire length of a protein antigen. Peptide libraries are commercially available and may be custom-made for particular antigens. Methods for contacting, pulsing or loading antigen-presenting cells are well known and incorporated by reference to Ngo, et al (2014), Peptide libraries may be obtained from JPT and are incorporated by reference to the website at https://www.jpt.com/products/peptrack/peptide-libraries.

A “peripheral blood mononuclear cell” or “PBMC” as used herein is any peripheral blood cell having a round nucleus. These cells consist of lymphocytes (T cells, B cells, NK cells) and monocytes. In humans, lymphocytes make up the majority of the PBMC population, followed by monocytes, and only a small percentage of dendritic cells.

The term “precursor cell” as used herein refers to a cell which can differentiate or otherwise be transformed into a particular kind of cell. For example, a “T-cell precursor cell” can differentiate into a T-cell and a “dendritic precursor cell” can differentiate into a dendritic cell.

A “subject” or “host” or “patient” as used herein is a vertebrate, preferably a mammal, more preferably a human. Mammals include, but are not limited to humans, simians, equines, bovines, porcines, canines, felines, murines, other farm animals, sport animals, or pets. Humans include those in need of virus- or other antigen-specific T-cells, such as those with lymphocytopenia, those who have undergone immune system ablation, those undergoing transplantation and/or immunosuppressive regimens, those having naïve or developing immune systems, such as neonates, or those undergoing cord blood or stem cell transplantation. In a typical embodiment, the term “patient” as used herein refers to a human.

A “T-cell population” or “T-cell subpopulation” is intended to include thymocytes, immature T lymphocytes, mature T lymphocytes, resting T lymphocytes and activated T-lymphocytes. The T-cell population or subpopulation can include αβ T-cells, including CD4+ T-cells, CD8+ T cells, γδ T-cells, Natural Killer T-cells, or any other subset of T-cells.

The term “tumor-associated antigen expression profile” or “tumor antigen expression profile” as used herein, refers to a profile of expression levels of tumor-associated antigens within a malignancy or tumor. Tumor-associated antigen expression may be assessed by any suitable method known in the art including, without limitation, quantitative real time polymerase chain reaction (qPCR), cell staining, or other suitable techniques. Non-limiting exemplary methods for determining a tumor-associated antigen expression profile can be found in Ding et al., Cancer Bio Med (2012) 9: 73-76; Qin et al., Leukemia Research (2009) 33(3) 384-390; Weber et al., Leukemia (2009) 23: 1634-1642; Liu et al., J. Immunol (2006) 176: 3374-3382; Schuster et al., Int J Cancer (2004) 108: 219-227.

The terms “tumor-associated antigen” or “TAA” as used herein is an antigen that is highly correlated with certain tumor cells. They are not usually found, or are found to a lesser extent, on normal cells.

The term “TVM composition” as used herein refers to a composition comprising a multi-tumor-associated antigen T-cell population, a multi-virus-associated antigen T-cell population, and a mesenchymal stem cell population. For purposes herein, when referring to combining T-cell subpopulations and mesenchymal stem cell populations to comprise the TVM composition, combining is intended to include the situation wherein the different cell types are physically combined into a single dosage form, that is, a single composition. In alternative embodiments, the cell subpopulations are kept physically separated but administrated concomitantly and collectively comprise the TVM composition.

The terms “viral-associated antigen” or “VAA” as used herein is a toxin or other substance given off by a virus which causes an immune response in its host. Viral antigens are protein in nature, typically strain-specific, and can be closely associated with the virus particle. A viral antigen is a protein encoded by the viral genome. A viral protein is an antigen specified by the viral genome that can be detected by a specific immunological response.

The term “VM composition” as used herein refers to a composition comprising a multi-virus-associated antigen T-cell population and a mesenchymal stem cell population. For purposes herein, when referring to combining T-cell subpopulations and mesenchymal stem cell populations to comprise the VM composition, combining is intended to include the situation wherein the different cell types are physically combined into a single dosage form, that is, a single composition. In alternative embodiments, the cell subpopulations are kept physically separated but administrated concomitantly and collectively comprise the VM composition.

### Tumor-Associated Antigens

The TVM compositions for administration provided herein include a T-cell subpopulation specific for one or more TAAs. The careful selection of antigens for TVM composition therapy is critical to success. Antigens used for immunotherapy should be intentionally selected based on either uniqueness to tumor cells, greater expression in tumor cells as compared to normal cells, or ability of normal cells with antigen expression to be adversely affected without significant compromise to normal cells or tissue.

Tumor-associated antigens (TAA) can be loosely categorized as oncofetal (typically only expressed in fetal tissues and in cancerous somatic cells), oncoviral (encoded by tumorigenic transforming viruses), overexpressed/accumulated (expressed by both normal and neoplastic tissue, with the level of expression highly elevated in neoplasia), cancer-testis (expressed only by cancer cells and adult reproductive tissues such as testis and placenta), lineage-restricted (expressed largely by a single cancer histotype), mutated (only expressed by cancer as a result of genetic mutation or alteration in transcription), post-translationally altered (tumor-associated alterations in glycosylation, etc.), or idiotypic (highly polymorphic genes where a tumor cell expresses a specific “clonotype”, i.e., as in B cell, T cell lymphoma/leukemia resulting from clonal aberrancies). Although they are preferentially expressed by tumor cells, TAAs are sometimes found in normal tissues. However, their expression differs from that of normal tissues by their degree of expression in the tumor, alterations in their protein structure in comparison with their normal counterparts or by their aberrant subcellular localization within malignant or tumor cells.

Examples of oncofetal tumor associated antigens include Carcinoembryonic antigen (CEA), immature laminin receptor, and tumor-associated glycoprotein (TAG) 72. Examples of overexpressed/accumulated include BING-4, calcium-activated chloride channel (CLCA) 2, Cyclin B1, 9D7, epithelial cell adhesion molecule (Ep-Cam), EphA3, Her2/neu, telomerase, mesothelin, orphan tyrosine kinase receptor (ROR1), stomach cancer-associated protein tyrosine phosphatase 1 (SAP-1), and survivin.

Examples of cancer-testis antigens include the b melanoma antigen (BAGE) family, cancer-associated gene (CAGE) family, G antigen (GAGE) family, melanoma antigen (MAGE) family, sarcoma antigen (SAGE) family and X antigen (XAGE) family, CT9, CT10, NY-ESO-1, L antigen (LAGE) 1, Melanoma antigen preferentially expressed in tumors (PRAME), and synovial sarcoma X (SSX) 2. Examples of lineage restricted tumor antigens include melanoma antigen recognized by T cells-1/2 (Melan-A/MART-1/2), Gp100/pmel17, tyrosine-related protein (TRP) 1 and 2, P. polypeptide, melanocortin 1 receptor (MC1R), and prostate-specific antigen. Examples of mutated tumor antigens include β-catenin, breast cancer antigen (BRCA) 1/2, cyclin-dependent kinase (CDK) 4, chronic myelogenous leukemia antigen (CML) 66, fibronectin, p53, Ras, and TGF-βRII. An example of a post-translationally altered tumor antigen is mucin (MUC) 1. Examples of idiotypic tumor antigens include immunoglobulin (Ig) and T cell receptor (TCR).

In some embodiments, the antigen associated with the disease or disorder is selected from the group consisting of CD19, CD20, CD22, hepatitis B surface antigen, anti-folate receptor, CD23, CD24, CD30, CD33, CD38, CD44, EGFR, EGP-2, EGP-4, 0EPHa2, ErbB2, 3, or 4, FBP, fetal acetylcholine receptor, HMW-MAA, IL-22R-alpha, IL-13R-alpha, kdr, kappa light chain, Lewis Y, MUC16 (CA-125), PSCA, NKG2D Ligands, oncofetal antigen, VEGF-R2, PSMA, estrogen receptor, progesterone receptor, ephrinB2, CD123, CS-1, c-Met and/or biotinylated molecules, and/or molecules expressed by HIV, HCV, HBV or other pathogens.

Exemplary tumor antigens include at least the following: carcinoembryonic antigen (CEA) for bowel cancers; CA-125 for ovarian cancer; MUC1 or epithelial tumor antigen (ETA) or CA15-3 for breast cancer; tyrosinase or melanoma-associated antigen (MAGE) for malignant melanoma; and abnormal products of ras, p53 for a variety of types of tumors; alphafetoprotein for hepatoma, ovarian, or testicular cancer; beta subunit of hCG for men with testicular cancer; prostate specific antigen for prostate cancer; beta 2 microglobulin for multiple myeloma and in some lymphomas; CA19-9 for colorectal, bile duct, and pancreatic cancer; chromogranin A for lung and prostate cancer; TA90 for melanoma, soft tissue sarcomas, and breast, colon, and lung cancer. Examples of TAAs are known in the art, for example in N. Vigneron, “Human Tumor Antigens and Cancer Immunotherapy,” BioMed Research International, vol. 2015, Article ID 948501, 17 pages, 2015. doi:10.1155/2015/948501; Ilyas et al., J Immunol. (2015) Dec. 1; 195(11): 5117-5122; Coulie et al., Nature Reviews Cancer (2014) volume 14, pages 135-146; Cheever et al., Clin Cancer Res. (2009) Sep. 1; 15(17):5323-37, which are incorporated by reference herein in its entirety.

Examples of oncoviral TAAs include human papilloma virus (HPV) L1, E6 and E7, Epstein-Barr Virus (EBV) Epstein-Barr nuclear antigen (EBNA), EBV viral capsid antigen (VCA) Igm or IgG, EBV early antigen (EA), latent membrane protein (LMP) 1 and 2, hepatitis B surface antigen (HBsAg), hepatitis B e antigen (HBeAg), hepatitis B core antigen (HBcAg), hepatitis B×antigen (HB×Ag), hepatitis C core antigen (HCV core Ag), Human T-Lymphotropic Virus Type 1 core antigen (HTLV-1 core antigen), HTLV-1 Tax antigen, HTLV-1 Group specific (Gag) antigens, HTLV-1 envelope (Env), HTLV-1 protease antigens (Pro), HTLV-1 Tof, HTLV-1 Rof, HTLV-1 polymerase (Pro) antigen, Human T-Lymphotropic Virus Type 2 core antigen (HTLV-2 core antigen), HTLV-2 Tax antigen, HTLV-2 Group specific (Gag) antigens, HTLV-2 envelope (Env), HTLV-2 protease antigens (Pro), HTLV-2 Tof, HTLV-2 Rof, HTLV-2 polymerase (Pro) antigen, latency-associated nuclear antigen (LANA), human herpesvirus-8 (HHV-8) K8.1, Merkel cell polyomavirus large T antigen (LTAg), and Merkel cell polyomavirus small T antigen (sTAg).

Elevated expression of certain types of glycolipids, for example gangliosides, is associated with the promotion of tumor survival in certain types of cancers. Examples of gangliosides include, for example, GM1b, GD1c, GM3, GM2, GM1a, GD1a, GT1a, GD3, GD2, GD1b, GT1b, GQ1b, GT3, GT2, GT1c, GQ1c, and GP1c. Examples of ganglioside derivatives include, for example, 9-O-Ac-GD3, 9-O-Ac-GD2, 5-N-de-GM3, N-glycolyl GM3, NeuGcGM3, and fucosyl-GM1. Exemplary gangliosides that are often present in higher levels in tumors, for example melanoma, small-cell lung cancer, sarcoma, and neuroblastoma, include GD3, GM2, and GD2.

In addition to the TAAs described above, another class of TAAs is tumor-specific neoantigens, which arise via mutations that alter amino acid coding sequences (non-synonymous somatic mutations). Some of these mutated peptides can be expressed, processed and presented on the cell surface, and subsequently recognized by T cells. Because normal tissues do not possess these somatic mutations, neoantigen-specific T cells are not subject to central and peripheral tolerance, and also lack the ability to induce normal tissue destruction. See, e.g., Lu & Robins, Cancer Immunotherapy Targeting Neoantigens, Seminars in Immunology, Volume 28, Issue 1, February 2016, Pages 22-27, incorporated herein by reference.

As a non-limiting example, Wilms tumor gene (WT1) is found in post-natal kidney, pancreas, fat, gonads and hematopoietic stem cells. In healthy hematopoietic stem cells WT1 encodes a transcription factor, which regulates cell proliferation, cell death and differentiation. WT1 is overexpressed in Wilms tumor, soft tissue sarcomas, rhabdomyosarcoma, ovarian, and prostate cancers. The WT1 gene was initially identified as a tumor suppressor gene due to its inactivation in Wilms' tumor (nephroblastoma), the most common pediatric kidney tumor. However, recent findings have shown that WT1 acts as an oncogene in ovarian and other tumors. In addition, several studies have reported that high expression of WT1 correlates with the aggressiveness of cancers and a poor outcome in leukemia, breast cancer, germ-cell tumor, prostate cancer, soft tissue sarcomas, rhabdomyosarcoma and head and neck squamous cell carcinoma.

There are several studies describing WT1 expression in ovarian cancers. A positive expression has been primarily observed in serous adenocarcinoma, and WT1 is more frequently expressed in high-grade serous carcinoma, which stands-out from other sub-types due to its aggressive nature and because it harbors unique genetic alterations. Patients with WT1-positive tumors tend to have a higher grade and stage of tumor.

Preferentially expressed antigen of melanoma (PRAME), initially identified in melanoma, has been associated with other tumors including neuroblastoma, osteosarcoma, soft tissue sarcomas, head and neck, lung and renal cancer including Wilms tumor. In neuroblastoma and osteosarcoma, PRAME expression was associated with advanced disease and a poor prognosis. PRAME is also highly expressed in leukemic cells and its expression levels are correlated with relapse and remission. The function in healthy tissue is not well understood, although studies suggest PRAME is involved in proliferation and survival in leukemia cells.

Survivin is highly expressed during normal fetal development but is absent in most mature tissues. It is thought to regulate apoptosis and proliferation of hematopoietic stem cells. Overexpression of survivin has been reported in almost all human malignancies including bladder cancer, lung cancer, breast cancer, stomach, esophagus, liver, ovarian cancers and hematological cancers. Survivin has been associated with chemotherapy resistance, increased tumor recurrence and decreased survival.

In some embodiments, the TVM composition includes one or more T-cell subpopulations targeting WT1, PRAME, Survivin, NY-ESO-1, MAGE-A3, MAGE-A4, Pr3, Cyclin A1, SSX2, Neutrophil Elastase (NE), or a combination thereof. In some embodiments, the TVM composition includes T-cell subpopulations targeting WT1, PRAME, and Survivin.

### Viral-Associated Antigens

The TVM and VM compositions for administration provided herein include a T-cell subpopulation specific for one or more VAAs. Patients receiving HSCT are particularly susceptible to viral infections. A virus is a sub-micrometer particle that has DNA or RNA packed in a shell called capsid. Viral antigens protrude from the capsid and often fulfill important function in docking to the host cell, fusion, and injection of viral DNA/RNA. Antibody-based immune responses form a first layer of protection of the host from viral infection; however, in many cases a vigorous cellular immune response mediated by T-cells and NK-cells is required for effective viral clearance.

A viral antigen is a toxin or other substance given off by a virus which causes an immune response in its host. Viral antigens are protein in nature, strain-specific, and closely associated with the virus particle. A viral antigen is a protein encoded by the viral genome. A viral protein is an antigen specified by the viral genome that can be detected by a specific immunological response.

Each virus has its own viral-associated antigens. Examples of antigens to cytomegalovirus (CMV) include immediate-early protein 1 (IE-1), immediate-early protein 2 (IE-2), 65 kDa phosphoprotein (pp65). Examples of antigens to Epstein-Barr Virus (EBV) include the Epstein-Barr Nuclear Antigen (EBNA) family, which includes EBNA-leader protein (EBNA-LP), EBNA1, EBNA2, EBNA3a, EBNA3b, EBNA3c; latent membrane protein (LMP) family, which includes LMP1 and LMP2; envelope glycoprotein GP350/GP340; secreted protein BARF1; mRNA export factor EB2 (BMLF1); DNA polymerase processivity factor (BMRF1) and trans-activator protein (BZLF1). Examples of antigens to human adenovirus (HAdV) include the hexon protein of Human adenovirus 3 (HAdV-3) and the penton protein of Human adenovirus 5 (HAdV-5). Examples of antigens to BK polyomavirus include capsid protein VP-1, capsid protein VP-2, large T antigen, and small T antigen. Examples of antigens to Human herpesvirus 6 (HHV-6) include proteins U14, U54 and U90. Examples of antigens to respiratory syncytial virus (RSV) include the fusion glycoprotein (F), major surface glycoprotein G, small hydrophobic protein (SH), and nucleocapsid (N) protein. Examples of antigens to human influenza include matrix protein (MP) 1, matrix protein (MP) 2, nucleocapsid protein (NP) 1, neuroaminidase, hemagglutinin (HA). Examples of antigens to human papillomavirus (HPV) include protein E4, protein E5, protein E6, protein E7, late major capsid protein (L) 1, replication protein E1, and replication protein E2. Examples of antigens to human immunodeficiency virus (HIV) include envelope glycoprotein gp160 (Env), Gag polyprotein, Nef protein, and Pol polyprotein.

In some embodiments, the TVM or VM composition includes one or more T-cell subpopulations specific to the viral-associated antigens IE-1, pp65, EBNA1, LMP1, LMP2, Hexon, Penton, LT, VP-1, MP1, NP1, N, F, U14 and U90, or a combination thereof. In some embodiments, the TVM or VM compositions includes one or more T-cell subpopulations specific to at least one of the viral-associated antigens of CMV selected from IE-1 and pp65; at least one of the viral-associated antigens of EBV selected from EBNA1, LMP1, LMP2, BARF1 and BZLF1; at least one of the viral-associated antigens of AdV selected from Hexon and Penton; at least one of the viral-associated antigens of BK virus selected from LT and VP-1; at least one of the viral-associated antigens of parainfluenza selected from MP1 and NP1; at least one of the viral-associated antigens of RSV selected from N and F; and at least one of the viral-associated antigens from HHV6 selected from U14 and U90.

### Generation of Targeted Tumor-Associated Antigen Peptides for Use in Activating T-Cell Subpopulations

T-cell subpopulations targeting TAAs can be prepared by pulsing antigen presenting cells or artificial antigen presenting cells with a single peptide or epitope, several peptides or epitopes, or with overlapping peptide libraries of the selected antigen, that for example, include peptides that are about 7, 8, 9, 10, 11, 12, 13, 14, 15, 16 or more amino acids long and overlapping one another by 5, 6, 7, 8, 9, 10, 11 or more amino acids, in certain aspects. GMP-quality overlapping peptide libraries directed to a number of tumor-associated antigens are commercially available, for example, through JPT Technologies and/or Miltenyi Biotec. In particular embodiments, the peptides are 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and there is overlap of 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length.

The TAA-targeting T-cell component of the TVM can be prepared by using a multi-TAA priming and expanding approach wherein the T-cells are primed with a mastermix of one or more antigenic peptides from two or more TAAs. Alternatively, the TAA targeting T cell component of the TVM can be prepared by separately priming and expanding a T-cell subpopulation to each targeted TAA, and then combining the separately primed and activated T-cell subpopulations.

In some embodiments, the T-cell subpopulation is specific to one or more known epitopes of the targeted TAA. Much work has been done to determine specific epitopes of TAAs and the HLA alleles they are associated with. Non-limiting examples of specific epitopes of TAAs and the alleles they are associated with can be found in Kessler et al., J Exp Med. 2001 Jan. 1; 193(1):73-88; Oka et al. Immunogenetics. 2000 February; 51(2):99-107; Ohminami et al., Blood. 2000 Jan. 1; 95(1):286-93; Schmitz et al., Cancer Res. 2000 Sep. 1; 60(17):4845-9 and Bachinsky et al., Cancer Immun. 2005 Mar. 22; 5:6, which are each incorporated herein by reference.

In some embodiments, the TAA peptides used to prime and expand a T-cell subpopulation includes specifically selected HLA-restricted peptides generated by determining the HLA profile of the donor source, and including peptides derived from the targeted TAA that best match the donor's HLA type. By including specifically selected donor HLA-restricted peptides in the peptide mix for priming and expanding T-cell subpopulations, a T-cell subpopulation can be generated that provides greater TAA targeted activity through more than one donor HLA, improving potential efficacy of the T-cell subpopulation. In addition, by generating a T-cell subpopulation with TAA targeted activity through more than one donor HLA allele, a single donor T-cell subpopulation may be included in a TVM composition for multiple recipients with different HLA profiles by matching one or more donor HLAs showing TAA-activity. In some embodiments, the TAA peptides used to prime and expand a T-cell subpopulation are derived from HLA-restricted peptides selected from at least one or more of an HLA-A restricted peptide, HLA-B restricted peptide, or HLA-DR restricted peptide. In some embodiments, the HLA-restricted epitopes are specific to at least one or more of a cell donor's HLA-A alleles, HLA-B alleles, or HLA-DR alleles. In some embodiments, the HLA-restricted epitopes are specific to at least both of the donor's HLA-A alleles, at least both of the donor's HLA-B alleles, and at least both of the donor's HLA-DR alleles. In some embodiments, the HLA-A alleles are selected from a group comprising HLA-A*01, HLA-A*02:01, HLA-A*03, HLA-A*11:01, HLA-A*24:02, HLA-A*26, or HLA-A*68:01. In some embodiments, the HLA-B alleles are selected from a group comprising HLA-B*07:02, HLA-B*08, HLA-B*15:01 (B62), HLA-B*18, HLA-B*27:05, HLA-B*35:01, or HLA-B*58:02. In some embodiments, the HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b). Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s0025100050595. In some embodiments, the mastermix of peptides includes both an overlapping peptide library and specifically selected HLA-restricted peptides generated by determining the HLA profile of the donor source.

This focused approach to activation can increase the effectiveness of the activated T-cell subpopulation, and ultimately, the TVM composition

WT-1 Antigenic Peptides

In some embodiments, the TVM composition includes WT-1 specific T-cells. WT1 specific T-cells can be generated as described below using one or more antigenic peptides to WT1. In some embodiments, the WT1 specific T-cells are generated using one or more antigenic peptides to WT1, or a modified or heteroclitic peptide derived from a WT1 peptide. In some embodiments, WT1 specific T-cells are generated using a WT1 antigen library comprising a pool of peptides (for example 15mers) containing amino acid overlap (for example 11 amino acids of overlap) between each sequence formed by scanning the protein amino acid sequence SEQ. ID. No. 1 (UniProtKB—P19544 (WT1_HUMAN)):

MGSDVRDLNALLPAVPSLGGGGGCALPVSGAAQWAPVLDFAPPGASAYGS
LGGPAPPPAPPPPPPPPPHSFIKQEPSWGGAEPHEEQCLSAFTVHFSGQF
TGTAGACRYGPFGPPPPSQASSGQARMFPNAPYLPSCLESQPAIRNQGYS
TVTEDGTPSYGHTPSHHAAQFPNHSFKHEDPMGQQGSLGEQQYSVPPPVY
GCHTPTDSCTGSQALLLRTPYSSDNLYQMTSQLECMTWNQMNLGATLKGV
AAGSSSSVKWTEGQSNHSTGYESDNHTTPILCGAQYRIHTHGVERGIQDV
RRVPGVAPTLVRSASETSEKRPFMCAYPGCNKRYFKLSHLQMHSRKHTGE
KPYQCDFKDCERRFSRSDQLKRHQRRHTGVKPFQCKTCQRKFSRSDHLKT
HTRTHTGKTSEKPFSCRWPSCQKKFARSDELVRHHNMHQRNMTKLQLAL

The antigenic library is commercially available, for example, from JPT (Product Code: PM-WT1: Pep Mix Human (WT1/WT33)). In some embodiments, the WT1 specific T-cells are generated using a commercially available overlapping antigenic library made up of WT1 peptides.

In some embodiments, the WT1 specific T-cells are generated using one or more antigenic peptides to WT1, or a modified or heteroclitic peptide derived from a WT1 peptide,

In some embodiments, the WT1 specific T-cells are generated with peptides that recognize class I MHC molecules. In some embodiments, the WT1 specific T-cells are generated with peptides that recognize class II MHC molecules. In some embodiments, the WT1 specific T-cells are generated with peptides that recognize both class I and class II MHC molecules.

In some embodiments, the WT1 peptides used to prime and expand a T-cell subpopulation includes specifically selected HLA-restricted peptides generated by determining the HLA profile of the donor source, and including peptides derived from WT1 that best match the donor's HLA. In some embodiments, the WT1 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting WT1 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 1-7, the HLA-B peptides are selected from the peptides of Tables 8-14, and the HLA-DR peptides are selected from the peptides of Tables 15-20. For example, if the donor cell source has an HLA profile that is HLA-A*01/*02:01; HLA-B*15:01/*18; and HLA-DRB1*0101/*0301, then the WT1 peptides used to prime and expand the WT1 specific T-cell subpopulation are restricted to the specific HLA profile, and may include the peptides identified in Table 1 (Seq. ID. Nos. 2-11) for HLA-A*01; Table 2 (Seq. ID. No. 12-21) for HLA-A*02:01; Table 10 (Seq. ID. No. 92-101) for HLA-B*15:01; Table 11 (Seq. ID. No. 102-111) for HLA-B*18; Table 15 (Seq. ID. No. 142-151) for HLA-DRB1*0101; and Table 16 (Seq. ID. No. 152-159) for HLA-DRB1*0301. In some embodiments, the mastermix of peptides includes both an overlapping peptide library and specifically selected HLA-restricted peptides generated by determining the HLA profile of the donor source.

In some embodiments, the WT1 HLA-restricted epitopes are specific to at least both of the donor's HLA-A alleles, at least both of the donor's HLA-B alleles, and at least both of the donor's HLA-DR alleles. In some embodiments, the WT1 HLA-A alleles are selected from a group comprising HLA-A*01, HLA-A*02:01, HLA-A*03, HLA-A*11:01, HLA-A*24:02, HLA-A*26, or HLA-A*68:01, and the corresponding WT1 HLA-restricted peptides are selected for: HLA-A*01 from Table 1; HLA-A*02:01 from Table 2; HLA-A*03 from Table 3; HLA-A*11:01 from Table 4; HLA-A*24:02 from Table 5; HLA-A*26 from Table 6; or HLA-A*68:01 from Table 7; or any combination thereof. In some embodiments, the WT1 HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding WT1 HLA-restricted peptides are selected for: HLA-B*07:02 from Table 8; HLA-B*08 from Table 9; HLA-B*15:01 (B62) from Table 10; HLA-B*18 from Table 11; HLA-B*27:05 from Table 12; HLA-B*35:01 from Table 13, or HLA-B*58:02 from Table 14; or any combination thereof. In some embodiments, the WT1 HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding WT1 HLA-restricted peptides are selected for: HLA-DRB1*0101 from Table 15; HLA-DRB1*0301 (DR17) from Table 16; HLA-DRB1*0401 (DR4Dw4) from Table 17; HLA-DRB1*0701 from Table 18; HLA-DRB1*1101 from Table 19; or HLA-DRB1*1501 (DR2b) from Table 20; or any combination thereof.

TABLE 1
WT1 HLA-A*01 Epitope Peptides

| SEQ ID NO. | Sequence |
|---|---|
| 2 | TSEKRPFMCAY |
| 3 | STVTFDGTPSY |
| 4 | HTTPILCGAQY |
| 5 | ESQPAIRNQGY |
| 6 | GSQALLLRTPY |
| 7 | HSRKHTGEKPY |
| 8 | FTGTAGACRY |
| 9 | RTPYSSDNLY |
| 10 | TTPILCGAQY |
| 11 | VTFDGTPSY |

TABLE 2
WT1 HLA-A*02:01 Epitope Peptides

| SEQ ID NO. | Sequence |
|---|---|
| 12 | SLGGGGGCAL |
| 13 | NALLPAVPSL |
| 14 | AIRNQGYSTV |
| 15 | NMHQRNMTKL |
| 16 | ALLPAVPSL |
| 17 | DLNALLPAV |
| 18 | SLGEQQYSV |
| 19 | NLGATLKGV |
| 20 | NLYQMTSQL |
| 21 | ILCGAQYRI |

TABLE 3
WT1 HLA-A*03 Epitope Peptides

| SEQ ID NO. | Sequence |
|---|---|
| 22 | DVRRVPGVAP |
| 23 | ALLPAVPSLG |
| 24 | ALPVSGAAQW |
| 25 | AIRNQGYSTV |
| 26 | RHQRRHTGVK |
| 27 | GVFRGIQDVR |
| 28 | RVPGVAPTL |
| 29 | RIHTHGVFR |
| 30 | DVRRVPGVA |
| 31 | HQRRHTGVK |

TABLE 4
WT1 HLA-A*11:01 Epitope Peptides

| SEQ ID NO. | Sequence |
|---|---|
| 32 | CTGSQALLLR |
| 33 | GVFRGIQDVR |
| 34 | HTGVKPFQCK |
| 35 | RTHTGKTSEK |
| 36 | KTHTRTHTGK |
| 37 | RSASETSEKR |
| 38 | LSHLQMHSRK |
| 39 | FSCRWPSCQK |
| 40 | RSASETSEK |
| 41 | FSRSDQLKR |

TABLE 5
WT1 HLA-A*24:02 Epitope Peptides

| SEQ ID NO. | Sequence |
|---|---|
| 42 | AYPGCNKRYF |
| 43 | QYRIHTHGVF |
| 44 | AFTVHFSGQF |
| 45 | PPPPPPPHSF |
| 46 | PPPPPPHSFI |
| 47 | PYLPSCLESQ |
| 48 | DFKDCERRF |
| 49 | GCNKRYFKL |
| 50 | ALLPAVPSL |
| 51 | PPPPPPHSF |

TABLE 6
WT1 HLA-A*26 Epitopes Peptides

| SEQ ID NO. | Sequence |
|---|---|
| 52 | TVTFDGTPSY |
| 53 | DFAPPGASAY |
| 54 | EGQSNHSTGY |
| 55 | TTPILCGAQY |
| 56 | ETSEKRPFMC |
| 57 | DVRDLNALL |
| 58 | VTFDGTPSY |
| 59 | FTVHFSGQF |
| 60 | EKRPFMCAY |
| 61 | ETSEKRPFM |

TABLE 7
WT1 HLA-A*68:01 Epitope Peptides

| SEQ ID NO. | Sequence |
|---|---|
| 62 | GVFRGIQDVRR |
| 63 | TTPILCGAQYR |
| 64 | ELVRHHNMHQR |
| 65 | PSCLESQPAIR |
| 66 | CTGSQALLLR |
| 67 | GVFRGIQDVR |
| 68 | KTHTRTHTGK |
| 69 | LVRHHNMHQR |
| 70 | FTGTAGACR |
| 71 | RIHTHGVFR |

TABLE 8
WT1 HLA-B*07:02 Epitope Peptides

| SEQ ID NO. | Sequence |
|---|---|
| 72 | PPGASAYGSL |
| 73 | EPHEEQCLSA |
| 74 | LPSCLESQPA |
| 75 | PPPPPPHSFI |
| 76 | PPSQASSGQA |
| 77 | DPMGQQGSL |
| 78 | PPPPPHSFI |
| 79 | PPPPPPHSF |
| 80 | TPSHHAAQF |
| 81 | WPSCQKKFA |

TABLE 9
WT1 HLA-B*08 Epitope Peptides

| SEQ ID NO. | Sequence |
|---|---|
| 82 | KRYFKLSHL |
| 83 | GCNKRYFKL |
| 84 | KKFARSDEL |
| 85 | GATLKGVAA |
| 86 | RRFSRSDQL |
| 87 | MTKLQLAL |
| 88 | EPHEEQCL |
| 89 | ETSEKRPF |
| 90 | CNKRYFKL |
| 91 | RNMTKLQL |

TABLE 10
WT1 HLA-B*15:01 (B62) Epitope Peptides

| SEQ ID NO. | Sequence |
|---|---|
| 92 | QQYSVPPPVY |
| 93 | TVTFDGTPSY |
| 94 | QQGSLGEQQY |
| 95 | SQALLLRTPY |
| 96 | SQPAIRNQGY |
| 97 | FQCKTCQRKF |
| 98 | AQWAPVLDF |
| 99 | GQSNHSTGY |
| 100 | NQGYSTVTF |
| 101 | CLSAFTVHF |

TABLE 11
WT1 HLA-B*18 Epitope Peptides

| SEQ ID NO. | Sequence |
|---|---|
| 102 | HEEQCLSAF |
| 103 | SETSEKRPF |
| 104 | GEKPYQCDF |
| 105 | SEKPFSCRW |
| 106 | AEPHEEQCL |
| 107 | DVRDLNALL |
| 108 | QALLLRTPY |
| 109 | EEQCLSAF |
| 110 | ETSEKRPF |
| 111 | DELVRHHN |

TABLE 12
WT1 HLA-B*27:05 Epitope Peptides

| SEQ ID NO. | Sequence |
|---|---|
| 112 | RRVPGVAPTL |
| 113 | RRFSRSDQLK |
| 114 | CRWPSCQKKF |
| 115 | LRTPYSSDNL |
| 116 | RRFSRSDQL |
| 117 | KRYFKLSHL |
| 118 | RRHTGVKPF |
| 119 | FRGIQDVRR |
| 120 | CRWPSCQKK |
| 121 | ARSDELVRH |

TABLE 13
WT1 HLA-B*35:01 Epitope Peptides

| SEQ ID NO. | Sequence |
|---|---|
| 122 | PPGASAYGSL |
| 123 | PPPPPPPHSF |
| 124 | PPPPPPHSFI |
| 125 | TPYSSDNLY |
| 126 | QPAIRNQGY |
| 127 | DPMGQQGSL |
| 128 | TPILCGAQY |
| 129 | TPSHHAAQF |
| 130 | PPPPPPHSF |
| 131 | YPGCNKRYF |

TABLE 14
WT1 HLA-B*58:02 Epitope Peptides

| SEQ ID NO. | Sequence |
|---|---|
| 132 | ASETSEKRPF |
| 133 | QASSGQARMF |
| 134 | RTPYSSDNLY |
| 135 | DSCTGSQALL |
| 136 | ASSGQARMF |
| 137 | RVPGVAPTL |
| 138 | TSQLECMTW |
| 139 | HTHGVFRGI |
| 140 | RTPYSSDNL |
| 141 | RSDELVRHH |

TABLE 15
WT1 HLA-DRB1*0101 Epitope Peptides

| SEQ ID NO. | Sequence |
|---|---|
| 142 | ASAYGSLGGPAPPPA |
| 143 | GSDVRDLNALLPAVP |
| 144 | IQDVRRVPGVAPTLV |
| 145 | VRDLNALLPAVPSLG |
| 146 | GATLKGVAAGSSSSV |
| 147 | TVHFSGQFTGTAGAC |
| 148 | VRRVPGVAPTLVRSA |
| 149 | NKRYFKLSHLQMHSR |
| 150 | LPAVPSLGGGGGCAL |
| 151 | RDLNALLPAVPSLGG |

TABLE 16
WT1 HLA-DRB1*0301 Epitope Peptides

| SEQ ID NO. | Sequence |
|---|---|
| 152 | YSTVTFDGTPSYGHT |
| 153 | MGSDVRDLNALLPAV |
| 154 | YQCDFKDCERRFSRS |
| 155 | VPSLGGGGGCALPVS |
| 156 | VLDFAPPGASAYGSL |
| 157 | LYQMTSQLECMTWNQ |
| 158 | PTLVRSASETSEKRP |
| 159 | HHNMHQRNMTKLQLA |

TABLE 17
WT1 HLA-DRB1*0401 (DR4Dw4) Epitope Peptides

| SEQ ID NO. | Sequence |
|---|---|
| 160 | NKRYFKLSHLQMHSR |
| 161 | TVHFSGQFTGTAGAC |
| 162 | ARMFPNAPYLPSCLE |
| 163 | NQGYSTVTFDGTPSY |
| 164 | TPSYGHTPSHHAAQF |
| 165 | NHSFKHEDPMGQQGS |
| 166 | RTPYSSDNLYQMTSQ |
| 167 | SVKWTEGQSNHSTGY |
| 168 | STGYESDNHTTPILC |
| 169 | KRPFMCAYPGCNKRY |

TABLE 18
WT1 HLA-DRB1*0701 Epitope Peptides

| SEQ ID NO. | Sequence |
|---|---|
| 170 | TPSYGHTPSHHAAQF |
| 171 | TVTFDGTPSYGHTPS |
| 172 | LSAFTVHFSGQFTGT |
| 173 | TPTDSCTGSQALLLR |
| 174 | LKGVAAGSSSSVKWT |
| 175 | TVHFSGQFTGTAGAC |
| 176 | YSTVTFDGTPSYGHT |
| 177 | CGAQYRIHTHGVFRG |
| 178 | HGVFRGIQDVRRVPG |
| 179 | APTLVRSASETSEKR |

TABLE 19
WT1 HLA-DRB1*1101 Epitope Peptides

| SEQ ID NO. | Sequence |
|---|---|
| 180 | FRGIQDVRRVPGVAP |
| 181 | NKRYFKLSHLQMHSR |
| 182 | QCDFKDCERRFSRSD |
| 183 | STGYESDNHTTPILC |
| 184 | SCRWPSCQKKFARSD |
| 185 | AAQWAPVLDFAPPGA |
| 186 | ASAYGSLGGPAPPPA |
| 187 | PGVAPTLVRSASETS |
| 188 | QMNLGATLKGVAAGS |

TABLE 20
WT1 HLA-DRB1*1501 (DR2b) Epitope Peptides

| SEQ ID NO. | Sequence |
|---|---|
| 189 | WAPVLDFAPPGASAY |
| 190 | RPFMCAYPGCNKRYF |
| 191 | GSDVRDLNALLPAVP |
| 192 | NALLPAVPSLGGGGG |
| 193 | PPGASAYGSLGGPAP |
| 194 | EQCLSAFTVHFSGQF |
| 195 | TAGACRYGPFGPPPP |
| 196 | PSCLESQPAIRNQGY |
| 197 | WNQMNLGATLKGVAA |
| 198 | IQDVRRVPGVAPTLV |

PRAME Antigenic Peptides

In some embodiments, the TVM composition includes PRAME specific T-cells. PRAME specific T-cells can be generated as described below using one or more antigenic peptides to PRAME. In some embodiments, the PRAME specific T-cells are generated using one or more antigenic peptides to PRAME, or a modified or heteroclitic peptide derived from a PRAME peptide. In some embodiments, PRAME specific T-cells are generated using a PRAME antigen library comprising a pool of peptides (for example 15mers) containing amino acid overlap (for example 11 amino acids of overlap) between each Sequence formed by scanning the protein amino acid sequence SEQ. ID. No. 199 (UniProt KB—P78395) for human melanoma antigen preferentially expressed in tumors (PRAME):

MERRRLWGSIQSRYISMSVWTSPRRLVELAGQSLLKDEALAIAALELLP
RELFPPLFMAAFDGRHSQTLKAMVQAWPFTCLPLGVLMKGQHLHLETFK
AVLDGLDVLLAQEVRPRRWKLQVLDLRKNSHQDFWTVWSGNRASLYSFP
EPEAAQPMTKKRKVDGLSTEAEQPFIPVEVLVDLFLKEGACDELFSYLI
EKVKRKKNVLRLCCKKLKIFAMPMQDIKMILKMVQLDSIEDLEVTCTWK
LPTLAKFSPYLGQMINLRRLLLSHIHASSYISPEKEEQYIAQFTSQFLS
LQCLQALYVDSLFFLRGRLDQLLRHVMNPLETLSITNCRLSEGDVMHLS
QSPSVSQLSVLSLSGVMLTDVSPEPLQALLERASATLQDLVFDECGITD
DQLLALLPSLSHCSQLTTLSFYGNSISISALQSLLQHLIGLSNLTHVLY
PVPLESYEDIHGTLHLERLAYLHARLRELLCELGRPSMVWLSANPCPHC
GDRTFYDPEPILCPCFMPN

Overlapping antigenic libraries are commercially available, for example, from JPT (Product code: PM-OIP4 PepMix Human (Prame/OIP4)). In some embodiments, the PRAME specific T-cells are generated using a commercially available overlapping antigenic library made up of PRAME peptides.

In some embodiments, the PRAME specific T-cells are generated using one or more antigenic peptides to PRAME, or a modified or heteroclitic peptide derived from a PRAME peptide. In some embodiments, the PRAME specific T-cells are generated with peptides that recognize class I MHC molecules. In some embodiments, the PRAME specific T-cells are generated with peptides that recognize class II MHC molecules. In some embodiments, the PRAME specific T-cells are generated with peptides that recognize both class I and class II MHC molecules.

In some embodiments, the PRAME peptides used to prime and expand a T-cell subpopulation includes specifically selected HLA-restricted peptides generated by determining the HLA profile of the donor source, and including peptides derived from PRAME that best match the donor's HLA. In some embodiments, the PRAME peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting PRAME derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 21-27, the HLA-B peptides are selected from the peptides of Tables 28-34, and the HLA-DR peptides are selected from the peptides of Tables 35-40. For example, if the donor cell source has an HLA profile that is HLA-A*01/*02:01; HLA-B*15:01/*18; and HLA-DRB1*0101/*0301, then the PRAMS peptides used to prime and expand the PRAMS specific T-cell subpopulation are restricted to the specific HLA profile, and may include the peptides identified in Table 21 (Seq. ID. Nos. 200-209) for HLA-A*01; Table 22 (Seq. ID. No. 210-219) for HLA-A*02:01; Table 30 (Seq. ID. No. 289-298) for HLA-B*15:01; Table 31 (Seq. ID. No. 299-308) for HLA-B*18; Table 35 (Seq. ID. No. 339-348) for HLA-DRB1*0101; and Table 36 (Seq. ID. No. 349-358) for HLA-DRB1*0301. In some embodiments, the mastermix of peptides includes both an overlapping peptide library and specifically selected HLA-restricted peptides generated by determining the HLA profile of the donor source.

In some embodiments, the PRAME HLA-restricted epitopes are specific to at least both of the donor's HLA-A alleles, at least both of the donor's HLA-B alleles, and at least both of the donor's HLA-DR alleles. In some embodiments, the PRAMS HLA-A alleles are selected from a group comprising HLA-A*01, HLA-A*02:01, HLA-A*03, HLA-A*11:01, HLA-A*24:02, HLA-A*26, or HLA-A*68:01, and the corresponding PRAME HLA-restricted peptides are selected for: HLA-A*01 from Table 21; HLA-A*02:01 from Table 22; HLA-A*03 from Table 23; HLA-A*11:01 from Table 24; HLA-A*24:02 from Table 25; HLA-A*26 from Table 26; or HLA-A*68:01 from Table 27; or any combination thereof. In some embodiments, the PRAMS HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding PRAME HLA-restricted peptides are selected for: HLA-B*07:02 from Table 28; HLA-B*08 from Table 29; HLA-B*15:01 (B62) from Table 30; HLA-B*18 from Table 31; HLA-B*27:05 from Table 32; HLA-B*35:01 from Table 33, or HLA-B*58:02 from Table 34; or any combination thereof. In some embodiments, the PRAME HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding PRAME HLA-restricted peptides are selected for: HLA-DRB1*0101 from Table 35; HLA-DRB1*0301 (DR17) from Table 36; HLA-DRB1*0401 (DR4Dw4) from Table 37; HLA-DRB1*0701 from Table 38; HLA-DRB1*1101 from Table 39; or HLA-DRB1*1501 (DR2b) from Table 40; or any combination thereof.

TABLE 21
PRAME HLA-A*01 Epitope Peptides

| SEQ ID NO. | Sequence |
|---|---|
| 200 | LTDVSPEPLQA |
| 201 | ITDDQLLALLP |
| 202 | HGTLHLERLAY |
| 203 | GTLHLERLAY |
| 204 | CSQLTTLSFY |
| 205 | LSLQCLQALY |
| 206 | PTLAKFSPY |
| 207 | LSNLTHVLY |
| 208 | WSGNRASLY |
| 209 | LSHIHASSY |

TABLE 22
PRAME HLA-A*02:01 Epitope Peptides

| SEQ ID NO. | Sequence |
|---|---|
| 210 | ALLERASATL |
| 211 | ALAIAALELL |
| 212 | SLSGVMLTDV |
| 213 | ALYVDSLFFL |
| 214 | QLLALLPSL |
| 215 | SLLQHLIGL |
| 216 | RLRELLCEL |
| 217 | YLHARLREL |
| 218 | ALAIAALEL |
| 219 | FLRGRLDQL |

TABLE 23
PRAME HLA-A*03 Epitope Peptides

| SEQ ID NO. | Sequence |
|---|---|
| 220 | HLIGLSNLTH |
| 221 | RLWGSIQSRY |
| 222 | KVKRKKNVLR |
| 223 | VLYPVPLESY |
| 224 | CLPLGVLMK |
| 225 | ELAGQSLLK |
| 226 | KLQVLDLRK |
| 227 | RLSEGDVMH |
| 228 | YLIEKVKRK |
| 229 | NVLRLCCKK |

TABLE 24
PRAME HLA-A*11:01 Epitope Peptides

| SEQ ID NO. | Sequence |
|---|---|
| 230 | KVKRKKNVLR |
| 231 | PMQDIKMILK |
| 232 | CTWKLPTLAK |
| 233 | AIAALELLPR |
| 234 | AVLDGLDVLL |
| 235 | FSYLIEKVKR |
| 236 | ELAGQSLLK |
| 237 | EVLVDLFLK |
| 238 | ASSYISPEK |
| 239 | ELFSYLIEK |

TABLE 25
PRAME HLA-A*24:02 Epitope Peptides

| SEQ ID NO. | Sequence |
|---|---|
| 240 | QYIAQFTSQF |
| 241 | AYLHARLREL |
| 242 | LFPPLFMAAF |
| 243 | KFSPYLGQMI |
| 244 | FFLRGRLDQL |
| 245 | VSPEPLQALL |
| 246 | SYEDIHGTL |
| 247 | PYLGQMINL |
| 248 | LYVDSLFFL |
| 249 | TFYDPEPIL |

TABLE 26
PRAME HLA-A*26 Epitope Peptides

| SEQ ID NO. | Sequence |
|---|---|
| 250 | ETFKAVLDGL |
| 251 | DVSPEPLQAL |
| 252 | ETLSITNCRL |
| 253 | EGACDELFSY |
| 254 | EKEEQYIAQF |
| 255 | SVSQLSVLSL |
| 256 | EVRPRRWKL |
| 257 | ETFKAVLDG |
| 258 | EVLVDLFLK |

TABLE 27
PRAME HLA-A*68:01 Epitope Peptides

| SEQ ID NO. | Sequence |
|---|---|
| 259 | DVLLAQEVRPR |
| 260 | EAAQPMTKKR |
| 261 | KVKRKKNVLR |
| 262 | EAAQPMTKK |
| 263 | EVLVDLFLK |
| 264 | ELFSYLIEK |
| 265 | ETLSITNCR |
| 266 | DVLLAQEVR |
| 267 | DSLFFLRGR |
| 268 | IAALELLPR |

TABLE 28
PRAME HLA-B*07:02 Epitope Peptides

| SEQ ID NO. | Sequence |
|---|---|
| 269 | RPRRWKLQVL |
| 270 | SPSVSQLSVL |
| 271 | LPSLSHCSQL |
| 272 | MPMQDIKMIL |
| 273 | LPRELFPPL |
| 274 | QPFIPVEVL |
| 275 | IPVEVLVDL |
| 276 | SPEPLQALL |
| 277 | RPRRWKLQV |
| 278 | RPSMVWLSA |

TABLE 29
PRAME HLA-B*08 Epitope Peptides

| SEQ ID NO. | Sequence |
|---|---|
| 279 | TKKRKVDGL |
| 280 | FLRGRLDQL |
| 281 | KVKRKKNVL |
| 282 | EVRPRRWKL |
| 283 | PRRWKLQVL |
| 284 | VLRLCCKKL |
| 285 | YLHARLREL |
| 286 | RLRELLCEL |
| 287 | HARLRELL |
| 288 | VKRKKNVL |

TABLE 30
PRAME HLA-B*15:01 (B62) Epitope Peptides

| SEQ ID NO. | Sequence |
|---|---|
| 289 | VLYPVPLESY |
| 290 | RLWGSIQSRY |
| 291 | GLSNLTHVLY |
| 292 | RLCCKKLKIF |
| 293 | LLSHIHASSY |
| 294 | TLHLERLAY |
| 295 | GQHLHLETF |
| 296 | SLQCLQALY |
| 297 | ALYVDSLFF |
| 298 | SQLTTLSFY |

TABLE 31
PRAME HLA-B*18 Epitope Peptides

| SEQ ID NO. | Sequence |
|---|---|
| 299 | DEALAIAAL |
| 300 | LELLPRELF |
| 301 | KEGACDELF |
| 302 | PEPILCPCF |
| 303 | VEVLVDLF |
| 304 | EEQYIAQF |
| 305 | LELLPREL |
| 306 | RELFPPLF |
| 307 | SEGDVMHL |
| 308 | LERASATL |

TABLE 32
PRAME HLA-B*27:05 Epitope Peptides

| SEQ ID NO. | Sequence |
|---|---|
| 309 | RRLWGSIQSR |
| 310 | RRWKLQVLDL |
| 311 | ERLAYLHARL |
| 312 | ARLRELLCEL |
| 313 | KRKKNVLRL |
| 314 | RRLLLSHIH |
| 315 | GRLDQLLRH |
| 316 | PRRWKLQVL |
| 317 | LRLCCKKLK |
| 318 | ERLAYLHAR |

TABLE 33
PRAME HLA-B*35:01 Epitope Peptides

| SEQ ID NO. | Sequence |
|---|---|
| 319 | RPRRWKLQVL |
| 320 | SPSVSQLSVL |
| 321 | LPRELFPPLF |
| 322 | IPVEVLVDLF |
| 323 | MPMQDIKMIL |
| 324 | LPTLAKFSPY |
| 325 | IPVEVLVDL |
| 326 | LPRELFPPL |
| 327 | SPEPLQALL |
| 328 | QPFIPVEVL |

TABLE 34
PRAME HLA-B*58:02 Epitope Peptides

| SEQ ID NO. | Sequence |
|---|---|
| 329 | MSVWTSPRRL |
| 330 | AALELLPREL |
| 331 | KAVLDGLDVL |
| 332 | LAQEVRPRRW |
| 333 | ESYEDIHGTL |
| 334 | LSLQCLQALY |
| 335 | VSPEPLQALL |
| 336 | LSHCSQLTTL |
| 337 | KAMVQAWPF |
| 338 | KVKRKKNVL |

TABLE 35
PRAME HLA-DRB1*0101 Epitope Peptides

| SEQ ID NO. | Sequence |
|---|---|
| 339 | PRRLVELAGQSLLKD |
| 340 | LDGLDVLLAQEVRPR |
| 341 | FLSLQCLQALYVDSL |
| 342 | RHVMNPLETLSITNC |
| 343 | QLSVLSLSGVMLTDV |
| 344 | RRLWGSIQSRYISMS |
| 345 | EEQYIAQFTSQFLSL |
| 346 | DDQLLALLPSLSHCS |
| 347 | GVMLTDVSPEPLQAL |
| 348 | GQSLLKDEALAIAAL |

TABLE 36
PRAME HLA-DRB1*0301 (DR17) Epitope Peptides

| SEQ ID NO. | Sequence |
|---|---|
| 349 | ECGITDDQLLALLPS |
| 350 | LKMVQLDSIEDLEVT |
| 351 | LQALYVDSLFFLRGR |
| 352 | RRLVELAGQSLLKDE |
| 353 | IAALELLPRELFPPL |
| 354 | LGQMINLRRLLLSHI |
| 355 | FWTVWSGNRASLYSF |
| 356 | SSYISPEKEEQYIAQ |
| 357 | LAYLHARLRELLCEL |
| 358 | GQSLLKDEALAIAAL |

TABLE 37
PRAME HLA-DRB1*0401 (DR4Dw4)
Epitope Peptides

| SEQ ID NO. | Sequence |
|---|---|
| 359 | RRLWGSIQSRYISMS |
| 360 | RRLVELAGQSLLKDE |
| 361 | SYLIEKVKRKKNVLR |
| 362 | LGQMINLRRLLLSHI |
| 363 | EQYIAQFTSQFLSLQ |
| 364 | RGRLDQLLRHVMNPL |
| 365 | RHVMNPLETLSITNC |
| 366 | EGDVMHLSQSPSVSQ |
| 367 | LALLPSLSHCSQLTT |
| 368 | SISISALQSLLQHLI |

TABLE 38
PRAME HLA-DRB1*0701 Epitope Peptides

| SEQ ID NO. | Sequence |
|---|---|
| 369 | RRLWGSIQSRYISMS |
| 370 | IEDLEVTCTWKLPTL |
| 371 | GDVMHLSQSPSVSQL |
| 372 | MVQLDSIEDLEVTCT |
| 373 | LSFYGNSISISALQS |
| 374 | MAAFDGRHSQTLKAM |
| 375 | EEQYIAQFTSQFLSL |
| 376 | EQYIAQFTSQFLSLQ |
| 377 | RHVMNPLETLSITNC |
| 378 | LQALLERASATLQDL |

TABLE 39
PRAME HLA-DRB1*1101 Epitope Peptides

| SEQ ID NO. | Sequence |
|---|---|
| 379 | TWKLPTLAKFSPYLG |
| 380 | QSRYISMSVWTSPRR |
| 381 | AQPMTKKRKVDGLST |
| 382 | TSQFLSLQCLQALYV |
| 383 | MSVWTSPRRLVELAG |
| 384 | IAALELLPRELFPPL |
| 385 | CLPLGVLMKGQHLHL |
| 386 | QDFWTVWSGNRASLY |
| 387 | SYLIEKVKRKKNVLR |
| 388 | MQDIKMILKMVQLDS |

TABLE 40
PRAME HLA-DRB1*1501 (DR2b) Epitope Peptides

| SEQ ID NO. | Sequence |
|---|---|
| 389 | HLHLETFKAVLDGLD |
| 390 | PVPLESYEDIHGTLH |
| 391 | YISMSVWTSPRRLVE |
| 392 | PLFMAAFDGRHSQTL |
| 393 | LPTLAKFSPYLGQMI |
| 394 | EQYIAQFTSQFLSLQ |
| 395 | LTTLSFYGNSISISA |
| 396 | LAKFSPYLGQMINLR |
| 397 | MERRRLWGSIQSRYI |
| 398 | GSIQSRYISMSVWTS |

Survivin Antigenic Peptides

In some embodiments, the TVM composition includes survivin specific T-cells. survivin specific T-cells can be generated as described below using one or more antigenic peptides to Survivin. In some embodiments, the Survivin specific T-cells are generated using one or more antigenic peptides to Survivin, or a modified or heteroclitic peptide derived from a survivin peptide. In some embodiments, survivin specific T-cells are generated using a survivin antigen library comprising a pool of peptides (for example 15mers) containing amino acid overlap (for example 11 amino acids of overlap) between each sequence formed by scanning the protein amino acid sequence SEQ. ID. No. 399 (UniProt KB—O15392) for human baculoviral inhibitor of apoptosis repeat-containing 5 (Survivin):

MGAPTLPPAWQPFLKDHRISTFKNWPFLEGCACTPERMAEAGFIHCPTEN
EPDLQCFFCFKELEGWEPDDDPIEEHKKHSSGCAFLSVKKQFEELTLGEF
LKLDRERAKNKIAKETNNKKKEFEETAKKVRRAIEQLAAMD

Overlapping antigenic libraries are commercially available, for example, from JPT, for example, from JPT (Product Code: PM-Survivin (PepMix Human (Survivin)). In some embodiments, the survivin specific T-cells are generated using a commercially available overlapping antigenic library made up of survivin peptides.

In some embodiments, the survivin specific T-cells are generated using one or more antigenic peptides to survivin, or a modified or heteroclitic peptide derived from a Survivin peptide,

In some embodiments, the survivin specific T-cells are generated with peptides that recognize class I MHC molecules. In some embodiments, the survivin specific T-cells are generated with peptides that recognize class II MHC molecules. In some embodiments, the Survivin specific T-cells are generated with peptides that recognize both class I and class II MHC molecules.

In some embodiments, the survivin peptides used to prime and expand a T-cell subpopulation includes specifically selected HLA-restricted peptides generated by determining the HLA profile of the donor source, and including peptides derived from survivin that best match the donor's HLA. In some embodiments, the survivin peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting survivin derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 41-47, the HLA-B peptides are selected from the peptides of Tables 48-54, and the HLA-DR peptides are selected from the peptides of Tables 55-60. For example, if the donor cell source has an HLA profile that is HLA-A*01/*02:01; HLA-B*15:01/*18; and HLA-DRB1*0101/*0301, then the survivin peptides used to prime and expand the survivin specific T-cell subpopulation are restricted to the specific HLA profile, and may include the peptides identified in Table 41 (Seq. ID. Nos. 400-409) for HLA-A*01; Table 42 (Seq. ID. No. 410-419) for HLA-A*02:01; Table 50 (Seq. ID. No. 490-500) for HLA-B*15:01; Table 51 (Seq. ID. No. 501-510) for HLA-B*18; Table 55 (Seq. ID. No. 541-550) for HLA-DRB1*0101; and Table 56 (Seq. ID. No. 551-560) for HLA-DRB1*0301. In some embodiments, the mastermix of peptides includes both an overlapping peptide library and specifically selected HLA-restricted peptides generated by determining the HLA profile of the donor source.

In some embodiments, the survivin HLA-restricted epitopes are specific to at least both of the donor's HLA-A alleles, at least both of the donor's HLA-B alleles, and at least both of the donor's HLA-DR alleles. In some embodiments, the survivin HLA-A alleles are selected from a group comprising HLA-A*01, HLA-A*02:01, HLA-A*03, HLA-A*11:01, HLA-A*24:02, HLA-A*26, or HLA-A*68:01, and the corresponding survivin HLA-restricted peptides are selected for: HLA-A*01 from Table 41; HLA-A*02:01 from Table 42; HLA-A*03 from Table 43; HLA-A*11:01 from Table 44; HLA-A*24:02 from Table 45; HLA-A*26 from Table 46; or HLA-A*68:01 from Table 47; or any combination thereof. In some embodiments, the survivin HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding survivin HLA-restricted peptides are selected for: HLA-B*07:02 from Table 48; HLA-B*08 from Table 49; HLA-B*15:01 (B62) from Table 50; HLA-B*18 from Table 51; HLA-B*27:05 from Table 52; HLA-B*35:01 from Table 53, or HLA-B*58:02 from Table 54; or any combination thereof. In some embodiments, the survivin HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding survivin HLA-restricted peptides are selected for: HLA-DRB1*0101 from Table 55; HLA-DRB1*0301 (DR17) from Table 56; HLA-DRB1*0401 (DR4Dw4) from Table 57; HLA-DRB1*0701 from Table 58; HLA-DRB1*1101 from Table 59; or HLA-DRB1*1501 (DR2b) from Table 60; or any combination thereof.

TABLE 41
Survivin HLA-A*01 Epitope Peptides

| SEQ ID NO. | Sequence |
|---|---|
| 400 | PTENEPDLAQC |
| 401 | KLDRERAKNKI |
| 402 | LKDHRISTFKN |
| 403 | STFKNWPFLEG |
| 404 | DDDPIEEHKKH |
| 405 | PTENEPDLAQ |
| 406 | PTENEPDLA |
| 407 | LTLGEFLKL |
| 408 | LGEFLKLDR |
| 409 | KLDRERAKN |

TABLE 42
Survivin HLA-A*02:01 Epitope Peptides

| SEQ ID NO. | Sequence |
|---|---|
| 410 | TLPPAWQPFL |
| 411 | ELTLGEFLKL |
| 412 | FLKDHRISTF |
| 413 | LTLGEFLKL |
| 414 | KVRRAIEQL |
| 415 | RAIEQLAAM |
| 416 | STFKNWPFL |
| 417 | FLKDHRIST |
| 418 | SVKKQFEEL |
| 419 | TLGEFLKLD |

TABLE 43
Survivin HLA-A*03 Epitope Peptides

| SEQ ID NO. | Sequence |
|---|---|
| 420 | KLDRERAKNK |
| 421 | FLKDHRISTF |
| 422 | FLKLDRERAK |
| 423 | KIAKETNNKK |
| 424 | DLAQCFFCFK |
| 425 | ELTLGEFLK |
| 426 | KIAKETNNK |
| 427 | KVRRAIEQL |
| 428 | SGCAFLSVK |
| 429 | KLDRERAKN |

TABLE 44
Survivin HLA-A*11:01 Epitope Peptides

| SEQ ID NO. | Sequence |
|---|---|
| 430 | SSGCAFLSVK |
| 431 | DLAQCFFCFK |
| 432 | SGCAFLSVKK |
| 433 | TLGEFLKLDR |
| 434 | STFKNWPFLE |
| 435 | KLDRERAKNK |
| 436 | KIAKETNNKK |
| 437 | SSGCAFLSV |
| 438 | GCAFLSVKK |
| 439 | ELTLGEFLK |

TABLE 45
Survivin HLA-A24:02 Epitope Peptides

| SEQ ID NO. | Sequence |
|---|---|
| 440 | QFEELTLGEF |
| 441 | TLPPAWQPFL |
| 442 | PDLAQCFFCF |
| 443 | PTLPPAWQPF |
| 444 | NEPDLAQCFF |
| 445 | LSVKKQFEEL |
| 446 | ELTLGEFLKL |
| 447 | AFLSVKKQF |
| 448 | LTLGEFLKL |
| 449 | TLPPAWQPF |

TABLE 46
Survivin HLA-A*26 Epitope Peptides

| SEQ ID NO. | Sequence |
|---|---|
| 450 | ELTLGEFLKL |
| 451 | ENEPDLAQCF |
| 452 | ETAKKVRRAI |
| 453 | ETNNKKKEFE |
| 454 | ETNNKKKEF |
| 455 | ETAKKVRRA |
| 456 | KVRRAIEQL |
| 457 | STFKNWPFL |
| 458 | EELTLGEFL |
| 459 | SVKKQFEEL |

TABLE 47
Survivin HLA-A*68:01 Epitope Peptides

| SEQ ID NO. | Sequence |
|---|---|
| 460 | LTLGEFLKLDR |
| 461 | PAWQPFLKDHR |
| 462 | SSGCAFLSVKK |
| 463 | EFEETAKKVRR |
| 464 | ETAKKVRRAIE |
| 465 | DLAQCFFCFK |
| 466 | EETAKKVRR |
| 467 | ERAKNKIAK |
| 468 | ETAKKVRRA |
| 469 | ELTLGEFLK |

TABLE 48
Survivin HLA-B*07:02 Epitope Peptides

| SEQ ID NO. | Sequence |
|---|---|
| 470 | LPPAWQPFL |
| 471 | CPTENEPDL |
| 472 | EPDLAQCFF |
| 473 | APTLPPAWQ |
| 474 | QPFLKDHRI |
| 475 | KHSSGCAFL |
| 476 | LTLGEFLKL |
| 477 | WPFLEGCACT |
| 478 | TPERMAEAGF |
| 479 | CPTENEPDLA |

TABLE 49
Survivin HLA-B*08 Epitope Peptides

| SEQ ID NO. | Sequence |
|---|---|
| 480 | RAKNKIAKE |
| 481 | QPFLKDHRI |
| 482 | SVKKQFEEL |
| 483 | NNKKKEFEE |
| 484 | TAKKVRRAI |
| 485 | AKKVRRAI |
| 486 | FLSVKKQF |
| 487 | RAKNKIAK |
| 488 | RERAKNKI |
| 489 | VKKQFEEL |

Table 50
Survivin HLA-B*15:01 (B62) Epitope Peptides

| SEQ ID NO. | Sequence |
|---|---|
| 490 | FLKDHRISTF |
| 491 | KQFEELTLGE |
| 492 | TLPPAWQPFL |
| 493 | ELEGWEPDDD |
| 495 | TLGEFLKLDR |
| 496 | TLPPAWQPF |
| 497 | DLAQCFFCF |
| 498 | KQFEELTLG |
| 499 | FLKDHRIST |
| 500 | KVRRAIEQL |

TABLE 51
Survivin HLA-B*18 Epitope Peptides

| SEQ ID NO. | Sequence |
|---|---|
| 501 | EELTLGEFL |
| 502 | FEELTLGEF |
| 503 | NEPDLAQCF |
| 504 | PERMAEAGF |
| 505 | DLAQCFFCF |
| 506 | KELEGWEPD |
| 507 | EELTLGEF |
| 508 | EEHKKHSS |
| 509 | KELEGWEP |
| 510 | KQFEELTL |

TABLE 52
Survivin HLA-B*27:05 Epitope Peptides

| SEQ ID NO. | Sequence |
|---|---|
| 511 | RRAIEQLAAM |
| 512 | GEFLKLDRER |
| 513 | ERMAEAGFIH |
| 514 | ERAKNKIAKE |
| 515 | KIAKETNNKK |
| 516 | ERAKNKIAK |
| 517 | DRERAKNKI |
| 518 | KEFEETAKK |
| 519 | ERMAEAGFI |
| 520 | GCAFLSVKK |

TABLE 53
Survivin HLA-B*35:01 Epitope Peptides

| SEQ ID NO. | Sequence |
|---|---|
| 521 | TPERMAEAGF |
| 522 | LPPAWQPFLK |
| 523 | EPDDDPIEEH |
| 524 | LSVKKQFEEL |
| 525 | LPPAWQPFL |
| 526 | CPTENEPDL |
| 527 | EPDLAQCFF |
| 528 | QPFLKDHRI |
| 529 | TPERMAEAG |
| 530 | EPDDDPIEE |

TABLE 54
Survivin HLA-B*58:02 Epitope Peptides

| SEQ ID NO. | Sequence |
|---|---|
| 531 | ETAKKVRRAI |
| 532 | PTLPPAWQPF |
| 533 | ISTFKNWPFL |
| 534 | LSVKKQFEEL |
| 535 | TAKKVRRAI |
| 536 | RAIEQLAAM |
| 537 | KVRRAIEQL |
| 538 | ISTFKNWPF |
| 539 | LTLGEFLKL |
| 540 | GAPTLPPAW |

TABLE 55
Survivin HLA-DRB1*0101 Epitope Peptides

| SEQ ID NO. | Sequence |
|---|---|
| 541 | FFCFKELEGWEPDDD |
| 542 | FKNWPFLEGCACTPE |
| 543 | LGEFLKLDRERAKNK |
| 544 | NWPFLEGCACTPERM |
| 545 | KKQFEELTLGEFLKL |
| 546 | CTPERMAEAGFIHCP |
| 547 | FEELTLGEFLKLDRE |
| 548 | MGAPTLPPAWQPFLK |
| 549 | KKKEFEETAKKVRRA |
| 550 | AKKVRRAIEQLAAMD |

TABLE 56
Survivin HLA-DRB1*0301 (DR17) Epitope Peptides

| SEQ ID NO. | Sequence |
|---|---|
| 551 | GEFLKLDRERAKNKI |
| 552 | WQPFLKDHRISTFKN |
| 553 | APTLPPAWQPFLKDH |
| 554 | DHRISTFKNWPFLEG |
| 555 | FEELTLGEFLKLDRE |
| 556 | PIENEPDLAQCFFCF |
| 557 | QPFLKDHRISTFKNW |
| 558 | GCAFLSVKKQFEELT |
| 559 | ELTLGEFLKLDRERA |
| 560 | AKKVRRAIEQLAAMD |

TABLE 57
Survivin HLA-DRB1*0401 (DR4Dw4) Epitope Peptides

| SEQ ID NO. | Sequence |
|---|---|
| 561 | WQPFLKDHRISTFKN |
| 562 | LGEFLKLDRERAKNK |
| 563 | APTLPPAWQPFLKDH |
| 564 | KNKIAKETNNKKKEF |
| 565 | DHRISTFKNWPFLEG |
| 566 | GEFLKLDRERAKNKI |
| 567 | FLKLDRERAKNKIAK |
| 568 | AKKVRRAIEQLAAMD |
| 569 | FLKDHRISTFKNWPF |
| 570 | RMAEAGFIHCPTENE |

TABLE 58
Survivin HLA-DRB1*0701 Epitope Peptides

| SEQ ID NO. | Sequence |
|---|---|
| 571 | AKKVRRAIEQLAAMD |
| 572 | APTLPPAWQPFLKDH |
| 573 | DHRISTFKNWPFLEG |
| 574 | LEGCACTPERMAEAG |
| 575 | EAGFIHCPTENEPDL |
| 576 | KKEFEETAKKVRRAI |
| 577 | AQCFFCFKELEGWEP |
| 578 | QCFFCFKELEGWEPD |
| 579 | LEGWEPDDDPIEEHK |
| 580 | KKQFEELTLGEFLKL |

TABLE 59
Survivin HLA-DRB1*1101 Epitope Peptides

| SEQ ID NO. | Sequence |
|---|---|
| 581 | LGEFLKLDRERAKNK |
| 582 | GCAFLSVKKQFEELT |
| 583 | FFCFKELEGWEPDDD |
| 584 | DDPIEEHKKHSSGCA |
| 585 | KKEFEETAKKVRRAI |
| 586 | PPAWQPFLKDHRIST |
| 587 | WQPFLKDHRISTFKN |
| 588 | AWQPFLKDHRISTFK |
| 589 | AQCFFCFKELEGWEP |
| 590 | ISTFKNWPFLEGCAC |

TABLE 60
Survivin HLA-DRB1*1501 (DR2b) Epitope Peptides

| SEQ ID NO. | Sequence |
|---|---|
| 591 | LGEFLKLDRERAKNK |
| 592 | GCAFLSVKKQFEELT |
| 593 | FFCFKELEGWEPDDD |
| 594 | DDPIEEHKKHSSGCA |
| 595 | KKEFEETAKKVRRAI |
| 596 | PPAWQPFLKDHRIST |
| 597 | WQPFLKDHRISTFKN |
| 598 | AWQPFLKDHRISTFK |
| 599 | AQCFFCFKELEGWEP |
| 600 | ISTFKNWPFLEGCAC |

NY-ESO-1 Antigenic Peptides

In some embodiments, the TVM composition includes NY-ESO-1 (cancer/testis antigen 1) specific T-cells. NY-ESO-1 specific T-cells can be generated as described below using one or more antigenic peptides to NY-ESO-1. In some embodiments, the NY-ESO-1 specific T-cells are generated using one or more antigenic peptides to NY-ESO-1, or a modified or heteroclitic peptide derived from a NY-ESO-1 peptide. In some embodiments, NY-ESO-1 specific T-cells are generated using a NY-ESO-1 antigen library comprising a pool of peptides (for example 15mers) containing amino acid overlap (for example 11 amino acids of overlap) between each sequence formed by scanning the protein amino acid sequence SEQ. ID. No. 601 (UniProt KB—P78358) for NY-ESO-1:

MQAEGRGTGGSTGDADGPGGPGIPDGPGGNAGGPGEAGATGGRGPRGAG
AARASGPGGGAPRGPHGGAASGLNGCCRCGARGPESRLLEFYLAMPFAT
PMEAELARRSLAQDAPPLPVPGVLLKEFTVSGNILTIRLTAADHRQLQL
SISSCLQQLSLLMWITQCFLPVFLAQPPSGQRR.

Overlapping antigenic libraries are commercially available, for example, from JPT, for example, from JPT (Product Code: PM-NYE (PepMix Human (NY-ESO-1)). In some embodiments, the NY-ESO-1 specific T-cells are generated using a commercially available overlapping antigenic library made up of NY-ESO-1 peptides.

In some embodiments, the NY-ESO-1 specific T-cells are generated using one or more antigenic peptides to NY-ESO-1, or a modified or heteroclitic peptide derived from a NY-ESO-1 peptide. In some embodiments, the NY-ESO-1 specific T-cells are generated with peptides that recognize class I MHC molecules. In some embodiments, the NY-ESO-1 specific T-cells are generated with peptides that recognize class II MHC molecules. In some embodiments, the NY-ESO-1 specific T-cells are generated with peptides that recognize both class I and class II MHC molecules.

In some embodiments, the NY-ESO-1 peptides used to prime and expand a T-cell subpopulation includes specifically selected HLA-restricted peptides generated by determining the HLA profile of the donor source, and including peptides derived from NY-ESO-1 that best match the donor's HLA. In some embodiments, the NY-ESO-1 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting NY-ESO-1 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 61-67, the HLA-B peptides are selected from the peptides of Tables 68-74, and the HLA-DR peptides are selected from the peptides of Tables 75-80. For example, if the donor cell source has an HLA profile that is HLA-A*01/*02:01; HLA-B*15:01/*18; and HLA-DRB1*0101/*0301, then the NY-ESO-1 peptides used to prime and expand the NY-ESO-1 specific T-cell subpopulation are restricted to the specific HLA profile, and may include the peptides identified in Table 61 (Seq. ID. Nos. 602-611) for HLA-A*01; Table 62 (Seq. ID. Nos. 612-621) for HLA-A*02:01; Table 70 (Seq. ID. Nos. 692-701) for HLA-B*15:01; Table 71 (Seq. ID. Nos. 702-711) for HLA-B*18; Table 75 (Seq. ID. Nos. 742-751) for HLA-DRB1*0101; and Table 76 (Seq. ID. Nos. 752-761) for HLA-DRB1*0301. In some embodiments, the mastermix of peptides includes both an overlapping peptide library and specifically selected HLA-restricted peptides generated by determining the HLA profile of the donor source.

In some embodiments, the NY-ESO-1 HLA-restricted epitopes are specific to at least both of the donor's HLA-A alleles, at least both of the donor's HLA-B alleles, and at least both of the donor's HLA-DR alleles. In some embodiments, the NY-ESO-1 HLA-A alleles are selected from a group comprising HLA-A*01, HLA-A*02:01, HLA-A*03, HLA-A*11:01, HLA-A*24:02, HLA-A*26, or HLA-A*68:01, and the corresponding NY-ESO-1 HLA-restricted peptides are selected for: HLA-A*01 from Table 61; HLA-A*02:01 from Table 62; HLA-A*03 from Table 63; HLA-A*11:01 from Table 64; HLA-A*24:02 from Table 65; HLA-A*26 from Table 66; or HLA-A*68:01 from Table 67; or any combination thereof. In some embodiments, the NY-ESO-1 HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding NY-ESO-1 HLA-restricted peptides are selected for: HLA-B*07:02 from Table 68; HLA-B*08 from Table 69; HLA-B*15:01 (B62) from Table 70; HLA-B*18 from Table 71; HLA-B*27:05 from Table 72; HLA-B*35:01 from Table 73, or HLA-B*58:02 from Table 74; or any combination thereof. In some embodiments, the NY-ESO-1 HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding NY-ESO-1 HLA-restricted peptides are selected for: HLA-DRB1*0101 from Table 75; HLA-DRB1*0301 (DR17) from Table 76; HLA-DRB1*0401 (DR4Dw4) from Table 77; HLA-DRB1*0701 from Table 78; HLA-DRB1*1101 from Table 79; or HLA-DRB1*1501 (DR2b) from Table 80; or any combination thereof.

TABLE 61
NYESO1 HLA-A*01 Epitope Peptides

| SEQ ID NO. | Sequence |
|---|---|
| 602 | RGPESRLLEFY |
| 603 | AADHRQLQLSI |
| 604 | EAELARRSLAQ |
| 605 | GPESRLLEFY |
| 606 | AQDAPPLPVP |
| 607 | AADHRQLQLS |
| 608 | EAELARRSLA |
| 609 | PESRLLEFY |
| 610 | AQDAPPLPV |
| 611 | AADHRQLQL |

TABLE 62
NYESO1 HLA-A*02:01 Epitope Peptides

| SEQ ID NO. | Sequence |
|---|---|
| 612 | LLMWITQCFL |
| 613 | DAPPLPVPGV |
| 614 | RLLEFYLAMP |
| 615 | FTVSGNILTI |
| 616 | QLQLSISSCL |
| 617 | SLAQDAPPL |
| 618 | SISSCLQQL |
| 619 | RLLEFYLAM |
| 620 | TVSGNILTI |
| 621 | LMWITQCFL |

TABLE 63
NYESO1 HLA-A*03 Epitope Peptides

| SEQ ID NO. | Sequence |
|---|---|
| 622 | PLPVPGVLLK |
| 623 | RLLEFYLAMP |
| 624 | ELARRSLAQD |
| 625 | TIRLTAADHR |
| 626 | RLTAADHRQL |
| 627 | QLSISSCLQQ |
| 628 | FLAQPPSGQR |
| 629 | TIRLTAADH |
| 630 | RLLEFYLAM |
| 631 | ELARRSLAQ |

TABLE 64
NYESO1 HLA-A*11:01 Epitope Peptides

| SEQ ID NO. | Sequence |
|---|---|
| 632 | ATPMEAELAR |
| 633 | PLPVPGVLLK |
| 634 | ASGPGGGAPR |
| 635 | TVSGNILTIR |
| 636 | GVLLKEFTVS |
| 637 | ASGLNGCCR |
| 638 | LPVPGVLLK |
| 639 | VSGNILTIR |
| 640 | FTVSGNILT |
| 641 | SSCLQQLSL |

TABLE 65
NYESO1 HLA-A*24:02 Epitope Peptides

| SEQ ID NO. | Sequence |
|---|---|
| 642 | PFATPMEAEL |
| 643 | PPLPVPGVLL |
| 644 | RGPESRLLEF |
| 645 | FYLAMPFATP |
| 646 | APPLPVPGVL |
| 647 | EFTVSGNIL |
| 648 | PPLPVPGVL |
| 649 | FYLAMPFAT |
| 650 | PLPVPGVLL |
| 651 | SCLQQLSLL |

TABLE 66
NYESO1 HLA-A*26 Epitope Peptides

| SEQ ID NO. | Sequence |
|---|---|
| 652 | PVPGVLLKEF |
| 653 | FTVSGNILTI |
| 654 | LSISSCLQQL |
| 655 | WITQCFLPVF |
| 656 | EFTVSGNIL |
| 657 | ITQCFLPVF |
| 658 | ESRLLEFYL |
| 659 | EAELARRSL |
| 660 | SISSCLQQL |
| 661 | TVSGNILTI |

TABLE 67
NYESO1 HLA-A*68:01 Epitope Peptides

| SEQ ID NO. | Sequence |
|---|---|
| 662 | ATPMEAELARR |
| 663 | FTVSGNILTIR |
| 664 | EAGATGGRGPR |
| 665 | LTIRLTAADHR |
| 666 | RASGPGGGAPR |
| 667 | TVSGNILTIR |
| 668 | ASGPGGGAPR |
| 669 | ATPMEAELAR |
| 670 | VSGNILTIR |
| 671 | PMEAELARR |

TABLE 68
NYESO1 HLA-B*07:02 Epitope Peptides

| SEQ ID NO. | Sequence |
|---|---|
| 672 | APRGPHGGAA |
| 673 | APPLPVPGVL |
| 674 | PPLPVPGVLL |
| 675 | GPHGGAASGL |
| 676 | GPRGAGAARA |
| 677 | APRGPHGGA |
| 678 | IPDGPGGNA |
| 679 | APPLPVPGV |
| 680 | PPLPVPGVL |
| 681 | GPGGPGIPD |

TABLE 69
NYESO1 HLA-B*08 Epitope Peptides

| SEQ ID NO. | Sequence |
|---|---|
| 682 | GPESRLLEF |
| 683 | AADHRQLQL |
| 684 | GARGPESRL |
| 685 | ESRLLEFYL |
| 686 | LLKEFTVSG |
| 687 | SLAQDAPPL |
| 688 | PLPVPGVLL |
| 689 | AELARRSL |
| 690 | LLKEFTVS |
| 691 | PLPVPGVL |

TABLE 70
NYESO1 HLA-B*15:01 (B62) Epitope Peptides

| SEQ ID NO. | Sequence |
|---|---|
| 692 | SLLMWITQCF |
| 693 | PVPGVLLKEF |
| 694 | LLEFYLAMPF |
| 695 | RLLEFYLAMP |
| 696 | VLLKEFTVSG |
| 697 | MQAEGRGTGG |
| 698 | ILTIRLTAAD |
| 699 | RQLQLSISSC |
| 700 | LLMWITQCF |
| 701 | LLKEFTVSG |

TABLE 71
NYESO1 HLA-B*18 Epitope Peptides

| SEQ ID NO. | Sequence |
|---|---|
| 702 | PESRLLEFY |
| 703 | LEFYLAMPF |
| 704 | MEAELARRS |
| 705 | ESRLLEFYL |
| 706 | VPGVLLKEF |
| 707 | ITQCFLPVF |
| 708 | PESRLLEF |
| 709 | AELARRSL |
| 710 | PGVLLKEF |
| 711 | MEAELARR |

TABLE 72
NYESO1 HLA-B*27:05 Epitope Peptides

| SEQ ID NO. | Sequence |
|---|---|
| 712 | SRLLEFYLAM |
| 713 | RGPESRLLEF |
| 714 | RSLAQDAPPL |
| 715 | GPHGGAASGL |
| 716 | RRSLAQDAPP |
| 717 | ARGPESRLL |
| 718 | IRLTAADHR |
| 719 | GARGPESRL |
| 720 | GRGTGGSTG |
| 721 | GATGGRGPR |

TABLE 73
NYESO1 HLA-B*35:01 Epitope Peptides

| SEQ ID NO. | Sequence |
|---|---|
| 722 | PPLPVPGVLL |
| 723 | GPESRLLEFY |
| 724 | GPHGGAASGL |
| 725 | APPLPVPGVL |
| 726 | MPFATPMEAE |
| 727 | PPLPVPGVL |
| 728 | GPESRLLEF |
| 729 | VPGVLLKEF |
| 730 | LQLSISSCL |
| 731 | LPVFLAQPP |

TABLE 74
NYESO1 HLA-B*58:02 Epitope Peptides

| SEQ ID NO. | Sequence |
|---|---|
| 732 | RSLAQDAPPL |
| 733 | GARGPESRLL |
| 734 | FTVSGNILTI |
| 735 | LSISSCLQQL |
| 736 | SSCLQQLSLL |
| 737 | VSGNILTIRL |
| 738 | ISSCLQQLSL |
| 739 | EAELARRSL |
| 740 | LTAADHRQL |
| 741 | ESRLLEFYL |

TABLE 75
NYESO1 HLA-DRB1*0101 Epitope Peptides

| SEQ ID NO. | Sequence |
|---|---|
| 742 | EFYLAMPFATPMEAE |
| 743 | SRLLEFYLAMPFATP |
| 744 | ATPMEAELARRSLAQ |
| 745 | GPGIPDGPGGNAGGP |
| 746 | LEFYLAMPFATPMEA |
| 747 | MPFATPMEAELARRS |
| 748 | LLMWITQCFLPVFLA |
| 749 | TQCFLPVFLAQPPSG |
| 750 | QCFLPVFLAQPPSGQ |
| 751 | YLAMPFATPMEAELA |

TABLE 76
NYESO1 HLA-DRB1*0301 (DR17) Epitope Peptides

| SEQ ID NO. | Sequence |
|---|---|
| 752 | LSLLMWITQCFLPVF |
| 753 | AMPFATPMEAELARR |
| 754 | QLSLLMWITQCFLPV |
| 755 | RRSLAQDAPPLPVPG |
| 756 | QLSISSCLQQLSLLM |
| 757 | SRLLEFYLAMPFATP |
| 758 | PLPVPGVLLKEFTVS |
| 759 | TIRLTAADHRQLQLS |
| 760 | HRQLQLSISSCLQQL |
| 761 | LMWITQCFLPVFLAQ |

TABLE 77
NYESO1 HLA-DRB1*0401 (DR4Dw4) Epitope Peptides

| SEQ ID NO. | Sequence |
|---|---|
| 762 | TIRLTAADHRQLQLS |
| 763 | LSLLMWITQCFLPVF |
| 764 | LLEFYLAMPFATPME |
| 765 | LKEFTVSGNILTIRL |
| 766 | ASGLNGCCRCGARGP |
| 767 | YLAMPFATPMEAELA |
| 768 | ATPMEAELARRSLAQ |
| 769 | PGVLLKEFTVSGNIL |
| 770 | GVLLKEFTVSGNILT |
| 771 | SGNILTIRLTAADHR |

TABLE 78
NYESO1 HLA-DRB1*0701 Epitope Peptides

| SEQ ID NO. | Sequence |
|---|---|
| 772 | HRQLQLSISSCLQQL |
| 773 | AMPFATPMEAELARR |
| 774 | VLLKEFTVSGNILTI |
| 775 | LKEFTVSGNILTIRL |
| 776 | FTVSGNILTIRLTAA |
| 777 | TIRLTAADHRQLQLS |
| 778 | QLSLLMWITQCFLPV |
| 779 | LSLLMWITQCFLPVF |
| 780 | YLAMPFATPMEAELA |
| 781 | SGNILTIRLTAADHR |

TABLE 79
NYESO1 HLA-DRB1*1101 Epitope Peptides

| SEQ ID NO. | Sequence |
|---|---|
| 782 | LEFYLAMPFATPMEA |
| 783 | TQCFLPVFLAQPPSG |
| 784 | ASGLNGCCRCGARGP |
| 785 | SGNILTIRLTAADHR |
| 786 | TIRLTAADHRQLQLS |
| 787 | MPFATPMEAELARRS |
| 788 | ATPMEAELARRSLAQ |
| 789 | TPMEAELARRSLAQD |
| 790 | PMEAELARRSLAQDA |
| 791 | LPVPGVLLKEFTVSG |

TABLE 80
NYESO1 HLA-DRB1*1501 (DR2b) Epitope Peptides

| SEQ ID NO. | Sequence |
|---|---|
| 792 | SRLLEFYLAMPFATP |
| 793 | QCFLPVFLAQPPSGQ |
| 794 | ESRLLEFYLAMPFAT |
| 795 | YLAMPFATPMEAELA |
| 796 | PGVLLKEFTVSGNIL |
| 797 | GVLLKEFTVSGNILT |
| 798 | QLSLLMWITQCFLPV |
| 799 | MWITQCFLPVFLAQP |
| 800 | LLEFYLAMPFATPME |
| 801 | LKEFTVSGNILTIRL |

MAGE A3 Antigenic Peptides

In some embodiments, the TVM composition includes MAGE-A3 (Melanoma-associated antigen 3) specific T-cells. MAGE-A3 specific T-cells can be generated as described below using one or more antigenic peptides to MAGE-A3. In some embodiments, the MAGE-A3 specific T-cells are generated using one or more antigenic peptides to MAGE-A3, or a modified or heteroclitic peptide derived from a MAGE-A3 peptide. In some embodiments, MAGE-A3 specific T-cells are generated using a MAGE-A3 antigen library comprising a pool of peptides (for example 15mers) containing amino acid overlap (for example 11 amino acids of overlap) between each sequence formed by scanning the protein amino acid sequence SEQ. ID. No. 802 (UniProt KB—P43357) for MAGE-A3:

MPLEQRSQHCKPEEGLEARGEALGLVGAQAPATEEQEAASSSSTLVEV
TLGEVPAAESPDPPQSPQGASSLPTTMNYPLWSQSYEDSSNQEEEGPS
TFPDLESEFQAALSRKVAELVHFLLLKYRAREPVTKAEMLGSVVGNWQ
YFFPVILLIIVLAIIAREGDCAPEEKIWEELSVLEVFEGREDSILGDP
KKLLTQHFVQENYLEYRQVPGSDPACYEFLWGPRALVETSYVKVLHHM
VKISGGPHISYPPLHEWVLREGEE.

Overlapping antigenic libraries are commercially available, for example, from JPT, for example, from JPT (Product Code: PM-MAGEA3 (PepMix Human (MAGE-A3)). In some embodiments, the MAGE-A3 specific T-cells are generated using a commercially available overlapping antigenic library made up of MAGE-A3 peptides.

In some embodiments, the MAGE-A3 specific T-cells are generated using one or more antigenic peptides to MAGE-A3, or a modified or heteroclitic peptide derived from a MAGE-A3 peptide. In some embodiments, the MAGE-A3 specific T-cells are generated with peptides that recognize class I MHC molecules. In some embodiments, the MAGE-A3 specific T-cells are generated with peptides that recognize class II MHC molecules. In some embodiments, the MAGE-A3 specific T-cells are generated with peptides that recognize both class I and class II MHC molecules.

In some embodiments, the MAGE-A3 peptides used to prime and expand a T-cell subpopulation includes specifically selected HLA-restricted peptides generated by determining the HLA profile of the donor source, and including peptides derived from MAGE-A3 that best match the donor's HLA. In some embodiments, the MAGE-A3 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting MAGE-A3 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 81-87, the HLA-B peptides are selected from the peptides of Tables 88-94, and the HLA-DR peptides are selected from the peptides of Tables 95-100. For example, if the donor cell source has an HLA profile that is HLA-A*01/*02:01; HLA-B*15:01/*18; and HLA-DRB1*0101/*0301, then the MAGE-A3 peptides used to prime and expand the MAGE-A3 specific T-cell subpopulation are restricted to the specific HLA profile, and may include the peptides identified in Table 81 (Seq. ID. Nos. 803-812) for HLA-A*01; Table 82 (Seq. ID. Nos. 813-822) for HLA-A*02:01; Table 90 (Seq. ID. Nos. 893-902) for HLA-B*15:01; Table 91 (Seq. ID. Nos. 903-912) for HLA-B*18; Table 95 (Seq. ID. Nos. 943-952) for HLA-DRB1*0101; and Table 96 (Seq. ID. Nos. 953-962) for HLA-DRB1*0301. In some embodiments, the mastermix of peptides includes both an overlapping peptide library and specifically selected HLA-restricted peptides generated by determining the HLA profile of the donor source.

In some embodiments, the MAGE-A3 HLA-restricted epitopes are specific to at least both of the donor's HLA-A alleles, at least both of the donor's HLA-B alleles, and at least both of the donor's HLA-DR alleles. In some embodiments, the MAGE-A3 HLA-A alleles are selected from a group comprising HLA-A*01, HLA-A*02:01, HLA-A*03, HLA-A*11:01, HLA-A*24:02, HLA-A*26, or HLA-A*68:01, and the corresponding MAGE-A3 HLA-restricted peptides are selected for: HLA-A*01 from Table 81; HLA-A*02:01 from Table 82; HLA-A*03 from Table 83; HLA-A*11:01 from Table 84; HLA-A*24:02 from Table 85; HLA-A*26 from Table 86; or HLA-A*68:01 from Table 87; or any combination thereof. In some embodiments, the MAGE-A3 HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding MAGE-A3 HLA-restricted peptides are selected for: HLA-B*07:02 from Table 88; HLA-B*08 from Table 89; HLA-B*15:01 (B62) from Table 90; HLA-B*18 from Table 91; HLA-B*27:05 from Table 92; HLA-B*35:01 from Table 93, or HLA-B*58:02 from Table 94; or any combination thereof. In some embodiments, the MAGE-A3 HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding MAGE-A3 HLA-restricted peptides are selected for: HLA-DRB1*0101 from Table 95; HLA-DRB1*0301 (DR17) from Table 96; HLA-DRB1*0401 (DR4Dw4) from Table 97; HLA-DRB1*0701 from Table 98; HLA-DRB1*1101 from Table 99; or HLA-DRB1*1501 (DR2b) from Table 100; or any combination thereof.

TABLE 81
MAGEA3 HLA-A*01 Epitope Peptides

| SEQ ID NO. | Sequence |
|---|---|
| 803 | LMEVDPIGHLY |
| 804 | AELVHFLLLKY |
| 805 | QHFVQENYLEY |
| 806 | ASSLPTTMNY |
| 807 | ELVHFLLLKY |
| 808 | LTQHFVQENY |
| 809 | EVDPIGHLY |
| 810 | SSLPTTMNY |
| 811 | LVHFLLLKY |
| 812 | GSVVGNWQY |

TABLE 82
MAGEA3 HLA-A*02:01 Epitope Peptides

| SEQ ID NO. | Sequence |
|---|---|
| 813 | TLVEVTLGEV |
| 814 | ALVETSYVKV |
| 815 | GLLIIVLAII |
| 816 | AALSRKVAEL |
| 817 | LVFGIELMEV |
| 818 | ALSRKVAEL |
| 819 | LLIIVLAII |
| 820 | GLLIIVLAI |
| 821 | FLWGPRALV |
| 822 | KIWEELSVL |

TABLE 83
MAGEA3 HLA-A*03 Epitope Peptides

| SEQ ID NO. | Sequence |
|---|---|
| 823 | KYRAREPVTK |
| 824 | YVKVLHHMVK |
| 825 | QVPGSDPACY |
| 826 | LLGDNQIMPK |
| 827 | KLLTQHFVQE |
| 828 | FLWGPRALVE |
| 829 | ALVETSYVK |
| 830 | ALGLVGAQA |
| 831 | ELVHFLLLK |
| 832 | YRAREPVTK |

TABLE 84
MAGEA3 HLA-A*11:01 Epitope Peptides

| SEQ ID NO. | Sequence |
|---|---|
| 833 | ESEFQAALSR |
| 834 | YVKVLHHMVK |
| 835 | AELVHFLLLK |
| 836 | LIIVLAIIAR |
| 837 | ASSSSTLVEV |
| 838 | STLVEVTLGE |
| 839 | ELVHFLLLK |
| 840 | SVLEVFEGR |
| 841 | DSILGDPKK |
| 842 | ALVETSYVK |

TABLE 85
MAGEA3 HLA-A*24:02 Epitope Peptides

| SEQ ID NO. | Sequence |
|---|---|
| 843 | SYPPLHEWVL |
| 844 | LYIFATCLGL |
| 845 | VFEGREDSIL |
| 846 | KVAELVHFLL |
| 847 | TFPDLESEF |
| 848 | VFEGREDSI |
| 849 | EFLWGPRAL |
| 850 | VAELVHFLL |
| 851 | IFSKASSSL |
| 852 | AELVHFLLL |

TABLE 86
MAGEA3 HLA-A*26 Epitope Peptides

| SEQ ID NO. | Sequence |
|---|---|
| 853 | ELVHFLLLKY |
| 854 | EKIWEELSVL |
| 855 | EVFEGREDSI |
| 856 | EVTLGEVPAA |
| 857 | EVDPIGHLY |
| 858 | LVHFLLLKY |
| 859 | EVFEGREDS |
| 860 | KVAELVHFL |
| 861 | EPVTKAEML |
| 862 | SVVGNWQYF |

TABLE 87
MAGEA3 HLA-A*68:01 Epitope Peptides

| SEQ ID NO. | Sequence |
|---|---|
| 863 | LLIIVLAIIAR |
| 864 | ELVHFLLLKYR |
| 865 | ELSVLEVFEGR |
| 866 | LIIVLAIIAR |
| 867 | ESEFQAALSR |
| 868 | IIVLAIIAR |
| 869 | ELVHFLLLK |
| 870 | IVLAIIARE |
| 871 | SVLEVFEGR |
| 872 | DSILGDPKK |

TABLE 88
MAGEA3 HLA-B*07:02 Epitope Peptides

| SEQ ID NO. | Sequence |
|---|---|
| 873 | APEEKIWEEL |
| 874 | SPQGASSLPT |
| 875 | APATEEQEAA |
| 876 | DPIGHLYIFA |
| 877 | GPHISYPPL |
| 878 | LPTTMNYPL |
| 879 | EPVTKAEML |
| 880 | YPPLHEWVL |
| 881 | APATEEQEA |
| 882 | MPKAGLLII |

TABLE 89
MAGEA3 HLA-B*08 Epitope Peptides

| SEQ ID NO. | Sequence |
|---|---|
| 883 | ALSRKVAEL |
| 884 | EPVTKAEML |
| 885 | GLEARGEAL |
| 886 | LLKYRAREP |
| 887 | QIMPKAGLL |
| 888 | EARGEALGL |
| 889 | MPKAGLLII |
| 890 | LLKYRARE |
| 891 | QIMPKAGL |
| 892 | EEKIWEEL |

TABLE 90
MAGEA3 HLA-B*15:01 (B62) Epitope Peptides

| SEQ ID NO. | Sequence |
|---|---|
| 893 | NQEEEGPSTF |
| 894 | ELVHFLLLKY |
| 895 | QVPGSDPACY |
| 896 | SVVGNWQYFF |
| 897 | TQHFVQENY |
| 898 | LVHFLLLKY |
| 899 | FVQENYLEY |
| 900 | WQYFFPVIF |
| 901 | EVDPIGHLY |
| 902 | VVGNWQYFF |

TABLE 91
MAGEA3 HLA-B*18 Epitope Peptides

| SEQ ID NO. | Sequence |
|---|---|
| 903 | EELSVLEVF |
| 904 | QEEEGPSTF |
| 905 | LESEFQAAL |
| 906 | PEEKIWEEL |
| 907 | AELVHFLLL |
| 908 | VETSYVKVL |
| 909 | EEEGPSTF |
| 910 | EEKIWEEL |
| 911 | AELVHFLL |
| 912 | LEARGEAL |

TABLE 92
MAGEA3 HLA-B*27:05 Epitope Peptides

| SEQ ID NO. | Sequence |
|---|---|
| 913 | AREPVTKAEM |
| 914 | SRKVAELVHF |
| 915 | SEFQAALSRK |
| 916 | RALVETSYVK |
| 917 | YRAREPVTK |
| 918 | PRALVETSY |
| 919 | SRKVAELVH |
| 920 | YFFPVIFSK |
| 921 | KAGLLIIVL |
| 922 | DSILGDPKK |

TABLE 93
MAGEA3 HLA-B*35:01 Epitope Peptides

| SEQ ID NO. | Sequence |
|---|---|
| 923 | APEEKIWEEL |
| 924 | GPRALVETSY |
| 925 | DPKKLLTQHF |
| 926 | EPVTKAEML |
| 927 | LPTTMNYPL |
| 928 | VPGSDPACY |
| 929 | YPPLHEWVL |
| 930 | GPHISYPPL |
| 931 | DPIGHLYIF |
| 932 | MPKAGLLII |

TABLE 94
MAGEA3 HLA-B*58:02 Epitope Peptides

| SEQ ID NO. | Sequence |
|---|---|
| 933 | KVAELVHFLL |
| 934 | KASSSLQLVF |
| 935 | SSSTLVEVTL |
| 936 | FSKASSSLQL |
| 937 | KAGLLIIVL |
| 938 | KVAELVHFL |
| 939 | SSTLVEVTL |
| 940 | SSLQLVFGI |
| 941 | KVLHHMVKI |
| 942 | SSLPTTMNY |

TABLE 95
MAGEA3 HLA-DRB1*0101 Epitope Peptides

| SEQ ID NO. | Sequence |
|---|---|
| 943 | PACYEFLWGPRALVE |
| 944 | YLEYRQVPGSDPACY |
| 945 | AGLLIIVLAIIAREG |
| 946 | GEALGLVGAQAPATE |
| 947 | QYFFPVIFSKASSSL |
| 948 | SSSLQLVFGIELMEV |
| 949 | EVTLGEVPAAESPDP |
| 950 | HHMVKISGGPHISYP |
| 951 | HFLLLKYRAREPVTK |
| 952 | ETSYVKVLHHMVKIS |

TABLE 96
MAGEA3 HLA-DRB1*0301 (DR17) Epitope Peptides

| SEQ ID NO. | Sequence |
|---|---|
| 953 | EDSILGDPKKLLTQH |
| 954 | IELMEVDPIGHLYIF |
| 955 | YDGLLGDNQIMPKAG |
| 956 | FPDLESEFQAALSRK |
| 957 | GPSTFPDLESEFQAA |
| 958 | LGSVVGNWQYFFPVI |
| 959 | ASSLPTTMNYPLWSQ |
| 960 | VAELVHFLLLKYRAR |
| 961 | CLGLSYDGLLGDNQI |
| 962 | SRKVAELVHFLLLKY |

TABLE 97
MAGEA3 HLA-DRB1*0401 (DR4Dw4) Epitope Peptides

| SEQ ID NO. | Sequence |
|---|---|
| 963 | PSTFPDLESEFQAAL |
| 964 | ESEFQAALSRKVAEL |
| 965 | QYFFPVIFSKASSSL |
| 966 | PVIFSKASSSLQLVF |
| 967 | ETSYVKVLHHMVKIS |
| 968 | FPDLESEFQAALSRK |
| 969 | SRKVAELVHFLLLKY |
| 970 | LMEVDPIGHLYIFAT |
| 971 | TSYVKVLHHMVKISG |
| 972 | WQYFFPVIFSKASSS |

TABLE 98
MAGEA3 HLA-DRB1*0701 Epitope Peptides

| SEQ ID NO. | Sequence |
|---|---|
| 973 | ESEFQAALSRKVAEL |
| 974 | ASSLPTTMNYPLWSQ |
| 975 | ATCLGLSYDGLLGDN |
| 976 | QYFFPVIFSKASSSL |
| 977 | FPVIFSKASSSLQLV |
| 978 | PVIFSKASSSLQLVF |
| 979 | GHLYIFATCLGLSYD |
| 980 | LEVFEGREDSILGDP |
| 981 | PRALVETSYVKVLHH |
| 982 | HISYPPLHEWVLREG |

TABLE 99
MAGEA3 HLA-DRB1*1101 Epitope Peptides

| SEQ ID NO. | Sequence |
|---|---|
| 983 | VKVLHHMVKISGGPH |
| 984 | WQYFFPVIFSKASSS |
| 985 | PACYEFLWGPRALVE |
| 986 | ETSYVKVLHHMVKIS |
| 987 | SRKVAELVHFLLLKY |
| 988 | ELVHFLLLKYRAREP |
| 989 | QYFFPVIFSKASSSL |
| 990 | YLEYRQVPGSDPACY |
| 991 | TSYVKVLHHMVKISG |
| 992 | SEFQAALSRKVAELV |

TABLE 100
MAGEA3 HLA-DRB1*1501 (DR2b) Epitope Peptides

| SEQ ID NO. | Sequence |
|---|---|
| 993 | GSVVGNWQYFFPVIF |
| 994 | HFLLLKYRAREPVTK |
| 995 | IGHLYIFATCLGLSY |
| 996 | VAELVHFLLLKYRAR |
| 997 | SSSLQLVFGIELMEV |
| 998 | GIELMEVDPIGHLYI |
| 999 | TCLGLSYDGLLGDNQ |
| 1000 | DNQIMPKAGLLIIVL |
| 1001 | AGLLIIVLAIIAREG |
| 1002 | LSVLEVFEGREDSIL |

MAGE A4 Antigenic Peptides

In some embodiments, the TVM composition includes MAGE-A4 (Melanoma-associated antigen 4) specific T-cells. MAGE-A4 specific T-cells can be generated as described below using one or more antigenic peptides to MAGE-A4. In some embodiments, the MAGE-A4 specific T-cells are generated using one or more antigenic peptides to MAGE-A4, or a modified or heteroclitic peptide derived from a MAGE-A4 peptide. In some embodiments, MAGE-A4 specific T-cells are generated using a MAGE-A4 antigen library comprising a pool of peptides (for example 15mers) containing amino acid overlap (for example 11 amino acids of overlap) between each sequence formed by scanning the protein amino acid sequence SEQ. ID. No. 1003 (UniProt KB—P43358) for MAGE-A4:

MSSEQKSQHCKPEEGVEAQEEALGLVGAQAPTTEEQEAAVSSSSPLVPGT
LEEVPAAESAGPPQSPQGASALPTTISFTCWRQPNEGSSSQEEEGPSTSP
DAESLFREALSNKVDELAHFLLRKYRAKELVTKAEMLERVIKNYKRCFPV
IFGKASESLKMIFGIDVKEVDPASNTYTLVTCLGLSYDGLLGNNQIFPKT
GLLIIVLGTIAMEGDSASEEEIWEELGVMGVYDGREHTVYGEPRKLLTQD
WVQENYLEYRQVPGSNPARYEFLWGPRALAETSYVKVLEHVVRVNARVRI
AYPSLREAALLEEEEGV

Overlapping antigenic libraries are commercially available, for example, from JPT, for example, from JPT (Product Code: PM-MAGEA4 (PepMix Human (MAGE-A4)). In some embodiments, the MAGE-A4 specific T-cells are generated using a commercially available overlapping antigenic library made up of MAGE-A4 peptides.

In some embodiments, the MAGE-A4 specific T-cells are generated using one or more antigenic peptides to MAGE-A4, or a modified or heteroclitic peptide derived from a MAGE-A4 peptide. In some embodiments, the MAGE-A4 specific T-cells are generated with peptides that recognize class I MHC molecules. In some embodiments, the MAGE-A4 specific T-cells are generated with peptides that recognize class II MHC molecules. In some embodiments, the MAGE-A4 specific T-cells are generated with peptides that recognize both class I and class II MHC molecules.

In some embodiments, the MAGE-A4 peptides used to prime and expand a T-cell subpopulation includes specifically selected HLA-restricted peptides generated by determining the HLA profile of the donor source, and including peptides derived from MAGE-A4 that best match the donor's HLA. In some embodiments, the MAGE-A4 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting MAGE-A4 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 101-107, the HLA-B peptides are selected from the peptides of Tables 108-114, and the HLA-DR peptides are selected from the peptides of Tables 115-120. For example, if the donor cell source has an HLA profile that is HLA-A*01/*02:01; HLA-B*15:01/*18; and HLA-DRB1*0101/*0301, then the MAGE-A4 peptides used to prime and expand the MAGE-A4 specific T-cell subpopulation are restricted to the specific HLA profile, and may include the peptides identified in Table 101 (Seq. ID. Nos. 1004-1013) for HLA-A*01; Table 102 (Seq. ID. Nos. 1014-1023) for HLA-A*02:01; Table 110 (Seq. ID. Nos. 1093-1102) for HLA-B*15:01; Table 111 (Seq. ID. Nos. 1103-1112) for HLA-B*18; Table 115 (Seq. ID. Nos. 1143-1152) for HLA-DRB1*0101; and Table 116 (Seq. ID. Nos. 1153-1162) for HLA-DRB1*0301. In some embodiments, the mastermix of peptides includes both an overlapping peptide library and specifically selected HLA-restricted peptides generated by determining the HLA profile of the donor source.

In some embodiments, the MAGE-A4 HLA-restricted epitopes are specific to at least both of the donor's HLA-A alleles, at least both of the donor's HLA-B alleles, and at least both of the donor's HLA-DR alleles. In some embodiments, the MAGE-A4 HLA-A alleles are selected from a group comprising HLA-A*01, HLA-A*02:01, HLA-A*03, HLA-A*11:01, HLA-A*24:02, HLA-A*26, or HLA-A*68:01, and the corresponding MAGE-A4 HLA-restricted peptides are selected for: HLA-A*01 from Table 101; HLA-A*02:01 from Table 102; HLA-A*03 from Table 103; HLA-A*11:01 from Table 104; HLA-A*24:02 from Table 105; HLA-A*26 from Table 106; or HLA-A*68:01 from Table 107; or any combination thereof. In some embodiments, the MAGE-A4 HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding MAGE-A4 HLA-restricted peptides are selected for: HLA-B*07:02 from Table 108; HLA-B*08 from Table 109; HLA-B*15:01 (B62) from Table 110; HLA-B*18 from Table 111; HLA-B*27:05 from Table 112; HLA-B*35:01 from Table 113, or HLA-B*58:02 from Table 114; or any combination thereof. In some embodiments, the MAGE-A4 HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding MAGE-A4 HLA-restricted peptides are selected for: HLA-DRB1*0101 from Table 115; HLA-DRB1*0301 (DR17) from Table 116; HLA-DRB1*0401 (DR4Dw4) from Table 117; HLA-DRB1*0701 from Table 118; HLA-DRB1*1101 from Table 119; or HLA-DRB1*1501 (DR2b) from Table 120; or any combination thereof.

TABLE 101
MAGEA4 HLA-A*01 Epitope Peptides

| SEQ ID NO. | Sequence |
|---|---|
| 1004 | YTLVTCLGLSY |
| 1005 | VKEVDPASNTY |
| 1006 | IWEELGVMGVY |
| 1007 | QDWVQENYLEY |
| 1008 | VYDGREHTVY |
| 1009 | WEELGVMGVY |
| 1010 | LTQDWVQENY |
| 1011 | EVDPASNTY |
| 1012 | TQDWVQENY |
| 1013 | MLERVIKNY |

TABLE 102
MAGEA4 HLA-A*02:01 Epitope Peptides

| SEQ ID NO. | Sequence |
|---|---|
| 1014 | ALAETSYVKV |
| 1015 | GLLIIVLGTI |
| 1016 | MIFGIDVKEV |
| 1017 | PLVPGTLEEV |
| 1018 | VIFGKASESL |
| 1019 | ALSNKVDEL |
| 1020 | LLIIVLGTI |
| 1021 | ALLEEEEGV |
| 1022 | KVLEHVVRV |
| 1023 | QIFPKTGLL |

TABLE 103
MAGEA4 HLA-A*03 Epitope Peptides

| SEQ ID NO. | Sequence |
|---|---|
| 1024 | SLFREALSNK |
| 1025 | KYRAKELVTK |
| 1026 | RVRIAYPSLR |
| 1027 | TLVTCLGLSY |
| 1028 | QVPGSNPARY |
| 1029 | HVVRVNARVR |
| 1030 | ALAETSYVK |
| 1031 | FLLRKYRAK |
| 1032 | ALGLVGAQA |
| 1033 | ELAHFLLRK |

TABLE 104
MAGEA4 HLA-A*11:01 Epitope Peptides

| SEQ ID NO. | Sequence |
|---|---|
| 1034 | TSPDAESLFR |
| 1035 | YVKVLEHVVR |
| 1036 | SSEQKSQHCK |
| 1037 | LVTKAEMLER |
| 1038 | RVRIAYPSLR |
| 1039 | VTKAEMLER |
| 1040 | ELAHFLLRK |
| 1041 | GVMGVYDGR |
| 1042 | TTISFTCWR |
| 1043 | ALAETSYVK |

TABLE 105
MAGEA4 HLA-A*24:02 Epitope Peptides

| SEQ ID NO. | Sequence |
|---|---|
| 1044 | AYPSLREAAL |
| 1045 | TYTLVTCLGL |
| 1046 | NYKRCFPVIF |
| 1047 | IFPKTGLLII |
| 1048 | KVDELAHFLL |
| 1049 | VYGEPRKLL |
| 1050 | NYKRCFPVI |
| 1051 | EFLWGPRAL |
| 1052 | IFGKASESL |

TABLE 106
MAGEA4 HLA-A*26 Epitope Peptides

| SEQ ID NO. | Sequence |
|---|---|
| 1053 | EMLERVIKNY |
| 1054 | EGVEAQEEAL |
| 1055 | ELAHFLLRKY |
| 1056 | EALSNKVDEL |
| 1057 | DWVQENYLEY |
| 1058 | ETSYVKVLEH |
| 1059 | EVDPASNTY |
| 1060 | LVTCLGLSY |
| 1061 | ELVTKAEML |
| 1062 | WVQENYLEY |

TABLE 107
MAGEA4 HLA-A*68:01 Epitope Peptides

| SEQ ID NO. | Sequence |
|---|---|
| 1063 | ELAHFLLRKYR |
| 1064 | STSPDAESLFR |
| 1065 | ELVTKAEMLER |
| 1066 | YVKVLEHVVR |
| 1067 | PTTISFTCWR |
| 1068 | LVTKAEMLER |
| 1069 | ELAHFLLRK |
| 1070 | TTISFTCWR |
| 1071 | GVMGVYDGR |
| 1072 | QVPGSNPAR |

TABLE 108
MAGEA4 HLA-B*07:02 Epitope Peptides

| SEQ ID NO. | Sequence |
|---|---|
| 1073 | YPSLREAALL |
| 1074 | SPQGASALPT |
| 1075 | VPGTLEEVPA |
| 1076 | APTTEEQEAA |
| 1077 | DPASNTYTLV |
| 1078 | PPQSPQGASA |
| 1079 | YPSLREAAL |
| 1080 | DPASNTYTL |
| 1081 | APTTEEQEA |
| 1082 | FPKTGLLII |

TABLE 109
MAGEA4 HLA-B*08 Epitope Peptides

| SEQ ID NO. | Sequence |
|---|---|
| 1083 | LRKYRAKEL |
| 1084 | ALSNKVDEL |
| 1085 | ELVTKAEML |
| 1086 | YPSLREAAL |
| 1087 | QIFPKTGLL |
| 1088 | VIKNYKRCF |
| 1089 | SLREAALL |
| 1090 | SLKMIFGI |
| 1091 | QIFPKTGL |
| 1092 | FPKTGLLI |

TABLE 110
MAGEA4 HLA-B*15:01 (B62) Epitope Peptides

| SEQ ID NO. | Sequence |
|---|---|
| 1093 | TLVTCLGLSY |
| 1094 | RVNARVRIAY |
| 1095 | ELAHFLLRKY |
| 1096 | QVPGSNPARY |
| 1097 | RVIKNYKRCF |
| 1098 | MLERVIKNY |
| 1099 | TQDWVQENY |
| 1100 | LVTCLGLSY |
| 1101 | WVQENYLEY |
| 1102 | EVDPASNTY |

TABLE 111
MAGEA4 HLA-B*18 Epitope Peptides

| SEQ ID NO. | Sequence |
|---|---|
| 1103 | AESLFREAL |
| 1104 | SEEEIWEEL |
| 1105 | EELGVMGVY |
| 1106 | AETSYVKVL |
| 1107 | DELAHFLL |
| 1108 | EEEIWEEL |
| 1109 | LERVIKNY |
| 1110 | SESLKMIF |
| 1111 | VEAQEEAL |
| 1112 | DGREHTVY |

TABLE 112
MAGEA4 HLA-B*27:05 Epitope Peptides

| SEQ ID NO. | Sequence |
|---|---|
| 1113 | KRCFPVIFGK |
| 1114 | ARYEFLWGPR |
| 1115 | ARVRIAYPSL |
| 1116 | YRAKELVTK |
| 1117 | ERVIKNYKR |
| 1118 | VRIAYPSLR |
| 1119 | LRKYRAKEL |
| 1120 | RCFPVIFGK |
| 1121 | PRALAETSY |
| 1122 | KMIFGIDVK |

TABLE 113
MAGEA4 HLA-B*35:01 Epitope Peptides

| SEQ ID NO. | Sequence |
|---|---|
| 1123 | YPSLREAALL |
| 1124 | GPRALAETSY |
| 1125 | DPASNTYTL |
| 1126 | YPSLREAAL |
| 1127 | VPGSNPARY |
| 1128 | FPKTGLLII |
| 1129 | LPTTISFTC |
| 1130 | KVDELABFL |
| 1131 | MLERVIKNY |
| 1132 | LGLSYDGLL |

TABLE 114
MAGEA4 HLA-B*58:02 Epitope Peptides

| SEQ ID NO. | Sequence |
|---|---|
| 1133 | RVIKNYKRCF |
| 1134 | KASESLKMIF |
| 1135 | SSSPLVPGTL |
| 1136 | KAEMLERVI |
| 1137 | KTGLLIIVL |
| 1138 | KVDELAHFL |
| 1139 | KASESLKMI |
| 1140 | PSLREAALL |
| 1141 | SSPLVPGTL |
| 1142 | LAHFLLRKY |

TABLE 115
MAGEA4 HLA-DRB1*0101 Epitope Peptides

| SEQ ID NO. | Sequence |
|---|---|
| 1143 | PARYEFLWGPRALAE |
| 1144 | TGLLIIVLGTIAMEG |
| 1145 | YLEYRQVPGSNPARY |
| 1146 | KRCFPVIFGKASESL |
| 1147 | EEALGLVGAQAPTTE |
| 1148 | SESLKMIFGIDVKEV |
| 1149 | GLLIIVLGTIAMEGD |
| 1150 | PGTLEEVPAAESAGP |
| 1151 | HFLLRKYRAKELVTK |
| 1152 | EEIWEELGVMGVYDG |

TABLE 116
MAGEA4 HLA-DRB1*0301 (DR17) Epitope Peptides

| SEQ ID NO. | Sequence |
|---|---|
| 1153 | GPSTSPDAESLFREA |
| 1154 | EHTVYGEPRKLLTQD |
| 1155 | LERVIKNYKRCFPVI |
| 1156 | VVRVNARVRIAYPSL |
| 1157 | KMIFGIDVKEVDPAS |
| 1158 | KAEMLERVIKNYKRC |
| 1159 | CLGLSYDGLLGNNQI |
| 1160 | RKLLTQDWVQENYLE |
| 1161 | ALSNKVDELAHFLLR |
| 1162 | TYTLVTCLGLSYDGL |

TABLE 117
MAGEA4 HLA-DRB1*0401 (DR4Dw4) Epitope Peptides

| SEQ ID NO. | Sequence |
|---|---|
| 1163 | ESLFREALSNKVDEL |
| 1164 | SNKVDELAHFLLRKY |
| 1165 | VKEVDPASNTYTLVT |
| 1166 | GLLIIVLGTIAMEGD |
| 1167 | EHTVYGEPRKLLTQD |
| 1168 | VKVLEHVVRVNARVR |
| 1169 | KRCFPVIFGKASESL |
| 1170 | PVIFGKASESLKMIF |
| 1171 | SNTYTLVTCLGLSYD |
| 1172 | GLSYDGLLGNNQIFP |

TABLE 118
MAGEA4 HLA-DRB1*0701 Epitope Peptides

| SEQ ID NO. | Sequence |
|---|---|
| 1173 | ESLFREALSNKVDEL |
| 1174 | VTCLGLSYDGLLGNN |
| 1175 | NQIFPKTGLLIIVLG |
| 1176 | ASALPTTISFTCWRQ |
| 1177 | FPVIFGKASESLKMI |
| 1178 | GLLIIVLGTIAMEGD |
| 1179 | PRALAETSYVKVLEH |
| 1180 | ETSYVKVLEHVVRVN |
| 1181 | RIAYPSLREAALLEE |
| 1182 | EQEAAVSSSSPLVPG |

TABLE 119
MAGEA4 HLA-DRB1*1101 Epitope Peptides

| SEQ ID NO. | Sequence |
|---|---|
| 1183 | VKVLEHVVRVNARVR |
| 1184 | RYEFLWGPRALAETS |
| 1185 | PARYEFLWGPRALAE |
| 1186 | ELAHFLLRKYRAKEL |
| 1187 | KRCFPVIFGKASESL |
| 1188 | YLEYRQVPGSNPARY |
| 1189 | TSYVKVLEHVVRVNA |
| 1190 | SNKVDELAHFLLRKY |
| 1191 | KMIFGIDVKEVDPAS |
| 1192 | AEMLERVIKNYKRCF |

TABLE 120
MAGEA4 HLA-DRB1*1501 (DR2b) Epitope Peptides

| SEQ ID NO. | Sequence |
|---|---|
| 1193 | HFLLRKYRAKELVTK |
| 1194 | LGVMGVYDGREHTVY |
| 1195 | EAAVSSSSPLVPGTL |
| 1196 | ASALPTTISFTCWRQ |
| 1197 | ERVIKNYKRCFPVIF |
| 1198 | SESLKMIFGIDVKEV |
| 1199 | TCLGLSYDGLLGNNQ |
| 1200 | NNQIFPKTGLLIIVL |
| 1201 | GLLIIVLGTIAMEGD |
| 1202 | LIIVLGTIAMEGDSA |

SSX2 Antigenic Peptides

In some embodiments, the TVM composition includes SSX2 (Synovial sarcoma, X breakpoint 2) specific T-cells. SSX2 specific T-cells can be generated as described below using one or more antigenic peptides to SSX2. In some embodiments, the SSX2 specific T-cells are generated using one or more antigenic peptides to SSX2, or a modified or heteroclitic peptide derived from a SSX2 peptide. In some embodiments, SSX2 specific T-cells are generated using a SSX2 antigen library comprising a pool of peptides (for example 15mers) containing amino acid overlap (for example 11 amino acids of overlap) between each sequence formed by scanning the protein amino acid sequence SEQ. ID. No. 1203 (UniProt KB—Q16385) for SSX2:

MNGDDAFARRPTVGAQIPEKIQKAFDDIAKYFSKEEWEKMKASEKIFYV
YMKRKYEAMTKLGFKATLPPFMCNKRAEDFQGNDLDNDPNRGNQVERPQ
MTFGRLQGISPKIMPKKPAEEGNDSEEVPEASGPQNDGKELCPPGKPTT
SEKIHERSGPKRGEHAWTHRLRERKQLVIYEEISDPEEDDE.

Overlapping antigenic libraries are commercially available, for example, from JPT, for example, from JPT (Product Code: PM-SSX2 (PepMix Human (SSX2)). In some embodiments, the SSX2 specific T-cells are generated using a commercially available overlapping antigenic library made up of SSX2 peptides.

In some embodiments, the SSX2 specific T-cells are generated using one or more antigenic peptides to SSX2, or a modified or heteroclitic peptide derived from a SSX2 peptide. In some embodiments, the SSX2 specific T-cells are generated with peptides that recognize class I MHC molecules. In some embodiments, the SSX2 specific T-cells are generated with peptides that recognize class II MHC molecules. In some embodiments, the SSX2 specific T-cells are generated with peptides that recognize both class I and class II MHC molecules.

In some embodiments, the SSX2 peptides used to prime and expand a T-cell subpopulation includes specifically selected HLA-restricted peptides generated by determining the HLA profile of the donor source, and including peptides derived from SSX2 that best match the donor's HLA. In some embodiments, the SSX2 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting SSX2 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 121-127, the HLA-B peptides are selected from the peptides of Tables 128-134, and the HLA-DR peptides are selected from the peptides of Tables 135-140. For example, if the donor cell source has an HLA profile that is HLA-A*01/*02:01; HLA-B*15:01/*18; and HLA-DRB1*0101/*0301, then the SSX2 peptides used to prime and expand the SSX2 specific T-cell subpopulation are restricted to the specific HLA profile, and may include the peptides identified in Table 121 (Seq. ID. Nos. 1204-1213) for HLA-A*01; Table 122 (Seq. ID. Nos. 1214-1223) for HLA-A*02:01; Table 130 (Seq. ID. Nos. 1294-1303) for HLA-B*15:01; Table 131 (Seq. ID. Nos. 1304-1313) for HLA-B*18; Table 135 (Seq. ID. Nos. 1344-1353) for HLA-DRB1*0101; and Table 136 (Seq. ID. Nos. 1354-1363) for HLA-DRB1*0301. In some embodiments, the mastermix of peptides includes both an overlapping peptide library and specifically selected HLA-restricted peptides generated by determining the HLA profile of the donor source.

In some embodiments, the SSX2 HLA-restricted epitopes are specific to at least both of the donor's HLA-A alleles, at least both of the donor's HLA-B alleles, and at least both of the donor's HLA-DR alleles. In some embodiments, the SSX2 HLA-A alleles are selected from a group comprising HLA-A*01, HLA-A*02:01, HLA-A*03, HLA-A*11:01, HLA-A*24:02, HLA-A*26, or HLA-A*68:01, and the corresponding SSX2 HLA-restricted peptides are selected for: HLA-A*01 from Table 121; HLA-A*02:01 from Table 122; HLA-A*03 from Table 123; HLA-A*11:01 from Table 124; HLA-A*24:02 from Table 125; HLA-A*26 from Table 126; or HLA-A*68:01 from Table 127; or any combination thereof. In some embodiments, the SSX2 HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding SSX2 HLA-restricted peptides are selected for: HLA-B*07:02 from Table 128; HLA-B*08 from Table 129; HLA-B*15:01 (B62) from Table 130; HLA-B*18 from Table 131; HLA-B*27:05 from Table 132; HLA-B*35:01 from Table 133, or HLA-B*58:02 from Table 134; or any combination thereof. In some embodiments, the SSX2 HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding SSX2 HLA-restricted peptides are selected for: HLA-DRB1*0101 from Table 135; HLA-DRB1*0301 (DR17) from Table 136; HLA-DRB1*0401 (DR4Dw4) from Table 137; HLA-DRB1*0701 from Table 138; HLA-DRB1*1101 from Table 139; or HLA-DRB1*1501 (DR2b) from Table 140; or any combination thereof.

TABLE 121
SSX2 HLA-A*01 Epitope Peptides

| SEQ ID NO. | Sequence |
|---|---|
| 1204 | RLRERKQLVIY |
| 1205 | EKMKASEKIFY |
| 1206 | KIFYVYMKRKY |
| 1207 | IQKAFDDIAKY |
| 1208 | MKASEKIFYVY |
| 1209 | LRERKQLVIY |
| 1210 | IFYVYMKRKY |
| 1211 | ASEKIFYVY |
| 1212 | KAFDDIAKY |
| 1213 | FYVYMKRKY |

TABLE 122
SSX2 HLA-A*02:01 Epitope Peptides

| SEQ ID NO. | Sequence |
|---|---|
| 1214 | RLRERKQLVI |
| 1215 | QMTFGRLQGI |
| 1216 | RLQGISPKI |
| 1217 | KASEKIFYV |
| 1218 | RLRERKQLV |
| 1219 | QIPEKIQKA |
| 1220 | MTFGRLQGI |
| 1221 | TKLGFKATL |
| 1222 | DAFARRPTV |
| 1223 | KIQKAFDDI |

TABLE 123
SSX2 HLA-A*03 Epitope Peptides

| SEQ ID NO. | Sequence |
|---|---|
| 1224 | RLRERKQLVI |
| 1225 | KIFYVYMKRK |
| 1226 | KIHERSGPK |
| 1227 | QVERPQMTF |
| 1228 | TLPPFMCNK |
| 1229 | GISPKIMPK |
| 1230 | TVGAQIPEK |
| 1231 | AQIPEKIQK |
| 1232 | KRKYEAMTK |
| 1233 | ARRPTVGAQI |

TABLE 124
SSX2 HLA-A*11:01 Epitope Peptides

| SEQ ID NO. | Sequence |
|---|---|
| 1234 | ATLPPFMCNK |
| 1235 | PTVGAQIPEK |
| 1236 | PTTSEKIHER |
| 1237 | FSKEEWEKMK |
| 1238 | ASGPQNDGK |
| 1239 | TVGAQIPEK |
| 1240 | GISPKIMPK |
| 1241 | TTSEKIHER |
| 1242 | WTHRLRERK |
| 1243 | AQIPEKIQK |

TABLE 125
SSX2 HLA-A*24:02 Epitope Peptides

| SEQ ID NO. | Sequence |
|---|---|
| 1244 | KYEAMTKLGF |
| 1245 | TVGAQIPEKI |
| 1246 | QIPEKIQKAF |
| 1247 | VYMKRKYEAM |
| 1248 | GRLQGISPKI |
| 1249 | AFDDIAKYF |
| 1250 | VGAQIPEKI |
| 1251 | GFKATLPPF |
| 1252 | GKPTTSEKI |
| 1253 | KQLVIYEEI |

TABLE 126
SSX2 HLA-A*26 Epitope Peptides

| SEQ ID NO. | Sequence |
|---|---|
| 1254 | EVPEASGPQN |
| 1255 | ERPQMTFGRL |
| 1256 | LVIYEEISDP |
| 1257 | EKMKASEKIF |
| 1258 | MTKLGFKATL |
| 1259 | KAFDDIAKY |
| 1260 | EKIFYVYMK |
| 1261 | QVERPQMTF |
| 1262 | EAMTKLGFK |
| 1263 | MTFGRLQGI |

TABLE 127
SSX2 HLA-A*68:01 Epitope Peptides

| SEQ ID NO. | Sequence |
|---|---|
| 1264 | ATLPPFMCNKR |
| 1265 | KIQKAFDDIAK |
| 1266 | QVERPQMTFGR |
| 1267 | GAQIPEKIQK |
| 1268 | DPNRGNQVER |
| 1269 | EASGPQNDGK |
| 1270 | TTSEKIHER |
| 1271 | EAMTKLGFK |
| 1272 | TVGAQIPEK |
| 1273 | GISPKIMPK |

TABLE 128
SSX2 HLA-B*07:02 Epitope Peptides

| SEQ ID NO. | Sequence |
|---|---|
| 1274 | SPKIMPKKPA |
| 1275 | GPKRGEHAWT |
| 1276 | LPPFMCNKRA |
| 1277 | RPQMTFGRL |
| 1278 | GPQNDGKEL |
| 1279 | IPEKIQKAF |
| 1280 | PPGKPTTSE |
| 1281 | FARRPTVGA |
| 1282 | AEDFQGNDL |
| 1283 | MPKKPAEEG |

TABLE 129
SSX2 HLA-B*08 Epitope Peptides

| SEQ ID NO. | Sequence |
|---|---|
| 1284 | WEKNIKASEK |
| 1285 | YMKRKYEAM |
| 1286 | SPKIMPKKP |
| 1287 | GPQNDGKEL |
| 1288 | RLRERKQL |
| 1289 | KLGFKATL |
| 1290 | EAMTKLGF |
| 1291 | IQKAFDDI |
| 1292 | GPKRGEHA |
| 1293 | GFKATLPPF |

TABLE 130
SSX2 HLA-B*15:01 (B62) Epitope Peptides

| SEQ ID NO. | Sequence |
|---|---|
| 1294 | NQVERPQMTF |
| 1295 | AQIPEKIQKA |
| 1296 | KASEKIFYVY |
| 1297 | RLRERKQLVI |
| 1298 | RLQGISPKIM |
| 1299 | ELCPPGKPTT |
| 1300 | KAFDDIAKY |
| 1301 | AQIPEKIQK |
| 1302 | PQNDGKELC |
| 1303 | RLRERKQLV |

TABLE 131
SSX2 HLA-B*18 Epitope Peptides

| SEQ ID NO. | Sequence |
|---|---|
| 1304 | RERKQLVIY |
| 1305 | YEAMTKLGF |
| 1306 | AEDFQGNDL |
| 1307 | GEHAWTHRL |
| 1308 | EEISDPEED |
| 1309 | SEKIFYVY |
| 1310 | PEKIQKAF |
| 1311 | VERPQMTF |
| 1312 | EEWEKMKA |
| 1313 | EEVPEASG |

TABLE 132
SSX2 HLA-B*27:05 Epitope Peptides

| SEQ ID NO. | Sequence |
|---|---|
| 1314 | GRLQGISPKI |
| 1315 | KRKYEAMTKL |
| 1316 | KRGEHAWTHR |
| 1317 | ERPQMTFGRL |
| 1318 | ERSGPKRGEH |
| 1319 | GRLQGISPK |
| 1320 | KRKYEAMTK |
| 1321 | HRLRERKQL |
| 1322 | RRPTVGAQI |
| 1323 | LRERKQLVI |

TABLE 133
SSX2 HLA-B*35:01 Epitope Peptides

| SEQ ID NO. | Sequence |
|---|---|
| 1324 | THRLRERKQL |
| 1325 | KRKYEAMTKL |
| 1326 | ERPQMTFGRL |
| 1327 | GRLQGISPKI |
| 1328 | IHERSGPKRG |
| 1329 | LRERKQLVI |
| 1330 | RRPTVGAQI |
| 1331 | AFDDIAKYF |
| 1332 | TKLGFKATL |
| 1333 | AEDFQGNDL |

TABLE 134
SSX2 HLA-B*58:02 Epitope Peptides

| SEQ ID NO. | Sequence |
|---|---|
| 1334 | KAFDDIAKYF |
| 1335 | KASEKIFYVY |
| 1336 | RAEDFQGNDL |
| 1337 | MTKLGFKATL |
| 1338 | ASEKIFYVYM |
| 1339 | IAKYFSKEEW |
| 1340 | KAFDDIAKY |
| 1341 | FSKEEWEKM |
| 1342 | ASEKIFYVY |
| 1343 | QVERPQMTF |

TABLE 135
SSX2 HLA-DRB1*0101 Epitope Peptides

| SEQ ID NO. | Sequence |
|---|---|
| 1344 | QMTFGRLQGISPKIM |
| 1345 | RPTVGAQIPEKIQKA |
| 1346 | FGRLQGISPKIMPKK |
| 1347 | DDAFARRPTVGAQIP |
| 1348 | KEEWEKMKASEKIFY |
| 1349 | KRKYEAMTKLGFKAT |
| 1350 | KLGFKATLPPFMCNK |
| 1351 | QKAFDDIAKYFSKEE |
| 1352 | PPFMCNKRAEDFQGN |
| 1353 | QGISPKIMPKKPAEE |

TABLE 136
SSX2 HLA-DRB1*0301 (DR17) Epitope Peptides

| SEQ ID NO. | Sequence |
|---|---|
| 1354 | GNDLDNDPNRGNQVE |
| 1355 | GAQIPEKIQKAFDDI |
| 1356 | WEKMKASEKIFYVYM |
| 1357 | EKIFYVYMKRKYEAM |
| 1358 | KIQKAFDDIAKYFSK |
| 1359 | THRLRERKQLVIYEE |
| 1360 | IQKAFDDIAKYFSKE |
| 1361 | YVYMKRKYEAMTKLG |
| 1362 | PPFMCNKRAEDFQGN |
| 1363 | CNKRAEDFQGNDLDN |

TABLE 137
SSX2 HLA-DRB1*0401 (DR4Dw4) Epitope Peptides

| SEQ ID NO. | Sequence |
|---|---|
| 1364 | GNDLDNDPNRGNQVE |
| 1365 | QKAFDDIAKYFSKEE |
| 1366 | AKYFSKEEWEKMKAS |
| 1367 | PEKIQKAFDDIAKYF |
| 1368 | SEKIFYVYMKRKYEA |
| 1369 | YEAMTKLGFKATLPP |
| 1370 | FGRLQGISPKIMPKK |
| 1371 | SEEVPEASGPQNDGK |
| 1372 | QLVIYEEISDPEEDD |
| 1373 | MNGDDAFARRPTVGA |

TABLE 138
SSX2 HLA-DRB1*0701 Epitope Peptides

| SEQ ID NO. | Sequence |
|---|---|
| 1374 | KLGFKATLPPFMCNK |
| 1375 | PEKIQKAFDDIAKYF |
| 1376 | CPPGKPTTSEKIHER |
| 1377 | DDAFARRPTVGAQIP |
| 1378 | FGRLQGISPKIMPKK |
| 1379 | PKRGEHAWTHRLRER |
| 1380 | LVIYEEISDPEEDDE |
| 1381 | WEKMKASEKIFYVYM |
| 1382 | AKYFSKEEWEKMKAS |
| 1383 | EWEKMKASEKIFYVY |

TABLE 139
SSX2 HLA-DRB1*1101 Epitope Peptides

| SEQ ID NO. | Sequence |
|---|---|
| 1384 | QKAFDDIAKYFSKEE |
| 1385 | KIFYVYMKRKYEAMT |
| 1386 | KRKYEAMTKLGFKAT |
| 1387 | QMTFGRLQGISPKIM |
| 1388 | EHAWTHRLRERKQLV |
| 1389 | EKIFYVYMKRKYEAM |
| 1390 | IFYVYMKRKYEAMTK |
| 1391 | GISPKIMPKKPAEEG |
| 1392 | LPPFMCNKRAEDFQG |
| 1393 | DDAFARRPTVGAQIP |

TABLE 140
SSX2 HLA-DRB1*1501 (DR2b) Epitope Peptides

| SEQ ID NO. | Sequence |
|---|---|
| 1394 | RKQLVIYEEISDPEE |
| 1395 | FDDIAKYFSKEEWEK |
| 1396 | VYMKRKYEAMTKLGF |
| 1397 | MTKLGFKATLPPFMC |
| 1398 | KATLPPFMCNKRAED |
| 1399 | DDAFARRPTVGAQIP |
| 1400 | QMTFGRLQGISPKIM |
| 1401 | EKIQKAFDDIAKYFS |
| 1402 | YFSKEEWEKMKASEK |
| 1403 | ASEKIFYVYMKRKYE |

PR3 Antigenic Peptides

In some embodiments, the TVM composition includes PR3 (leukocyte proteinase 3) specific T-cells. PR3 specific T-cells can be generated as described below using one or more antigenic peptides to PR3. In some embodiments, the PR3 specific T-cells are generated using one or more antigenic peptides to PR3, or a modified or heteroclitic peptide derived from a PR3 peptide. In some embodiments, PR3 specific T-cells are generated using a PR3 antigen library comprising a pool of peptides (for example 15mers) containing amino acid overlap (for example 11 amino acids of overlap) between each sequence formed by scanning the protein amino acid sequence SEQ. ID. No. 1404 (UniProt KB—P24158) for PR3:

MAHRPPSPALASVLLALLLSGAARAAEIVGGHEAQPHSRPYMASLQMRG
NPGSHFCGGTLIHPSFVLTAAHCLRDIPQRLVNVVLGAHNVRTQEPTQQ
HFSVAQVFLNNYDAENKLNDVLLIQLSSPANLSASVATVQLPQQDQPVP
HGTQCLAMGWGRVGAHDPPAQVLQELNVTVVTFFCRPHNICTFVPRRKA
GICFGDSGGPLICDGIIQGIDSFVIWGCATRLFPDFFTRVALYVDWIRS
TLRRVEAKGRP.

In some embodiments, the PR3 specific T-cells are generated using one or more antigenic peptides to PR3, or a modified or heteroclitic peptide derived from a PR3 peptide. In some embodiments, the PR3 specific T-cells are generated with peptides that recognize class I MHC molecules. In some embodiments, the PR3 specific T-cells are generated with peptides that recognize class II MHC molecules. In some embodiments, the PR3 specific T-cells are generated with peptides that recognize both class I and class II MHC molecules.

In some embodiments, the PR3 peptides used to prime and expand a T-cell subpopulation includes specifically selected HLA-restricted peptides generated by determining the HLA profile of the donor source, and including peptides derived from PR3 that best match the donor's HLA. In some embodiments, the PR3 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting PR3 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 141-147, the HLA-B peptides are selected from the peptides of Tables 148-154, and the HLA-DR peptides are selected from the peptides of Tables 155-160. For example, if the donor cell source has an HLA profile that is HLA-A*01/*02:01; HLA-B*15:01/*18; and HLA-DRB1*0101/*0301, then the PR3 peptides used to prime and expand the PR3 specific T-cell subpopulation are restricted to the specific HLA profile, and may include the peptides identified in Table 141 (Seq. ID. Nos. 1405-1414) for HLA-A*01; Table 142 (Seq. ID. Nos. 1415-1424) for HLA-A*02:01; Table 150 (Seq. ID. Nos. 1495-1504) for HLA-B*15:01; Table 151 (Seq. ID. Nos. 1505-1514) for HLA-B*18; Table 155 (Seq. ID. Nos. 1545-1554) for HLA-DRB1*0101; and Table 156 (Seq. ID. Nos. 1555-1564) for HLA-DRB1*0301. In some embodiments, the mastermix of peptides includes both an overlapping peptide library and specifically selected HLA-restricted peptides generated by determining the HLA profile of the donor source.

In some embodiments, the PR3 HLA-restricted epitopes are specific to at least both of the donor's HLA-A alleles, at least both of the donor's HLA-B alleles, and at least both of the donor's HLA-DR alleles. In some embodiments, the PR3 HLA-A alleles are selected from a group comprising HLA-A*01, HLA-A*02:01, HLA-A*03, HLA-A*11:01, HLA-A*24:02, HLA-A*26, or HLA-A*68:01, and the corresponding PR3 HLA-restricted peptides are selected for: HLA-A*01 from Table 141; HLA-A*02:01 from Table 142; HLA-A*03 from Table 143; HLA-A*11:01 from Table 144; HLA-A*24:02 from Table 145; HLA-A*26 from Table 146; or HLA-A*68:01 from Table 147; or any combination thereof. In some embodiments, the PR3 HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding PR3 HLA-restricted peptides are selected for: HLA-B*07:02 from Table 148; HLA-B*08 from Table 149; HLA-B*15:01 (B62) from Table 150; HLA-B*18 from Table 151; HLA-B*27:05 from Table 152; HLA-B*35:01 from Table 153, or HLA-B*58:02 from Table 154; or any combination thereof. In some embodiments, the PR3 HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding PR3 HLA-restricted peptides are selected for: HLA-DRB1*0101 from Table 155; HLA-DRB1*0301 (DR17) from Table 156; HLA-DRB1*0401 (DR4Dw4) from Table 157; HLA-DRB1*0701 from Table 158; HLA-DRB1*1101 from Table 159; or HLA-DRB1*1501 (DR2b) from Table 160; or any combination thereof.

TABLE 141
Pr3 HLA-A*01 Epitope Peptides

| SEQ ID NO. | Sequence |
|---|---|
| 1405 | FPDFFTRVALY |
| 1406 | GHEAQPHSRPY |
| 1407 | FSVAQVFLNNY |
| 1408 | SVAQVFLNNY |
| 1409 | HEAQPHSRPY |
| 1410 | LRDIPQRLVN |
| 1411 | DFFTRVALY |
| 1412 | EAQPHSRPY |
| 1413 | VAQVFLNNY |
| 1414 | YVDWIRSTLRR |

TABLE 142
Pr3 HLA-A*02:01 Epitope Peptides

| SEQ ID NO. | Sequence |
|---|---|
| 1415 | ALASVLLALL |
| 1416 | KLNDVLLIQL |
| 1417 | VLQELNVTVV |
| 1418 | LICDGIIQGI |
| 1419 | LIHPSFVLTA |
| 1420 | RLFPDFFTRV |
| 1421 | ALYVDWIRST |
| 1422 | NLSASVATV |
| 1423 | LLALLLSGA |
| 1424 | CLAMGWGRV |

TABLE 143
Pr3 HLA-A*03 Epitope Peptides

| SEQ ID NO. | Sequence |
|---|---|
| 1425 | FLNNYDAENK |
| 1426 | TLRRVEAKGR |
| 1427 | QVLQELNVTV |
| 1428 | IVGGHEAQPH |
| 1429 | RLVNVVLGAH |
| 1430 | ALLLSGAAR |
| 1431 | FVIWGCATR |
| 1432 | RLFPDFFTR |
| 1433 | VVLGAHNVR |
| 1434 | ELNVTVVTF |

TABLE 144
Pr3 HLA-A*11:01 Epitope Peptides

| SEQ ID NO. | Sequence |
|---|---|
| 1435 | FVLTAAHCLR |
| 1436 | NVTVVTFFCR |
| 1437 | YVDWIRSTLR |
| 1438 | RVALYVDWIR |
| 1439 | STLRRVEAK |
| 1440 | VVLGAHNVR |
| 1441 | ASVLLALLL |
| 1442 | SVLLALLLS |
| 1443 | VTVVTFFCR |
| 1444 | SVAQVFLNN |

TABLE 145
Pr3 HLA-A*24:02 Epitope Peptides

| SEQ ID NO. | Sequence |
|---|---|
| 1445 | LYVDWIRSTL |
| 1446 | CFGDSGGPLI |
| 1447 | TFVPRRKAGI |
| 1448 | SFVLTAAHCL |
| 1449 | PSPALASVLL |
| 1450 | VIWGCATRLF |
| 1451 | TFFCRPHNI |
| 1452 | CFGDSGGPL |
| 1453 | HFSVAQVFL |
| 1454 | NKLNDVLLI |

TABLE 146
Pr3 HLA-A*26 Epitope Peptides

| SEQ ID NO. | Sequence |
|---|---|
| 1455 | SVAQVFLNNY |
| 1456 | DGIIQGIDSF |
| 1457 | DVLLIQLSSP |
| 1458 | ELNVTVVTFF |
| 1459 | FVIWGCATRL |
| 1460 | DFFTRVALY |
| 1461 | ELNVTVVTF |
| 1462 | DVLLIQLSS |
| 1463 | YVDWIRSTL |
| 1464 | EAQPHSRPY |

TABLE 147
Pr3 HLA-A*68:01 Epitope Peptides

| SEQ ID NO. | Sequence |
|---|---|
| 1465 | DSFVIWGCATR |
| 1466 | LLALLLSGAAR |
| 1467 | GTQCLAMGWGR |
| 1468 | STLRRVEAKGR |
| 1469 | LALLLSGAAR |
| 1470 | YVDWIRSTLR |
| 1471 | NVVLGAHNVR |
| 1472 | NVTVVTFFCR |
| 1473 | RVALYVDWIR |
| 1474 | ATRLFPDFFTR |

TABLE 148
Pr3 HLA-B*07:02 Epitope Peptides

| SEQ ID NO. | Sequence |
|---|---|
| 1475 | FPDFFTRVAL |
| 1476 | PPSPALASVL |
| 1477 | IPQRLVNVVL |
| 1478 | QPVPHGTQCL |
| 1479 | VPHGTQCLAM |
| 1480 | DPPAQVLQEL |
| 1481 | SPALASVLL |
| 1482 | PPAQVLQEL |
| 1483 | AHRPPSPAL |
| 1484 | HPSFVLTAA |

TABLE 149
Pr3 HLA-B*08 Epitope Peptides

| SEQ ID NO. | Sequence |
|---|---|
| 1485 | ENKLNDVLL |
| 1486 | VPRRKAGIC |
| 1487 | CLRDIPQRL |
| 1488 | SPALASVLL |
| 1489 | PQRLVNVVL |
| 1490 | SASVATVQL |
| 1491 | VPRRKAGI |
| 1492 | ENKLNDVL |
| 1493 | VDWIRSTL |
| 1494 | DFFTRVAL |

TABLE 150
Pr3 HLA-B*15:01 (B62) Epitope Peptides

| SEQ ID NO. | Sequence |
|---|---|
| 1495 | QQHFSVAQVF |
| 1496 | SVAQVFLNNY |
| 1497 | ELNVTVVTFF |
| 1498 | RLFPDFFTRV |
| 1499 | RLVNVVLGAH |
| 1500 | KLNDVLLIQL |
| 1501 | ELNVTVVTF |
| 1502 | TQEPTQQHF |
| 1503 | GIIQGIDSF |
| 1504 | ALASVLLAL |

TABLE 151
Pr3 HLA-B*18 Epitope Peptides

| SEQ ID NO. | Sequence |
|---|---|
| 1505 | AENKLNDVL |
| 1506 | DFFTRVALY |
| 1507 | ATRLFPDFF |
| 1508 | GGTLIHPSF |
| 1509 | QEPTQQHF |
| 1510 | LNVTVVTF |
| 1511 | QELNVTVV |
| 1512 | NDVLLIQL |
| 1513 | PRRKAGICF |
| 1514 | CATRLFPDF |

TABLE 152
Pr3 HLA-B*27:05 Epitope Peptides

| SEQ ID NO. | Sequence |
|---|---|
| 1515 | TRLFPDFFTR |
| 1516 | SRPYMASLQM |
| 1517 | VRTQEPTQQH |
| 1518 | ARAAEIVGGH |
| 1519 | CRPHNICTF |
| 1520 | PRRKAGICF |
| 1521 | GIIQGIDSF |
| 1522 | MRGNPGSHF |
| 1523 | LRRVEAKGR |
| 1524 | RRVEAKGRP |

TABLE 153
Pr3 HLA-B*35:01 Epitope Peptides

| SEQ ID NO. | Sequence |
|---|---|
| 1525 | FPDFFTRVAL |
| 1526 | IPQRLVNVVL |
| 1527 | PPSPALASVL |
| 1528 | DPPAQVLQEL |
| 1529 | QPVPHGTQCL |
| 1530 | VPRRKAGICF |
| 1531 | SPALASVLL |
| 1532 | PPAQVLQEL |
| 1533 | GPLICDGII |
| 1534 | RPYMASLQM |

TABLE 154
Pr3 HLA-B*58:02 Epitope Peptides

| SEQ ID NO. | Sequence |
|---|---|
| 1535 | RTQEPTQQHF |
| 1536 | LTAAHCLRDI |
| 1537 | PSPALASVLL |
| 1538 | LSGAARAAEI |
| 1539 | GSHFCGGTLI |
| 1540 | ASVLLALLL |
| 1541 | EAQPHSRPY |
| 1542 | HSRPYMASL |
| 1543 | RVALYVDWI |
| 1544 | CATRLFPDF |

TABLE 155
Pr3 HLA-DRB1*0101 Epitope Peptides

| SEQ ID NO. | Sequence |
|---|---|
| 1545 | AMGWGRVGAHDPPAQ |
| 1546 | SVLLALLLSGAARAA |
| 1547 | NDVLLIQLSSPANLS |
| 1548 | HPSFVLTAAHCLRDI |
| 1549 | DGIIQGIDSFVIWGC |
| 1550 | GICFGDSGGPLICDG |
| 1551 | ASVLLALLLSGAARA |
| 1552 | LLALLLSGAARAAEI |
| 1553 | ARAAEIVGGHEAQPH |
| 1554 | SLQMRGNPGSHFCGG |

TABLE 156
Pr3 HLA-DRB1*0301 (DR17) Epitope Peptides

| SEQ ID NO. | Sequence |
|---|---|
| 1555 | VFLNNYDAENKLNDV |
| 1556 | SPALASVLLALLLSG |
| 1557 | VLLIQLSSPANLSAS |
| 1558 | GGPLICDGIIQGIDS |
| 1559 | AAHCLRDIPQRLVNV |
| 1560 | ATRLFPDFFTRVALY |
| 1561 | SLQMRGNPGSHFCGG |
| 1562 | HFSVAQVFLNNYDAE |
| 1563 | PAQVLQELNVTVVTF |
| 1564 | RVALYVDWIRSTLRR |

TABLE 157
Pr3 HLA-DRB1*0401 (DR4Dw4) Epitope Peptides

| SEQ ID NO. | Sequence |
|---|---|
| 1565 | TRLFPDFFTRVALYV |
| 1566 | SVLLALLLSGAARAA |
| 1567 | AHCLRDIPQRLVNVV |
| 1568 | VNVVLGAHNVRTQEP |
| 1569 | DVLLIQLSSPANLSA |
| 1570 | PAQVLQELNVTVVTF |
| 1571 | VTVVTFFCRPHNICT |
| 1572 | DGIIQGIDSFVIWGC |
| 1573 | QQHFSVAQVFLNNYD |
| 1574 | VTFFCRPHNICTFVP |

TABLE 158
Pr3 HLA-DRB1*0701 Epitope Peptides

| SEQ ID NO. | Sequence |
|---|---|
| 1575 | DGIIQGIDSFVIWGC |
| 1576 | HPSFVLTAAHCLRDI |
| 1577 | GICFGDSGGPLICDG |
| 1578 | YVDWIRSTLRRVEAK |
| 1579 | AHCLRDIPQRLVNVV |
| 1580 | VLLIQLSSPANLSAS |
| 1581 | PAQVLQELNVTVVTF |
| 1582 | LQELNVTVVTFFCRP |
| 1583 | CDGIIQGIDSFVIWG |
| 1584 | SFVIWGCATRLFPDF |

TABLE 159
Pr3 HLA-DRB1*1101 Epitope Peptides

| SEQ ID NO. | Sequence |
|---|---|
| 1585 | HNICTFVPRRKAGIC |
| 1586 | FVIWGCATRLFPDFF |
| 1587 | AMGWGRVGAHDPPAQ |
| 1588 | VVTFFCRPHNICTFV |
| 1589 | DWIRSTLRRVEAKGR |
| 1590 | PDFFTRVALYVDWIR |
| 1591 | ASVLLALLLSGAARA |
| 1592 | SVLLALLLSGAARAA |
| 1593 | SRPYMASLQMRGNPG |
| 1594 | LNNYDAENKLNDVLL |

TABLE 160
Pr3 HLA-DRB1*1501 (DR2b) Epitope Peptides

| SEQ ID NO. | Sequence |
|---|---|
| 1595 | QVFLNNYDAENKLND |
| 1596 | PHNICTFVPRRKAGI |
| 1597 | TRLFPDFFTRVALYV |
| 1598 | VNVVLGAHNVRTQEP |
| 1599 | LIQLSSPANLSASVA |
| 1600 | PAQVLQELNVTVVTF |
| 1601 | NVTVVTFFCRPHNIC |
| 1602 | VTVVTFFCRPHNICT |
| 1603 | DGIIQGIDSFVIWGC |
| 1604 | IQGIDSFVIWGCATR |

Cyclin-A1 Antigenic Peptides

In some embodiments, the TVM composition includes Cyclin-A1 specific T-cells. Cyclin-A1 specific T-cells can be generated as described below using one or more antigenic peptides to Cyclin-A1. In some embodiments, the Cyclin-A1 specific T-cells are generated using one or more antigenic peptides to Cyclin-A1, or a modified or heteroclitic peptide derived from a Cyclin-A1 peptide. In some embodiments, Cyclin-A1 specific T-cells are generated using a Cyclin-A1 antigen library comprising a pool of peptides (for example 15mers) containing amino acid overlap (for example 11 amino acids of overlap) between each sequence formed by scanning the protein amino acid sequence SEQ. ID. No. 1605 (UniProt KB—P78396) for Cyclin-A1:

METGFPAIMYPGSFIGGWGEEYLSWEGPGLPDFVFQQQPVESEAMHCSNP
KSGVVLATVARGPDACQILTRAPLGQDPPQRTVLGLLTANGQYRRTCGQG
ITRIRCYSGSENAFPPAGKKALPDCGVQEPPKQGFDIYMDELEQGDRDSC
SVREGMAFEDVYEVDTGTLKSDLHFLLDFNTVSPMLVDSSLLSQSEDISS
LGTDVINVTEYAEEIYQYLREAEIRHRPKAHYMKKQPDITEGMRTILVDW
LVEVGEEYKLRAETLYLAVNFLDRFLSCMSVLRGKLQLVGTAAMLLASKY
EEIYPPEVDEFVYITDDTYTKRQLLKMEHLLLKVLAFDLTVPTTNQFLLQ
YLRRQGVCVRTENLAKYVAELSLLEADPFLKYLPSLIAAAAFCLANYTVN
KHFWPETLAAFTGYSLSEIVPCLSELHKAYLDIPHRPQQAIREKYKASKY
LCVSLMEPPAVLLLQ.

In some embodiments, the Cyclin-A1 specific T-cells are generated using one or more antigenic peptides to Cyclin-A1, or a modified or heteroclitic peptide derived from a Cyclin-A1 peptide. In some embodiments, the Cyclin-A1 specific T-cells are generated with peptides that recognize class I MHC molecules. In some embodiments, the Cyclin-A1 specific T-cells are generated with peptides that recognize class II MHC molecules. In some embodiments, the Cyclin-A1 specific T-cells are generated with peptides that recognize both class I and class II MHC molecules.

In some embodiments, the Cyclin-A1 peptides used to prime and expand a T-cell subpopulation includes specifically selected HLA-restricted peptides generated by determining the HLA profile of the donor source, and including peptides derived from Cyclin-A1 that best match the donor's HLA. In some embodiments, the Cyclin-A1 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting Cyclin-A1 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 161-167, the HLA-B peptides are selected from the peptides of Tables 168-174, and the HLA-DR peptides are selected from the peptides of Tables 175-180. For example, if the donor cell source has an HLA profile that is HLA-A*01/*02:01; HLA-B*15:01/*18; and HLA-DRB1*0101/*0301, then the Cyclin-A1 peptides used to prime and expand the Cyclin-A1 specific T-cell subpopulation are restricted to the specific HLA profile, and may include the peptides identified in Table 161 (Seq. ID. Nos. 1606-1615) for HLA-A*01; Table 162 (Seq. ID. Nos. 1616-1626) for HLA-A*02:01; Table 170 (Seq. ID. Nos. 1698-1707) for HLA-B*15:01; Table 171 (Seq. ID. Nos. 1708-1717) for HLA-B*18; Table 175 (Seq. ID. Nos. 1747-1756) for HLA-DRB1*0101; and Table 176 (Seq. ID. Nos. 1757-1766) for HLA-DRB1*0301. In some embodiments, the mastermix of peptides includes both an overlapping peptide library and specifically selected HLA-restricted peptides generated by determining the HLA profile of the donor source.

In some embodiments, the Cyclin-A HLA-restricted epitopes are specific to at least both of the donor's HLA-A alleles, at least both of the donor's HLA-B alleles, and at least both of the donor's HLA-DR alleles. In some embodiments, the Cyclin-A HLA-A alleles are selected from a group comprising HLA-A*01, HLA-A*02:01, HLA-A*03, HLA-A*11:01, HLA-A*24:02, HLA-A*26, or HLA-A*68:01, and the corresponding Cyclin-A HLA-restricted peptides are selected for: HLA-A*01 from Table 161; HLA-A*02:01 from Table 162; HLA-A*03 from Table 163; HLA-A*11:01 from Table 164; HLA-A*24:02 from Table 165; HLA-A*26 from Table 166; or HLA-A*68:01 from Table 167; or any combination thereof. In some embodiments, the Cyclin-A HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding Cyclin-A HLA-restricted peptides are selected for: HLA-B*07:02 from Table 168; HLA-B*08 from Table 169; HLA-B*15:01 (B62) from Table 170; HLA-B*18 from Table 171; HLA-B*27:05 from Table 172; HLA-B*35:01 from Table 173, or HLA-B*58:02 from Table 174; or any combination thereof. In some embodiments, the Cyclin-A HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding Cyclin-A HLA-restricted peptides are selected for: HLA-DRB1*0101 from Table 175; HLA-DRB1*0301 (DR17) from Table 176; HLA-DRB1*0401 (DR4Dw4) from Table 177; HLA-DRB1*0701 from Table 178; HLA-DRB1*1101 from Table 179; or HLA-DRB1*1501 (DR2b) from Table 180; or any combination thereof.

TABLE 161
Cyclin A1 HLA-A*01 Epitope Peptides

| SEQ ID NO. | Sequence |
|---|---|
| 1606 | VTEYAEEIYQY |
| 1607 | VREGMAFEDVY |
| 1608 | WPETLAAFTGY |
| 1609 | GTAAMLLASKY |
| 1610 | GTDVINVTEY |
| 1611 | LLEADPFLKY |
| 1612 | PTTNQFLLQY |
| 1613 | IREKYKASKY |
| 1614 | TTNQFLLQY |
| 1615 | PPEVDEFVY |
| 1616 | AAMLLASKY |

TABLE 162
Cyclin A1 HLA-A*02:01 Epitope Peptides

| SEQ ID NO. | Sequence |
|---|---|
| 1617 | TILVDWLVEV |
| 1618 | GMAFEDVYEV |
| 1619 | SLMEPPAVLL |
| 1620 | NLAKYVAEL |
| 1621 | SLSEIVPCL |
| 1622 | VLRGKLQLV |
| 1623 | SLLEADPFL |
| 1624 | SLGTDVINV |
| 1625 | TLYLAVNFL |
| 1626 | KMEHLLLKV |

TABLE 163
Cyclin A1 HLA-A*03 Epitope Peptides

| SEQ ID NO. | Sequence |
|---|---|
| 1627 | AIREKYKASK |
| 1628 | SLLEADPFLK |
| 1629 | IVPCLSELHK |
| 1630 | LLKMEHLLLK |
| 1631 | YLRRQGVCVR |
| 1632 | CVRTENLAK |
| 1633 | CLANYTVNK |
| 1634 | FVYITDDTY |
| 1635 | ALPDCGVQE |
| 1636 | FIGGWGEEY |
| 1637 | SVLRGKLQL |

TABLE 164
Cyclin A1 HLA-A*11:01 Epitope Peptides

| SEQ ID NO. | Sequence |
|---|---|
| 1638 | DTYTKRQLLK |
| 1639 | GTAAMLLASK |
| 1640 | IVPCLSELHK |
| 1641 | SLLEADPFLK |
| 1642 | LLKMEHLLLK |
| 1643 | GVVLATVAR |
| 1644 | CVRTENLAK |
| 1645 | RTCGQGITR |
| 1646 | LVEVGEEYK |
| 1647 | YLAVNFLDR |

TABLE 165
Cyclin A1 HLA-A*24:02 Epitope Peptides

| SEQ ID NO. | Sequence |
|---|---|
| 1648 | EYLSWEGPGL |
| 1649 | EYAEEIYQYL |
| 1650 | PFLKYLPSLI |
| 1651 | IYQYLREAEI |
| 1652 | IYPPEVDEF |
| 1653 | KYVAELSLL |
| 1654 | VYEVDTGTL |
| 1655 | HYMKKQPDI |
| 1656 | EYKLRAETL |
| 1657 | CYSGSENAF |

TABLE 166
Cyclin A1 HLA-A*26 Epitope Peptides

| SEQ ID NO. | Sequence |
|---|---|
| 1658 | DVYEVDTGTL |
| 1659 | ETLYLAVNFL |
| 1660 | EIYPPEVDEF |
| 1661 | DPFLKYLPSL |
| 1662 | EIVPCLSEL |
| 1663 | ETGFPAIMY |
| 1664 | ETLAAFTGY |
| 1665 | EYAEEIYQY |
| 1666 | DTGTLKSDL |
| 1667 | EVDEFVYIT |

TABLE 167
Cyclin A1 HLA-A*68:01 Epitope Peptides

| SEQ ID NO. | Sequence |
|---|---|
| 1668 | DVYEVDTGTLK |
| 1669 | DIPHRPQQAIR |
| 1670 | DTYTKRQLLK |
| 1671 | TAAMLLASK |
| 1672 | ITDDTYTKR |
| 1673 | GVVLATVAR |
| 1674 | EVGEEYKLR |
| 1675 | RTCGQGITR |
| 1676 | DACQILTRA |
| 1677 | KAYLDIPHR |

TABLE 168
Cyclin A1 HLA-B*07:02 Epitope Peptides

| SEQ ID NO. | Sequence |
|---|---|
| 1678 | DPPQRTVLGL |
| 1679 | SPMLVDSSLL |
| 1680 | DPFLKYLPSL |
| 1681 | NPKSGVVLAT |
| 1682 | LPSLIAAAAF |
| 1683 | PPQRTVLGL |
| 1684 | FPPAGKKAL |
| 1685 | GPGLPDFVF |
| 1686 | IPHRPQQAI |
| 1687 | VPTTNQFLL |

TABLE 169
Cyclin A1 HLA-B*08 Epitope Peptides

| SEQ ID NO. | Sequence |
|---|---|
| 1688 | EYKLRAETL |
| 1689 | LLKVLAFDL |
| 1690 | TLKSDLHFL |
| 1691 | LLKMEHLLL |
| 1692 | HLLLKVLAF |
| 1693 | VLRGKLQL |
| 1694 | FLKYLPSL |
| 1695 | NPKSGVVL |
| 1696 | PPAGKKAL |
| 1697 | LAKYVAEL |

TABLE 170
Cyclin A1 HLA-B*15:01 (B62) Epitope Peptides

| SEQ ID NO. | Sequence |
|---|---|
| 1698 | CVRTENLAKY |
| 1699 | GQGITRIRCY |
| 1700 | LLASKYEEIY |
| 1701 | LLEADPFLKY |
| 1702 | WLVEVGEEY |
| 1703 | PQQAIREKY |
| 1704 | CLSELHKAY |
| 1705 | GLLTANGQY |
| 1706 | HLLLKVLAF |
| 1707 | VQEPPKQGF |

TABLE 171
Cyclin A1 HLA-B*18 Epitope Peptides

| SEQ ID NO. | Sequence |
|---|---|
| 1708 | MEHLLLKVL |
| 1709 | WEGPGLPDF |
| 1710 | LEADPFLKY |
| 1711 | REKYKASKY |
| 1712 | PEVDEFVY |
| 1713 | YEVDTGTL |
| 1714 | QEPPKQGF |
| 1715 | TEYAEEIY |
| 1716 | AEEIYQYL |
| 1717 | TEGMRTIL |

TABLE 172
Cyclin A1 HLA-B*27:05 Epitope Peptides

| SEQ ID NO. | Sequence |
|---|---|
| 1718 | RRTCGQGITR |
| 1719 | DRFLSCMSVL |
| 1720 | HRPQQAIREK |
| 1721 | HRPKAHYMKK |
| 1722 | IREKYKASKY |
| 1723 | KRQLLKMEHL |
| 1724 | LRRQGVCVR |
| 1725 | IREKYKASK |
| 1726 | KRQLLKMEH |
| 1727 | LREAEIRHR |

TABLE 173
Cyclin A1 HLA-B*35:01 Epitope Peptides

| SEQ ID NO. | Sequence |
|---|---|
| 1728 | PPQRTVLGLL |
| 1729 | EPPKQGFDIY |
| 1730 | SPMLVDSSLL |
| 1731 | DPFLKYLPSL |
| 1732 | LPSLIAAAAF |
| 1733 | FPPAGKKAL |
| 1734 | DPPQRTVLGL |
| 1735 | RPQQAIREKY |
| 1736 | FPAIMYPGSF |

TABLE 174
Cyclin A1 HLA-B*58:02 Epitope Peptides

| SEQ ID NO. | Sequence |
|---|---|
| 1737 | KSDLHFLLDF |
| 1738 | KASKYLCVSL |
| 1739 | RTCGQGITRI |
| 1740 | LAVNFLDRFL |
| 1741 | YSLSEIVPCL |
| 1742 | CSNPKSGVVL |
| 1743 | SSLLSQSEDI |
| 1744 | VSLMEPPAVL |
| 1745 | LSLLEADPF |
| 1746 | PSLIAAAAF |

TABLE 175
Cyclin A1 HLA-DRB1*0101 Epitope Peptides

| SEQ ID NO. | Sequence |
|---|---|
| 1747 | VAELSLLEADPFLKY |
| 1748 | EHLLLKVLAFDLTVP |
| 1749 | LKYLPSLIAAAAFCL |
| 1750 | LLDFNTVSPMLVDSS |
| 1751 | SEDISSLGTDVINVT |
| 1752 | DPFLKYLPSLIAAAA |
| 1753 | NGQYRRTCGQGITRI |
| 1754 | RGKLQLVGTAAMLLA |
| 1755 | ASKYEEIYPPEVDEF |
| 1756 | YLCVSLMEPPAVLLL |

TABLE 176
Cyclin A1 HLA-DRB1*0301 (DR17) Epitope Peptides

| SEQ ID NO. | Sequence |
|---|---|
| 1757 | TGTLKSDLHFLLDFN |
| 1758 | LSLLEADPFLKYLPS |
| 1759 | DCGVQEPPKQGFDIY |
| 1760 | DLHFLLDFNTVSPML |
| 1761 | EAEIRHRPKAHYMKK |
| 1762 | VINVTEYAEEIYQYL |
| 1763 | QPDITEGMRTILVDW |
| 1764 | DWLVEVGEEYKLRAE |
| 1765 | VPCLSELHKAYLDIP |
| 1766 | GSFIGGWGEEYLSWE |

TABLE 177
Cyclin A1 HLA-DRB1*0401 (DR4Dw4) Epitope Peptides

| SEQ ID NO. | Sequence |
|---|---|
| 1767 | DLHFLLDFNTVSPML |
| 1768 | EFVYITDDTYTKRQL |
| 1769 | HKAYLDIPHRPQQAI |
| 1770 | FEDVYEVDTGTLKSD |
| 1771 | TGTLKSDLHFLLDFN |
| 1772 | VSPMLVDSSLLSQSE |
| 1773 | SEDISSLGTDVINVT |
| 1774 | YLAVNFLDRFLSCMS |
| 1775 | VNFLDRFLSCMSVLR |
| 1776 | RGKLQLVGTAAMLLA |

TABLE 178
Cyclin A1 HLA-DRB1*0701 Epitope Peptides

| SEQ ID NO. | Sequence |
|---|---|
| 1777 | VDEFVYITDDTYTKR |
| 1778 | NGQYRRTCGQGITRI |
| 1779 | ENAFPPAGKKALPDC |
| 1780 | FEDVYEVDTGTLKSD |
| 1781 | FLLDFNTVSPMLVDS |
| 1782 | SEDISSLGTDVINVT |
| 1783 | EEIYQYLREAEIRHR |
| 1784 | RGKLQLVGTAAMLLA |
| 1785 | KLQLVGTAAMLLASK |
| 1786 | ASKYEEIYPPEVDEF |

TABLE 179
Cyclin A1 HLA-DRB1*1101 Epitope Peptides

| SEQ ID NO. | Sequence |
|---|---|
| 1787 | YLAVNFLDRFLSCMS |
| 1788 | LANYTVNKHFWPETL |
| 1789 | HKAYLDIPHRPQQAI |
| 1790 | ETGFPAIMYPGSFIG |
| 1791 | LVEVGEEYKLRAETL |
| 1792 | ASKYLCVSLMEPPAV |
| 1793 | KRQLLKMEHLLLKVL |
| 1794 | LKMEHLLLKVLAFDL |
| 1795 | NQFLLQYLRRQGVCV |
| 1796 | CVRTENLAKYVAELS |

TABLE 180
Cyclin A1 HLA-DRB1*1501 (DR2b) Epitope Peptides

| SEQ ID NO. | Sequence |
|---|---|
| 1797 | YLAVNFLDRFLSCMS |
| 1798 | LANYTVNKHFWPETL |
| 1799 | HKAYLDIPHRPQQAI |
| 1800 | ETGFPAIMYPGSFIG |
| 1801 | LVEVGEEYKLRAETL |
| 1802 | ASKYLCVSLMEPPAV |
| 1803 | KRQLLKMEHLLLKVL |
| 1804 | LKMEHLLLKVLAFDL |
| 1805 | NQFLLQYLRRQGVCV |
| 1806 | CVRTENLAKYVAELS |

Neutrophil Elastase Antigenic Peptides

In some embodiments, the TVM composition includes neutrophil elastase specific T-cells. neutrophil elastase specific T-cells can be generated as described below using one or more antigenic peptides to neutrophil elastase. In some embodiments, the neutrophil elastase specific T-cells are generated using one or more antigenic peptides to neutrophil elastase, or a modified or heteroclitic peptide derived from a neutrophil elastase peptide. In some embodiments, neutrophil elastase specific T-cells are generated using a neutrophil elastase antigen library comprising a pool of peptides (for example 15mers) containing amino acid overlap (for example 11 amino acids of overlap) between each sequence formed by scanning the protein amino acid sequence SEQ. ID. No. 1807 (UniProt KB—P08246) for neutrophil elastase:

MTLGRRLACLFLACVLPALLLGGTALASEIVGGRRARPHAWPFMVSLQLR
GGHFCGATLIAPNFVMSAAHCVANVNVRAVRVVLGAHNLSRREPTRQVFA
VQRIFENGYDPVNLLNDIVILQLNGSATINANVQVAQLPAQGRRLGNGVQ
CLAMGWGLLGRNRGIASVLQELNVTVVTSLCRRSNVCTLVRGRQAGVCFG
DSGSPLVCNGLIFIGIASFVRGGCASGLYPDAFAPVAQFVNWIDSIIQRS
EDNPCPHPRDPDPASRTH.

In some embodiments, the neutrophil elastase specific T-cells are generated using one or more antigenic peptides to neutrophil elastase, or a modified or heteroclitic peptide derived from a neutrophil elastase peptide. In some embodiments, the neutrophil elastase specific T-cells are generated with peptides that recognize class I MHC molecules. In some embodiments, the neutrophil elastase specific T-cells are generated with peptides that recognize class II MHC molecules. In some embodiments, the neutrophil elastase specific T-cells are generated with peptides that recognize both class I and class II MHC molecules.

In some embodiments, the neutrophil elastase peptides used to prime and expand a T-cell subpopulation includes specifically selected HLA-restricted peptides generated by determining the HLA profile of the donor source, and including peptides derived from neutrophil elastase that best match the donor's HLA. In some embodiments, the neutrophil elastase peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting neutrophil elastase derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 181-187, the HLA-B peptides are selected from the peptides of Tables 188-194, and the HLA-DR peptides are selected from the peptides of Tables 195-200. For example, if the donor cell source has an HLA profile that is HLA-A*01/*02:01; HLA-B*15:01/*18; and HLA-DRB1*0101/*0301, then the neutrophil elastase peptides used to prime and expand the neutrophil elastase specific T-cell subpopulation are restricted to the specific HLA profile, and may include the peptides identified in Table 181 (Seq. ID. Nos. 1808-1817) for HLA-A*01; Table 182 (Seq. ID. Nos. 1818-1827) for HLA-A*02:01; Table 190 (Seq. ID. Nos. 1989-1907) for HLA-B*15:01; Table 191 (Seq. ID. Nos. 1908-1917) for HLA-B*18; Table 195 (Seq. ID. Nos. 1948-1957) for HLA-DRB1*0101; and Table 196 (Seq. ID. Nos. 1958-1967) for HLA-DRB1*0301. In some embodiments, the mastermix of peptides includes both an overlapping peptide library and specifically selected HLA-restricted peptides generated by determining the HLA profile of the donor source.

In some embodiments, the neutrophil elastase HLA-restricted epitopes are specific to at least both of the donor's HLA-A alleles, at least both of the donor's HLA-B alleles, and at least both of the donor's HLA-DR alleles. In some embodiments, the neutrophil elastase HLA-A alleles are selected from a group comprising HLA-A*01, HLA-A*02:01, HLA-A*03, HLA-A*11:01, HLA-A*24:02, HLA-A*26, or HLA-A*68:01, and the corresponding neutrophil elastase HLA-restricted peptides are selected for: HLA-A*01 from Table 181; HLA-A*02:01 from Table 182; HLA-A*03 from Table 183; HLA-A*11:01 from Table 184; HLA-A*24:02 from Table 185; HLA-A*26 from Table 186; or HLA-A*68:01 from Table 187; or any combination thereof. In some embodiments, the neutrophil elastase HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding neutrophil elastase HLA-restricted peptides are selected for: HLA-B*07:02 from Table 188; HLA-B*08 from Table 189; HLA-B*15:01 (B62) from Table 190; HLA-B*18 from Table 191; HLA-B*27:05 from Table 192; HLA-B*35:01 from Table 193, or HLA-B*58:02 from Table 194; or any combination thereof. In some embodiments, the neutrophil elastase HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding neutrophil elastase HLA-restricted peptides are selected for: HLA-DRB1*0101 from Table 195; HLA-DRB1*0301 (DR17) from Table 196; HLA-DRB1*0401 (DR4Dw4) from Table 197; HLA-DRB1*0701 from Table 198; HLA-DRB1*1101 from Table 199; or HLA-DRB1*1501 (DR2b) from Table 200; or any combination thereof.

TABLE 181
Neutrophil Elastase HLA-A*01 Epitope Peptides

| SEQ ID NO. | Sequence |
|---|---|
| 1808 | FVRGGCASGLY |
| 1809 | FAVQRIFENGY |
| 1810 | AVQRIFENGY |
| 1811 | GYDPVNLLND |
| 1812 | VRGGCASGLY |
| 1813 | ASEIVGGRRA |
| 1814 | FGDSGSPLVC |
| 1815 | VQRIFENGY |
| 1816 | RGGCASGLY |
| 1817 | LNDIVILQL |

TABLE 182
Neutrophil Elastase HLA-A*02:01 Epitope Peptides

| SEQ ID NO. | Sequence |
|---|---|
| 1818 | LLNDIVILQL |
| 1819 | RLACLFLACV |
| 1820 | GLYPDAFAPV |
| 1821 | FLACVLPAL |
| 1822 | NLLNDIVIL |
| 1823 | VLQELNVTV |
| 1824 | ALLLGGTAL |
| 1825 | GIASVLQEL |
| 1826 | TLGRRLACL |
| 1827 | LLLGGTALA |

TABLE 183
Neutrophil Elastase HLA-A*03 Epitope Peptides

| SEQ ID NO. | Sequence |
|---|---|
| 1828 | VVLGAHNLSR |
| 1829 | LVRGRQAGVC |
| 1830 | AVQRIFENGY |
| 1831 | NVRAVRVVL |
| 1832 | GLIHGIASF |
| 1833 | ALLLGGTAL |
| 1834 | AVRVVLGAH |
| 1835 | NLSRREPTR |
| 1836 | SLQLRGGHF |
| 1837 | FVRGGCASG |

TABLE 184
Neutrophil Elastase HLA-A*11:01 Epitope Peptides

| SEQ ID NO. | Sequence |
|---|---|
| 1838 | VVLGAHNLSR |
| 1839 | PTRQVFAVQR |
| 1840 | NVTVVTSLCR |
| 1841 | ALASEIVGGR |
| 1842 | QVAQLPAQGR |
| 1843 | RSNVCTLVR |
| 1844 | VTVVTSLCR |
| 1845 | ASEIVGGRR |
| 1846 | AMGWGLLGR |
| 1847 | NVCTLVRGR |

TABLE 185
Neutrophil Elastase HLA-A*24:02 Epitope Peptides

| SEQ ID NO. | Sequence |
|---|---|
| 1848 | LFLACVLPAL |
| 1849 | VSLQLRGGHF |
| 1850 | VNLLNDIVIL |
| 1851 | QCLAMGWGLL |
| 1852 | GSPLVCNGLI |
| 1853 | CFGDSGSPL |
| 1854 | QFVNWIDSI |
| 1855 | NGYDPVNLL |
| 1856 | GYDPVNLLN |
| 1857 | LYPDAFAPV |

TABLE 186
Neutrophil Elastase HLA-A*26 Epitope Peptides

| SEQ ID NO. | Sequence |
|---|---|
| 1858 | ELNVTVVTSL |
| 1859 | ENGYDPVNLL |
| 1860 | MTLGRRLACL |
| 1861 | AVQRIFENGY |
| 1862 | DIVILQLNGS |
| 1863 | FVRGGCASGL |
| 1864 | DAFAPVAQF |
| 1865 | RVVLGAHNL |
| 1866 | QVFAVQRIF |
| 1867 | EIVGGRRAR |

TABLE 187
Neutrophil Elastase HLA-A*68:01 Epitope Peptides

| SEQ ID NO. | Sequence |
|---|---|
| 1868 | CVANVNVRAVR |
| 1869 | FVNWIDSIIQR |
| 1870 | QVAQLPAQGRR |
| 1871 | PTRQVFAVQR |
| 1872 | VANVNVRAVR |
| 1873 | TVVTSLCRR |
| 1874 | LASEIVGGR |
| 1875 | NVCTLVRGR |
| 1876 | NWIDSIIQR |
| 1877 | EIVGGRRAR |

TABLE 188
Neutrophil Elastase HLA-B*07:02 Epitope Peptides

| SEQ ID NO. | Sequence |
|---|---|
| 1878 | APVAQFVNWI |
| 1879 | CPHPRDPDPA |
| 1880 | LPALLLGGTA |
| 1881 | WPFMVSLQL |
| 1882 | YPDAFAPVA |
| 1883 | APNFVMSAA |
| 1884 | EPTRQVFAV |
| 1885 | RPHAWPFMV |
| 1886 | ACVLPALLL |
| 1887 | NVRAVRVVL |

TABLE 189
Neutrophil Elastase HLA-B*08 Epitope Peptides

| SEQ ID NO. | Sequence |
|---|---|
| 1888 | TLGRRLACL |
| 1889 | SLQLRGGHF |
| 1890 | GLLGRNRGI |
| 1891 | GRRLACLFL |
| 1892 | FLACVLPAL |
| 1893 | ALLLGGTAL |
| 1894 | NLLNDIVIL |
| 1895 | VRAVRVVL |
| 1896 | SPLVCNGL |
| 1897 | CLFLACVL |

TABLE 190
Neutrophil Elastase HLA-B*15:01 (B62) Epitope
Peptides

| SEQ ID NO. | Sequence |
|---|---|
| 1898 | RQVFAVQRIF |
| 1899 | TLGRRLACLF |
| 1900 | AVQRIFENGY |
| 1901 | ALLLGGTALA |
| 1902 | ILQLNGSATI |
| 1903 | RLGNGVQCLA |
| 1904 | VQRIFENGY |
| 1905 | GLIHGIASF |
| 1906 | SLQLRGGHF |
| 1907 | QVFAVQRIF |

TABLE 191
Neutrophil Elastase HLA-B*18 Epitope Peptides

| SEQ ID NO. | Sequence |
|---|---|
| 1908 | DAFAPVAQF |
| 1909 | LGRRLACLF |
| 1910 | NVRAVRVVL |
| 1911 | ASGLYPDAF |
| 1912 | ACLFLACVL |
| 1913 | GATLIAPNF |
| 1914 | REPTRQVF |
| 1915 | QELNVTVV |
| 1916 | ANVQVAQL |
| 1917 | ACVLPALL |

TABLE 192
Neutrophil Elastase HLA-B*27:05 Epitope Peptides

| SEQ ID NO. | Sequence |
|---|---|
| 1918 | RRLGNGVQCL |
| 1919 | RRARPHAWPF |
| 1920 | GRNRGIASVL |
| 1921 | RRSNVCTLVR |
| 1922 | VRVVLGAHNL |
| 1923 | RREPTRQVF |
| 1924 | GRRLACLFL |
| 1925 | VRGGCASGL |
| 1926 | TRQVFAVQR |
| 1927 | PRDPDPASR |

TABLE 193
Neutrophil Elastase HLA-B*35:01 Epitope Peptides

| SEQ ID NO. | Sequence |
|---|---|
| 1928 | APVAQFVNWI |
| 1929 | LFLACVLPAL |
| 1930 | VNVRAVRVVL |
| 1931 | YPDAFAPVAQ |
| 1932 | WPFMVSLQL |
| 1933 | DPVNLLNDI |
| 1934 | SPLVCNGLI |
| 1935 | ACLFLACVL |
| 1936 | NLLNDIVIL |
| 1937 | RNRGIASVL |

TABLE 194
Neutrophil Elastase HLA-B*58:02 Epitope Peptides

| SEQ ID NO. | Sequence |
|---|---|
| 1938 | RARPHAWPFM |
| 1939 | VSLQLRGGHF |
| 1940 | GSPLVCNGLI |
| 1941 | LACVLPALLL |
| 1942 | RVVLGAHNL |
| 1943 | QVFAVQRIF |
| 1944 | GATLIAPNF |
| 1945 | ASGLYPDAF |
| 1946 | GATLIAPNF |
| 1947 | ASGLYPDAF |

TABLE 195
Neutrophil Elastase HLA-DRB1*0101 Epitope Peptides

| SEQ ID NO. | Sequence |
|---|---|
| 1948 | PHAWPFMVSLQLRGG |
| 1949 | LNDIVILQLNGSATI |
| 1950 | CLFLACVLPALLLGG |
| 1951 | APNFVMSAAHCVANV |
| 1952 | ASFVRGGCASGLYPD |
| 1953 | VLPALLLGGTALASE |
| 1954 | ALASEIVGGRRARPH |
| 1955 | RAVRVVLGAHNLSRR |
| 1956 | YDPVNLLNDIVILQL |
| 1957 | NVQVAQLPAQGRRLG |

TABLE 196
Neutrophil Elastase HLA-DRB1*0301 (DR17) Epitope
Peptides

| SEQ ID NO. | Sequence |
|---|---|
| 1958 | VFAVQRIFENGYDPV |
| 1959 | PVNLLNDIVILQLNG |
| 1960 | ASGLYPDAFAPVAQF |
| 1961 | CLFLACVLPALLLGG |
| 1962 | IFENGYDPVNLLNDI |
| 1963 | IASVLQELNVTVVTS |
| 1964 | ACLFLACVLPALLLG |
| 1965 | VRVVLGAHNLSRREP |
| 1966 | VNLLNDIVILQLNGS |
| 1967 | NDIVILQLNGSATIN |

TABLE 197
Neutrophil Elastase HLA-DRB1*0401 (DR4Dw4)
Epitope Peptides

| SEQ ID NO. | Sequence |
|---|---|
| 1968 | PDAFAPVAQFVNWID |
| 1969 | PNFVMSAAHCVANVN |
| 1970 | NRGIASVLQELNVTV |
| 1971 | IASVLQELNVTVVTS |
| 1972 | VTVVTSLCRRSNVCT |
| 1973 | RSNVCTLVRGRQAGV |
| 1974 | AQFVNWIDSIIQRSE |
| 1975 | PHAWPFMVSLQLRGG |
| 1976 | AMGWGLLGRNRGIAS |
| 1977 | GVCFGDSGSPLVCNG |

TABLE 198
Neutrophil Elastase HLA-DRB1*0701 Epitope Peptides

| SEQ ID NO. | Sequence |
|---|---|
| 1978 | GVCFGDSGSPLVCNG |
| 1979 | PHAWPFMVSLQLRGG |
| 1980 | VFAVQRIFENGYDPV |
| 1981 | ILQLNGSATINANVQ |
| 1982 | APNFVMSAAHCVANV |
| 1983 | IVILQLNGSATINAN |
| 1984 | LQELNVTVVTSLCRR |
| 1985 | VAQFVNWIDSIIQRS |
| 1986 | YDPVNLLNDIVILQL |
| 1987 | VNLLNDIVILQLNGS |

TABLE 199
Neutrophil Elastase HLA-DRB1*1101 Epitope Peptides

| SEQ ID NO. | Sequence |
|---|---|
| 1988 | AMGWGLLGRNRGIAS |
| 1989 | RSNVCTLVRGRQAGV |
| 1990 | VTVVTSLCRRSNVCT |
| 1991 | ASEIVGGRRARPHAW |
| 1992 | VAQLPAQGRRLGNGV |
| 1993 | PNFVMSAAHCVANVN |
| 1994 | AVRVVLGAHNLSRRE |
| 1995 | VLGAHNLSRREPTRQ |
| 1996 | TRQVFAVQRIFENGY |
| 1997 | GWGLLGRNRGIASVL |

TABLE 200
Neutrophil Elastase HLA-DRB1*1501(DR2b) Epitope
Peptides

| SEQ ID NO. | Sequence |
|---|---|
| 1998 | PALLLGGTALASEIV |
| 1999 | VRVVLGAHNLSRREP |
| 2000 | RIFENGYDPVNLLND |
| 2001 | ILQLNGSATINANVQ |
| 2002 | VQCLAMGWGLLGRNR |
| 2003 | WGLLGRNRGIASVLQ |
| 2004 | IASVLQELNVTVVTS |
| 2005 | ELNVTVVTSLCRRSN |
| 2006 | VTSLCRRSNVCTLVR |
| 2007 | NGLIHGIASFVRGGC |

### Generation of Targeted Virus-Associated Antigen Peptides for Use in Activating T-Cell Subpopulations

T-cell subpopulations targeting one or multiple VAAs can be prepared by pulsing antigen presenting cells or artificial antigen presenting cells with a selected single peptide or epitope, several peptides or epitopes, or with peptide libraries of the selected viral-associated antigens, that for example, include peptides that are about 7, 8, 9, 10, 11, 12, 13, 14, 15, 16 or more amino acids long and overlapping one another by 5, 6, 7, 8, or 9 amino acids, in certain aspects. GMP-quality overlapping peptide libraries directed to a number of viral-associated antigens are commercially available, for example, through JPT Technologies and/or Miltenyi Biotec. In particular embodiments, the peptides are 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and there is overlap of 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length.

The VAA-targeting T-cell component of the TVM or VM can be prepared by using a multi-VAA priming and expanding approach wherein the T-cells are primed with a mastermix of one or more antigenic peptides from two or more VAAs. Alternatively, the VAA targeting T cell component of the TVM or VM can be prepared by separately priming and expanding a T-cell subpopulation to each targeted VAA, and then combining the separately primed and activated T-cell subpopulations.

In some embodiments, the T-cell subpopulation is specific to one or more known epitopes of multiple VAA. Much work has been done to determine specific epitopes of VAAs and the HLA alleles they are associated with. Non-limiting examples of specific epitopes of VAAs and the alleles they are associated with can be found in Kuzushima et al., Blood (2003) 101:1460-1468; Kondo et al., Blood (2004) 103(2): 630-638; Hanley et al., Blood (2009) 114(9): 1958-1967; and Hanley et al., Cytotherapy (2011) 13: 976-986, which are each incorporated herein by reference.

In some embodiments, the VAA peptides used to prime and expand a T-cell subpopulation includes specifically selected HLA-restricted peptides generated by determining the HLA profile of the donor source, and including peptides derived from the targeted VAA that best match the donor's HLA type. By including specifically selected donor HLA-restricted peptides in the peptide mix for priming and expanding T-cell subpopulations, a T-cell subpopulation can be generated that provides greater VAA targeted activity through more than one donor HLA, improving potential efficacy of the T-cell subpopulation. In addition, by generating a T-cell subpopulation with VAA targeted activity through more than one donor HLA allele, a single donor T-cell subpopulation may be included in a TVM or VM composition for multiple recipients with different HLA profiles by matching one or more donor HLAs showing VAA-activity. In some embodiments, the VAA peptides used to prime and expand a T-cell subpopulation are derived from HLA-restricted peptides selected from at least one or more of an HLA-A restricted peptide, HLA-B restricted peptide, or HLA-DR restricted peptide. In some embodiments, the HLA-restricted epitopes are specific to at least one or more of a cell donor's HLA-A alleles, HLA-B alleles, or HLA-DR alleles. In some embodiments, the HLA-restricted epitopes are specific to at least both of the donor's HLA-A alleles, at least both of the donor's HLA-B alleles, and at least both of the donor's HLA-DR alleles. In some embodiments, the HLA-A alleles are selected from a group comprising HLA-A*01, HLA-A*02:01, HLA-A*03, HLA-A*11:01, HLA-A*24:02, HLA-A*26, or HLA-A*68:01. In some embodiments, the HLA-B alleles are selected from a group comprising HLA-B*07:02, HLA-B*08, HLA-B*15:01 (B62), HLA-B*18, HLA-B*27:05, HLA-B*35:01, or HLA-B*58:02. In some embodiments, the HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b). Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595. In some embodiments, the mastermix of peptides includes both an overlapping peptide library and specifically selected HLA-restricted peptides generated by determining the HLA profile of the donor source.

This focused approach to activation can increase the effectiveness of the activated T-cell subpopulation, and ultimately, the TVM or VM composition

Epstein-Barr Virus (EBV) Strain B95-8 LMP1 Antigenic Peptides

In some embodiments, the TVM or VM composition includes Epstein-Barr Virus (EBV) Strain B95-8 LMP1 specific T-cells. LMP1 specific T-cells can be generated as described below using one or more antigenic peptides to LMP1. In some embodiments, the LMP1 specific T-cells are generated using one or more antigenic peptides to LMP1, or a modified or heteroclitic peptide derived from a LMP1 peptide. In some embodiments, LMP1 specific T-cells are generated using a LMP1 antigen library comprising a pool of peptides (for example 15mers) containing amino acid overlap (for example 11 amino acids of overlap) between each sequence formed by scanning the protein amino acid sequence SEQ. ID. No. 2008 (UniProt KB—P03230) for EBV Strain B95-8 LMP1:

MEHDLERGPPGPRRPPRGPPLSSSLGLALLLLLLALLFWLYIVMSDWTG
GALLVLYSFALMLIIIILIIFIFRRDLLCPLGALCILLLMITLLLIALW
NLHGQALFLGIVLFIFGCLLVLGIWIYLLEMLWRLGATIWQLLAFFLAF
FLDLILLIIALYLQQNWWTLLVDLLWLLLFLAILIWMYYHGQRHSDEHH
HDDSLPHPQQATDDSGHESDSNSNEGRHHLLVSGAGDGPPLCSQNLGAP
GGGPDNGPQDPDNTDDNGPQDPDNTDDNGPHDPLPQDPDNTDDNGPQDP
DNTDDNGPHDPLPHSPSDSAGNDGGPPQLTEEVENKGGDQGPPLMTDGG
GGHSHDSGHGGGDPHLPTLLLGSSGSGGDDDDPHGPVQLSYYD.

In some embodiments, the LMP1 specific T-cells are generated using one or more antigenic peptides to LMP1, or a modified or heteroclitic peptide derived from a LMP1 peptide. In some embodiments, the LMP1 specific T-cells are generated with peptides that recognize class I MHC molecules. In some embodiments, the LMP1 specific T-cells are generated with peptides that recognize class II MHC molecules. In some embodiments, the LMP1 specific T-cells are generated with peptides that recognize both class I and class II MHC molecules.

In some embodiments, the LMP1 peptides used to prime and expand a T-cell subpopulation includes specifically selected HLA-restricted peptides generated by determining the HLA profile of the donor source, and including peptides derived from LMP1 that best match the donor's HLA. In some embodiments, the LMP1 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting LMP1 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 201-207, the HLA-B peptides are selected from the peptides of Tables 208-214, and the HLA-DR peptides are selected from the peptides of Tables 215-220. For example, if the donor cell source has an HLA profile that is HLA-A*01/*02:01; HLA-B*15:01/*18; and HLA-DRB1*0101/*0301, then the LMP1 peptides used to prime and expand the LMP1 specific T-cell subpopulation are restricted to the specific HLA profile, and may include the peptides identified in Table 201 (Seq. ID. Nos. 2009-2013) for HLA-A*01; Table 202 (Seq. ID. Nos. 2014-2018) for HLA-A*02:01; Table 210 (Seq. ID. Nos. 2054-2058) for HLA-B*15:01; Table 211 (Seq. ID. Nos. 2059-2063) for HLA-B*18; Table 215 (Seq. ID. Nos. 2079-2083) for HLA-DRB1*0101; and Table 216 (Seq. ID. Nos. 2084-2088) for HLA-DRB1*0301. In some embodiments, the mastermix of peptides includes both an overlapping peptide library and specifically selected HLA-restricted peptides generated by determining the HLA profile of the donor source.

In some embodiments, the LMP1 HLA-restricted epitopes are specific to at least both of the donor's HLA-A alleles, at least both of the donor's HLA-B alleles, and at least both of the donor's HLA-DR alleles. In some embodiments, the LMP1 HLA-A alleles are selected from a group comprising HLA-A*01, HLA-A*02:01, HLA-A*03, HLA-A*11:01, HLA-A*24:02, HLA-A*26, or HLA-A*68:01, and the corresponding LMP1 HLA-restricted peptides are selected for: HLA-A*01 from Table 201; HLA-A*02:01 from Table 202; HLA-A*03 from Table 203; HLA-A*11:01 from Table 204; HLA-A*24:02 from Table 205; HLA-A*26 from Table 206; or HLA-A*68:01 from Table 207; or any combination thereof. In some embodiments, the LMP1 HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding LMP1 HLA-restricted peptides are selected for: HLA-B*07:02 from Table 208; HLA-B*08 from Table 209; HLA-B*15:01 (B62) from Table 210; HLA-B*18 from Table 211; HLA-B*27:05 from Table 212; HLA-B*35:01 from Table 213, or HLA-B*58:02 from Table 214; or any combination thereof. In some embodiments, the LMP1 HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding LMP1 HLA-restricted peptides are selected for: HLA-DRB1*0101 from Table 215; HLA-DRB1*0301 (DR17) from Table 216; HLA-DRB1*0401 (DR4Dw4) from Table 217; HLA-DRB1*0701 from Table 218; HLA-DRB1*1101 from Table 219; or HLA-DRB1*1501 (DR2b) from Table 220; or any combination thereof.

TABLE 201
EBV Strain B95-8 LMP1 HLA-A*01 Epitope Peptides

| SEQ ID NO. | Sequence |
|---|---|
| 2009 | LLALLFWLY |
| 2010 | WTGGALLVLY |
| 2011 | LLLLALLFWLY |
| 2012 | MSDWTGGALLV |
| 2013 | DWTGGALLVLY |

TABLE 202
EBV Strain B95-8 LMP1 HLA-A*02:01 Epitope Peptides

| SEQ ID NO. | Sequence |
|---|---|
| 2014 | ALLLLLLAL |
| 2015 | LLLLLLALL |
| 2016 | YLLEMLWRL |
| 2017 | GLALLLLLL |
| 2018 | LLLALLFWL |

TABLE 203
EBV Strain B95-8 LMP1 HLA-A*03 Epitope Peptides

| SEQ ID NO. | Sequence |
|---|---|
| 2019 | ALFLGIVLF |
| 2020 | QLLAFFLAF |
| 2021 | LLLLLALLF |
| 2022 | MLWRLGATI |
| 2023 | QLILEVENK |

TABLE 204
EBV Strain B95-8 LMP1 HLA-A*11:01 Epitope Peptides

| SEQ ID NO. | Sequence |
|---|---|
| 2024 | SSLGLALLL |
| 2025 | IILIIFIFR |
| 2026 | SSSLGLALLL |
| 2027 | IIILIIFIFR |
| 2028 | ESDSNSNEGR |

TABLE 205
EBV Strain B95-8 LMP1 HLA-A*24:02 Epitope Peptides

| SEQ ID NO. | Sequence |
|---|---|
| 2029 | LYSFALMLI |
| 2030 | FFLDLILLI |
| 2031 | IFIFRRDLL |
| 2032 | IYLLEMLWRL |
| 2033 | LYLQQNWWTL |

TABLE 206
EBV Strain B95-8 LMP1 HLA-A*26 Epitope Peptides

| SEQ ID NO. | Sequence |
|---|---|
| 2034 | DLILLIIAL |
| 2035 | ATIWQLLAF |
| 2036 | LIIIILIIF |
| 2037 | EVENKGGDQ |
| 2038 | LVDLLWLLLF |

TABLE 207
EBV Strain B95-8 LMP1 HLA-A*68:01 Epitope Peptides

| SEQ ID NO. | Sequence |
|---|---|
| 2039 | IIIILIIFIFR |
| 2040 | IILIIFIFR |
| 2041 | IIILIIFIFR |
| 2042 | ILIIFIFRR |
| 2043 | DLERGPPGPR |

TABLE 208
EBV Strain B95-8 LMP1 HLA-B*07:02 Epitope Peptides

| SEQ ID NO. | Sequence |
|---|---|
| 2044 | DPHLPTLLL |
| 2045 | PPLSSSLGL |
| 2046 | GPPLCSQNL |
| 2047 | GPPLSSSLGL |
| 2048 | CPLGALCILL |

TABLE 209
EBV Strain B95-8 LMP1 HLA-B*08 Epitope Peptides

| SEQ ID NO. | Sequence |
|---|---|
| 2049 | FIFRRDLL |
| 2050 | SNEGRHHLL |
| 2051 | SLGLALLLL |
| 2052 | ILLLMITLL |
| 2053 | DLILLIIAL |

TABLE 210
EBV Strain B95-8 LMP1 HLA-B*15:01 (B62) Epitope
Peptides

| SEQ ID NO. | Sequence |
|---|---|
| 2054 | ALFLGIVLF |
| 2055 | CLLVLGIWIY |
| 2056 | LLLALLFWLY |
| 2057 | MLIIIILIIF |
| 2058 | DLILLIIALY |

TABLE 211
EBV Strain B95-8 LMP1 HLA-B*18 Epitope Peptides

| SEQ ID NO. | Sequence |
|---|---|
| 2059 | DEHHHDDSL |
| 2060 | DLILLIIAL |
| 2061 | NEGRHHLL |
| 2062 | DLLWLLLF |
| 2063 | EEVENKGG |

TABLE 212
EBV Strain B95-8 LMP1 HLA-B*27:05
Epitope Peptides

| SEQ ID NO. | Sequence |
|---|---|
| 2064 | WRLGATIWQL |
| 2065 | PRGPPLSSSL |
| 2066 | RRPPRGPPL |
| 2067 | ERGPPGPRR |
| 2068 | FRRDLLCPL |

TABLE 213
EBV Strain B95-8 LMP1 HLA-B*35:01
Epitope Peptides

| SEQ ID NO. | Sequence |
|---|---|
| 2069 | PPLSSSLGL |
| 2070 | DPHLPTLLL |
| 2071 | GPPLCSQNL |
| 2072 | CPLGALCILL |
| 2073 | DPHGPVQLSY |

TABLE 214
EBV Strain B95-8 LMP1 HLA-B*58:02
Epitope Peptides

| SEQ ID NO. | Sequence |
|---|---|
| 2074 | SSLGLALLL |
| 2075 | ITLLLIALW |
| 2076 | LSSSLGLAL |
| 2077 | SSSLGLALLL |
| 2078 | NSNEGRHHLL |

TABLE 215
EBV Strain B95-8 LMP1 HLA-DRB1*0101
Epitope Peptides

| SEQ ID NO. | Sequence |
|---|---|
| 2079 | LALLLLLLALLFWLY |
| 2080 | RDLLCPLGALCILLL |
| 2081 | LIALWNLHGQALFLG |
| 2082 | GATIWQLLAFFLAFF |
| 2083 | LGIVLFIFGCLLVLG |

TABLE 216
EBV Strain B95-8 LMP1 HLA-DRB1*0301
(DR17) Epitope Peptides

| SEQ ID NO. | Sequence |
|---|---|
| 2084 | WWTLLVDLLWLLLFL |
| 2085 | IFIFRRDLLCPLGAL |
| 2086 | IILIIFIFRRDLLCP |
| 2087 | ILIIFIFRRDLLCPL |
| 2088 | GLALLLLLLALLFWL |

TABLE 217
EBV Strain B95-8 LMP1 HLA-DRB1*0401
(DR4Dw4) Epitope Peptides

| SEQ ID NO. | Sequence |
|---|---|
| 2089 | LFWLYIVMSDWTGGA |
| 2090 | LYIVMSDWTGGALLV |
| 2091 | GGALLVLYSFALMLI |
| 2092 | IILIIFIFRRDLLCP |
| 2093 | ILIIFIFRRDLLCPL |

TABLE 218
EBV Strain B95-8 LMP1 HLA-DRB1*0701
Epitope Peptides

| SEQ ID NO. | Sequence |
|---|---|
| 2094 | HDPLPHSPSDSAGND |
| 2095 | GGALLVLYSFALMLI |
| 2096 | LVLYSFALMLIIIIL |
| 2097 | LCILLLMITLLLIAL |
| 2098 | LWRLGATIWQLLAFF |

TABLE 219
EBV Strain B95-8 LMP1 HLA-DRB1*1101
Epitope Peptides

| SEQ ID NO. | Sequence |
|---|---|
| 2099 | IYLLEMLWRLGATIW |
| 2100 | FWLYIVMSDWTGGAL |
| 2101 | ATIWQLLAFFLAFFL |
| 2102 | IILIIFIFRRDLLCP |
| 2103 | QNWWTLLVDLLWLLL |

TABLE 220
EBV Strain B95-8 LMP1 HLA-DRB1*1501
(DR2b) Epitope Peptides

| SEQ ID NO. | Sequence |
|---|---|
| 2104 | WQLLAFFLAFFLDLI |
| 2105 | LLWLLLFLAILIWMY |
| 2106 | LLALLFWLYIVMSDW |
| 2107 | LLVLYSFALMLIIII |
| 2108 | GIVLFIFGCLLVLGI |

Epstein-Barr Virus (EBV) Strain B95-8 LMP2 Antigenic Peptides

In some embodiments, the TVM or VM composition includes Epstein-Barr Virus (EBV) Strain B95-8 LMP2 specific T-cells. LMP2 specific T-cells can be generated as described below using one or more antigenic peptides to LMP2. In some embodiments, the LMP2 specific T-cells are generated using one or more antigenic peptides to LMP2, or a modified or heteroclitic peptide derived from a LMP2 peptide. In some embodiments, LMP2 specific T-cells are generated using a LMP2 antigen library comprising a pool of peptides (for example 15mers) containing amino acid overlap (for example 11 amino acids of overlap) between each sequence formed by scanning the protein amino acid sequence SEQ. ID. No. 2109 (UniProt KB—P13285) for EBV Strain B95-8 LMP2:

MGSLEMVPMGAGPPSPGGDPDGYDGGNNSQYPSASGSSGNTPTPPNDEER
ESNEEPPPPYEDPYWGNGDRHSDYQPLGTQDQSLYLGLQHDGNDGLPPPP
YSPRDDSSQHIYEEAGRGSMNPVCLPVIVAPYLFWLAAIAASCETASVST
VVTATGLALSLLLLAAVASSYAAAQRKLLTPVTVLTAVVTFFAICLTWRI
EDPPENSLLFALLAAAGGLQGIYVLVMLVLLILAYRRRWRRLTVCGGIMF
LACVLVLIVDAVLQLSPLLGAVTVVSMTLLLLAFVLWLSSPGGLGTLGAA
LLTLAAALALLASLILGTLNLTTMELLMLLWTLVVLLICSSCSSCPLSKI
LLARLFLYALALLLLASALIAGGSILQTNEKSLSSTEFIPNLFCMLLLIV
AGILFILAILTEWGSGNRTYGPVFMCLGGLLTMVAGAVWLTVMSNTLLSA
WILTAGFLIFLIGFALFGVIRCCRYCCYYCLTLESEERPPTPYRNTV.

In some embodiments, the LMP2 specific T-cells are generated using one or more antigenic peptides to LMP2, or a modified or heteroclitic peptide derived from a LMP2 peptide. In some embodiments, the LMP2 specific T-cells are generated with peptides that recognize class I MHC molecules. In some embodiments, the LMP2 specific T-cells are generated with peptides that recognize class II MHC molecules. In some embodiments, the LMP2 specific T-cells are generated with peptides that recognize both class I and class II MHC molecules.

In some embodiments, the LMP2 peptides used to prime and expand a T-cell subpopulation includes specifically selected HLA-restricted peptides generated by determining the HLA profile of the donor source, and including peptides derived from LMP2 that best match the donor's HLA. In some embodiments, the LMP2 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting LMP2 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 221-227, the HLA-B peptides are selected from the peptides of Tables 228-234, and the HLA-DR peptides are selected from the peptides of Tables 235-240. For example, if the donor cell source has an HLA profile that is HLA-A*01/*02:01; HLA-B*15:01/*18; and HLA-DRB1*0101/*0301, then the LMP2 peptides used to prime and expand the LMP2 specific T-cell subpopulation are restricted to the specific HLA profile, and may include the peptides identified in Table 221 (Seq. ID. Nos. 2009-2013) for HLA-A*01; Table 222 (Seq. ID. Nos. 2115-2119) for HLA-A*02:01; Table 230 (Seq. ID. Nos. 2155-2159) for HLA-B*15:01; Table 231 (Seq. ID. Nos. 2160-2164) for HLA-B*18; Table 235 (Seq. ID. Nos. 2180-2184) for HLA-DRB1*0101; and Table 236 (Seq. ID. Nos. 2185-2189) for HLA-DRB1*0301. In some embodiments, the mastermix of peptides includes both an overlapping peptide library and specifically selected HLA-restricted peptides generated by determining the HLA profile of the donor source.

In some embodiments, the LMP2 HLA-restricted epitopes are specific to at least both of the donor's HLA-A alleles, at least both of the donor's HLA-B alleles, and at least both of the donor's HLA-DR alleles. In some embodiments, the LMP2 HLA-A alleles are selected from a group comprising HLA-A*01, HLA-A*02:01, HLA-A*03, HLA-A*11:01, HLA-A*24:02, HLA-A*26, or HLA-A*68:01, and the corresponding LMP2 HLA-restricted peptides are selected for: HLA-A*01 from Table 221; HLA-A*02:01 from Table 222; HLA-A*03 from Table 223; HLA-A*11:01 from Table 224; HLA-A*24:02 from Table 225; HLA-A*26 from Table 226; or HLA-A*68:01 from Table 227; or any combination thereof. In some embodiments, the LMP2 HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding LMP2 HLA-restricted peptides are selected for: HLA-B*07:02 from Table 228; HLA-B*08 from Table 229; HLA-B*15:01 (B62) from Table 230; HLA-B*18 from Table 231; HLA-B*27:05 from Table 232; HLA-B*35:01 from Table 233, or HLA-B*58:02 from Table 234; or any combination thereof. In some embodiments, the LMP2 HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding LMP2 HLA-restricted peptides are selected for: HLA-DRB1*0101 from Table 235; HLA-DRB1*0301 (DR17) from Table 236; HLA-DRB1*0401 (DR4Dw4) from Table 237; HLA-DRB1*0701 from Table 238; HLA-DRB1*1101 from Table 239; or HLA-DRB1*1501 (DR2b) from Table 240; or any combination thereof.

TABLE 221
EBV Strain B95-8 LMP2 HLA-A*01
Epitope Peptides

| SEQ ID NO. | Sequence |
|---|---|
| 2110 | RDDSSQHIY |
| 2111 | ESEERPPTPY |
| 2112 | GYDGGNNSQY |
| 2113 | GNDGLPPPPY |
| 2114 | LTEWGSGNRTY |

TABLE 222
EBV Strain B95-8 LMP2 HLA-A*02:01
Epitope Peptides

| SEQ ID NO. | Sequence |
|---|---|
| 2115 | LLLAFVLWL |
| 2116 | FLLMLLWTL |
| 2117 | LLASLILGTL |
| 2118 | LLARLFLYAL |
| 2119 | FLIGFALFGV |

TABLE 223
EBV Strain B95-8 LMP2 HLA-A*03
Epitope Peptides

| SEQ ID NO. | Sequence |
|---|---|
| 2120 | LLAAVASSY |
| 2121 | ALIAGGSIL |
| 2122 | SLLLLAAVA |
| 2123 | LLLAAVASSY |
| 2124 | QLSPLLGAVT |

TABLE 224
EBV Strain B95-8 LMP2 HLA-A*11:01
Epitope Peptides

| SEQ ID NO. | Sequence |
|---|---|
| 2125 | SSYAAAQRK |
| 2126 | GSILQTNFK |
| 2127 | SSCSSCPLSK |
| 2128 | ASSYAAAQRK |
| 2129 | AVLQLSPLLG |

TABLE 225
EBV Strain B95-8 LMP2 HLA-A*24:02
Epitope Peptides

| SEQ ID NO. | Sequence |
|---|---|
| 2130 | TYGPVFMCL |
| 2131 | PYLFWLAAI |
| 2132 | SYAAAQRKLL |
| 2133 | IYVLVMLVLL |
| 2134 | MFLACVLVLI |

TABLE 226
EBV Strain B95-8 LMP2 HLA-A*26
Epitope Peptides

| SEQ ID NO. | Sequence |
|---|---|
| 2135 | PVFMCLGGL |
| 2136 | DAVLQLSPL |
| 2137 | TVVSMTLLLL |
| 2138 | TVVTATGLAL |
| 2139 | VTVLTAVVTF |

TABLE 227
EBV Strain B95-8 LMP2 HLA-A*68:01
Epitope Peptides

| SEQ ID NO. | Sequence |
|---|---|
| 2140 | AVASSYAAAQR |
| 2141 | VTFFAICLTWR |
| 2142 | LVLLILAYR |
| 2143 | PLSKILLAR |
| 2144 | VASSYAAAQR |

TABLE 228
EBV Strain B95-8 LMP2 HLA-B*07:02
Epitope Peptides

| SEQ ID NO. | Sequence |
|---|---|
| 2145 | LPVIVAPYL |
| 2146 | APYLFWLAA |
| 2147 | IPNLFCMLLL |
| 2148 | QPLGTQDQSL |
| 2149 | SPGGLGTLGA |

TABLE 229
EBV Strain B95-8 LMP2 HLA-B*08
Epitope Peptides

| SEQ ID NO. | Sequence |
|---|---|
| 2150 | CPLSKILL |
| 2151 | ILLARLFL |
| 2152 | AAAQRKLL |
| 2153 | AYRRRWRRL |
| 2154 | LARLFLYAL |

TABLE 230
EBV Strain B95-8 LMP2 HLA-B*15:01
(B62) Epitope Peptides

| SEQ ID NO. | Sequence |
|---|---|
| 2155 | MLVLLILAY |
| 2156 | CLPVIVAPY |
| 2157 | LLAAVASSY |
| 2158 | LLLAAVASSY |
| 2159 | RLTVCGGIMF |

TABLE 231
EBV Strain B95-8 LMP2 HLA-B*18
Epitope Peptides

| SEQ ID NO. | Sequence |
|---|---|
| 2160 | SEERPPTPY |
| 2161 | IEDPPFNSL |
| 2162 | EERPPTPY |
| 2163 | TEFIPNLF |
| 2164 | NEEPPPPY |

TABLE 232
EBV Strain B95-8 LMP2 HLA-B*27:05
Epitope Peptides

| SEQ ID NO. | Sequence |
|---|---|
| 2165 | ARLFLYALAL |
| 2166 | GRGSMNPVCL |
| 2167 | RRLTVCGGIM |
| 2168 | GGLQGIYVL |
| 2169 | CRYCCYYCL |

TABLE 233
EBV Strain B95-8 LMP2 HLA-B*35:01
Epitope Peptides

| SEQ ID NO. | Sequence |
|---|---|
| 2170 | LPVIVAPYL |
| 2171 | SPGGDPDGY |
| 2172 | QPLGTQDQSL |
| 2173 | PPFNSLLFAL |
| 2174 | GPVFMCLGGL |

TABLE 234
EBV Strain B95-8 LMP2 HLA-B*58:02
Epitope Peptides

| SEQ ID NO. | Sequence |
|---|---|
| 2175 | KSLSSTEFI |
| 2176 | SSCPLSKIL |
| 2177 | LSKILLARLF |
| 2178 | SSYAAAQRKL |
| 2179 | LSSPGGLGTL |

TABLE 235
EBV Strain B95-8 LMP2 HLA-DRB1*0101
Epitope Peptides

| SEQ ID NO. | Sequence |
|---|---|
| 2180 | QTNFKSLSSTEFIPN |
| 2181 | ALSLLLLAAVASSYA |
| 2182 | PGGLGTLGAALLTLA |
| 2183 | CMLLLIVAGILFILA |
| 2184 | AGFLIFLIGFALFGV |

TABLE 236
EBV Strain B95-8 LMP2 HLA-DRB1*0301
(DR17) Epitope Peptides

| SEQ ID NO. | Sequence |
|---|---|
| 2185 | YLGLQHDGNDGLPPP |
| 2186 | VLVLIVDAVLQLSPL |
| 2187 | AVWLTVMSNTLLSAW |
| 2188 | YQPLGTQDQSLYLGL |
| 2189 | VLVMLVLLILAYRRR |

TABLE 237
EBV Strain B95-8 LMP2 HLA-DRB1*0401
(DR4Dw4) Epitope Peptides

| SEQ ID NO. | Sequence |
|---|---|
| 2190 | HSDYQPLGTQDQSLY |
| 2191 | QSLYLGLQHDGNDGL |
| 2192 | QHIYEEAGRGSMNPV |
| 2193 | ASSYAAAQRKLLTPV |
| 2194 | GAVWLTVMSNTLLSA |

TABLE 238
EBV Strain B95-8 LMP2 HLA-DRB1*0701
Epitope Peptides

| SEQ ID NO. | Sequence |
|---|---|
| 2195 | ASCFTASVSTVVTAT |
| 2196 | ACVLVLIVDAVLQLS |
| 2197 | VTFFAICLTWRIEDP |
| 2198 | GAVWLTVMSNTLLSA |
| 2199 | LSAWILTAGFLIFLI |

TABLE 239
EBV Strain B95-8 LMP2 HLA-DRB1*1101
Epitope Peptides

| SEQ ID NO. | Sequence |
|---|---|
| 2200 | QSLYLGLQHDGNDGL |
| 2201 | LTEWGSGNRTYGPVF |
| 2202 | PNLFCMLLLIVAGIL |
| 2203 | NRTYGPVFMCLGGLL |
| 2204 | VTVLTAVVTFFAICL |

TABLE 240
EBV Strain B95-8 LMP2 HLA-DRB1*1501
(DR2b) Epitope Peptides

| SEQ ID NO. | Sequence |
|---|---|
| 2205 | LTAVVTFFAICLTWR |
| 2206 | FLLMLLWTLVVLLIC |
| 2207 | LIFLIGFALFGVIRC |
| 2208 | LAILTEWGSGNRTYG |
| 2209 | LIGFALFGVIRCCRY |

Epstein-Barr Virus (EBV) Strain B95-8 EBNA1 Antigenic Peptides

In some embodiments, the TVM or VM composition includes Epstein-Barr Virus (EBV) Strain B95-8 EBNA1 specific T-cells. EBNA1 specific T-cells can be generated as described below using one or more antigenic peptides to EBNA1. In some embodiments, the EBNA1 specific T-cells are generated using one or more antigenic peptides to EBNA1, or a modified or heteroclitic peptide derived from a EBNA1 peptide. In some embodiments, EBNA1 specific T-cells are generated using a EBNA1 antigen library comprising a pool of peptides (for example 15mers) containing amino acid overlap (for example 11 amino acids of overlap) between each sequence formed by scanning the protein amino acid sequence SEQ. ID. No. 2210 (UniProt KB—P03211) for EBV Strain B95-8 EBNA1:

MSDEGPGTGPGNGLGEKGDTSGPEGSGGSGPQRRGGDNHGRGRGRGRGRG
GGRPGAPGGSGSGPRHRDGVRRPQKRPSCIGCKGTHGGTGAGAGAGGAGA
GGAGAGGGAGAGGGAGGAGGAGGAGAGGGAGAGGGAGGAGGAGAGGGAGA
GGGAGGAGAGGGAGGAGGAGAGGGAGAGGGAGGAGAGGGAGGAGGAGAGG
GAGAGGAGGAGGAGAGGAGAGGGAGGAGGAGAGGAGAGGAGAGGAGAGGA
GGAGAGGAGGAGAGGAGGAGAGGGAGGAGAGGGAGGAGAGGAGGAGAGGA
GGAGAGGAGGAGAGGGAGAGGAGAGGGGRGRGGSGGRGRGGSGGRGRGGS
GGRRGRGRERARGGSRERARGRGRGRGEKRPRSPSSQSSSSGSPPRRPPP
GRRPFFHPVGEADYFEYHQEGGPDGEPDVPPGAIEQGPADDPGEGPSTGP
RGQGDGGRRKKGGWFGKHRGQGGSNPKFENIAEGLRALLARSHVERTTDE
GTWVAGVFVYGGSKTSLYNLRRGTALAIPQCRLTPLSRLPFGMAPGPGPQ
PGPLRESIVCYFMVFLQTHIFAEVLKDAIKDLVMTKPAPTCNIRVTVCSF
DDGVDLPPWFPPMVEGAAAEGDDGDDGDEGGDGDEGEEGQE.

In some embodiments, the EBNA1 specific T-cells are generated using one or more antigenic peptides to EBNA1, or a modified or heteroclitic peptide derived from a EBNA1 peptide. In some embodiments, the EBNA1 specific T-cells are generated with peptides that recognize class I MHC molecules. In some embodiments, the EBNA1 specific T-cells are generated with peptides that recognize class II MHC molecules. In some embodiments, the EBNA1 specific T-cells are generated with peptides that recognize both class I and class II MHC molecules.

In some embodiments, the EBNA1 peptides used to prime and expand a T-cell subpopulation includes specifically selected HLA-restricted peptides generated by determining the HLA profile of the donor source, and including peptides derived from EBNA1 that best match the donor's HLA. In some embodiments, the EBNA1 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting EBNA1 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 241-247, the HLA-B peptides are selected from the peptides of Tables 248-254, and the HLA-DR peptides are selected from the peptides of Tables 255-260. For example, if the donor cell source has an HLA profile that is HLA-A*01/*02:01; HLA-B*15:01/*18; and HLA-DRB1*0101/*0301, then the EBNA1 peptides used to prime and expand the EBNA1 specific T-cell subpopulation are restricted to the specific HLA profile, and may include the peptides identified in Table 241 (Seq. ID. Nos. 2211-2215) for HLA-A*01; Table 242 (Seq. ID. Nos. 2216-2220) for HLA-A*02:01; Table 250 (Seq. ID. Nos. 2256-2260) for HLA-B*15:01; Table 251 (Seq. ID. Nos. 2261-2265) for HLA-B*18; Table 255 (Seq. ID. Nos. 2281-2285) for HLA-DRB1*0101; and Table 256 (Seq. ID. Nos. 2286-2290) for HLA-DRB1*0301. In some embodiments, the mastermix of peptides includes both an overlapping peptide library and specifically selected HLA-restricted peptides generated by determining the HLA profile of the donor source.

In some embodiments, the EBNA1 HLA-restricted epitopes are specific to at least both of the donor's HLA-A alleles, at least both of the donor's HLA-B alleles, and at least both of the donor's HLA-DR alleles. In some embodiments, the EBNA1 HLA-A alleles are selected from a group comprising HLA-A*01, HLA-A*02:01, HLA-A*03, HLA-A*11:01, HLA-A*24:02, HLA-A*26, or HLA-A*68:01, and the corresponding EBNA1 HLA-restricted peptides are selected for: HLA-A*01 from Table 241; HLA-A*02:01 from Table 242; HLA-A*03 from Table 243; HLA-A*11:01 from Table 244; HLA-A*24:02 from Table 245; HLA-A*26 from Table 246; or HLA-A*68:01 from Table 247; or any combination thereof. In some embodiments, the EBNA1 HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding EBNA1 HLA-restricted peptides are selected for: HLA-B*07:02 from Table 248; HLA-B*08 from Table 249; HLA-B*15:01 (B62) from Table 250; HLA-B*18 from Table 251; HLA-B*27:05 from Table 252; HLA-B*35:01 from Table 253, or HLA-B*58:02 from Table 254; or any combination thereof. In some embodiments, the EBNA1 HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding EBNA1 HLA-restricted peptides are selected for: HLA-DRB1*0101 from Table 255; HLA-DRB1*0301 (DR17) from Table 256; HLA-DRB1*0401 (DR4Dw4) from Table 257; HLA-DRB1*0701 from Table 258; HLA-DRB1*1101 from Table 259; or HLA-DRB1*1501 (DR2b) from Table 260; or any combination thereof.

TABLE 241
EBV Strain B95-8 EBNA1 HLA-A*01 Epitope Peptides

| SEQ ID NO. | Sequence |
|---|---|
| 2211 | VGEADYFEY |
| 2212 | TTDEGTWVA |
| 2213 | TWVAGVFVY |
| 2214 | GTWVAGVFVY |
| 2215 | FVYGGSKTSLY |

TABLE 242
EBV Strain B95-8 EBNA1 HLA-A*02:01 Epitope Peptides

| SEQ ID NO. | Sequence |
|---|---|
| 2216 | NIAEGLRAL |
| 2217 | ALAIPQCRL |
| 2218 | VLKDAIKDL |
| 2219 | FLQTHIFAEV |
| 2220 | AIPQCRLTPL |

TABLE 243
EBV Strain B95-8 EBNA1 HLA-A*03 Epitope Peptides

| SEQ ID NO. | Sequence |
|---|---|
| 2221 | AIKDLVMTK |
| 2222 | GVFVYGGSK |
| 2223 | ALLARSHVER |
| 2224 | RLTPLSRLPF |
| 2225 | GLRALLARSH |

TABLE 244
EBV Strain B95-8 EBNA1 HLA-A*11:01 Epitope Peptides

| SEQ ID NO. | Sequence |
|---|---|
| 2226 | GVFVYGGSK |
| 2227 | GSGSGPRHR |
| 2228 | QTHIFAEVLK |
| 2229 | ALLARSHVER |
| 2230 | GSKTSLYNLR |

TABLE 245
EBV Strain B95-8 EBNA1 HLA-A*24:02 Epitope Peptides

| SEQ ID NO. | Sequence |
|---|---|
| 2231 | VYGGSKTSL |
| 2232 | LYNLRRGTAL |
| 2233 | KFENIAEGL |
| 2234 | IFAEVLKDAI |
| 2235 | YFMVFLQTHI |

TABLE 246
EBV Strain B95-8 EBNA1 HLA-A*26 Epitope Peptides

| SEQ ID NO. | Sequence |
|---|---|
| 2236 | MVFLQTHIF |
| 2237 | DGVDLPPWF |
| 2238 | EVLKDAIKDL |
| 2239 | ENIAEGLRAL |
| 2240 | DVPPGAIEQG |

TABLE 247
EBV Strain B95-8 EBNA1 HLA-A*68:01 Epitope Peptides

| SEQ ID NO. | Sequence |
|---|---|
| 2241 | RALLARSHVER |
| 2242 | GTALAIPQCR |
| 2243 | QTHIFAEVLK |
| 2244 | DAIKDLVMTK |
| 2245 | MTKPAPTCNIR |

TABLE 248
EBV Strain B95-8 EBNA1 HLA-B*07:02 Epitope Peptides

| SEQ ID NO. | Sequence |
|---|---|
| 2246 | IPQCRLTPL |
| 2247 | GPGPQPGPL |
| 2248 | EPDVPPGAI |
| 2249 | GPGTGPGNGL |
| 2250 | RPPPGRRPFF |

TABLE 249
EBV Strain B95-8 EBNA1 HLA-B*08 Epitope Peptides

| SEQ ID NO. | Sequence |
|---|---|
| 2251 | NLRRGTAL |
| 2252 | GPRHRDGV |
| 2253 | VLKDAIKDL |
| 2254 | IPQCRLTPL |
| 2255 | GRRKKGGWF |

TABLE 250
EBV Strain B95-8 EBNA1 HLA-B*15:01 (B62)
Epitope Peptides

| SEQ ID NO. | Sequence |
|---|---|
| 2256 | PLRESIVCY |
| 2257 | RLTPLSRLPF |
| 2258 | GQGGSNPKF |
| 2259 | PVGEADYFEY |
| 2260 | MVFLQTHIF |

TABLE 251
EBV Strain B95-8 EBNA1 HLA-B*18 Epitope Peptides

| SEQ ID NO. | Sequence |
|---|---|
| 2261 | RESIVCYF |
| 2262 | GEADYFEY |
| 2263 | FENIAEGL |
| 2264 | AEGLRALL |
| 2265 | DGVDLPPWF |

TABLE 252
EBV Strain B95-8 EBNA1 HLA-B*27:05 Epitope Peptides

| SEQ ID NO. | Sequence |
|---|---|
| 2266 | RRKKGGWFGK |
| 2267 | GRGGSGGRGR |
| 2268 | GRGGSGGRR |
| 2269 | RRGGDNHGR |
| 2270 | CRLTPLSRL |

TABLE 253
EBV Strain B95-8 EBNA1 HLA-B*35:01 Epitope Peptides

| SEQ ID NO. | Sequence |
|---|---|
| 2271 | HPVGEADYF |
| 2272 | IPQCRLTPL |
| 2273 | GPGPQPGPL |
| 2274 | GPLRESIVCY |
| 2275 | GPGTGPGNGL |

TABLE 254
EBV Strain B95-8 EBNA1 HLA-B*58:02 Epitope Peptides

| SEQ ID NO. | Sequence |
|---|---|
| 2276 | GSNPKFENI |
| 2277 | GSKTSLYNL |
| 2278 | IAEGLRALL |
| 2279 | ESIVCYFMVF |
| 2280 | MTKPAPTCNI |

TABLE 255
EBV Strain B95-8 EBNA1 HLA-DRB1*0101
Epitope Peptides

| SEQ ID NO. | Sequence |
|---|---|
| 2281 | CYFMVFLQTHIFAEV |
| 2282 | TSLYNLRRGTALAIP |
| 2283 | RLPFGMAPGPGPQPG |
| 2284 | AEGLRALLARSHVER |
| 2285 | AGVFVYGGSKTSLYN |

TABLE 256
EBV Strain B95-8 EBNA1 HLA-DRB1*0301
(DR17) Epitope Peptides

| SEQ ID NO. | Sequence |
|---|---|
| 2286 | FAEVLKDAIKDLVMT |
| 2287 | FENIAEGLRALLARS |
| 2288 | QCRLTPLSRLPFGMA |
| 2289 | RPFFHPVGEADYFEY |
| 2290 | GVFVYGGSKTSLYNL |

TABLE 257
EBV Strain B95-8 EBNA1 HLA-DRB1*0401
(DR4Dw4) Epitope Peptides

| SEQ ID NO. | Sequence |
|---|---|
| 2291 | VCYFMVFLQTHIFAE |
| 2292 | KTSLYNLRRGTALAI |
| 2293 | AGVFVYGGSKTSLYN |
| 2294 | THIFAEVLKDAIKDL |
| 2295 | FENIAEGLRALLARS |

TABLE 258
EBV Strain B95-8 EBNA1 HLA-DRB1*0701
Epitope Peptides

| SEQ ID NO. | Sequence |
|---|---|
| 2296 | VFVYGGSKTSLYNLR |
| 2297 | RPFFHPVGEADYFEY |
| 2298 | NPKFENIAEGLRALL |
| 2299 | RSHVERTTDEGTWVA |
| 2300 | CYFMVFLQTHIFAEV |

TABLE 259
EBV Strain B95-8 EBNA1 HLA-DRB1*1101
Epitope Peptides

| SEQ ID NO. | Sequence |
|---|---|
| 2301 | KTSLYNLRRGTALAI |
| 2302 | KGGWFGKHRGQGGSN |
| 2303 | THIFAEVLKDAIKDL |
| 2304 | PPWFPPMVEGAAAEG |
| 2305 | QCRLTPLSRLPFGMA |

TABLE 260
EBV Strain B95-8 EBNA1 HLA-DRB1*1501
(DR2b) Epitope Peptides

| SEQ ID NO. | Sequence |
|---|---|
| 2306 | RESIVCYFMVFLQTH |
| 2307 | ESIVCYFMVFLQTHI |
| 2308 | VAGVFVYGGSKTSLY |
| 2309 | GVDLPPWFPPMVEGA |
| 2310 | LYNLRRGTALAIPQC |

Epstein-Barr Virus (EBV) Strain B95-8 EBNA2 Antigenic Peptides

In some embodiments, the TVM or VM composition includes Epstein-Barr Virus (EBV) Strain B95-8 EBNA2 specific T-cells. EBNA2 specific T-cells can be generated as described below using one or more antigenic peptides to EBNA2. In some embodiments, the EBNA2 specific T-cells are generated using one or more antigenic peptides to EBNA2, or a modified or heteroclitic peptide derived from a EBNA2 peptide. In some embodiments, EBNA2 specific T-cells are generated using a EBNA2 antigen library comprising a pool of peptides (for example 15mers) containing amino acid overlap (for example 11 amino acids of overlap) between each sequence formed by scanning the protein amino acid sequence SEQ. ID. No. 2311 (UniProt KB—P03211) for EBV Strain B95-8 EBNA2:

MPTFYLALHGGQTYHLIVDTDSLGNPSLSVIPSNPYQEQLSDTPLIPLTI
FVGENTGVPPPLPPPPPPPPPPPPPPPPPPPPPPPPPPSPPPPPPPPPPP
QRRDAWTQEPSPLDRDPLGYDVGHGPLASAMRMLWMANYIVRQSRGDRGL
ILPQGPQTAPQARLVQPHVPPLRPTAPTILSPLSQPRLTPPQPLMMPPRP
TPPTPLPPATLTVPPRPTRPTTLPPTPLLTVLQRPTELQPTPSPPRMHLP
VLHVPDQSMHPLTHQSTPNDPDSPEPRSPTVFYNIPPMPLPPSQLPPPAA
PAQPPPGVINDQQLHHLPSGPPWWPPICDPPQPSKTQGQSRGQSRGRGRG
RGRGRGKGKSRDKQRKPGGPWRPEPNTSSPSMPELSPVLGLHQGQGAGDS
PTPGPSNAAPVCRNSHTATPNVSPIHEPESHNSPEAPILFPDDWYPPSID
PADLDESWDYIFETTESPSSDEDYVEGPSKRPRPSIQ.

In some embodiments, the EBNA2 specific T-cells are generated using one or more antigenic peptides to EBNA2, or a modified or heteroclitic peptide derived from a EBNA2 peptide. In some embodiments, the EBNA2 specific T-cells are generated with peptides that recognize class I MHC molecules. In some embodiments, the EBNA2 specific T-cells are generated with peptides that recognize class II MHC molecules. In some embodiments, the EBNA2 specific T-cells are generated with peptides that recognize both class I and class II MHC molecules.

In some embodiments, the EBNA2 peptides used to prime and expand a T-cell subpopulation includes specifically selected HLA-restricted peptides generated by determining the HLA profile of the donor source, and including peptides derived from EBNA2 that best match the donor's HLA. In some embodiments, the EBNA2 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting EBNA2 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 261-267, the HLA-B peptides are selected from the peptides of Tables 268-274, and the HLA-DR peptides are selected from the peptides of Tables 275-280. For example, if the donor cell source has an HLA profile that is HLA-A*01/*02:01; HLA-B*15:01/*18; and HLA-DRB1*0101/*0301, then the EBNA2 peptides used to prime and expand the EBNA2 specific T-cell subpopulation are restricted to the specific HLA profile, and may include the peptides identified in Table 261 (Seq. ID. Nos. 2312-2316) for HLA-A*01; Table 262 (Seq. ID. Nos. 2317-2321) for HLA-A*02:01; Table 270 (Seq. ID. Nos. 2357-2361) for HLA-B*15:01; Table 271 (Seq. ID. Nos. 2362-2366) for HLA-B*18; Table 275 (Seq. ID. Nos. 2382-2386) for HLA-DRB1*0101; and Table 276 (Seq. ID. Nos. 2387-2391) for HLA-DRB1*0301. In some embodiments, the mastermix of peptides includes both an overlapping peptide library and specifically selected HLA-restricted peptides generated by determining the HLA profile of the donor source.

In some embodiments, the EBNA2 HLA-restricted epitopes are specific to at least both of the donor's HLA-A alleles, at least both of the donor's HLA-B alleles, and at least both of the donor's HLA-DR alleles. In some embodiments, the EBNA2 HLA-A alleles are selected from a group comprising HLA-A*01, HLA-A*02:01, HLA-A*03, HLA-A*11:01, HLA-A*24:02, HLA-A*26, or HLA-A*68:01, and the corresponding EBNA2 HLA-restricted peptides are selected for: HLA-A*01 from Table 261; HLA-A*02:01 from Table 262; HLA-A*03 from Table 263; HLA-A*11:01 from Table 264; HLA-A*24:02 from Table 265; HLA-A*26 from Table 266; or HLA-A*68:01 from Table 267; or any combination thereof. In some embodiments, the EBNA2 HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding EBNA2 HLA-restricted peptides are selected for: HLA-B*07:02 from Table 268; HLA-B*08 from Table 269; HLA-B*15:01 (B62) from Table 270; HLA-B*18 from Table 271; HLA-B*27:05 from Table 272; HLA-B*35:01 from Table 273, or HLA-B*58:02 from Table 274; or any combination thereof. In some embodiments, the EBNA2 HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding EBNA2 HLA-restricted peptides are selected for: HLA-DRB1*0101 from Table 275; HLA-DRB1*0301 (DR17) from Table 276; HLA-DRB1*0401 (DR4Dw4) from Table 277; HLA-DRB1*0701 from Table 278; HLA-DRB1*1101 from Table 279; or HLA-DRB1*1501 (DR2b) from Table 280; or any combination thereof.

TABLE 261
EBV Strain B95-8 EBNA2 HLA-A*01 Epitope Peptides

| SEQ ID NO. | Sequence |
|---|---|
| 2312 | PLDRDPLGY |
| 2313 | PADLDESWDY |
| 2314 | TTESPSSDEDY |
| 2315 | SPEPRSPTVFY |
| 2316 | PSPLDRDPLGY |

TABLE 262
EBV Strain B95-8 EBNA2 HLA-A*02:01
Epitope Peptides

| SEQ ID NO. | Sequence |
|---|---|
| 2317 | HLIVDTDSL |
| 2318 | SLGNPSLSV |
| 2319 | TLPPTPLLTV |
| 2320 | VINDQQLHHL |
| 2321 | ALHGGQTYHL |

TABLE 263
EBV Strain B95-8 EBNA2 HLA-A*03
Epitope Peptides

| SEQ ID NO. | Sequence |
|---|---|
| 2322 | RGRGRGRGK |
| 2323 | PLDRDPLGY |
| 2324 | YLALHGGQTY |
| 2325 | RLTPPQPLMIVI |
| 2326 | SVIPSNPYQE |

TABLE 264
EBV Strain B95-8 EBNA2 HLA-A*11:01
Epitope Peptides

| SEQ ID NO. | Sequence |
|---|---|
| 2327 | PSNAAPVCR |
| 2328 | WTQEPSPLDR |
| 2329 | PTPLLTVLQR |
| 2330 | LVQPHVPPLR |
| 2331 | MLWMANYIVR |

TABLE 265
EBV Strain B95-8 EBNA2 HLA-A*24:02
Epitope Peptides

| SEQ ID NO. | Sequence |
|---|---|
| 2332 | GYDVGHGPL |
| 2333 | FYNIPPMPL |
| 2334 | NSPEAPILF |
| 2335 | TTLPPTPLL |
| 2336 | TVLQRPILL |

TABLE 266
EBV Strain B95-8 EBNA2 HLA-A*26
Epitope Peptides

| SEQ ID NO. | Sequence |
|---|---|
| 2337 | LVQPHVPPL |
| 2338 | ETTESPSSD |
| 2339 | DTPLIPLTIF |
| 2340 | DTDSLGNPSL |
| 2341 | DVGHGPLASA |

TABLE 267
EBV Strain B95-8 EBNA2 HLA-A*68:01
Epitope Peptides

| SEQ ID NO. | Sequence |
|---|---|
| 2342 | PTILSPLSQPR |
| 2343 | PATLTVPPR |
| 2344 | PTPLLTVLQR |
| 2345 | MLWMANYIVR |
| 2346 | LVQPHVPPLR |

TABLE 268
EBV Strain B95-8 EBNA2 HLA-B*07:02
Epitope Peptides

| SEQ ID NO. | Sequence |
|---|---|
| 2347 | TPSPPRMHL |
| 2348 | PPTPLLTVL |
| 2349 | EPSPLDRDPL |
| 2350 | PPRPTPPTPL |
| 2351 | PPRPTRPTTL |

TABLE 269
EBV Strain B95-8 EBNA2 HLA-B*08 Epitope Peptides

| SEQ ID NO. | Sequence |
|---|---|
| 2352 | SPLDRDPL |
| 2353 | SKRPRPSI |
| 2354 | PPRMHLPVL |
| 2355 | SRGDRGLIL |
| 2356 | RGKGKSRDK |

TABLE 270
EBV Strain B95-8 EBNA2 HLA-B*15:01 (B62)
Epitope Peptides

| SEQ ID NO. | Sequence |
|---|---|
| 2357 | PLDRDPLGY |
| 2358 | YLALHGGQTY |
| 2359 | SLSVIPSNPY |
| 2360 | DLDESWDYIF |
| 2361 | PLPPATLTVP |

TABLE 271
EBV Strain B95-8 EBNA2 HLA-B*18 Epitope Peptides

| SEQ ID NO. | Sequence |
|---|---|
| 2362 | PEPRSPTVF |
| 2363 | DESWDYIF |
| 2364 | QEQLSDTPL |
| 2365 | PELSPVLGL |
| 2366 | DEDYVEGP |

TABLE 272
EBV Strain B95-8 EBNA2 HLA-B*27:05
Epitope Peptides

| SEQ ID NO. | Sequence |
|---|---|
| 2367 | GRGKGKSRDK |
| 2368 | PRLTPPQPLM |
| 2369 | QRKPGGPWR |
| 2370 | PRPTPPTPL |
| 2371 | PRPTRPTTL |

TABLE 273
EBV Strain B95-8 EBNA2 HLA-B*35:01
Epitope Peptides

| SEQ ID NO. | Sequence |
|---|---|
| 2372 | EPRSPTVFY |
| 2373 | VPDQSMHPL |
| 2374 | LPPTPLLTVL |
| 2375 | SPPRMHLPVL |
| 2376 | SPLDRDPLGY |

TABLE 274
EBV Strain B95-8 EBNA2 HLA-B*58:02
Epitope Peptides

| SEQ ID NO. | Sequence |
|---|---|
| 2377 | RSPTVFYNI |
| 2378 | NSPEAPILF |
| 2379 | LALHGGQTY |
| 2380 | PSGPPWWPPI |
| 2381 | PSMPELSPVL |

TABLE 275
EBV Strain B95-8 EBNA2 HLA-DRB1*0101
Epitope Peptides

| SEQ ID NO. | Sequence |
|---|---|
| 2382 | QPHVPPLRPTAPTIL |
| 2383 | SNPYQEQLSDTPLIP |
| 2384 | PTVFYNIPPMPLPPS |
| 2385 | DQQLHHLPSGPPWWP |
| 2386 | RGLILPQGPQTAPQA |

TABLE 276
EBV Strain B95-8 EBNA2 HLA-DRB1*0301 (DR17)
Epitope Peptides

| SEQ ID NO. | Sequence |
|---|---|
| 2387 | PPGVINDQQLHHLPS |
| 2388 | PTILSPLSQPRLTPP |
| 2389 | TYHLIVDTDSLGNPS |
| 2390 | HLIVDTDSLGNPSLS |
| 2391 | ASAMRMLWMANYIVR |

TABLE 277
EBV Strain B95-8 EBNA2 HLA-DRB1*0401 (DR4Dw4)
Epitope Peptides

| SEQ ID NO. | Sequence |
|---|---|
| 2392 | MPTFYLALHGGQTYH |
| 2393 | GGPWRPEPNTSSPSM |
| 2394 | PTILSPLSQPRLTPP |
| 2395 | PTPLLTVLQRPTELQ |
| 2396 | DQSMHPLTHQSTPND |

TABLE 278
EBV Strain B95-8 EBNA2 HLA-DRB1*0701
Epitope Peptides

| SEQ ID NO. | Sequence |
|---|---|
| 2397 | NPSLSVIPSNPYQEQ |
| 2398 | PADLDESWDYIFETT |
| 2399 | DYIFETTESPSSDED |
| 2400 | YLALHGGQTYHLIVD |
| 2401 | YHLIVDTDSLGNPSL |

TABLE 279
EBV Strain B95-8 EBNA2 HLA-DRB1*1101
Epitope Peptides

| SEQ ID NO. | Sequence |
|---|---|
| 2402 | GQTYHLIVDTDSLGN |
| 2403 | TPLLTVLQRPTELQP |
| 2404 | PQPLMMPPRPTPPTP |
| 2405 | DQSMHPLTHQSTPND |
| 2406 | VINDQQLHHLPSGPP |

TABLE 280
EBV Strain B95-8 EBNA2 HLA-DRB1*1501 (DR2b)
Epitope Peptides

| SEQ ID NO. | Sequence |
|---|---|
| 2407 | LIPLTIFVGENTGVP |
| 2408 | YHLIVDTDSLGNPSL |
| 2409 | TIFVGENTGVPPPLP |
| 2410 | MLWMANYIVRQSRGD |
| 2411 | QPRLTPPQPLMMPPR |

Human Papillomavirus (HPV) Strain 16 E6 Antigenic Peptides

In some embodiments, the TVM or VM composition includes Human Papillomavirus (HPV) Strain 16 E6 specific T-cells. E6 specific T-cells can be generated as described below using one or more antigenic peptides to E6. In some embodiments, the E6 specific T-cells are generated using one or more antigenic peptides to E6, or a modified or heteroclitic peptide derived from a E6 peptide. In some embodiments, E6 specific T-cells are generated using a E6 antigen library comprising a pool of peptides (for example 15mers) containing amino acid overlap (for example 11 amino acids of overlap) between each sequence formed by scanning the protein amino acid sequence SEQ. ID. No. 2412 (UniProt KB—P03126) for HPV Strain 16-8 E6:

MHQKRTAMFQDPQERPRKLPQLCTELQTTIHDIILECVYCKQQLLRREVYD
FAFRDLCIVYRDGNPYAVCDKCLKFYSKISEYRHYCYSLYGTTLEQQYNKP
LCDLLIRCINCQKPLCPEEKQRHLDKKQRFHNIRGRWTGRCMSCCRSSRTR
RETQL.

In some embodiments, the E6 specific T-cells are generated using one or more antigenic peptides to E6, or a modified or heteroclitic peptide derived from a E6 peptide. In some embodiments, the E6 specific T-cells are generated with peptides that recognize class I MHC molecules. In some embodiments, the E6 specific T-cells are generated with peptides that recognize class II MHC molecules. In some embodiments, the E6 specific T-cells are generated with peptides that recognize both class I and class II MHC molecules.

In some embodiments, the E6 peptides used to prime and expand a T-cell subpopulation includes specifically selected HLA-restricted peptides generated by determining the HLA profile of the donor source, and including peptides derived from E6 that best match the donor's HLA. In some embodiments, the E6 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting E6 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 281-287, the HLA-B peptides are selected from the peptides of Tables 288-294, and the HLA-DR peptides are selected from the peptides of Tables 295-280. For example, if the donor cell source has an HLA profile that is HLA-A*01/*02:01; HLA-B*15:01/*18; and HLA-DRB1*0101/*0301, then the E6 peptides used to prime and expand the E6 specific T-cell subpopulation are restricted to the specific HLA profile, and may include the peptides identified in Table 281 (Seq. ID. Nos. 2413-2417) for HLA-A*01; Table 282 (Seq. ID. Nos. 2418-2422) for HLA-A*02:01; Table 290 (Seq. ID. Nos. 2458-2462) for HLA-B*15:01; Table 291 (Seq. ID. Nos. 2463-2467) for HLA-B*18; Table 295 (Seq. ID. Nos. 2483-2487) for HLA-DRB1*0101; and Table 296 (Seq. ID. Nos. 2488-2492) for HLA-DRB1*0301. In some embodiments, the mastermix of peptides includes both an overlapping peptide library and specifically selected HLA-restricted peptides generated by determining the HLA profile of the donor source.

In some embodiments, the E6 HLA-restricted epitopes are specific to at least both of the donor's HLA-A alleles, at least both of the donor's HLA-B alleles, and at least both of the donor's HLA-DR alleles. In some embodiments, the E6 HLA-A alleles are selected from a group comprising HLA-A*01, HLA-A*02:01, HLA-A*03, HLA-A*11:01, HLA-A*24:02, HLA-A*26, or HLA-A*68:01, and the corresponding E6 HLA-restricted peptides are selected for: HLA-A*01 from Table 281; HLA-A*02:01 from Table 282; HLA-A*03 from Table 283; HLA-A*11:01 from Table 284; HLA-A*24:02 from Table 285; HLA-A*26 from Table 286; or HLA-A*68:01 from Table 287; or any combination thereof. In some embodiments, the E6 HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding E6 HLA-restricted peptides are selected for: HLA-B*07:02 from Table 288; HLA-B*08 from Table 289; HLA-B*15:01 (B62) from Table 290; HLA-B*18 from Table 291; HLA-B*27:05 from Table 292; HLA-B*35:01 from Table 293, or HLA-B*58:02 from Table 294; or any combination thereof. In some embodiments, the E6 HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding E6 HLA-restricted peptides are selected for: HLA-DRB1*0101 from Table 295; HLA-DRB1*0301 (DR17) from Table 296; HLA-DRB1*0401 (DR4Dw4) from Table 297; HLA-DRB1*0701 from Table 298; HLA-DRB1*1101 from Table 299; or HLA-DRB1*1501 (DR2b) from Table 300; or any combination thereof.

TABLE 281
HPV Strain 16 E6 HLA-A*01 Epitope Peptides

| SEQ ID NO. | Sequence |
|---|---|
| 2413 | YAVCDKCLKFY |
| 2414 | SEYRHYCYSLY |
| 2415 | CKQQLLRREVY |
| 2416 | IHDIILECVY |
| 2417 | YSKISEYRHY |

TABLE 282
HPV Strain 16 E6 HLA-A*02:01 Epitope Peptides

| SEQ ID NO. | Sequence |
|---|---|
| 2418 | TIHDIILECV |
| 2419 | QLCIELQTTI |
| 2420 | PLCDLLIRCI |
| 2421 | KLPQLCTEL |
| 2422 | QLCIELQTT |

TABLE 283
HPV Strain 16 E6 HLA-A*03 Epitope Peptides

| SEQ ID NO. | Sequence |
|---|---|
| 2423 | LLIRCINCQK |
| 2424 | DIILECVYCK |
| 2425 | CVYCKQQLLR |
| 2426 | SLYGTTLEQQ |
| 2427 | IVYRDGNPY |

TABLE 284
HPV Strain 16 E6 HLA-A*11:01 Epitope Peptides

| SEQ ID NO. | Sequence |
|---|---|
| 2428 | CVYCKQQLLR |
| 2429 | GTTLEQQYNK |
| 2430 | DIILECVYCK |
| 2431 | AFRDLCIVYR |
| 2432 | WTGRCMSCCR |

TABLE 285
HPV Strain 16 E6 HLA-A*24:02 Epitope Peptides

| SEQ ID NO. | Sequence |
|---|---|
| 2433 | QYNKPLCDLL |
| 2434 | QDPQERPRKL |
| 2435 | LCPEEKQRHL |
| 2436 | VYDFAFRDL |
| 2437 | PYAVCDKCL |

TABLE 286
HPV Strain 16 E6 HLA-A*26 Epitope Peptides

| SEQ ID NO. | Sequence |
|---|---|
| 2438 | EVYDFAFRDL |
| 2439 | AVCDKCLKFY |
| 2440 | EYRHYCYSLY |
| 2441 | DIILECVYCK |
| 2442 | CIVYRDGNPY |

TABLE 287
HPV Strain 16 E6 HLA-A*68:01 Epitope Peptides

| SEQ ID NO. | Sequence |
|---|---|
| 2443 | FAFRDLCIVYR |
| 2444 | RTAMFQDPQER |
| 2445 | CVYCKQQLLRR |
| 2446 | MSCCRSSRTRR |
| 2447 | DLLIRCINCQK |

TABLE 288
HPV Strain 16 E6 HLA-B*07:02 Epitope Peptides

| SEQ ID NO. | Sequence |
|---|---|
| 2448 | RPRKLPQLCT |
| 2449 | NPYAVCDKCL |
| 2450 | LPQLCTELQT |
| 2451 | QERPRKLPQL |
| 2452 | DPQERPRKL |

TABLE 289
HPV Strain 16 E6 HLA-B*08 Epitope Peptides

| SEQ ID NO. | Sequence |
|---|---|
| 2453 | CPEEKQRHL |
| 2454 | DPQERPRKL |
| 2455 | DKKQRFHNI |
| 2456 | ERPRKLPQL |
| 2457 | CVYCKQQLL |

TABLE 290
HPV Strain 16 E6 HLA-B*15:01 (B62)
Epitope Peptides

| SEQ ID NO. | Sequence |
|---|---|
| 2458 | KQQLLRREVY |
| 2459 | AVCDKCLKFY |
| 2460 | QLLRREVYDF |
| 2461 | QLCTELQTTI |
| 2462 | FAFRDLCIVY |

TABLE 291
HPV Strain 16 E6 HLA-B*18 Epitope Peptides

| SEQ ID NO. | Sequence |
|---|---|
| 2463 | LEQQYNKPL |
| 2464 | QQLLRREVY |
| 2465 | DPQERPRKL |
| 2466 | AFRDLCIVY |
| 2467 | REVYDFAF |

TABLE 292
HPV Strain 16 E6 HLA-B*27:05 Epitope Peptides

| SEQ ID NO. | Sequence |
|---|---|
| 2468 | RREVYDFAFR |
| 2469 | QRHLDKKQRF |
| 2470 | IRCINCQKPL |
| 2471 | LRREVYDFAF |
| 2472 | RKLPQLCTEL |

TABLE 293
HPV Strain 16 E6 HLA-B*35:01 Epitope Peptides

| SEQ ID NO. | Sequence |
|---|---|
| 2473 | NPYAVCDKCL |
| 2474 | FAFRDLCIVY |
| 2475 | LQTTIHDIIL |
| 2476 | KPLCDLLIRC |
| 2477 | ECVYCKQQLL |

TABLE 294
HPV Strain 16 E6 HLA-B*58:02 Epitope Peptides

| SEQ ID NO. | Sequence |
|---|---|
| 2478 | YSKISEYRHY |
| 2479 | EVYDFAFRDL |
| 2480 | SSRTRRETQL |
| 2481 | FAFRDLCIVY |
| 2482 | YAVCDKCLKF |

TABLE 295
HPV Strain 16 E6 HLA-DRB1*0101 Epitope Peptides

| SEQ ID NO. | Sequence |
|---|---|
| 2483 | IRCINCQKPLCPEEK |
| 2484 | CIVYRDGNPYAVCDK |
| 2485 | RHYCYSLYGTTLEQQ |
| 2486 | NKPLCDLLIRCINCQ |
| 2487 | DLLIRCINCQKPLCP |

TABLE 296
HPV Strain 16 E6 HLA-DRB1*0301 (DR17)
Epitope Peptides

| SEQ ID NO. | Sequence |
|---|---|
| 2488 | RTAMFQDPQERPRKL |
| 2489 | LCIVYRDGNPYAVCD |
| 2490 | EKQRHLDKKQRFHNI |
| 2491 | GTTLEQQYNKPLCDL |
| 2492 | NPYAVCDKCLKFYSK |

TABLE 297
HPV Strain 16 E6 HLA-DRB1*0401 (DR4Dw4)
Epitope Peptides

| SEQ ID NO. | Sequence |
|---|---|
| 2493 | PRKLPQLCTELQTTI |
| 2494 | LPQLCTELQTTIHDI |
| 2495 | FRDLCIVYRDGNPYA |
| 2496 | LCIVYRDGNPYAVCD |
| 2497 | REVYDFAFRDLCIVY |

TABLE 298
HPV Strain 16 E6 HLA-DRB1*0701 Epitope Peptides

| SEQ ID NO. | Sequence |
|---|---|
| 2498 | YSLYGTTLEQQYNKP |
| 2499 | CTELQTTIHDIILEC |
| 2500 | QTTIHDIILECVYCK |
| 2501 | LKFYSKISEYRHYCY |
| 2502 | GTTLEQQYNKPLCDL |

TABLE 299
HPV Strain 16 E6 HLA-DRB1*1101 Epitope Peptides

| SEQ ID NO. | Sequence |
|---|---|
| 2503 | FRDLCIVYRDGNPYA |
| 2504 | GNPYAVCDKCLKFYS |
| 2505 | REVYDFAFRDLCIVY |
| 2506 | DLLIRCINCQKPLCP |
| 2507 | LKFYSKISEYRHYCY |

TABLE 300
HPV Strain 16 E6 HLA-DRB1*1501 (DR2b)
Epitope Peptides

| SEQ ID NO. | Sequence |
|---|---|
| 2508 | DKCLKFYSKISEYRH |
| 2509 | RREVYDFAFRDLCIV |
| 2510 | IRCINCQKPLCPEEK |
| 2511 | LDKKQRFHNIRGRWT |
| 2512 | LECVYCKQQLLRREV |

Human Papillomavirus (HPV) Strain 16 E7 Antigenic Peptides

In some embodiments, the TVM or VM composition includes Human Papillomavirus (HPV) Strain 16 E7 specific T-cells. E7 specific T-cells can be generated as described below using one or more antigenic peptides to E7. In some embodiments, the E7 specific T-cells are generated using one or more antigenic peptides to E7, or a modified or heteroclitic peptide derived from a E7 peptide. In some embodiments, E7 specific T-cells are generated using a E7 antigen library comprising a pool of peptides (for example 15mers) containing amino acid overlap (for example 11 amino acids of overlap) between each sequence formed by scanning the protein amino acid sequence SEQ. ID. No. 2513 (UniProt KB—P03129) for HPV Strain 16-8 E7:

MEIGDTPTLHEYMLDLQPETTDLYCYEQLNDSSEEEDEIDGPAGQAEPDR
AHYNIVTFCCKCDSTLRLCVQSTHVDIRTLEDLLMGTLGIVCPICSQKP.

In some embodiments, the E7 specific T-cells are generated using one or more antigenic peptides to E7, or a modified or heteroclitic peptide derived from a E7 peptide. In some embodiments, the E7 specific T-cells are generated with peptides that recognize class I MHC molecules. In some embodiments, the E7 specific T-cells are generated with peptides that recognize class II MHC molecules. In some embodiments, the E7 specific T-cells are generated with peptides that recognize both class I and class II MHC molecules.

In some embodiments, the E7 peptides used to prime and expand a T-cell subpopulation includes specifically selected HLA-restricted peptides generated by determining the HLA profile of the donor source, and including peptides derived from E7 that best match the donor's HLA. In some embodiments, the E7 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting E7 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 301-307, the HLA-B peptides are selected from the peptides of Tables 308-314, and the HLA-DR peptides are selected from the peptides of Tables 315-320. For example, if the donor cell source has an HLA profile that is HLA-A*01/*02:01; HLA-B*15:01/*18; and HLA-DRB1*0101/*0301, then the E7 peptides used to prime and expand the E7 specific T-cell subpopulation are restricted to the specific HLA profile, and may include the peptides identified in Table 301 (Seq. ID. Nos. 2514-2518) for HLA-A*01; Table 302 (Seq. ID. Nos. 2519-2523) for HLA-A*02:01; Table 310 (Seq. ID. Nos. 2559-2563) for HLA-B*15:01; Table 311 (Seq. ID. Nos. 2564-2568) for HLA-B*18; Table 315 (Seq. ID. Nos. 2584-2588) for HLA-DRB1*0101; and Table 316 (Seq. ID. Nos. 2589-2593) for HLA-DRB1*0301. In some embodiments, the mastermix of peptides includes both an overlapping peptide library and specifically selected HLA-restricted peptides generated by determining the HLA profile of the donor source.

In some embodiments, the E7 HLA-restricted epitopes are specific to at least both of the donor's HLA-A alleles, at least both of the donor's HLA-B alleles, and at least both of the donor's HLA-DR alleles. In some embodiments, the E7 HLA-A alleles are selected from a group comprising HLA-A*01, HLA-A*02:01, HLA-A*03, HLA-A*11:01, HLA-A*24:02, HLA-A*26, or HLA-A*68:01, and the corresponding E7 HLA-restricted peptides are selected for: HLA-A*01 from Table 301; HLA-A*02:01 from Table 302; HLA-A*03 from Table 303; HLA-A*11:01 from Table 304; HLA-A*24:02 from Table 305; HLA-A*26 from Table 306; or HLA-A*68:01 from Table 307; or any combination thereof. In some embodiments, the E7 HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding E7 HLA-restricted peptides are selected for: HLA-B*07:02 from Table 308; HLA-B*08 from Table 309; HLA-B*15:01 (B62) from Table 310; HLA-B*18 from Table 311; HLA-B*27:05 from Table 312; HLA-B*35:01 from Table 313, or HLA-B*58:02 from Table 314; or any combination thereof. In some embodiments, the E7 HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding E7 HLA-restricted peptides are selected for: HLA-DRB1*0101 from Table 315; HLA-DRB1*0301 (DR17) from Table 316; HLA-DRB1*0401 (DR4Dw4) from Table 317; HLA-DRB1*0701 from Table 318; HLA-DRB1*1101 from Table 319; or HLA-DRB1*1501 (DR2b) from Table 320; or any combination thereof.

TABLE 301
HPV Strain 16 E7 HLA-A*01 Epitope Peptides

| SEQ ID NO. | Sequence |
|---|---|
| 2514 | MHGDTPTLHEY |
| 2515 | HGDTPTLHEY |
| 2516 | QPETTDLYCY |
| 2517 | DLQPETTDLY |
| 2518 | QAEPDRAHY |

TABLE 302
HPV Strain 16 E7 HLA-A*02:01 Epitope Peptides

| SEQ ID NO. | Sequence |
|---|---|
| 2519 | DLLMGTLGIV |
| 2520 | TLEDLLMGTL |
| 2521 | LLMGTLGIV |
| 2522 | TLHEYMLDL |
| 2523 | DLQPETTDL |

TABLE 303
HPV Strain 16 E7 HLA-A*03 Epitope Peptides

| SEQ ID NO. | Sequence |
|---|---|
| 2524 | TLRLCVQSTH |
| 2525 | GIVCPICSQK |
| 2526 | DLQPETTDLY |
| 2527 | LLMGTLGIVC |
| 2528 | IVCPICSQK |

TABLE 304
HPV Strain 16 E7 HLA-A*11:01 Epitope Peptides

| SEQ ID NO. | Sequence |
|---|---|
| 2529 | CVQSTHVDIR |
| 2530 | GIVCPICSQK |
| 2531 | SSEEEDEIDG |
| 2532 | IVCPICSQK |
| 2533 | STLRLCVQS |

TABLE 305
HPV Strain 16 E7 HLA-A*24:02 Epitope Peptides

| SEQ ID NO. | Sequence |
|---|---|
| 2534 | TFCCKCDSTL |
| 2535 | VDIRTLEDLL |
| 2536 | AEPDRAHYNI |
| 2537 | TDLYCYEQL |
| 2538 | GTLGIVCPI |

TABLE 306
HPV Strain 16 E7 HLA-A*26 Epitope Peptides

| SEQ ID NO. | Sequence |
|---|---|
| 2539 | DTPTLHEYML |
| 2540 | HVDIRTLEDL |
| 2541 | DRAHYNIVTF |
| 2542 | STHVDIRTL |
| 2543 | ETTDLYCYE |

TABLE 307
HPV Strain 16 E7 HLA-A*68:01 Epitope Peptides

| SEQ ID NO. | Sequence |
|---|---|
| 2544 | TFCCKCDSTLR |
| 2545 | ETTDLYCYEQL |
| 2546 | CVQSTHVDIR |
| 2547 | IVCPICSQK |
| 2548 | PAGQAEPDR |

TABLE 308
HPV Strain 16 E7 HLA-B*07:02 Epitope Peptides

| SEQ ID NO. | Sequence |
|---|---|
| 2549 | EPDRAHYNIV |
| 2550 | GPAGQAEPDR |
| 2551 | CCKCDSTLRL |
| 2552 | TPTLHEYML |
| 2553 | EIDGPAGQA |

TABLE 309
HPV Strain 16 E7 HLA-B*08 Epitope Peptides

| SEQ ID NO. | Sequence |
|---|---|
| 2554 | DIRTLEDLL |
| 2555 | TLHEYMLDL |
| 2556 | TPTLHEYML |
| 2557 | DLQPETTDL |
| 2558 | CCKCDSTL |

TABLE 310
HPV Strain 16 E7 HLA-B*15:01 (B62) Epitope Peptides

| SEQ ID NO. | Sequence |
|---|---|
| 2559 | DLQPETTDLY |
| 2560 | GQAEPDRAHY |
| 2561 | TLRLCVQSTH |
| 2562 | LLMGTLGIVC |
| 2563 | LQPETTDLY |

TABLE 311
HPV Strain 16 E7 HLA-B*18 Epitope Peptides

| SEQ ID NO. | Sequence |
|---|---|
| 2564 | LEDLLMGTL |
| 2565 | PETTDLYCY |
| 2566 | DEIDGPAGQ |
| 2567 | DIRTLEDLL |
| 2568 | AEPDRAHY |

TABLE 312
HPV Strain 16 E7 HLA-B*27:05 Epitope Peptides

| SEQ ID NO. | Sequence |
|---|---|
| 2569 | DRAHYNIVTF |
| 2570 | LDLQPETTDL |
| 2571 | LRLCVQSTH |
| 2572 | IRTLEDLLM |
| 2573 | RAHYNIVTF |

TABLE 313
HPV Strain 16 E7 HLA-B*35:01 Epitope Peptides

| SEQ ID NO. | Sequence |
|---|---|
| 2574 | QPETTDLYCY |
| 2575 | TPTLHEYML |
| 2576 | EPDRAHYNI |
| 2577 | FCCKCDSTL |
| 2578 | LEDLLMGTL |

TABLE 314
HPV Strain 16 E7 HLA-B*58:02 Epitope Peptides

| SEQ ID NO. | Sequence |
|---|---|
| 2579 | QSTHVDIRTL |
| 2580 | RAHYNIVTF |
| 2581 | DSSEEEDEI |
| 2582 | GTLGIVCPI |
| 2583 | DTPTLHEYM |

TABLE 315
HPV Strain 16 E7 HLA-DRB1*0101 Epitope Peptides

| SEQ ID NO. | Sequence |
|---|---|
| 2584 | MGTLGIVCPICSQKP |
| 2585 | HVDIRTLEDLLMGTL |
| 2586 | DLLMGTLGIVCPICS |
| 2587 | IRTLEDLLMGTLGIV |
| 2588 | TLEDLLMGTLGIVCP |

TABLE 316
HPV Strain 16 E7 HLA-DRB1*0301 (DR17) Epitope
Peptides

| SEQ ID NO. | Sequence |
|---|---|
| 2589 | TFCCKCDSTLRLCVQ |
| 2590 | MLDLQPETTDLYCYE |
| 2591 | TTDLYCYEQLNDSSE |
| 2592 | IRTLEDLLMGTLGIV |
| 2593 | LHEYMLDLQPETTDL |

TABLE 317
HPV Strain 16 E7 HLA-DRB1*0401 (DR4Dw4) Epitope
Peptides

| SEQ ID NO. | Sequence |
|---|---|
| 2594 | LHEYMLDLQPETTDL |
| 2595 | TDLYCYEQLNDSSEE |
| 2596 | RAHYNIVTFCCKCDS |
| 2597 | HEYMLDLQPETTDLY |
| 2598 | MLDLQPETTDLYCYE |

TABLE 318
HPV Strain 16 E7 HLA-DRB1*0701 Epitope Peptides

| SEQ ID NO. | Sequence |
|---|---|
| 2599 | IVTFCCKCDSTLRLC |
| 2600 | MLDLQPETTDLYCYE |
| 2601 | THVDIRTLEDLLMGT |
| 2602 | HVDIRTLEDLLMGTL |
| 2603 | YEQLNDSSEEEDEID |

TABLE 319
HPV Strain 16 E7 HLA-DRB1*1101 Epitope Peptides

| SEQ ID NO. | Sequence |
|---|---|
| 2604 | YNIVTFCCKCDSTLR |
| 2605 | DLLMGTLGIVCPICS |
| 2606 | MGTLGIVCPICSQKP |
| 2607 | TDLYCYEQLNDSSEE |
| 2608 | LYCYEQLNDSSEEED |

TABLE 320
HPV Strain 16 E7 HLA-DRB1*1501 (DR2b) Epitope
Peptides

| SEQ ID NO. | Sequence |
|---|---|
| 2609 | TTDLYCYEQLNDSSE |
| 2610 | TPTLHEYMLDLQPET |
| 2611 | EDLLMGTLGIVCPIC |
| 2612 | MGTLGIVCPICSQKP |
| 2613 | HYNIVTFCCKCDSTL |

Human Papillomavirus (HPV) Strain 16 E7 Antigenic Peptides

In some embodiments, the TVM or VM composition includes Human Papillomavirus (HPV) Strain 16 E7 specific T-cells. E7 specific T-cells can be generated as described below using one or more antigenic peptides to E7. In some embodiments, the E7 specific T-cells are generated using one or more antigenic peptides to E7, or a modified or heteroclitic peptide derived from a E7 peptide. In some embodiments, E7 specific T-cells are generated using a E7 antigen library comprising a pool of peptides (for example 15mers) containing amino acid overlap (for example 11 amino acids of overlap) between each sequence formed by scanning the protein amino acid sequence SEQ. ID. No. 2513 (UniProt KB—P03129) for HPV Strain 16-8 E7:

MHGDTPTLHEYMLDLQPETTDLYCYEQLNDSSEEEDEIDGPAGQAEPDRA
HYNIVTFCCKCDSTLRLCVQSTHVDIRTLEDLLMGTLGIVCPICSQKP.

In some embodiments, the E7 specific T-cells are generated using one or more antigenic peptides to E7, or a modified or heteroclitic peptide derived from a E7 peptide. In some embodiments, the E7 specific T-cells are generated with peptides that recognize class I MHC molecules. In some embodiments, the E7 specific T-cells are generated with peptides that recognize class II MHC molecules. In some embodiments, the E7 specific T-cells are generated with peptides that recognize both class I and class II MHC molecules.

In some embodiments, the E7 peptides used to prime and expand a T-cell subpopulation includes specifically selected HLA-restricted peptides generated by determining the HLA profile of the donor source, and including peptides derived from E7 that best match the donor's HLA. In some embodiments, the E7 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting E7 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 301-307, the HLA-B peptides are selected from the peptides of Tables 308-314, and the HLA-DR peptides are selected from the peptides of Tables 315-320. For example, if the donor cell source has an HLA profile that is HLA-A*01/*02:01; HLA-B*15:01/*18; and HLA-DRB1*0101/*0301, then the E7 peptides used to prime and expand the E7 specific T-cell subpopulation are restricted to the specific HLA profile, and may include the peptides identified in Table 301 (Seq. ID. Nos. 2514-2518) for HLA-A*01; Table 302 (Seq. ID. Nos. 2519-2523) for HLA-A*02:01; Table 310 (Seq. ID. Nos. 2559-2563) for HLA-B*15:01; Table 311 (Seq. ID. Nos. 2564-2568) for HLA-B*18; Table 315 (Seq. ID. Nos. 2584-2588) for HLA-DRB1*0101; and Table 316 (Seq. ID. Nos. 2589-2593) for HLA-DRB1*0301. In some embodiments, the mastermix of peptides includes both an overlapping peptide library and specifically selected HLA-restricted peptides generated by determining the HLA profile of the donor source.

In some embodiments, the HPV E7 HLA-restricted epitopes are specific to at least both of the donor's HLA-A alleles, at least both of the donor's HLA-B alleles, and at least both of the donor's HLA-DR alleles. In some embodiments, the HPV E7 HLA-A alleles are selected from a group comprising HLA-A*01, HLA-A*02:01, HLA-A*03, HLA-A*11:01, HLA-A*24:02, HLA-A*26, or HLA-A*68:01, and the corresponding HPV E7 HLA-restricted peptides are selected for: HLA-A*01 from Table 301; HLA-A*02:01 from Table 302; HLA-A*03 from Table 303; HLA-A*11:01 from Table 304; HLA-A*24:02 from Table 305; HLA-A*26 from Table 306; or HLA-A*68:01 from Table 307; or any combination thereof. In some embodiments, the HPV E7 HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding HPV E7 HLA-restricted peptides are selected for: HLA-B*07:02 from Table 308; HLA-B*08 from Table 309; HLA-B*15:01 (B62) from Table 310; HLA-B*18 from Table 311; HLA-B*27:05 from Table 312; HLA-B*35:01 from Table 313, or HLA-B*58:02 from Table 314; or any combination thereof. In some embodiments, the HPV E7 HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding HPV E7 HLA-restricted peptides are selected for: HLA-DRB1*0101 from Table 315; HLA-DRB1*0301 (DR17) from Table 316; HLA-DRB1*0401 (DR4Dw4) from Table 317; HLA-DRB1*0701 from Table 318; HLA-DRB1*1101 from Table 319; or HLA-DRB1*1501 (DR2b) from Table 320; or any combination thereof.

TABLE 301
HPV Strain 16 E7 HLA-A*01 Epitope Peptides

| SEQ ID NO. | Sequence |
|---|---|
| 2514 | MHGDTPTLHEY |
| 2515 | HGDTPTLHEY |
| 2516 | QPETTDLYCY |
| 2517 | DLQPETTDLY |
| 2518 | QAEPDRAHY |

TABLE 302
HPV Strain 16 E7 HLA-A*02:01 Epitope Peptides

| SEQ ID NO. | Sequence |
|---|---|
| 2519 | DLLMGTLGIV |
| 2520 | TLEDLLMGTL |
| 2521 | LLMGTLGIV |
| 2522 | TLHEYMLDL |
| 2523 | DLQPETTDL |

TABLE 303
HPV Strain 16 E7 HLA-A*03 Epitope Peptides

| SEQ ID NO. | Sequence |
|---|---|
| 2524 | TLRLCVQSTH |
| 2525 | GIVCPICSQK |
| 2526 | DLQPETTDLY |
| 2527 | LLMGTLGIVC |
| 2528 | IVCPICSQK |

TABLE 304
HPV Strain 16 E7 HLA-A*11:01 Epitope Peptides

| SEQ ID NO. | Sequence |
|---|---|
| 2529 | CVQSTHVDIR |
| 2530 | GIVCPICSQK |
| 2531 | SSEEEDEIDG |
| 2532 | IVCPICSQK |
| 2533 | STLRLCVQS |

TABLE 305
HPV Strain 16 E7 HLA-A*24:02 Epitope Peptides

| SEQ ID NO. | Sequence |
|---|---|
| 2534 | TFCCKCDSTL |
| 2535 | VDIRTLEDLL |
| 2536 | AEPDRAHYNI |
| 2537 | TDLYCYEQL |
| 2538 | GTLGIVCPI |

TABLE 306
HPV Strain 16 E7 HLA-A*26 Epitope Peptides

| SEQ ID NO. | Sequence |
|---|---|
| 2539 | DTPTLHEYML |
| 2540 | HVDIRTLEDL |
| 2541 | DRAHYNIVTF |
| 2542 | STHVDIRTL |
| 2543 | ETTDLYCYE |

TABLE 307
HPV Strain 16 E7 HLA-A*68:01 Epitope
Peptides

| SEQ ID NO. | Sequence |
|---|---|
| 2544 | TFCCKCDSTLR |
| 2545 | ETTDLYCYEQL |
| 2546 | CVQSTHVDIR |
| 2547 | IVCPICSQK |
| 2548 | PAGQAEPDR |

TABLE 308
HPV Strain 16 E7 HLA-B*07:02 Epitope Peptides

| SEQ ID NO. | Sequence |
|---|---|
| 2549 | EPDRAHYNIV |
| 2550 | GPAGQAEPDR |
| 2551 | CCKCDSTLRL |
| 2552 | TPTLHEYML |
| 2553 | EIDGPAGQA |

TABLE 309
HPV Strain 16 E7 HLA-B*08 Epitope Peptides

| SEQ ID NO. | Sequence |
|---|---|
| 2554 | DIRTLEDLL |
| 2555 | TLHEYMLDL |
| 2556 | TPTLHEYML |
| 2557 | DLQPETTDL |
| 2558 | CCKCDSTL |

TABLE 310
HPV Strain 16 E7 HLA-B*15:01 (B62) Epitope
Peptides

| SEQ ID NO. | Sequence |
|---|---|
| 2559 | DLQPETTDLY |
| 2560 | GQAEPDRAHY |
| 2561 | TLRLCVQSTH |
| 2562 | LLMGTLGIVC |
| 2563 | LQPETTDLY |

TABLE 311
HPV Strain 16 E7 HLA-B*18 Epitope Peptides

| SEQ ID NO. | Sequence |
|---|---|
| 2564 | LEDLLMGTL |
| 2565 | PETTDLYCY |
| 2566 | DEIDGPAGQ |
| 2567 | DIRTLEDLL |
| 2568 | AEPDRAHY |

TABLE 312
HPV Strain 16 E7 HLA-B*27:05 Epitope Peptides

| SEQ ID NO. | Sequence |
|---|---|
| 2569 | DRAHYNIVTF |
| 2570 | LDLQPETTDL |
| 2571 | LRLCVQSTH |
| 2572 | IRTLEDLLM |
| 2573 | RAHYNIVTF |

TABLE 313
HPV Strain 16 E7 HLA-B*35:01 Epitope Peptides

| SEQ ID NO. | Sequence |
|---|---|
| 2574 | QPETTDLYCY |
| 2575 | TPTLHEYML |
| 2576 | EPDRAHYNI |
| 2577 | FCCKCDSTL |
| 2578 | LEDLLMGTL |

TABLE 314
HPV Strain 16 E7 HLA-B*58:02 Epitope Peptides

| SEQ ID NO. | Sequence |
|---|---|
| 2579 | QSTHVDIRTL |
| 2580 | RAHYNIVTF |
| 2581 | DSSEEEDEI |
| 2582 | GTLGIVCPI |
| 2583 | DTPTLHEYM |

TABLE 315
HPV Strain 16 E7 HLA-DRB1*0101 Epitope Peptides

| SEQ ID NO. | Sequence |
|---|---|
| 2584 | MGTLGIVCPICSQKP |
| 2585 | HVDIRTLEDLLMGTL |
| 2586 | DLLMGTLGIVCPICS |
| 2587 | IRTLEDLLMGTLGIV |
| 2588 | TLEDLLMGTLGIVCP |

TABLE 316
HPV Strain 16 E7 HLA-DRB1*0301 (DR17) Epitope
Peptides

| SEQ ID NO. | Sequence |
|---|---|
| 2589 | TFCCKCDSTLRLCVQ |
| 2590 | MLDLQPETTDLYCYE |
| 2591 | TTDLYCYEQLNDSSE |
| 2592 | IRTLEDLLMGTLGIV |
| 2593 | LHEYMLDLQPETTDL |

TABLE 317
HPV Strain 16 E7 HLA-DRB1*0401 (DR4Dw4) Epitope
Peptides

| SEQ ID NO. | Sequence |
|---|---|
| 2594 | LHEYMLDLQPETTDL |
| 2595 | TDLYCYEQLNDSSEE |
| 2596 | RAHYNIVTFCCKCDS |
| 2597 | HEYMLDLQPETTDLY |
| 2598 | MLDLQPETTDLYCYE |

TABLE 318
HPV Strain 16 E7 HLA-DRB1*0701 Epitope Peptides

| SEQ ID NO. | Sequence |
|---|---|
| 2599 | IVTFCCKCDSTLRLC |
| 2600 | MLDLQPETTDLYCYE |
| 2601 | THVDIRTLEDLLMGT |
| 2602 | HVDIRTLEDLLMGTL |
| 2603 | YEQLNDSSEEEDEID |

TABLE 319
HPV Strain 16 E7 HLA-DRB1*1101 Epitope Peptides

| SEQ ID NO. | Sequence |
|---|---|
| 2604 | YNIVTFCCKCDSTLR |
| 2605 | DLLMGTLGIVCPICS |
| 2606 | MGTLGIVCPICSQKP |
| 2607 | TDLYCYEQLNDSSEE |
| 2608 | LYCYEQLNDSSEEED |

TABLE 320
HPV Strain 16 E7 HLA-DRB1*1501 (DR2b) Epitope
Peptides

| SEQ ID NO. | Sequence |
|---|---|
| 2609 | TTDLYCYEQLNDSSE |
| 2610 | TPTLHEYMLDLQPET |
| 2611 | EDLLMGTLGIVCPIC |
| 2612 | MGTLGIVCPICSQKP |
| 2613 | HYNIVTFCCKCDSTL |

### Human Cytomegalovirus (HCMV) Strain HHV-5 Antigenic Peptides

In some embodiments, the TVM or VM composition includes Human Cytomegalovirus (HCMV) pp65 specific T-cells. pp65 specific T-cells can be generated as described below using one or more antigenic peptides to pp65. In some embodiments, the pp65 specific T-cells are generated using one or more antigenic peptides to pp65, or a modified or heteroclitic peptide derived from a pp65 peptide. In some embodiments, pp65 specific T-cells are generated using a pp65 antigen library comprising a pool of peptides (for example 15mers) containing amino acid overlap (for example 11 amino acids of overlap) between each sequence formed by scanning the protein amino acid sequence SEQ. ID. No. 2614 (UniProt KB—P06725) for HCMV Strain HHV-5 pp65:

MESRGRRCPEMISVLGPISGHVLKAVFSRGDTPVLPHETRLLQTGIHVRVS
QPSLILVSQYTPDSTPCHRGDNQLQVQHTYFTGSEVENVSVNVHNPTGRSI
CPSQEPMSIYVYALPLKMLNIPSINVHHYPSAAERKHRHLPVADAVIHASG
KQMWQARLTVSGLAWTRQQNQWKEPDVYYTSAFVFPTKDVALRHVVCAHEL
VCSMENTRATKMQVIGDQYVKVYLESFCEDVPSGKLFMHVTLGSDVEEDLT
MTRNPQPFMRPHERNGFTVLCPKNMIIKPGKISHIMLDVAFTSHEHEGLLC
PKSIPGLSISGNLLMNGQQIFLEVQAIRETVELRQYDPVAALFFFDIDLLL
QRGPQYSEHPTFTSQYRIQGKLEYRHTWDRHDEGAAQGDDDVWTSGSDSDE
ELVTTERKTPRVTGGGAMAGASTSAGRKRKSASSATACTSGVMTRGRLKAE
STVAPEEDTDEDSDNEIHNPAVFTWPPWQAGILARNLVPMVATVQGQNLKY
QEFFWDANDIYRIFAELEGVWQPAAQPKRRRHRQDALPGPCIASTPKKHRG

In some embodiments, the pp65 specific T-cells are generated using one or more antigenic peptides to pp65, or a modified or heteroclitic peptide derived from a pp65 peptide. In some embodiments, the pp65 specific T-cells are generated with peptides that recognize class I MHC molecules. In some embodiments, the pp65 specific T-cells are generated with peptides that recognize class II MHC molecules. In some embodiments, the pp65 specific T-cells are generated with peptides that recognize both class I and class II MHC molecules.

In some embodiments, the pp65 peptides used to prime and expand a T-cell subpopulation includes specifically selected HLA-restricted peptides generated by determining the HLA profile of the donor source, and including peptides derived from pp65 that best match the donor's HLA. In some embodiments, the pp65 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting pp65 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 321-327, the HLA-B peptides are selected from the peptides of Tables 328-334, and the HLA-DR peptides are selected from the peptides of Tables 325-340. For example, if the donor cell source has an HLA profile that is HLA-A*01/*02:01; HLA-B*15:01/*18; and HLA-DRB1*0101/*0301, then the pp65 peptides used to prime and expand the pp65 specific T-cell subpopulation are restricted to the specific HLA profile, and may include the peptides identified in Table 321 (Seq. ID. Nos. 2615-2619) for HLA-A*01; Table 322 (Seq. ID. Nos. 2620-2624) for HLA-A*02:01; Table 330 (Seq. ID. Nos. 2660-2664) for HLA-B*15:01; Table 331 (Seq. ID. Nos. 2665-2669) for HLA-B*18; Table 335 (Seq. ID. Nos. 2685-2689) for HLA-DRB1*0101; and Table 336 (Seq. ID. Nos. 2690-2694) for HLA-DRB1*0301.

In some embodiments, the HCMV pp65 HLA-restricted epitopes are specific to at least both of the donor's HLA-A alleles, at least both of the donor's HLA-B alleles, and at least both of the donor's HLA-DR alleles. In some embodiments, the HCMV pp65 HLA-A alleles are selected from a group comprising HLA-A*01, HLA-A*02:01, HLA-A*03, HLA-A*11:01, HLA-A*24:02, HLA-A*26, or HLA-A*68:01, and the corresponding HCMV pp65 HLA-restricted peptides are selected for: HLA-A*01 from Table 321; HLA-A*02:01 from Table 322; HLA-A*03 from Table 323; HLA-A*11:01 from Table 324; HLA-A*24:02 from Table 325; HLA-A*26 from Table 326; or HLA-A*68:01 from Table 327; or any combination thereof. In some embodiments, the HCMV pp65 HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding HCMV pp65 HLA-restricted peptides are selected for: HLA-B*07:02 from Table 328; HLA-B*08 from Table 329; HLA-B*15:01 (B62) from Table 330; HLA-B*18 from Table 331; HLA-B*27:05 from Table 332; HLA-B*35:01 from Table 333, or HLA-B*58:02 from Table 334; or any combination thereof. In some embodiments, the HCMV pp65 HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding HCMV pp65 HLA-restricted peptides are selected for: HLA-DRB1*0101 from Table 335; HLA-DRB1*0301 (DR17) from Table 336; HLA-DRB1*0401 (DR4Dw4) from Table 337; HLA-DRB1*0701 from Table 338; HLA-DRB1*1101 from Table 339; or HLA-DRB1*1501 (DR2b) from Table 340; or any combination thereof.

TABLE 321
HCMV pp65 HLA-A*01 Epitope Peptides

| SEQ ID NO. | Sequence |
|---|---|
| 2615 | YSEHPTFTSQY |
| 2616 | ATKMQVIGDQY |
| 2617 | SQYRIQGKLEY |
| 2618 | PSQEPMSIYVY |
| 2619 | ATVQGQNLKY |

TABLE 322
HCMV pp65 HLA-A*02:01 Epitope Peptides

| SEQ ID NO. | Sequence |
|---|---|
| 2620 | LLCPKSIPGL |
| 2621 | ILARNLVPMV |
| 2622 | ALFFFDIDLL |
| 2623 | ALPGPCIAST |
| 2624 | VLGPISGHVL |

TABLE 323
HCMV pp65 HLA-A*03 Epitope Peptides

| SEQ ID NO. | Sequence |
|---|---|
| 2625 | DVYYTSAFVF |
| 2626 | DLLLQRGPQY |
| 2627 | RLLQTGIHVR |
| 2628 | SIYVYALPLK |
| 2629 | VLCPKNMIIK |

TABLE 324
HCMV pp65 HLA-A*11:01 Epitope Peptides

| SEQ ID NO. | Sequence |
|---|---|
| 2630 | ASTSAGRKRK |
| 2631 | GVWQPAAQPK |
| 2632 | SVNVHNPTGR |
| 2633 | SIYVYALPLK |
| 2634 | YTSAFVFPTK |

TABLE 325
HCMV pp65 HLA-A*24:02 Epitope Peptides

| SEQ ID NO. | Sequence |
|---|---|
| 2635 | QYVKVYLESF |
| 2636 | QYDPVAALFF |
| 2637 | VFPTKDVAL |
| 2638 | VYYTSAFVF |
| 2639 | VYALPLKML |

TABLE 326
HCMV pp65 HLA-A*26 Epitope Peptides

| SEQ ID NO. | Sequence |
|---|---|
| 2640 | DVYYTSAFVF |
| 2641 | DVAFTSHEHF |
| 2642 | EVENVSVNVH |
| 2643 | EPMSIYVYAL |
| 2644 | FVFPTKDVAL |

TABLE 327
HCMV pp65 HLA-A*68:01 Epitope Peptides

| SEQ ID NO. | Sequence |
|---|---|
| 2645 | ELVTTERKTPR |
| 2646 | NVHHYPSAAER |
| 2647 | FVFPTKDVALR |
| 2648 | TVLCPKNMIIK |
| 2649 | DSDEELVTTER |

TABLE 328
HCMV pp65 HLA-B*07:02 Epitope Peptides

| SEQ ID NO. | Sequence |
|---|---|
| 2650 | EPMSIYVYAL |
| 2651 | KPGKISHIML |
| 2652 | IPGLSISGNL |
| 2653 | TPVLPHETRL |
| 2654 | VPSGKLFMHV |

TABLE 329
HCMV pp65 HLA-B*08 Epitope Peptides

| SEQ ID NO. | Sequence |
|---|---|
| 2655 | RRRHRQDAL |
| 2656 | AAERKHRHL |
| 2657 | VLCPKNMII |
| 2658 | QPKRRRHRQ |
| 2659 | ALPLKMLNI |

TABLE 330
HCMV pp65 HLA-B*15:01 (B62) Epitope Peptides

| SEQ ID NO. | Sequence |
|---|---|
| 2660 | NQWKEPDVYY |
| 2661 | RQYDPVAALF |
| 2662 | SQEPMSIYVY |
| 2663 | NQLQVQHTYF |
| 2664 | NLLMNGQQIF |

TABLE 331
HCMV pp65 HLA-B*18 Epitope Peptides

| SEQ ID NO. | Sequence |
|---|---|
| 2665 | NEIHNPAVF |
| 2666 | QEPMSIYVY |
| 2667 | AERKHRHL |
| 2668 | DPVAALFF |
| 2669 | DEELVTTE |

TABLE 332
HCMV pp65 HLA-B*27:05 Epitope Peptides

| SEQ ID NO. | Sequence |
|---|---|
| 2670 | RRCPEMISVL |
| 2671 | YRIQGKLEYR |
| 2672 | KRRRHRQDAL |
| 2673 | VRVSQPSLIL |
| 2674 | RLLQTGIHVR |

TABLE 333
HCMV pp65 HLA-B*35:01 Epitope Peptides

| SEQ ID NO. | Sequence |
|---|---|
| 2675 | DPVAALFFF |
| 2676 | WPPWQAGIL |
| 2677 | IPSINVHHY |
| 2678 | KPGKISHIM |
| 2679 | CPSQEPMSI |

TABLE 334
HCMV pp65 HLA-B*58:02 Epitope Peptides

| SEQ ID NO. | Sequence |
|---|---|
| 2680 | RSICPSQEPM |
| 2681 | PVLPHETRLL |
| 2682 | SAAERKHRHL |
| 2683 | MSIYVYALPL |
| 2684 | GSDVEEDLTM |

TABLE 335
HCMV pp65 HLA-DRB1*0101 Epitope Peptides

| SEQ ID NO. | Sequence |
|---|---|
| 2685 | RNGFTVLCPKNMIIK |
| 2686 | HEHFGLLCPKSIPGL |
| 2687 | LRQYDPVAALFFFDI |
| 2688 | CPEMISVLGPISGHV |
| 2689 | ISVLGPISGHVLKAV |

TABLE 336
HCMV pp65 HLA-DRB1*0301 (DR17) Epitope Peptides

| SEQ ID NO. | Sequence |
|---|---|
| 2690 | HVTLGSDVEEDLTMT |
| 2691 | YQEFFWDANDIYRIF |
| 2692 | SGNLLMNGQQIFLEV |
| 2693 | QPFMRPHERNGFTVL |
| 2694 | AALFFFDIDLLLQRG |

TABLE 337
HCMV pp65 HLA-DRB1*0401 (DR4Dw4) Epitope Peptides

| SEQ ID NO. | Sequence |
|---|---|
| 2695 | TPVLPHETRLLQTGI |
| 2696 | PLKMLNIPSINVHHY |
| 2697 | FAELEGVWQPAAQPK |
| 2698 | KAVFSRGDTPVLPHE |
| 2699 | VSQYTPDSTPCHRGD |

TABLE 338
HCMV pp65 HLA-DRB1*0701 Epitope Peptides

| SEQ ID NO. | Sequence |
|---|---|
| 2700 | PLKMLNIPSINVHHY |
| 2701 | SAFVFPTKDVALRHV |
| 2702 | ATKMQVIGDQYVKVY |
| 2703 | IPGLSISGNLLMNGQ |
| 2704 | KAVFSRGDTPVLPHE |

TABLE 339
HCMV pp65 HLA-DRB1*1101 Epitope Peptides

| SEQ ID NO. | Sequence |
|---|---|
| 2705 | HPTFTSQYRIQGKLE |
| 2706 | TSAFVFPTKDVALRH |
| 2707 | TSGVMTRGRLKAEST |
| 2708 | NIPSINVHHYPSAAE |
| 2709 | LPVADAVIHASGKQM |

TABLE 340
HCMV pp65 HLA-DRB1*1501 (DR2b) Epitope Peptides

| SEQ ID NO. | Sequence |
|---|---|
| 2710 | TVELRQYDPVAALFF |
| 2711 | LILVSQYTPDSTPCH |
| 2712 | NPAVFTWPPWQAGIL |
| 2713 | PEMISVLGPISGHVL |
| 2714 | QEPMSIYVYALPLKM |

### Human Cytomegalovirus (HCMV) Strain HHV-5 Antigenic Peptides

In some embodiments, the TVM or VM composition includes Human Cytomegalovirus (HCMV) HHV-5 VIE1 specific T-cells. VIE1 specific T-cells can be generated as described below using one or more antigenic peptides to VIE1. In some embodiments, the VIE1 specific T-cells are generated using one or more antigenic peptides to VIE1, or a modified or heteroclitic peptide derived from a VIE1 peptide. In some embodiments, VIE1 specific T-cells are generated using a VIE1 antigen library comprising a pool of peptides (for example 15mers) containing amino acid overlap (for example 11 amino acids of overlap) between each sequence formed by scanning the protein amino acid sequence SEQ. ID. No. 2715 (UniProt KB—P03169) for HCMV Strain HHV-5 VIE1:

MESSAKRKMDPDNPDEGPSSKVPRPETPVTKATTFLQTMLRKEVNSQLSL
GDPLFPELAEESLKTFERVTEDCNENPEKDVLAELVKQIKVRVDMVRHRI
KEHMLKKYTQTEEKFTGAFNMMGGCLQNALDILDKVHEPFEEMKCIGLTM
QSMYENYIVPEDKREMWMACIKELHDVSKGAANKLGGALQAKARAKKDEL
RRKMMYMCYRNIEFFTKNSAFPKTTNGCSQAMAALQNLPQCSPDEIMAYA
QKIFKILDEERDKVLTHIDHIFMDILTTCVETMCNEYKVTSDACMMTMYG
GISLLSEFCRVLSCYVLEETSVMLAKRPLITKPEVISVMKRRIEEICMKV
FAQYILGADPLRVCSPSVDDLRAIAEESDEEEAIVAYTLATRGASSSDSL
VSPPESPVPATIPLSSVIVAENSDQEESEQSDEEEEEGAQEEREDTVSVK
SEPVSEIEEVAPEEEEDGAEEPTASGGKSTHPMVTRSKADQ

In some embodiments, the VIE1 specific T-cells are generated using one or more antigenic peptides to VIE1, or a modified or heteroclitic peptide derived from a VIE1 peptide. In some embodiments, the VIE1 specific T-cells are generated with peptides that recognize class I MHC molecules. In some embodiments, the VIE1 specific T-cells are generated with peptides that recognize class II MHC molecules. In some embodiments, the VIE1 specific T-cells are generated with peptides that recognize both class I and class II MHC molecules.

In some embodiments, the VIE1 peptides used to prime and expand a T-cell subpopulation includes specifically selected HLA-restricted peptides generated by determining the HLA profile of the donor source, and including peptides derived from VIE1 that best match the donor's HLA. In some embodiments, the VIE1 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting VIE1 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 341-347, the HLA-B peptides are selected from the peptides of Tables 348-354, and the HLA-DR peptides are selected from the peptides of Tables 355-360. For example, if the donor cell source has an HLA profile that is HLA-A*01/*02:01; HLA-B*15:01/*18; and HLA-DRB1*0101/*0301, then the VIE1 peptides used to prime and expand the VIE1 specific T-cell subpopulation are restricted to the specific HLA profile, and may include the peptides identified in Table 341 (Seq. ID. Nos. 2716-2720) for HLA-A*01; Table 342 (Seq. ID. Nos. 2721-2725) for HLA-A*02:01; Table 350 (Seq. ID. Nos. 2761-2765) for HLA-B*15:01; Table 351 (Seq. ID. Nos. 2766-2770) for HLA-B*18; Table 355 (Seq. ID. Nos. 2786-2790) for HLA-DRB1*0101; and Table 356 (Seq. ID. Nos. 2791-2795) for HLA-DRB1*0301. In some embodiments, the mastermix of peptides includes both an overlapping peptide library and specifically selected HLA-restricted peptides generated by determining the HLA profile of the donor source.

In some embodiments, the HCMV VIE1 HLA-restricted epitopes are specific to at least both of the donor's HLA-A alleles, at least both of the donor's HLA-B alleles, and at least both of the donor's HLA-DR alleles. In some embodiments, the HCMV VIE1 HLA-A alleles are selected from a group comprising HLA-A*01, HLA-A*02:01, HLA-A*03, HLA-A*11:01, HLA-A*24:02, HLA-A*26, or HLA-A*68:01, and the corresponding HCMV VIE1 HLA-restricted peptides are selected for: HLA-A*01 from Table 341; HLA-A*02:01 from Table 342; HLA-A*03 from Table 343; HLA-A*11:01 from Table 344; HLA-A*24:02 from Table 345; HLA-A*26 from Table 346; or HLA-A*68:01 from Table 347; or any combination thereof. In some embodiments, the HCMV VIE1 HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding HCMV VIE1 HLA-restricted peptides are selected for: HLA-B*07:02 from Table 348; HLA-B*08 from Table 349; HLA-B*15:01 (B62) from Table 350; HLA-B*18 from Table 351; HLA-B*27:05 from Table 352; HLA-B*35:01 from Table 353, or HLA-B*58:02 from Table 354; or any combination thereof. In some embodiments, the HCMV VIE1 HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding HCMV VIE1 HLA-restricted peptides are selected for: HLA-DRB1*0101 from Table 355; HLA-DRB1*0301 (DR17) from Table 356; HLA-DRB1*0401 (DR4Dw4) from Table 357; HLA-DRB1*0701 from Table 358; HLA-DRB1*1101 from Table 359; or HLA-DRB1*1501 (DR2b) from Table 360; or any combination thereof.

TABLE 341
HCMV IE-1 HLA-A*01 Epitope Peptides

| SEQ ID NO. | Sequence |
|---|---|
| 2716 | LSEFCRVLSCY |
| 2717 | ESDEEEAIVAY |
| 2718 | KKDELRRKMMY |
| 2719 | TSDACMMTMY |
| 2720 | IKEHMLKKY |

TABLE 342
HCMV IE-1 HLA-A*02:01 Epitope Peptides

| SEQ ID NO. | Sequence |
|---|---|
| 2721 | SLGDPLFPEL |
| 2722 | LITKPEVISV |
| 2723 | VLAELVKQI |
| 2724 | TMYGGISLL |
| 2725 | ILDEERDKV |

TABLE 343
HCMV IE-1 HLA-A*03 Epitope Peptides

| SEQ ID NO. | Sequence |
|---|---|
| 2726 | ALQAKARAKK |
| 2727 | CIKELHDVSK |
| 2728 | DVSKGAANK |
| 2729 | RIKEHMLKK |
| 2730 | KLGGALQAK |

TABLE 344
HCMV IE-1 HLA-A*11:01 Epitope Peptides

| SEQ ID NO. | Sequence |
|---|---|
| 2731 | TTFLQTMLRK |
| 2732 | STHPMVTRSK |
| 2733 | ATTFLQTMLR |
| 2734 | IMAYAQKIFK |
| 2735 | KSTHPMVTR |

TABLE 345
HCMV IE-1 HLA-A*24:02 Epitope Peptides

| SEQ ID NO. | Sequence |
|---|---|
| 2736 | LFPELAEESL |
| 2737 | MYMCYRNIEF |
| 2738 | AYAQKIFKI |
| 2739 | KYTQTEEKF |
| 2740 | QYILGADPL |

TABLE 346
HCMV IE-1 HLA-A*26 Epitope Peptides

| SEQ ID NO. | Sequence |
|---|---|
| 2741 | ETPVTKATTF |
| 2742 | DVSKGAANKL |
| 2743 | ELAEESLKTF |
| 2744 | EVISVMKRRI |
| 2745 | EVNSQLSLG |

TABLE 347
HCMV IE-1 HLA-A*68:01 Epitope Peptides

| SEQ ID NO. | Sequence |
|---|---|
| 2746 | EAIVAYTLATR |
| 2747 | EIMAYAQKIFK |
| 2748 | ETSVMLAKR |
| 2749 | DVSKGAANK |
| 2750 | EVISVMKRR |

TABLE 348
HCMV IE-1 HLA-B*07:02 Epitope Peptides

| SEQ ID NO. | Sequence |
|---|---|
| 2751 | TPVTKATTFL |
| 2752 | NPEKDVLAEL |
| 2753 | SPVPATIPL |
| 2754 | VPRPETPVT |
| 2755 | RPETPVTKA |

TABLE 349
HCMV IE-1 HLA-B*08 Epitope Peptides

| SEQ ID NO. | Sequence |
|---|---|
| 2756 | KARAKKDEL |
| 2757 | VMKRRIEEI |
| 2758 | SAKRKMDPD |
| 2759 | RHRIKEHML |
| 2760 | ENPEKDVL |

TABLE 350
HCMV IE-1 HLA-B*15:01 (B62) Epitope Peptides

| SEQ ID NO. | Sequence |
|---|---|
| 2761 | ELAEESLKTF |
| 2762 | SQLSLGDPLF |
| 2763 | KVLTHIDHIF |
| 2764 | VLTHIDHIF |
| 2765 | ILDKVHEPF |

TABLE 351
HCMV IE-1 HLA-B*18 Epitope Peptides

| SEQ ID NO. | Sequence |
|---|---|
| 2766 | DEEEAIVAY |
| 2767 | DELRRKMMY |
| 2768 | EEMKCIGL |
| 2769 | EEKFTGAF |
| 2770 | DEERDKVL |

TABLE 352
HCMV IE-1 HLA-B*27:05 Epitope Peptides

| SEQ ID NO. | Sequence |
|---|---|
| 2771 | HRIKEHMLKK |
| 2772 | ARAKKDELRR |
| 2773 | RRIEEICMK |
| 2774 | LRKEVNSQL |
| 2775 | CRVLSCYVL |

TABLE 353
HCMV IE-1 HLA-B*35:01 Epitope Peptides

| SEQ ID NO. | Sequence |
|---|---|
| 2776 | NPEKDVLAEL |
| 2777 | TPVTKATTFL |
| 2778 | VPEDKREMWM |
| 2779 | FPELAEESL |
| 2780 | SPVPATIPL |

TABLE 354
HCMV IE-1 HLA-B*58:02 Epitope Peptides

| SEQ ID NO. | Sequence |
|---|---|
| 2781 | KATTFLQTM |
| 2782 | KTTNGCSQAM |
| 2783 | TSVMLAKRPL |
| 2784 | KARAKKDEL |
| 2785 | KVLTHIDHI |

TABLE 355
HCMV IE-1 HLA-DRB1*0101 Epitope Peptides

| SEQ ID NO. | Sequence |
|---|---|
| 2786 | DHIFMDILTTCVETM |
| 2787 | DACMMTMYGGISLLS |
| 2788 | LSEFCRVLSCYVLEE |
| 2789 | VFAQYILGADPLRVC |
| 2790 | TGAFNMMGGCLQNAL |

TABLE 356
HCMV IE-1 HLA-DRB1*0301 (DR17) Epitope Peptides

| SEQ ID NO. | Sequence |
|---|---|
| 2791 | FERVTEDCNENPEKD |
| 2792 | NYIVPEDKREMWMAC |
| 2793 | FNMMGGCLQNALDIL |
| 2794 | QTMLRKEVNSQLSLG |
| 2795 | MKCIGLTMQSMYENY |

TABLE 357
HCMV IE-1 HLA-DRB1*0401 (DR4Dw4)
Epitope Peptides

| SEQ ID NO. | Sequence |
|---|---|
| 2796 | DHIFMDILTTCVETM |
| 2797 | LSEFCRVLSCYVLEE |
| 2798 | LSCYVLEETSVMLAK |
| 2799 | IVAYTLATRGASSSD |
| 2800 | ETPVTKATTFLQTML |

TABLE 358
HCMV IE-1 HLA-DRB1*0701 Epitope Peptides

| SEQ ID NO. | Sequence |
|---|---|
| 2801 | DHIFMDILTTCVETM |
| 2802 | CNEYKVTSDACMMTM |
| 2803 | LSEFCRVLSCYVLEE |
| 2804 | LSCYVLEETSVMLAK |
| 2805 | DPLFPELAEESLKTF |

TABLE 359
HCMV IE-1 HLA-DRB1*1101 Epitope Peptides

| SEQ ID NO. | Sequence |
|---|---|
| 2806 | IKELHDVSKGAANKL |
| 2807 | IVAYTLATRGASSSD |
| 2808 | KDVLAELVKQIKVRV |
| 2809 | KPEVISVMKRRIEEI |
| 2810 | PEVISVMKRRIEEIC |

TABLE 360
HCMV IE-1 HLA-DRB1*1501 (DR2b)
Epitope Peptides

| SEQ ID NO. | Sequence |
|---|---|
| 2811 | ACMMTMYGGISLLSE |
| 2812 | ISLLSEFCRVLSCYV |
| 2813 | EESLKTFERVTEDCN |
| 2814 | ETPVTKATTFLQTML |
| 2815 | NSQLSLGDPLFPELA |

### Human Adenovirus C Serotype 2 (HAdV-2) (Human Adenovirus 2) Hexon Protein

In some embodiments, the TVM or VM composition includes Human adenovirus C serotype 2 (HMV-2) Hexon protein CAPSH specific T-cells. CAPSH specific T-cells can be generated as described below using one or more antigenic peptides to CAPSH. In some embodiments, the CAPSH specific T-cells are generated using one or more antigenic peptides to CAPSH, or a modified or heteroclitic peptide derived from a CAPSH peptide. In some embodiments, CAPSH specific T-cells are generated using a CAPSH antigen library comprising a pool of peptides (for example 15mers) containing amino acid overlap (for example 11 amino acids of overlap) between each sequence formed by scanning the protein amino acid sequence SEQ. ID. No. 2816 (UniProt KB—P03277) for Human adenovirus C serotype 2 (HAdV-2) Hexon protein CAPSH:

MATPSMMPQWSYMHISGQDASEYLSPGLVQFARATETYFSLNNKFRNPTV
APTHDVTTDRSQRLTLRFIPVDREDTAYSYKARFTLAVGDNRVLDMASTY
FDIRGVLDRGPTFKPYSGTAYNALAPKGAPNSCEWEQTEDSGRAVAEDEE
EEDEDEEEEEEEQNARDQATKKTHVYAQAPLSGETITKSGLQIGSDNAET
QAKPVYADPSYQPEPQIGESQWNEADANAAGGRVLKKTTPMKPCYGSYAR
PTNPFGGQSVLVPDEKGVPLPKVDLQFFSNTTSLNDRQGNATKPKVVLYS
EDVNMETPDTHLSYKPGKGDENSKAMLGQQSMPNRPNYIAFRDNFIGLMY
YNSTGNMGVLAGQASQLNAVVDLQDRNTELSYQLLLDSIGDRTRYFSMWN
QAVDSYDPDVRIIENHGTEDELPNYCFPLGGIGVTDTYQAIKANGNGSGD
NGDTTWTKDETFATRNEIGVGNNFAMEINLNANLWRNFLYSNIALYLPDK
LKYNPTNVEISDNPNTYDYMNKRVVAPGLVDCYINLGARWSLDYMDNVNP
FNHHRNAGLRYRSMLLGNGRYVPFHIQVPQKFFAIKNLLLLPGSYTYEWN
FRKDVNMVLQSSLGNDLRVDGASIKFDSICLYATFFPMAHNTASTLEAML
RNDTNDQSFNDYLSAANMLYPIPANATNVPISIPSRNWAAFRGWAFTRLK
TKETPSLGSGYDPYYTYSGSIPYLDGTFYLNHTFKKVAITFDSSVSWPGN
DRLLTPNEFEIKRSVDGEGYNVAQCNMTKDWFLVQMLANYNIGYQGFYIP
ESYKDRMYSFFRNFQPMSRQVVDDTKYKEYQQVGILHQHNNSGFVGYLAP
TMREGQAYPANVPYPLIGKTAVDSITQKKFLCDRTLWRIPFSSNFMSMGA
LTDLGQNLLYANSAHALDMTFEVDPMDEPTLLYVLFEVFDVVRVHQPHRG
VIETVYLRTPFSAGNATT

In some embodiments, the CAPSH specific T-cells are generated using one or more antigenic peptides to CAPSH, or a modified or heteroclitic peptide derived from a CAPSH peptide. In some embodiments, the CAPSH specific T-cells are generated with peptides that recognize class I MHC molecules. In some embodiments, the CAPSH specific T-cells are generated with peptides that recognize class II MHC molecules. In some embodiments, the CAPSH specific T-cells are generated with peptides that recognize both class I and class II MHC molecules.

In some embodiments, the CAPSH peptides used to prime and expand a T-cell subpopulation includes specifically selected HLA-restricted peptides generated by determining the HLA profile of the donor source, and including peptides derived from CAPSH that best match the donor's HLA. In some embodiments, the CAPSH peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting CAPSH derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 361-367, the HLA-B peptides are selected from the peptides of Tables 368-374, and the HLA-DR peptides are selected from the peptides of Tables 375-380. For example, if the donor cell source has an HLA profile that is HLA-A*01/*02:01; HLA-B*15:01/*18; and HLA-DRB1*0101/*0301, then the CAPSH peptides used to prime and expand the CAPSH specific T-cell subpopulation are restricted to the specific HLA profile, and may include the peptides identified in Table 361 (Seq. ID. Nos. 2817-2821) for HLA-A*01; Table 362 (Seq. ID. Nos. 2822-2826) for HLA-A*02:01; Table 370 (Seq. ID. Nos. 2862-2866) for HLA-B*15:01; Table 371 (Seq. ID. Nos. 2867-2871) for HLA-B*18; Table 375 (Seq. ID. Nos. 2887-2891) for HLA-DRB1*0101; and Table 376 (Seq. ID. Nos. 2892-2896) for HLA-DRB1*0301. In some embodiments, the mastermix of peptides includes both an overlapping peptide library and specifically selected HLA-restricted peptides generated by determining the HLA profile of the donor source.

In some embodiments, the HAdV-2 Hexon protein HLA-restricted epitopes are specific to at least both of the donor's HLA-A alleles, at least both of the donor's HLA-B alleles, and at least both of the donor's HLA-DR alleles. In some embodiments, the HAdV-2 Hexon protein HLA-A alleles are selected from a group comprising HLA-A*01, HLA-A*02:01, HLA-A*03, HLA-A*11:01, HLA-A*24:02, HLA-A*26, or HLA-A*68:01, and the corresponding HAdV-2 Hexon protein HLA-restricted peptides are selected for: HLA-A*01 from Table 361; HLA-A*02:01 from Table 362; HLA-A*03 from Table 363; HLA-A*11:01 from Table 364; HLA-A*24:02 from Table 365; HLA-A*26 from Table 366; or HLA-A*68:01 from Table 367; or any combination thereof. In some embodiments, the HAdV-2 Hexon protein HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding HAdV-2 Hexon protein HLA-restricted peptides are selected for: HLA-B*07:02 from Table 368; HLA-B*08 from Table 369; HLA-B*15:01 (B62) from Table 370; HLA-B*18 from Table 371; HLA-B*27:05 from Table 372; HLA-B*35:01 from Table 373, or HLA-B*58:02 from Table 374; or any combination thereof. In some embodiments, the HAdV-2 Hexon protein HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding HAdV-2 Hexon protein HLA-restricted peptides are selected for: HLA-DRB1*0101 from Table 375; HLA-DRB1*0301 (DR17) from Table 376; HLA-DRB1*0401 (DR4Dw4) from Table 377; HLA-DRB1*0701 from Table 378; HLA-DRB1*1101 from Table 379; or HLA-DRB1*1501 (DR2b) from Table 380; or any combination thereof.

TABLE 361
HADV HEXON HLA-A*01 Epitope Peptides

| SEQ ID NO. | Sequence |
|---|---|
| 2817 | LLDSIGDRTRY |
| 2818 | ISDNPNTYDY |
| 2819 | PMDEPTLLY |
| 2820 | LQDRNTELSY |
| 2821 | GTEDELPNY |

TABLE 362
HADV HEXON HLA-A*02:01 Epitope Peptides

| SEQ ID NO. | Sequence |
|---|---|
| 2822 | LLYANSAHAL |
| 2823 | TLLYVLFEV |
| 2824 | TLAVGDNRV |
| 2825 | VLAGQASQL |
| 2826 | MLLGNGRYV |

TABLE 363
HADV HEXON HLA-A*03 Epitope Peptides

| SEQ ID NO. | Sequence |
|---|---|
| 2827 | DLRVDGASIK |
| 2828 | RVLKKTTPMK |
| 2829 | RVLDMASTY |
| 2830 | ALYLPDKLK |
| 2831 | VLDRGPTFK |

TABLE 364
HADV HEXON HLA-A*11:01 Epitope Peptides

| SEQ ID NO. | Sequence |
|---|---|
| 2832 | GVLDRGPTFK |
| 2833 | GVTDTYQAIK |
| 2834 | GVIETVYLR |
| 2835 | ASTLEAMLR |
| 2836 | NVPYPLIGK |

TABLE 365
HADV HEXON HLA-A*24:02 Epitope Peptides

| SEQ ID NO. | Sequence |
|---|---|
| 2837 | AYPANVPYPL |
| 2838 | EYLSPGLVQF |
| 2839 | PYLDGTFYL |
| 2840 | TYFSLNNKF |
| 2841 | TYFDIRGVL |

TABLE 366
HADV HEXON HLA-A*26 Epitope Peptides

| SEQ ID NO. | Sequence |
|---|---|
| 2842 | EVDPMDEPTL |
| 2843 | ETPSLGSGY |
| 2844 | ETPDTHLSY |
| 2845 | ETVYLRTPF |
| 2846 | EVFDVVRVH |

TABLE 367
HADV HEXON HLA-A*68:01 Epitope Peptides

| SEQ ID NO. | Sequence |
|---|---|
| 2847 | TAVDSITQK |
| 2848 | ETYFSLNNK |
| 2849 | NVPYPLIGK |
| 2850 | LTPNEFEIKR |
| 2851 | DVVRVHQPHR |

TABLE 368
HADV HEXON HLA-B*07:02 Epitope Peptides

| SEQ ID NO. | Sequence |
|---|---|
| 2852 | IPYLDGTFYL |
| 2853 | VPDEKGVPL |
| 2854 | NPFGGQSVL |
| 2855 | YPANVPYPL |
| 2856 | DPMDEPTLL |

TABLE 369
HADV HEXON HLA-B*08 Epitope Peptides

| SEQ ID NO. | Sequence |
|---|---|
| 2857 | GLRYRSMLL |
| 2858 | VPDEKGVPL |
| 2859 | GLRYRSML |
| 2860 | SYKARFTL |
| 2861 | ATKPKVVL |

TABLE 370
HADV HEXON HLA-B*15:01 (B62) Epitope Peptides

| SEQ ID NO. | Sequence |
|---|---|
| 2862 | LLLLPGSYTY |
| 2863 | YQGFYIPESY |
| 2864 | VQFARATETY |
| 2865 | ALYLPDKLKY |
| 2866 | RQVVDDTKY |

TABLE 371
HADV HEXON HLA-B*18 Epitope Peptides

| SEQ ID NO. | Sequence |
|---|---|
| 2867 | DEPTLLYVL |
| 2868 | AETQAKPVY |
| 2869 | DELPNYCF |
| 2870 | DENSKAML |
| 2871 | TEDELPNY |

TABLE 372
HADV HEXON HLA-B*27:05 Epitope Peptides

| SEQ ID NO. | Sequence |
|---|---|
| 2872 | DRSQRLTLRF |
| 2873 | GRVLKKTTPM |
| 2874 | LRVDGASIKF |
| 2875 | WRIPFSSNF |
| 2876 | DRLLTPNEF |

TABLE 373
HADV HEXON HLA-B*35:01 Epitope Peptides

| SEQ ID NO. | Sequence |
|---|---|
| 2877 | IPVDREDTAY |
| 2878 | MPNRPNYIAF |
| 2879 | IPESYKDRMY |
| 2880 | DPMDEPTLLY |
| 2881 | VPDEKGVPL |

TABLE 374
HADV HEXON HLA-B*58:02 Epitope Peptides

| SEQ ID NO. | Sequence |
|---|---|
| 2882 | RSQRLTLRFI |
| 2883 | VSWPGNDRLL |
| 2884 | DSIGDRTRYF |
| 2885 | DSYDPDVRII |
| 2886 | KTTPMKPCY |

TABLE 375
HADV HEXON HLA-DRB1*0101 Epitope Peptides

| SEQ ID NO. | Sequence |
|---|---|
| 2887 | GTAYNALAPKGAPNS |
| 2888 | VDCYINLGARWSLDY |
| 2889 | YVPFHIQVPQKFFAI |
| 2890 | QWSYMHISGQDASEY |
| 2891 | TGNMGVLAGQASQLN |

TABLE 376
HADV HEXON HLA-DRB1*0301 (DR17) Epitope Peptides

| SEQ ID NO. | Sequence |
|---|---|
| 2892 | EWNFRKDVNMVLQSS |
| 2893 | GASIKFDSICLYATF |
| 2894 | THDVTTDRSQRLTLR |
| 2895 | QSVLVPDEKGVPLPK |
| 2896 | GRAVAEDEEEEDEDE |

TABLE 377
HADV HEXON HLA-DRB1*0401 (DR4Dw4) Epitope Peptides

| SEQ ID NO. | Sequence |
|---|---|
| 2897 | TLRFIPVDREDTAYS |
| 2898 | VVLYSEDVNMETPDT |
| 2899 | DTTWTKDETFATRNE |
| 2900 | GNNFAMEINLNANLW |
| 2901 | PQKFFAIKNLLLLPG |

TABLE 378
HADV HEXON HLA-DRB1*0701 Epitope Peptides

| SEQ ID NO. | Sequence |
|---|---|
| 2902 | LMYYNSTGNMGVLAG |
| 2903 | PQKFFAIKNLLLLPG |
| 2904 | DPYYTYSGSIPYLDG |
| 2905 | FKKVAITFDSSVSWP |
| 2906 | LVQFARATETYFSLN |

TABLE 379
HADV HEXON HLA-DRB1*1101 Epitope Peptides

| SEQ ID NO. | Sequence |
|---|---|
| 2907 | YATFFPMAHNTASTL |
| 2908 | PNTYDYMNKRVVAPG |
| 2909 | FRNFQPMSRQVVDDT |
| 2910 | TLRFIPVDREDTAYS |
| 2911 | NVPYPLIGKTAVDSI |

TABLE 380
HADV HEXON HLA-DRB1*1501 (DR2b) Epitope Peptides

| SEQ ID NO. | Sequence |
|---|---|
| 2912 | PGLVDCYINLGARWS |
| 2913 | FDSICLYATFFPMAH |
| 2914 | YSFFRNFQPMSRQVV |
| 2915 | PSMMPQWSYMHISGQ |
| 2916 | KVDLQFFSNTTSLND |

### Human Adenovirus Serotype 2 (HAdV-2) (Human Adenovirus 2) Penton Protein

In some embodiments, the TVM or VM composition includes Human adenovirus C serotype 2 (HAAT-2) Penton protein CAPSP specific T-cells. CAPSP specific T-cells can be generated as described below using one or more antigenic peptides to CAPSP. In some embodiments, the CAPSP specific T-cells are generated using one or more antigenic peptides to CAPSP, or a modified or heteroclitic peptide derived from a CAPSP peptide. In some embodiments, CAPSP specific T-cells are generated using a CAPSP antigen library comprising a pool of peptides (for example 15mers) containing amino acid overlap (for example 11 amino acids of overlap) between each sequence formed by scanning the protein amino acid sequence SEQ. ID. No. 2917 (UniProt KB—P03276) for Human adenovirus C serotype 2 (HAdV-2) Penton protein CAPSP:

MQRAAMYEEGPPPSYESVVSAAPVAAALGSPFDAPLDPPFVPPRYLRPT
GGRNSIRYSELAPLFDTTRVYLVDNKSTDVASLNYQNDHSNFLTTVIQN
NDYSPGEASTQTINLDDRSHWGGDLKTILHTNMPNVNEFMFTNKFKARV
MVSRSLTKDKQVELKYEWVEFTLPEGNYSETMTIDLMNNAIVEHYLKVG
RQNGVLESDIGVKFDTRNFRLGFDPVTGLVMPGVYTNEAFHPDIILLPG
CGVDFTHSRLSNLLGIRKRQPFQEGFRITYDDLEGGNIPALLDVDAYQA
SLKDDTEQGGDGAGGGNNSGSGAEENSNAAAAAMQPVEDMNDHAIRGDT
FATRAEEKRAEAEAAAEAAAPAAQPEVEKPQKKPVIKPLTEDSKKRSYN
LISNDSTFTQYRSWYLAYNYGDPQTGIRSWTLLCTPDVTCGSEQVYWSL
PDMMQDPVTFRSTSQISNFPVVGAELLPVHSKSFYNDQAVYSQLIRQFT
SLTHVFNRFPENQILARPPAPTITTVSENVPALTDHGTLPLRNSIGGVQ
RVTITDARRRTCPYVYKALGIVSPRVLSSRTF

In some embodiments, the CAPSP specific T-cells are generated using one or more antigenic peptides to CAPSP, or a modified or heteroclitic peptide derived from a CAPSP peptide. In some embodiments, the CAPSP specific T-cells are generated with peptides that recognize class I MHC molecules. In some embodiments, the CAPSP specific T-cells are generated with peptides that recognize class II MHC molecules. In some embodiments, the CAPSP specific T-cells are generated with peptides that recognize both class I and class II MHC molecules.

In some embodiments, the CAPSP peptides used to prime and expand a T-cell subpopulation includes specifically selected HLA-restricted peptides generated by determining the HLA profile of the donor source, and including peptides derived from CAPSP that best match the donor's HLA. In some embodiments, the CAPSP peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting CAPSP derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 381-387, the HLA-B peptides are selected from the peptides of Tables 388-394, and the HLA-DR peptides are selected from the peptides of Tables 395-400. For example, if the donor cell source has an HLA profile that is HLA-A*01/*02:01; HLA-B*15:01/*18; and HLA-DRB1*0101/*0301, then the CAPSP peptides used to prime and expand the CAPSP specific T-cell subpopulation are restricted to the specific HLA profile, and may include the peptides identified in Table 381 (Seq. ID. Nos. 2918-2922) for HLA-A*01; Table 382 (Seq. ID. Nos. 2923-2927) for HLA-A*02:01; Table 390 (Seq. ID. Nos. 2963-2967) for HLA-B*15:01; Table 391 (Seq. ID. Nos. 2968-2972) for HLA-B*18; Table 395 (Seq. ID. Nos. 2988-2992) for HLA-DRB1*0101; and Table 396 (Seq. ID. Nos. 2993-2997) for HLA-DRB1*0301. In some embodiments, the mastermix of peptides includes both an overlapping peptide library and specifically selected HLA-restricted peptides generated by determining the HLA profile of the donor source.

In some embodiments, the HAdV-2 Penton protein HLA-restricted epitopes are specific to at least both of the donor's HLA-A alleles, at least both of the donor's HLA-B alleles, and at least both of the donor's HLA-DR alleles. In some embodiments, the HAdV-2 Penton protein HLA-A alleles are selected from a group comprising HLA-A*01, HLA-A*02:01, HLA-A*03, HLA-A*11:01, HLA-A*24:02, HLA-A*26, or HLA-A*68:01, and the corresponding HAdV-2 Penton protein HLA-restricted peptides are selected for: HLA-A*01 from Table 381; HLA-A*02:01 from Table 382; HLA-A*03 from Table 383; HLA-A*11:01 from Table 384; HLA-A*24:02 from Table 385; HLA-A*26 from Table 386; or HLA-A*68:01 from Table 387; or any combination thereof. In some embodiments, the HAdV-2 Penton protein HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding HAdV-2 Penton protein HLA-restricted peptides are selected for: HLA-B*07:02 from Table 388; HLA-B*08 from Table 389; HLA-B*15:01 (B62) from Table 390; HLA-B*18 from Table 391; HLA-B*27:05 from Table 392; HLA-B*35:01 from Table 393, or HLA-B*58:02 from Table 394; or any combination thereof. In some embodiments, the HAdV-2 Penton protein HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding HAdV-2 Penton protein HLA-restricted peptides are selected for: HLA-DRB1*0101 from Table 395; HLA-DRB1*0301 (DR17) from Table 396; HLA-DRB1*0401 (DR4Dw4) from Table 397; HLA-DRB1*0701 from Table 398; HLA-DRB1*1101 from Table 399; or HLA-DRB1*1501 (DR2b) from Table 400; or any combination thereof.

TABLE 381
HADV PENTON HLA-A*01 Epitope Peptides

| SEQ ID NO. | Sequence |
|---|---|
| 2918 | ITDARRRTCPY |
| 2919 | PLDPPFVPPRY |
| 2920 | STDVASLNY |
| 2921 | TKDKQVELKY |
| 2922 | LTEDSKKRSY |

TABLE 382
HADV PENTON HLA-A*02:01 Epitope Peptides

| SEQ ID NO. | Sequence |
|---|---|
| 2923 | DLEGGNIPAL |
| 2924 | SIRYSELAPL |
| 2925 | SLTKDKQVEL |
| 2926 | ILARPPAPTI |
| 2927 | AIVEHYLKV |

TABLE 383
HADV PENTON HLA-A*03 Epitope Peptides

| SEQ ID NO. | Sequence |
|---|---|
| 2928 | RVMVSRSLTK |
| 2929 | GVLESDIGVK |
| 2930 | ILLPGCGVDF |
| 2931 | RLSNLLGIRK |
| 2932 | DVDAYQASLK |

TABLE 384
HADV PENTON HLA-A*11:01 Epitope Peptides

| SEQ ID NO. | Sequence |
|---|---|
| 2933 | GVLESDIGVK |
| 2934 | RVMVSRSLTK |
| 2935 | LTKDKQVELK |
| 2936 | DVDAYQASLK |
| 2937 | DTFATRAEEK |

TABLE 385
HADV PENTON HLA-A*24:02 Epitope Peptides

| SEQ ID NO. | Sequence |
|---|---|
| 2938 | NYSETMTIDL |
| 2939 | TYDDLEGGNI |
| 2940 | RYSELAPLF |
| 2941 | KYEWVEFTL |
| 2942 | VYSQLIRQF |

TABLE 386
HADV PENTON HLA-A*26 Epitope Peptides

| SEQ ID NO. | Sequence |
|---|---|
| 2943 | DVTCGSEQVY |
| 2944 | EAFHPDIILL |
| 2945 | TTVSENVPAL |
| 2946 | DVDAYQASL |
| 2947 | TVSENVPAL |

TABLE 387
HADV PENTON HLA-A*68:01 Epitope Peptides

| SEQ ID NO. | Sequence |
|---|---|
| 2948 | DTFATRAEEKR |
| 2949 | PVEDMNDHAIR |
| 2950 | ELAPLFDTTR |
| 2951 | IVEHYLKVGR |
| 2952 | RVTITDARR |

TABLE 388
HADV PENTON HLA-B*07:02 Epitope Peptides

| SEQ ID NO. | Sequence |
|---|---|
| 2953 | PPSYESVVSA |
| 2954 | VPALTDHGTL |
| 2955 | DPPFVPPRYL |
| 2956 | PPFVPPRYL |
| 2957 | RPPAPTITT |

TABLE 389
HADV PENTON HLA-B*08 Epitope Peptides

| SEQ ID NO. | Sequence |
|---|---|
| 2958 | LTKDKQVEL |
| 2959 | DSKKRSYNL |
| 2960 | ELKYEWVEF |
| 2961 | QKKPVIKPL |
| 2962 | GIRKRQPF |

TABLE 390
HADV PENTON HLA-B*15:01 (B62) Epitope Peptides

| SEQ ID NO. | Sequence |
|---|---|
| 2963 | ILLPGCGVDF |
| 2964 | RQFTSLTHVF |
| 2965 | TQYRSWYLAY |
| 2966 | VLESDIGVKF |
| 2967 | PLFDTTRVY |

TABLE 391
HADV PENTON HLA-B*18 Epitope Peptides

| SEQ ID NO. | Sequence |
|---|---|
| 2968 | NEFMFTNKF |
| 2969 | LESDIGVKF |
| 2970 | TEDSKKRSY |
| 2971 | EEGPPPSY |
| 2972 | QEGFRITY |

TABLE 392
HADV PENTON HLA-B*27:05 Epitope Peptides

| SEQ ID NO. | Sequence |
|---|---|
| 2973 | FRSTSQISNF |
| 2974 | GRNSIRYSEL |
| 2975 | IRYSELAPLF |
| 2976 | QRVTITDARR |
| 2977 | RRTCPYVYK |

TABLE 393
HADV PENTON HLA-B*35:01 Epitope Peptides

| SEQ ID NO. | Sequence |
|---|---|
| 2978 | APLFDTTRVY |
| 2979 | IPALLDVDAY |
| 2980 | VPALTDHGTL |
| 2981 | FPVVGAELL |
| 2982 | LPVHSKSFY |

TABLE 394
HADV PENTON HLA-B*58:02 Epitope Peptides

| SEQ ID NO. | Sequence |
|---|---|
| 2983 | KARVMVSRSL |
| 2984 | KSTDVASLNY |
| 2985 | KSFYNDQAVY |
| 2986 | RSWYLAYNY |
| 2987 | RSTSQISNF |

TABLE 395
HADV PENTON HLA-DRB1*0101 Epitope Peptides

| SEQ ID NO. | Sequence |
|---|---|
| 2988 | HPDIILLPGCGVDFT |
| 2989 | RITYDDLEGGNIPAL |
| 2990 | IRSWTLLCTPDVTCG |
| 2991 | ISNFPVVGAELLPVH |
| 2992 | RLGFDPVTGLVMPGV |

TABLE 396
HADV PENTON HLA-DRB1*0301 (DR17) Epitope Peptides

| SEQ ID NO. | Sequence |
|---|---|
| 2993 | SRSLTKDKQVELKYE |
| 2994 | NGVLESDIGVKFDTR |
| 2995 | NFRLGFDPVTGLVMP |
| 2996 | IKPLTEDSKKRSYNL |
| 2997 | DIGVKFDTRNFRLGF |

TABLE 397
HADV PENTON HLA-DRB1*0401 (DR4Dw4) Epitope
Peptides

| SEQ ID NO. | Sequence |
|---|---|
| 2998 | SLNYQNDHSNFLTTV |
| 2999 | KRSYNLISNDSTFTQ |
| 3000 | IRSWTLLCTPDVTCG |
| 3001 | QAVYSQLIRQFTSLT |
| 3002 | IRQFTSLTHVFNRFP |

TABLE 398
HADV PENTON HLA-DRB1*0701 Epitope Peptides

| SEQ ID NO. | Sequence |
|---|---|
| 3003 | EGNYSETMTIDLMNN |
| 3004 | APLFDTTRVYLVDNK |
| 3005 | SLNYQNDHSNFLTTV |
| 3006 | DSTFTQYRSWYLAYN |
| 3007 | IRSWTLLCTPDVTCG |

TABLE 399
HADV PENTON HLA-DRB1*1101 Epitope Peptides

| SEQ ID NO. | Sequence |
|---|---|
| 3008 | VEHYLKVGRQNGVLE |
| 3009 | LSNLLGIRKRQPFQE |
| 3010 | IRQFTSLTHVFNRFP |
| 3011 | VNEFMFTNKFKARVM |
| 3012 | PPSYESVVSAAPVAA |

TABLE 400
HADV PENTON HLA-DRB1*1501 (DR2b) Epitope Peptides

| SEQ ID NO. | Sequence |
|---|---|
| 3013 | SQLIRQFTSLTHVFN |
| 3014 | NAIVEHYLKVGRQNG |
| 3015 | QTGIRSWTLLCTPDV |
| 3016 | TSQISNFPVVGAELL |
| 3017 | LLDVDAYQASLKDDT |

### BK Polyomavirus (BKPyV) (Human Polyomavirus 1) Large T Antigen

In some embodiments, the TVM or VM composition includes BK polyomavirus (BKPyV) (Human polyomavirus 1) Large T Antigen LT specific T-cells. LT specific T-cells can be generated as described below using one or more antigenic peptides to LT. In some embodiments, the LT specific T-cells are generated using one or more antigenic peptides to LT, or a modified or heteroclitic peptide derived from a LT peptide. In some embodiments, LT specific T-cells are generated using a LT antigen library comprising a pool of peptides (for example 15mers) containing amino acid overlap (for example 11 amino acids of overlap) between each sequence formed by scanning the protein amino acid sequence SEQ. ID. No. 3018 (UniProt KB—P03071) for BK polyomavirus (BKPyV) (Human polyomavirus 1) Large T Antigen LT:

MDKVLNREESMELMDLLGLERAAWGNLPLMRKAYLRKCKEFHPDKGGDED
KMKRMNTLYKKMEQDVKVAHQPDFGTWSSSEVPTYGTEEWESWWSSFNEK
WDEDLFCHEDMFASDEEATADSQHSTPPKKKRKVEDPKDFPSDLHQFLSQ
AVFSNRTLACFAVYTTKEKAQILYKKLMEKYSVTFISRHMCAGHNIIFFL
TPHRHRVSAINNFCQKLCTFSFLICKGVNKEYLLYSALTRDPYHTIEESI
QGGLKEHDFSPEEPEETKQVSWKLITEYAVETKCEDVFLLLGMYLEFQYN
VEECKKCQKKDQPYHFKYHEKHFANAIIFAESKNQKSICQQAVDTVLAKK
RVDTLHMTREEMLTERFNHILDKMDLIFGAHGNAVLEQYMAGVAWLHCLL
PKMDSVIFDFLHCIVFNVPKRRYWLFKGPIDSGKTTLAAGLLDLCGGKAL
NVNLPMERLTFELGVAIDQYMVVFEDVKGTGAESKDLPSGHGINNLDSLR
DYLDGSVKVNLEKKHLNKRTQIFPPGLVTMNEYPVPKTLQARFVRQIDFR
PKIYLRKSLQNSEFLLEKRILQSGMTLLLLLIWFRPVADFATDIQSRIVE
WKERLDSEISMYTFSRMKYNICMGKCILDITREEDSETEDSGHGSSTESQ
SQCSSQVSDTSAPAEDSQRSDPHSQELHLCKGFQCFKRPKTPPPK

In some embodiments, the LT specific T-cells are generated using one or more antigenic peptides to LT, or a modified or heteroclitic peptide derived from a LT peptide. In some embodiments, the LT specific T-cells are generated with peptides that recognize class I MHC molecules. In some embodiments, the LT specific T-cells are generated with peptides that recognize class II MHC molecules. In some embodiments, the LT specific T-cells are generated with peptides that recognize both class I and class II MHC molecules.

In some embodiments, the LT peptides used to prime and expand a T-cell subpopulation includes specifically selected HLA-restricted peptides generated by determining the HLA profile of the donor source, and including peptides derived from LT that best match the donor's HLA. In some embodiments, the LT peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting LT derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 401-407, the HLA-B peptides are selected from the peptides of Tables 408-414, and the HLA-DR peptides are selected from the peptides of Tables 415-420. For example, if the donor cell source has an HLA profile that is HLA-A*01/*02:01; HLA-B*15:01/*18; and HLA-DRB1*0101/*0301, then the LT peptides used to prime and expand the LT specific T-cell subpopulation are restricted to the specific HLA profile, and may include the peptides identified in Table 401 (Seq. ID. Nos. 3019-3023) for HLA-A*01; Table 402 (Seq. ID. Nos. 3024-3028) for HLA-A*02:01; Table 410 (Seq. ID. Nos. 3064-3068) for HLA-B*15:01; Table 411 (Seq. ID. Nos. 3069-3073) for HLA-B*18; Table 415 (Seq. ID. Nos. 3089-3093) for HLA-DRB1*0101; and Table 416 (Seq. ID. Nos. 3094-3098) for HLA-DRB1*0301. In some embodiments, the mastermix of peptides includes both an overlapping peptide library and specifically selected HLA-restricted peptides generated by determining the HLA profile of the donor source.

In some embodiments, the BKPyV HLA-restricted epitopes are specific to at least both of the donor's HLA-A alleles, at least both of the donor's HLA-B alleles, and at least both of the donor's HLA-DR alleles. In some embodiments, the BKPyV HLA-A alleles are selected from a group comprising HLA-A*01, HLA-A*02:01, HLA-A*03, HLA-A*11:01, HLA-A*24:02, HLA-A*26, or HLA-A*68:01, and the corresponding BKPyV HLA-restricted peptides are selected for: HLA-A*01 from Table 401; HLA-A*02:01 from Table 402; HLA-A*03 from Table 403; HLA-A*11:01 from Table 404; HLA-A*24:02 from Table 405; HLA-A*26 from Table 406; or HLA-A*68:01 from Table 407; or any combination thereof. In some embodiments, the BKPyV HLA-B alleles are selected from a group comprising HLA-B*07:02, HLA-B*08, HLA-B*15:01 (B62), HLA-B*18, HLA-B*27:05, HLA-B*35:01, or HLA-B*58:02, and the corresponding BKPyV HLA-restricted peptides are selected for: HLA-B*07:02 from Table 408; HLA-B*08 from Table 409; HLA-B*15:01 (B62) from Table 410; HLA-B*18 from Table 411; HLA-B*27:05 from Table 412; HLA-B*35:01 from Table 413, or HLA-B*58:02 from Table 414; or any combination thereof. In some embodiments, the BKPyV HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b) and the corresponding BKPyV HLA-restricted peptides are selected for: HLA-DRB1*0101 from Table 415; HLA-DRB1*0301 (DR17) from Table 416; HLA-DRB1*0401 (DR4Dw4) from Table 417; HLA-DRB1*0701 from Table 418; HLA-DRB1*1101 from Table 419; or HLA-DRB1*1501 (DR2b) from Table 420; or any combination thereof.

TABLE 401
BKPYV LARGE T ANTIGEN HLA-A*01 Epitope Peptides

| SEQ ID NO. | Sequence |
|---|---|
| 3019 | CEDVFLLLGMY |
| 3020 | YTTKEKAQILY |
| 3021 | KKDQPYHFKY |
| 3022 | RLDSEISMY |
| 3023 | QIDFRPKIY |

TABLE 402
BKPYV LARGE T ANTIGEN HLA-A*02:01 Epitope Peptides

| SEQ ID NO. | Sequence |
|---|---|
| 3024 | VLAKKRVDTL |
| 3025 | SICQQAVDTV |
| 3026 | YLDGSVKVNL |
| 3027 | TLAAGLLDL |
| 3028 | CLLPKMDSV |

TABLE 403
BKPYV LARGE T ANTIGEN HLA-A*03 Epitope Peptides

| SEQ ID NO. | Sequence |
|---|---|
| 3029 | DVGTGAESK |
| 3030 | FLICKGVNK |
| 3031 | RDYLDGSVK |
| 3032 | ILYKKLMEK |
| 3033 | AVDTVLAKK |

TABLE 404
BKPYV LARGE T ANTIGEN HLA-A*11:01 Epitope Peptides

| SEQ ID NO. | Sequence |
|---|---|
| 3034 | VTMNEYPVPK |
| 3035 | GSVKVNLEK |
| 3036 | CTFSFLICK |
| 3037 | STPPKKKRK |
| 3038 | AVDTVLAKK |

TABLE 405
BKPYV LARGE T ANTIGEN HLA-A*24:02 Epitope Peptides

| SEQ ID NO. | Sequence |
|---|---|
| 3039 | VYTTKEKAQI |
| 3040 | KYNICMGKCI |
| 3041 | AYLRKCKEF |
| 3042 | PYHTIEESI |
| 3043 | QYMAGVAWL |

TABLE 406
BKPYV LARGE T ANTIGEN HLA-A*26 Epitope Peptides

| SEQ ID NO. | Sequence |
|---|---|
| 3044 | ELGVAIDQY |
| 3045 | ETKCEDVFLL |
| 3046 | EDVFLLLGMY |
| 3047 | DVFLLLGMY |
| 3048 | ETKQVSWKL |

TABLE 407
BKPYV LARGE T ANTIGEN HLA-A*68:01 Epitope Peptides

| SEQ ID NO. | Sequence |
|---|---|
| 3049 | DTSAPAEDSQR |
| 3050 | LVTMNEYPVPK |
| 3051 | CTFSFLICK |
| 3052 | AVDTVLAKK |
| 3053 | PVPKTLQAR |

TABLE 408
BKPYV LARGE T ANTIGEN HLA-B*07:02 Epitope Peptides

| SEQ ID NO. | Sequence |
|---|---|
| 3054 | LPSGHGINNL |
| 3055 | LPMERLTFEL |
| 3056 | RPKIYLRKSL |
| 3057 | GPIDSGKTTL |
| 3058 | FPSDLHQFL |

TABLE 409
BKPYV LARGE T ANTIGEN HLA-B*08 Epitope Peptides

| SEQ ID NO. | Sequence |
|---|---|
| 3059 | LAKKRVDTL |
| 3060 | TTKEKAQIL |
| 3061 | VPKRRYWL |
| 3062 | FLLEKRIL |
| 3063 | PLMRKAYL |

TABLE 410
BKPYV LARGE T ANTIGEN HLA-B*15:01 (B62) Epitope
Peptides

| SEQ ID NO. | Sequence |
|---|---|
| 3064 | RQIDFRPKIY |
| 3065 | KLMEKYSVTF |
| 3066 | ILYKKLMEKY |
| 3067 | LLGMYLEFQY |
| 3068 | NLPLMRKAY |

TABLE 411
BKPYV LARGE T ANTIGEN HLA-B*18 Epitope Peptides

| SEQ ID NO. | Sequence |
|---|---|
| 3069 | REEMLTERF |
| 3070 | QELHLCKGF |
| 3071 | NEKWDEDLF |
| 3072 | EEMLTERF |
| 3073 | MEKYSVTF |

TABLE 412
BKPYV LARGE T ANTIGEN HLA-B*27:05 Epitope Peptides

| SEQ ID NO. | Sequence |
|---|---|
| 3074 | ARFVRQIDFR |
| 3075 | KRILQSGMTL |
| 3076 | HRVSAINNF |
| 3077 | ERFNHILDK |
| 3078 | KRMNTLYKK |

TABLE 413
BKPYV LARGE T ANTIGEN HLA-B*35:01 Epitope Peptides

| SEQ ID NO. | Sequence |
|---|---|
| 3079 | GPIDSGKTTL |
| 3080 | LPMERLTFEL |
| 3081 | LPSGHGINNL |
| 3082 | FPSDLHQFL |
| 3083 | LPLMRKAYL |

TABLE 414
BKPYV LARGE T ANTIGEN HLA-B*58:02 Epitope Peptides

| SEQ ID NO. | Sequence |
|---|---|
| 3084 | KSLQNSEFLL |
| 3085 | KAQILYKKLM |
| 3086 | KAYLRKCKEF |
| 3087 | KTTLAAGLL |
| 3088 | KALNVNLPM |

TABLE 415
BKPYV LARGE T ANTIGEN HLA-DRB1*0101 Epitope
Peptides

| SEQ ID NO. | Sequence |
|---|---|
| 3089 | MVVFEDVKGTGAESK |
| 3090 | CEDVFLLLGMYLEFQ |
| 3091 | AAGLLDLCGGKALNV |
| 3092 | RMKYNICMGKCILDI |
| 3093 | QILYKKLMEKYSVTF |

TABLE 416
BKPYV LARGE T ANTIGEN HLA-DRB1*0301 (DR17)
Epitope Peptides

| SEQ ID NO. | Sequence |
|---|---|
| 3094 | PKDFPSDLHQFLSQA |
| 3095 | YKKMEQDVKVAHQPD |
| 3096 | EDMFASDEEATADSQ |
| 3097 | YMVVFEDVKGTGAES |
| 3098 | WGNLPLMRKAYLRKC |

TABLE 417
BKPYV LARGE T ANTIGEN HLA-DRB1*0401 (DR4Dw4)
Epitope Peptides

| SEQ ID NO. | Sequence |
|---|---|
| 3099 | PKDFPSDLHQFLSQA |
| 3100 | NSEFLLEKRILQSGM |
| 3101 | VADFATDIQSRIVEW |
| 3102 | CKGFQCFKRPKTPPP |
| 3103 | DKVLNREESMELMDL |

TABLE 418
BKPYV LARGE T ANTIGEN HLA-DRB1*0701 Epitope
Peptides

| SEQ ID NO. | Sequence |
|---|---|
| 3104 | QVSWKLITEYAVETK |
| 3105 | VDTLHMTREEMLTER |
| 3106 | WESWWSSFNEKWDED |
| 3107 | TFELGVAIDQYMVVF |
| 3108 | QPDFGTWSSSEVPTY |

TABLE 419
BKPYV LARGE T ANTIGEN HLA-DRB1*1101 Epitope
Peptides

| SEQ ID NO. | Sequence |
|---|---|
| 3109 | IFFLTPHRHRVSAIN |
| 3110 | YLLYSALTRDPYHTI |
| 3111 | PYHFKYHEKHFANAI |
| 3112 | HCIVFNVPKRRYWLF |
| 3113 | YMVVFEDVKGTGAES |

TABLE 420
BKPYV LARGE T ANTIGEN HLA-DRB1*1501 (DR2b)
Epitope Peptides

| SEQ ID NO. | Sequence |
|---|---|
| 3114 | CQKLCTFSFLICKGV |
| 3115 | DQYMVVFEDVKGTGA |
| 3116 | LLLLIWFRPVADFAT |
| 3117 | PSDLHQFLSQAVFSN |
| 3118 | GHNIIFFLTPHRHRV |

Method of Treating a Patient in Conjunction with a Hematopoietic Stem Cell Transplant by Administering a TVM or VM Composition

The invention includes a method to treat a patient receiving a HSCT, typically a human, by administering an effective amount of a TVM or VM composition described herein concomitantly with the administration of the HSCT or following administration of the HSCT.

The dose administered may vary according to the decision of the healthcare practitioner. In some embodiments, the TVM or VM composition is administered to a patient, such as a human in a dose ranging from 1×10⁶ cells/m² to 1×10⁸ cells/m² of each multi-antigen specific T-cell subpopulation and 1×10⁶ cells/kg to 1×10⁷ cells/kg of a mesenchymal stem cell subpopulation. The dose can be a single dose, for example, comprising the combination of all of the T-cell and MSC subpopulations in the TVM or VM combined composition, or in multiple separate doses, wherein each dose comprises a separate T-cell and MSC subpopulation and the collective separate doses of T-cell and MSC subpopulations comprise the total TVM or VM composition. In some embodiments, each T-cell subpopulation dosage is 1×10⁶ cells/m², 2×10⁶ cells/m², 3×10⁶ cells/m², 4×10⁶ cells/m², 5×10⁶ cells/m², 6×10⁶ cells/m², 7×10⁶ cells/m², 8×10⁶ cells/m², 9×10⁶ cells/m², 1×10⁷ cells/m², 2×10⁷ cells/m², 3×10⁷ cells/m², 4×10⁷ cells/m², 5×10⁷ cells/m², 6×10⁷ cells/m², 7×10⁷ cells/m², 8×10⁷ cells/m², 9×10⁷ cells/m², or 1×10⁸ cells/m². In some embodiments, each MSC subpopulation dosage is 1×10⁶ cells/kg, 2×10⁶ cells/kg, 3×10⁶ cells/kg, 4×10⁶ cells/kg, or 5×10⁶ cells/kg, 6×10⁶ cells/kg, 7×10⁶ cells/kg, 8×10⁶ cells/kg, 9×10⁶ cells/kg, or 1×10⁷ cells/kg.

The TVM or VM composition may be administered by any suitable method. In some embodiments, the TVM or VM composition is administered to a patient, such as a human as an infusion and in a particular embodiment, an infusion with a total volume of 1 to 20 cc. In some embodiments, the TVM or VM composition is administered to a patient as a 1, 2, 3, 4, 5, 6, 7, 8, 9, 10, 11, 12, 13, 14, 15, 16, 17, 18, 19, or 20 cc infusion. In some embodiments, the TVM or VM composition when present as an infusion is administered to a patient over 10, 20, 30, 40, 50, 60 or more minutes to the patient in need thereof.

In some embodiments, a patient receiving an infusion has vital signs monitored before, during, and 1-hour post infusion of the TVM or VM composition. In certain embodiments, patients with stable disease (SD), partial response (PR), or complete response (CR) up to 6 weeks after initial infusion may be eligible to receive additional infusions, for example, 1, 2, 3, 4, 5, 6, 7, 8, 9 or 10 additional infusions several weeks apart, for example, up to about 1, 2, 3, 4, 5, 6, 7, 8, 9 or 10 weeks apart.

### Determining the Tumor-Associated Antigen Expression Profile

Determining a TAA expression profile can be performed by any method known in the art. Non-limiting exemplary methods for determining a tumor-associated antigen expression profile can be found in Ding et al., Cancer Bio Med (2012) 9: 73-76; Qin et al., Leukemia Research (2009) 33(3) 384-390; and Weber et al., Leukemia (2009) 23: 1634-1642. In some embodiments, TAA expression profiles are generated from a sample collected from a patient with a malignancy or tumor. In some embodiments, the sample is selected from a group consisting of blood, bone marrow, and tumor biopsy.

In some embodiments, the TAA expression profile is determined from a blood sample of a patient with a malignancy or tumor. In some embodiments, the TAA expression profile is determined from a bone marrow sample of a patient with a malignancy or tumor. In some embodiments, the TAA expression profile is determined from a tumor biopsy sample of a patient with a malignancy or tumor.

In some embodiments, genetic material is extracted from the sample collected from a patient with a malignancy or tumor. In some embodiments, the genetic material is selected from a group consisting of total RNA, messenger RNA and genomic DNA.

After extraction of genetic material, quantitative reverse transcriptase polymerase chain reaction (qPCR) is performed on the genetic material utilizing primers developed from TAAs of interest.

The patient's tumor cells can be checked for reactivity against activated T-cell subpopulations and/or the TVM composition of the present invention using any known methods, including cytotoxicity assays described herein.

### Hematological and Solid Tumors Targeted for Treatment

The TVM compositions described herein can be used to treat a patient with a solid or hematological malignancy who is undergoing HSCT in conjunction with the administration of the TVM composition.

Lymphoid neoplasms are broadly categorized into precursor lymphoid neoplasms and mature T-cell, B-cell or natural killer cell (NK) neoplasms. Chronic leukemias are those likely to exhibit primary manifestations in blood and bone marrow, whereas lymphomas are typically found in extramedullary sites, with secondary events in the blood or bone. Over 79,000 new cases of lymphoma were estimated in 2013. Lymphoma is a cancer of lymphocytes, which are a type of white blood cell. Lymphomas are categorized as Hodgkin's or non-Hodgkin's. Over 48,000 new cases of leukemias were expected in 2013.

In some embodiments, the disease or disorder is a hematological malignancy selected from a group consisting of leukemia, lymphoma and multiple myeloma.

In some embodiments, the methods described herein can be used to treat a leukemia. For example, the patient such as a human may be suffering from an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AML); large granular lymphocytic leukemia; or Adult T-cell chronic leukemia. In some embodiments, the patient suffers from an acute myelogenous leukemia, for example an undifferentiated AML (M0); myeloblastic leukemia (M1; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7).

In a particular embodiment, the hematological malignancy is a lymphoma or lymphocytic or myelocytic proliferation disorder or abnormality. In some embodiments, the lymphoma is a non-Hodgkin's lymphoma. In some embodiments, the lymphoma is a Hodgkin's lymphoma. In some embodiments, the hematological malignancy is a relapsed or refractory leukemia, lymphoma, or myeloma.

In some aspects, the methods described herein can be used to treat a patient such as a human, with a Non-Hodgkin's Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's Macroglobulinemia.

Alternatively, the methods described herein can be used to treat a patient, such as a human, with a Hodgkin's Lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL.

Alternatively, the methods described herein can be used to treat a patient, for example a human, with specific B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; Mediastinal large B cell lymphoma; Nodal marginal zone B cell lymphoma (NMZL); Splenic marginal zone lymphoma (SMZL); Intravascular large B-cell lymphoma; Primary effusion lymphoma; or Lymphomatoid granulomatosis; B-cell prolymphocytic leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T cell/histiocyte rich large B-cell lymphoma; DLBCL associated with chronic inflammation; Epstein-Barr virus (EBV)+ DLBCL of the elderly; Primary mediastinal (thymic) large B-cell lymphoma; Primary cutaneous DLBCL, leg type; ALK+ large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma; or B-cell lymphoma, unclassifiable, with features intermediate between diffuse large B-cell lymphoma and classical Hodgkin lymphoma.

Abnormal proliferation of T-cells, B-cells, and/or NK-cells can result in a wide range of cancers. A host, for example a human, afflicted with any of these disorders can be treated with an effective amount of the TAA-L composition as described herein to achieve a decrease in symptoms (a palliative agent) or a decrease in the underlying disease (a disease modifying agent).

Alternatively, the methods described herein can be used to treat a patient, such as a human, with a hematological malignancy, for example but not limited to T-cell or NK-cell lymphoma, for example, but not limited to: peripheral T-cell lymphoma; anaplastic large cell lymphoma, for example anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sézary syndrome, primary cutaneous anaplastic large cell lymphoma, primary cutaneous CD30+ T-cell lymphoproliferative disorder; primary cutaneous aggressive epidermotropic CD8+ cytotoxic T-cell lymphoma; primary cutaneous gamma-delta T-cell lymphoma; primary cutaneous small/medium CD4+ T-cell lymphoma, and lymphomatoid papulosis; Adult T-cell Leukemia/Lymphoma (ATLL); Blastic NK-cell Lymphoma; Enteropathy-type T-cell lymphoma; Hematosplenic gamma-delta T-cell Lymphoma; Lymphoblastic Lymphoma; Nasal NK/T-cell Lymphomas; Treatment-related T-cell lymphomas; for example lymphomas that appear after solid organ or bone marrow transplantation; T-cell prolymphocytic leukemia; T-cell large granular lymphocytic leukemia; Chronic lymphoproliferative disorder of NK-cells; Aggressive NK cell leukemia; Systemic EBV+ T-cell lymphoproliferative disease of childhood (associated with chronic active EBV infection); Hydroa vacciniforme-like lymphoma; Adult T-cell leukemia/lymphoma; Enteropathy-associated T-cell lymphoma; Hepatosplenic T-cell lymphoma; or Subcutaneous panniculitis-like T-cell lymphoma.

In some embodiments, the TVM composition disclosed herein is used to treat a patient with a selected hematopoietic malignancy either before or after hematopoietic stem cell transplantation (HSCT). In some embodiments, the TVM composition is used to treat a patient with a selected hematopoietic malignancy after HSCT. In some embodiments, the TVM composition is used to treat a patient with a selected hematopoietic malignancy up to about 30, 35, 40, 45, or 50 days after HSCT. In some embodiments, the TVM composition is used to treat a patient with a selected hematopoietic malignancy after neutrophil engraftment during the period following HSCT. In some embodiments, the TVM composition is used to treat a patient with a selected hematopoietic malignancy before HSCT, such as one week, two weeks, three weeks or more before HSCT.

In some aspects, the tumor is a solid tumor. In some embodiments, the solid tumor is Wilms Tumor. In some embodiments, the solid tumor is osteosarcoma. In some embodiments, the solid tumor is Ewing's sarcoma. In some embodiments, the solid tumor is neuroblastoma. In some embodiments, the solid tumor is soft tissue sarcoma. In some embodiments, the solid tumor is rhabdomyosarcoma. In some embodiments, the solid tumor is glioma. In some embodiments, the solid tumor is germ cell cancer. In some embodiments, the solid tumor is breast cancer. In some embodiments, the solid tumor is lung cancer. In some embodiments the solid tumor is ovarian cancer. In some embodiments, the solid tumor is renal cell carcinoma. In some embodiments, the solid tumor is colon cancer. In some embodiments, the solid tumor is melanoma. In some embodiments, the solid tumor is a relapsed or refractory solid tumor.

Non-limiting examples of tumors that can be treated according to the present invention include, but are not limited to, acoustic neuroma, adenocarcinoma, adrenal gland cancer, anal cancer, angiosarcoma (e.g., lymphangiosarcoma, lymphangioendotheli osarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast, triple negative breast cancer, HER2-negative breast cancer, HER2-positive breast cancer, male breast cancer, late-line metastatic breast cancer, progesterone receptor-negative breast cancer, progesterone receptor-positive breast cancer, recurrent breast cancer), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), glioblastoma multiforme, head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

Non-Cancer Disorders Targeted for Treatment with Hematopoietic Stem Cell Transplant

The VM compositions described herein can be used to treat a patient with a non-cancer disorder who is undergoing HSCT. In some embodiments, the disease or disorder is an autoimmune disease. In some embodiments, the disease or disorder is a metabolic disorder. In some embodiments, the disease or disorder is a primary immune deficiency disorder.

In some embodiments, the methods described herein can be used to treat a patient with an autoimmune disease. Non-limiting examples of autoimmune diseases that can be treated with HSCT include, but are not limited to, Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Diamond-Blackfan anemia, Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Hemophagocytic lymphohistiocytosis (HLH), Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and Wegener's granulomatosis (or Granulomatosis with Polyangiitis (GPA)).

In another embodiment, VM compositions and methods described herein can be used to treat a patient with a metabolic disorder undergoing a HSCT. Non-limiting examples of metabolic disorders that can be treated with HSCT include, but are not limited to, mucopolysaccharidosis, including MPS I (Hurler, Scheie, H-S syndrome), MPS II (Hunter syndrome), MPS III A-D (Sanfilippo A-D syndrome), MPS IV A-B (Morquio A-B syndrome), MPS VI (Maroteaux-Lamy syndrome), MPS VII (Sly syndrome); glycoproteinosis, including, but not limited to aspartylglucosaminuria, fucosidosis, α-Mannosidosis, β-Mannosidosis, Mucolipidosis III and IV (sialidosis), Schindler disease; Sphingolipidosis, including but not limited to Fabry's disease, Farber's (lipogranulomatosis, Gaucher's I-III, GM1 gangliosidosis, Niemann-Pick disease A and B, Tay-Sachs disease, Sandhoff s disease, Globoid leukodystrophy (Krabbe disease), metachromatic leukodystrophy (MLD), other lipidosis, including but not limited to Niemann-Pick disease C, Wolman disease, Ceroid lipofuscinosis Type III (Batten ds); glycogen storage disorders including but not limited to GSD type II (Pompe disease); multiple enzyme deficiency disorders, including but not limited to galactosialidosis, mucolipidosis type II (I-cell disease), and other mucolipidoses; lysosomal transport defects, including but not limited to cystinosis, sialic acid storage disease, Salla disease; peroxisomal storage disorders (PSD) including but not limited to adrenoleukodystrophy, adrenomyeloneuropathy. Inherited genetic disorders include, but are not limited to hemoglobinopathies including b-Thalassemia major, a-Thalassemia major, and Sickle cell anemia; hematopoietic diseases including osteopetrosis, Diamond-Blackfan syndrome, Shwachman-Diamond syndrome, Dyskeratosis congenita, Fanconi anemia, Congenital amegakaryocytic thrombocytopenia; haemoglobinopathies including severe SS anaemia, Congenital erythropoietic porphyria (CEP, Gunther's disease, Congenital dyserythropoietic anaemia (CDA) types I and II, Hereditary sideroblastic anaemia, Pyruvate kinase deficiency; Platelet disorders including Glanzmann's thrombasthenia.

In yet another embodiment, the VM compositions and methods described herein can be used to treat a patient with a primary immune deficiency disorder that is undergoing a HSCT. Non-limiting examples of primary immune deficiency disorder that can be treated with HSCT include, but are not limited to, Primary immune deficiencies include, but are not limited to, Wiskott-Aldrich syndrome, Epidermolysis bullosa, Severe congenital neutropenia, Thalassemia major, Leukocyte adhesion deficiency, chronic granulomatous disease, familial hemophagocytic lymphohistiocytosis, hyperimmunoglobulin M (HIgM) syndrome, severe combined immunodeficiency (SCID), and leukocyte adhesion deficiency type 1 (LAD1), Bare Lymphocyte Syndrome, CD40 Ligand Deficiency, Chediak-Higashi Syndrome, Combined Immunodeficiency Disease, hemophagocytosis, and leukocyte lymphoproliferative syndrome. T/B+ SCID, γc deficiency, JAK3 deficiency, interleukin 7 r deficiency, CD45 deficiency, CD3δ/CD3ε deficiency, TB-SCID, RAG 1/2 deficiency, DCLRE1C deficiency, ADA deficiency, reticular dysgenesis, Omenn syndrome, DNA ligase type IV deficiency, Cernunnos deficiency, CD40 ligand deficiency, CD40 deficiency, Purine nucleoside phosphorylase (PNP) deficiency, CD3γ deficiency, CD8 deficiency, ZAP-70 deficiency, Ca++ channel deficiency, MHC class I deficiency, MHC class II deficiency, Winged helix deficiency, CD25 deficiency, STAT5b deficiency, Itk deficiency, and DOCK8 deficiency, infantile agranulocytosis (Kostman's syndrome), lazy leukocyte syndrome (neutrophil actin deficiency), neutrophil membrane GP-180 deficiency, agammaglobulinemia, and X-linked lymphoproliferative syndrome.

### Administration of TVM and VM Compositions

Methods for administration of cells for adoptive cell therapy are known and may be used in connection with the provided methods and TVM or VM compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

The administration of the TVM or VM composition may vary. In one aspect, the TVM or VM composition may be administered to a patient such as a human at an interval selected from once every 1 week, 2 weeks, 3 weeks, 4 weeks, 5 weeks, 6 weeks, 7 weeks, 8 weeks, or more after the initial administration of the TVM or VM composition. In a typical embodiment, the TVM or VM composition is administered in an initial dose then at every 4 weeks thereafter. In some embodiments, the TVM or VM composition may be administered repetitively to 1, 2, 3, 4, 5, 6, or more times after the initial administration of the composition. In a typical embodiment, the TVM or VM composition is administered repetitively up to 10 more times after the initial administration of the TVM or VM composition. In an alternative embodiment, the TVM or VM composition is administered more than 10 times after the initial administration of the TVM or VM composition.

In some embodiments for the treatment of a patient undergoing HSCT with cancer, a TAA expression profile of the malignancy or tumor of the patient, for example, a human is performed prior to the initial administration of the TVM composition. In some embodiments, a TAA expression profile of the malignancy or tumor of the patient is performed prior to each subsequent administration of the TVM composition, allowing for the option to adjust the TVM composition. In some embodiments, the TVM composition of subsequent administrations remains the same as the initial administration. In some embodiments, the TVM composition of subsequent administrations is changed based on the change in the TAA expression profile of the malignancy or tumor of the patient.

In some embodiments, the TVM or VM compositions are administered to a subject in the form of a pharmaceutical composition, such as a composition comprising the cells or cell populations and a pharmaceutically acceptable carrier or excipient. The pharmaceutical compositions in some embodiments additionally comprise other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The choice of carrier in the pharmaceutical composition may be determined in part by the by the particular method used to administer the cell composition. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition.

In addition, buffering agents in some aspects are included in the composition. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins 21st ed. (May 1, 2005).

In some embodiments, the pharmaceutical composition comprises the TVM or VM composition in an amount that is effective to treat or prevent the disease or condition, such as a therapeutically effective or prophylactically effective amount. Thus, in some embodiments, the methods of administration include administration of the TVM or VM composition at effective amounts. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, the TVM or VM composition is administered at a desired dosage, which in some aspects includes a desired dose or number of cells and/or a desired number of T-cell subpopulations. Thus, the dosage of cells in some embodiments is based on a total number of cells (or number per m² body surface area or per kg body weight) and a desired amount of the individual populations or sub-types. In some embodiments, the dosage of cells is based on a desired total number (or number per m² body surface area or per kg of body weight) of cells in the individual populations or of individual cell types. In some embodiments, the dosage is based on a combination of such features, such as a desired number of total cells, and desired total number of cells in the individual populations.

In some embodiments, the TVM or VM composition is administered at or within a tolerated difference of a desired dose of total cells, such as a desired dose of T cells or MSCs. In some aspects, the desired dose is a desired number of cells, a desired number of cells per unit of body surface area or a desired number of cells per unit of body weight of the subject to whom the cells are administered, e.g., cells/m² or cells/kg. In some aspects, the desired dose is at or above a minimum number of cells or minimum number of cells per unit of body surface area or body weight. In some aspects, among the total cells, administered at the desired dose, the individual populations or sub-types are present at or near a desired output ratio as described herein, e.g., within a certain tolerated difference or error of such a ratio.

In some embodiments, the cells are administered at or within a tolerated difference of a desired dose. In some aspects, the desired dose is a desired number of cells, or a desired number of such cells per unit of body surface area or body weight of the subject to whom the cells are administered, e.g., cells/m² or cells/kg. In some aspects, the desired dose is at or above a minimum number of cells of the population, or minimum number of cells of the population per unit of body surface area or body weight.

Thus, in some embodiments, the dosage is based on a desired fixed dose of total cells and/or based on a desired fixed dose of two or more, e.g., each, of the individual T-cell and MSC subpopulations. Thus, in some embodiments, the dosage is based on a desired fixed or minimum dose of T-cell and MSC subpopulations and a desired ratio thereof.

In certain embodiments, TVM or VM composition is administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual T-cell subpopulations of cells is within a range of between at or about 10⁴ and at or about 10⁹ cells/meter² (m²) body surface area, such as between 10⁵ and 10⁶ cells/m² body surface area, for example, at or about 1×10⁵ cells/m², 1.5×10⁵ cells/m², 2×10⁵ cells/m², or 1×10⁶ cells/m² body surface area. For example, in some embodiments, the cells are administered at, or within a certain range of error of, between at or about 10⁴ and at or about 10⁹ T cells/meter² (m²) body surface area, such as between 10⁵ and 10⁶ T cells/m² body surface area, for example, at or about 1×10⁵ T cells/m², 1.5×10⁵ T cells/m², 2×10⁵ T cells/m², or 1×10⁶ T cells/m² body surface area.

In some embodiments, the cells are administered at or within a certain range of error of between at or about 10⁴ and at or about 10⁹ cells/meter² (m²) body weight, such as between 10⁵ and 10⁶ cells/m² body weight, for example, at or about 1×10⁵ cells/m², 1.5×10⁵ cells/m², 2×10⁵ cells/kg, or 1×10⁶ cells/m² body surface area.

### Product Release Testing and Characterization of T-Cell Subpopulations

Prior to infusion, the TVM or VM composition may be characterized for safety and release testing. Product release testing, also known as lot or batch release testing, is an important step in the quality control process of drug substances and drug products. This testing verifies that a T-cell or MSC subpopulation and/or TVM or VM composition meets a pre-determined set of specifications. Pre-determined release specifications for T-cell and MSC subpopulations and TVM or VM compositions include confirmation that the cell product is >70% viable, has <5.0 EU/ml of endotoxin, is negative for aerobic, anaerobic, fungal pathogens and mycoplasma, and slacks reactivity to allogeneic PHA blasts, for example, with less than 10% lysis to PHA blasts. The phenotype of the T-cell subpopulations comprising the TVM or VM composition may be determined with requirements for clearance to contain, in one non-limiting embodiment, <2% dendritic cells and <2% B cells. The HLA identity between the T-cell subpopulations comprising the TVM or VM composition and the donor is also confirmed. The phenotype of the MSC subpopulation comprising the TVM or VM composition may be determined by flow cytometry with requirements for clearance to contain, in one non-limiting embodiment >90% CD73+, CD90+, and CD105+ cells; <2% CD34+, CD45+, CD14+, and CD19+ cells; and <5% HLA-DR+ cells.

Antigen specificity of the T-cell subpopulations can be tested via an Interferon-γ Enzyme-Linked Immunospot (IFNγ ELISpot) assay. Other cytokines can also be utilized to measure antigen specificity including TNFα and IL-4. Pre-stimulated effector cells and target cells pulsed with the TAAs or VAAs of interest are incubated in a 96-well plate (pre-incubated with anti-INF- [symbol] antibody) at an E/T ratio of 1:2. They are compared with a no antigen control, an irrelevant peptide not used for T-cell generation, and SEB as a positive control. After washing, the plates are incubated with a biotinylated anti-IFN- [symbol] antibody. Spots are detected by incubating with streptavidin-coupled alkaline phosphastase and substrate. Spot forming cells (SFCs) are counted and evaluated using an automated plate reader.

The phenotype of the TVM or VM composition can be determined by extracellular antibody staining with anti-CD3, CD4, CD8, CD14, CD16, CD19, CD34, CD45, CD56, CD73, CD83, CD90, CD105, HLA-DR, TCRαβ, TCRγδ and analyzed on a flow cytometer. Annexin-V and PI antibodies can be used as viability controls, and data analyzed with FlowJo Flow Cytometry software (Treestar, Ashland, Oreg., USA).

The lytic capacity of T-cell subpopulations can be evaluated via ⁵¹Chromium (⁵¹Cr) and Europium (Eu)-release cytotoxicity assays to test recognition and lysis of target cells by the T-cell subpopulations comprising the TVM compositions.

Typically, activated primed T-cells (effector cells) can be tested against ⁵¹Cr-labeled target cells at effector-to-target ratios of, for example, 40:1, 20:1, 10:1, and 5:1. Cytolytic activity can be determined by measuring ⁵¹Cr release into the supernatant on a gamma-counter. Spontaneous release is assessed by incubating target cells alone, and maximum lysis by adding 1% Triton X-100. Specific lysis was calculated as: specific lysis (%)=(experimental release−spontaneous release)/(maximum release−spontaneous release)×100.

Europium-release assays can also be utilized to measure the lytic capacity of T-cell subpopulations comprising the TVM or VM compositions. This is a non-radioactive alternative to the conventional Chromium-51 (⁵¹Cr) release assay and works on the same principle as the radioactive assay. Target cells are first loaded with an acetoxymethyl ester of BATDA. The ligand penetrates the cell membrane quickly. Within the cell, the ester bonds are hydrolyzed to form a hydrophilic ligand (TDA), which no longer passes through the cell membrane. If cells are lysed by an effector cell, TDA is released outside the cell into the supernatant. Upon addition of Europium solution to the supernatant, Europium can form a highly fluorescent and stable chelate with the released TDA (EuTDA). The measured fluorescence signal correlates directly with the number of lysed cells in the cytotoxicity assay. Specific lysis was calculated as: specific lysis (%)=(experimental release−spontaneous release)/(maximum release−spontaneous release)×100.

### Monitoring

Following administration of the cells, the biological activity of the administered cell populations in some embodiments is measured, e.g., by any of a number of known methods. Parameters to assess include specific binding of a T-cell or other immune cell to antigen, in vivo, e.g., by imaging, or ex vivo, e.g., by ELISA or flow cytometry. In certain embodiments, the ability of the administered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004), all incorporated herein by reference. In certain embodiments, the biological activity of the cells is measured by assaying expression and/or secretion of one or more cytokines, such as IFNγ, IL-2, and TNF. In some aspects the biological activity is measured by assessing clinical outcome, such as reduction in tumor burden or load.

### Combination Therapies

In one aspect of the invention, TVM or VM compositions disclosed herein can be beneficially administered in combination with another therapeutic regimen for beneficial, additive, or synergistic effects.

In some embodiments, the TVM composition is administered in combination with another therapy to treat a hematological malignancy. In some embodiments, the TVM composition is administered in combination with another therapy to treat a solid tumor. The second therapy can be a pharmaceutical or a biologic agent (for example an antibody) to increase the efficacy of treatment with a combined or synergistic approach.

In some embodiments, the additional therapy is a monoclonal antibody (MAb). Some MAbs stimulate an immune response that destroys tumor cells. Similar to the antibodies produced naturally by B cells, these MAbs “coat” the tumor cell surface, triggering its destruction by the immune system. FDA-approved MAbs of this type include rituximab, which targets the CD20 antigen found on non-Hodgkin lymphoma cells, and alemtuzumab, which targets the CD52 antigen found on B-cell chronic lymphocyticleukemia (CLL) cells. Rituximab may also trigger cell death (apoptosis) directly. Another group of MAbs stimulates an antitumor immune response by binding to receptors on the surface of immune cells and inhibiting signals that prevent immune cells from attacking the body's own tissues, including tumor cells. Other MAbs interfere with the action of proteins that are necessary for tumor growth. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. Similarly, cetuximab and panitumumab target the epidermal growth factor receptor (EGFR). MAbs that bind to cell surface growth factor receptors prevent the targeted receptors from sending their normal growth-promoting signals. They may also trigger apoptosis and activate the immune system to destroy tumor cells. Another group of tumor therapeutic MAbs are the immunoconjugates. These MAbs, which are sometimes called immunotoxins or antibody-drug conjugates, consist of an antibody attached to a cell-killing substance, such as a plant or bacterial toxin, a chemotherapy drug, or a radioactive molecule. The antibody latches onto its specific antigen on the surface of a tumor cell, and the cell-killing substance is taken up by the cell. FDA-approved conjugated MAbs that work this way include 90Y-ibritumomab tiuxetan, which targets the CD20 antigen to deliver radioactive yttrium-90 to B-cell non-Hodgkin lymphoma cells; ¹³¹I-tositumomab, which targets the CD20 antigen to deliver radioactive ¹³¹I to non-Hodgkin lymphoma cells.

In some embodiments, the additional agent is an immune checkpoint inhibitor (ICI), for example, but not limited to PD-1 inhibitors, PD-L1 inhibitors, PD-L2 inhibitors, CTLA-4 inhibitors, LAG-3 inhibitors, TIM-3 inhibitors, and V-domain Ig suppressor of T-cell activation (VISTA) inhibitors, or combinations thereof.

In some embodiments, the immune checkpoint inhibitor is a PD-1 inhibitor that blocks the interaction of PD-1 and PD-L1 by binding to the PD-1 receptor, and in turn inhibits immune suppression. In some embodiments, the immune checkpoint inhibitor is a PD-1 immune checkpoint inhibitor selected from nivolumab (Opdivo®), pembrolizumab (Keytruda®), pidilizumab, AMP-224 (AstraZeneca and MedImmune), PF-06801591 (Pfizer), MEDI0680 (AstraZeneca), PDR001 (Novartis), REGN2810 (Regeneron), MGA012 (MacroGenics), BGB-A317 (BeiGene) SHR-12-1 (Jiangsu Hengrui Medicine Company and Incyte Corporation), TSR-042 (Tesaro), and the PD-L1/VISTA inhibitor CA-170 (Curis Inc.).

In some embodiments, the immune checkpoint inhibitor is the PD-1 immune checkpoint inhibitor nivolumab (Opdivo®) administered in an effective amount for the treatment of Hodgkin's lymphoma. In another aspect of this embodiment, the immune checkpoint inhibitor is the PD-1 immune checkpoint inhibitor pembrolizumab (Keytruda®) administered in an effective amount. In an additional aspect of this embodiment, the immune checkpoint inhibitor is the PD-1 immune checkpoint inhibitor pidilizumab (Medivation) administered in an effective amount for refractory diffuse large B-cell lymphoma (DLBCL).

In some embodiments, the immune checkpoint inhibitor is a PD-L1 inhibitor that blocks the interaction of PD-1 and PD-L1 by binding to the PD-L1 receptor, and in turn inhibits immune suppression. PD-L1 inhibitors include, but are not limited to, atezolizumab, durvalumab, KNO35CA-170 (Curis Inc.), and LY3300054 (Eli Lilly).

In some embodiments, the immune checkpoint inhibitor is the PD-L1 immune checkpoint inhibitor atezolizumab (Tecentriq®) administered in an effective amount. In another aspect of this embodiment, the immune checkpoint inhibitor is durvalumab (AstraZeneca and MedImmune) administered in an effective. In yet another aspect of the embodiment, the immune checkpoint inhibitor is KN035 (Alphamab). An additional example of a PD-L1 immune checkpoint inhibitor is BMS-936559 (Bristol-Myers Squibb), although clinical trials with this inhibitor have been suspended as of 2015.

In one aspect of this embodiment, the immune checkpoint inhibitor is a CTLA-4 immune checkpoint inhibitor that binds to CTLA-4 and inhibits immune suppression. CTLA-4 inhibitors include, but are not limited to, ipilimumab, tremelimumab (AstraZeneca and MedImmune), AGEN1884 and AGEN2041 (Agenus).

In some embodiments, the CTLA-4 immune checkpoint inhibitor is ipilimumab (Yervoy®) administered in an effective amount

In another embodiment, the immune checkpoint inhibitor is a LAG-3 immune checkpoint inhibitor. Examples of LAG-3 immune checkpoint inhibitors include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGD013 (MacroGenics). In yet another aspect of this embodiment, the immune checkpoint inhibitor is a TIM-3 immune checkpoint inhibitor. A specific TIM-3 inhibitor includes, but is not limited to, TSR-022 (Tesaro).

Other immune checkpoint inhibitors for use in combination with the invention described herein include, but are not limited to, B7-H3/CD276 immune checkpoint inhibitors such as MGA217, indoleamine 2,3-dioxygenase (IDO) immune checkpoint inhibitors such as Indoximod and INCB024360, killer immunoglobulin-like receptors (KIRs) immune checkpoint inhibitors such as Lirilumab (BMS-986015), carcinoembryonic antigen cell adhesion molecule (CEACAM) inhibitors (e.g., CEACAM-1, -3 and/or -5). Exemplary anti-CEACAM-1 antibodies are described in WO 2010/125571, WO 2013/082366 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep. 2; 5(9). pii: e12529 (DOI:10: 1371/journal.pone.0021146), or cross-reacts with CEACAM-1 and CEACAM-5 as described in, e.g., WO 2013/054331 and US 2014/0271618. Still other checkpoint inhibitors can be molecules directed to B and T lymphocyte attenuator molecule (BTLA), for example as described in Zhang et al., Monoclonal antibodies to B and T lymphocyte attenuator (BTLA) have no effect on in vitro B cell proliferation and act to inhibit in vitro T cell proliferation when presented in a cis, but not trans, format relative to the activating stimulus, Clin Exp Immunol. 2011 January; 163(1): 77-87.

Current chemotherapeutic drugs that may be used in combination with the TVM composition described herein include those used to treat AML including cytarabine (cytosine arabinoside or ara-C) and the anthracycline drugs (such as daunorubicin/daunomycin, idarubicin, and mitoxantrone). Some of the other chemo drugs that may be used to treat AML include: Cladribine (Leustatin®, 2-CdA), Fludarabine (Fludara®), Topotecan, Etoposide (VP-16), 6-thioguanine (6-TG), Hydroxyurea (Hydrea®), Corticosteroid drugs, such as prednisone or dexamethasone (Decadron®), Methotrexate (MTX), 6-mercaptopurine (6-MP), Azacitidine (Vidaza®), Decitabine (Dacogen®). Additional drugs include dasatinib and checkpoint inhibitors such as novolumab, Pembrolizumab, and atezolizumab.

Current chemotherapeutic drugs that may be used in combination with the TVM composition described herein include those used for CLL and other lymphomas including: purine analogs such as fludarabine (Fludara®), pentostatin (Nipent®), and cladribine (2-CdA, Leustatin®), and alkylating agents, which include chlorambucil (Leukeran®) and cyclophosphamide (Cytoxan®) and bendamustine (Treanda®). Other drugs sometimes used for CLL include doxorubicin (Adriamycin®), methotrexate, oxaliplatin, vincristine (Oncovin®), etoposide (VP-16), and cytarabine (ara-C). Other drugs include Rituximab (Rituxan), Obinutuzumab (Gazyva™), Ofatumumab (Arzerra®), Alemtuzumab (Campath®) and Ibrutinib (Imbruvica™)

Current chemotherapeutic drugs that may be used in combination with the TVM composition described herein include those used for CML including: Interferon, imatinib (Gleevec), the chemo drug hydroxyurea (Hydrea®), cytarabine (Ara-C), busulfan, cyclophosphamide (Cytoxan®), and vincristine (Oncovin®). Omacetaxine (Synribo®) is a chemo drug that was approved to treat CML that is resistant to some of the TKIs now in use.

Current chemotherapeutic drugs that may be used in combination with the TVM composition described herein include those used for CMML, for example, Deferasirox (Exjade®), cytarabine with idarubicin, cytarabine with topotecan, and cytarabine with fludarabine, Hydroxyurea (hydroxycarbamate, Hydrea®), azacytidine (Vidaza®) and decitabine (Dacogen®).

Current chemotherapeutic drugs that may be used in combination with the TVM composition described herein include those used for multiple myeloma include Pomalidomide (Pomalyst®), Carfilzomib (Kyprolis™), Everolimus (Afinitor®), dexamethasone (Decadron), prednisone and methylprednisolone (Solu-medrol®) and hydrocortisone.

Current chemotherapeutic drugs that may be used in combination with the TVM composition described herein include those used for Hodgkin's disease include Brentuximab vedotin (Adcetris™): anti-CD-30, Rituximab, Adriamycin® (doxorubicin), Bleomycin, Vinblastine, Dacarbazine (DTIC).

Current chemotherapeutic drugs that may be used in combination with the TVM composition described herein include those used for Non-Hodgkin's disease include Rituximab (Rituxan®), Ibritumomab (Zevalin®), tositumomab (Bexxar®), Alemtuzumab (Campath®) (CD52 antigen), Ofatumumab (Arzerra®), Brentuximab vedotin (Adcetris®) and Lenalidomide (Revlimid®).

Current chemotherapeutic drugs that may be used in combination with the TVM composition described herein include those used for:

B-Cell Lymphoma, for Example:

Diffuse large B-cell lymphoma: CHOP (cyclophosphamide, doxorubicin, vincristine, and prednisone), plus the monoclonal antibody rituximab (Rituxan). This regimen, known as R-CHOP, is usually given for about 6 months.

Primary Mediastinal B-Cell Lymphoma: R-CHOP.

Follicular lymphoma: rituximab (Rituxan) combined with chemo, using either a single chemo drug (such as bendamustine or fludarabine) or a combination of drugs, such as the CHOP or CVP (cyclophosphamide, vincristine, prednisone regimens. The radioactive monoclonal antibodies, ibritumomab (Zevalin) and tositumomab (Bexxar) are also possible treatment options. For patients who may not be able to tolerate more intensive chemo regimens, rituximab alone, milder chemo drugs (such as chlorambucil or cyclophosphamide).

Chronic Lymphocytic Leukemia/Small Lymphocytic Lymphoma: R-CHOP.

Mantle cell lymphoma: fludarabine, cladribine, or pentostatin; bortezomib (Velcade) and lenalidomide (Revlimid) and ibrutinib (Imbruvica).

Extranodal marginal zone B-cell lymphoma—mucosa-associated lymphoid tissue (MALT) lymphoma: rituximab; chlorambucil or fludarabine or combinations such as CVP, often along with rituximab.

Nodal marginal zone B-cell lymphoma: rituximab (Rituxan) combined with chemo, using either a single chemo drug (such as bendamustine or fludarabine) or a combination of drugs, such as the CHOP or CVP (cyclophosphamide, vincristine, prednisone regimens. The radioactive monoclonal antibodies, ibritumomab (Zevalin) and tositumomab (Bexxar) are also possible treatment options. For patients who may not be able to tolerate more intensive chemo regimens, rituximab alone, milder chemo drugs (such as chlorambucil or cyclophosphamide).

Splenic marginal zone B-cell lymphoma: rituximab; patients with Hep C—anti-virals.

Burkitt lymphoma: methotrexate; hyper-CVAD—cyclophosphamide, vincristine, doxorubicin (also known as Adriamycin), and dexamethasone. Course B consists of methotrexate and cytarabine; CODOX-M—cyclophosphamide, doxorubicin, high-dose methotrexate/ifosfamide, etoposide, and high-dose cytarabine; etoposide, vincristine, doxorubicin, cyclophosphamide, and prednisone (EPOCH)

Lymphoplasmacytic lymphoma—rituximab.

Hairy cell leukemia—cladribine (2-CdA) or pentostatin; rituximab; interferon-alfa

T-cell lymphomas, for example:

Precursor T-lymphoblastic lymphoma/leukemia—cyclophosphamide, doxorubicin (Adriamycin), vincristine, L-asparaginase, methotrexate, prednisone, and, sometimes, cytarabine (ara-C). Because of the risk of spread to the brain and spinal cord, a chemo drug such as methotrexate is also given into the spinal fluid.

Skin lymphomas: Gemcitabine Liposomal doxorubicin (Doxil); Methotrexate; Chlorambucil; Cyclophosphamide; Pentostatin; Etoposide; Temozolomide; Pralatrexate; R-CHOP.

Angioimmunoblastic T-cell lymphoma: prednisone or dexamethasone.

Extranodal natural killer/T-cell lymphoma, nasal type: CHOP.

Anaplastic large cell lymphoma: CHOP; pralatrexate (Folotyn), targeted drugs such as bortezomib (Velcade) or romidepsin (Istodax), or immunotherapy drugs such as alemtuzumab (Campath) and denileukin diftitox (Ontak).

Primary central nervous system (CNS) lymphoma—methotrexate; rituximab.

A more general list of suitable chemotherapeutic agents includes, but are not limited to, radioactive molecules, toxins, also referred to as cytotoxins or cytotoxic agents, which includes any agent that is detrimental to the viability of cells, agents, and liposomes or other vesicles containing chemotherapeutic compounds. Examples of suitable chemotherapeutic agents include but are not limited to 1-dehydrotestosterone, 5-fluorouracil decarbazine, 6-mercaptopurine, 6-thioguanine, actinomycin D, adriamycin, aldesleukin, alkylating agents, allopurinol sodium, altretamine, amifostine, anastrozole, anthramycin (AMC)), anti-mitotic agents, cis-dichlorodiamine platinum (II) (DDP) cisplatin), diamino dichloro platinum, anthracyclines, antibiotics, antis, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunorubicin HCl, daunorucbicin citrate, denileukin diftitox, Dexrazoxane, Dibromomannitol, dihydroxy anthracin dione, Docetaxel, dolasetron mesylate, doxorubicin HCl, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCl, glucocorticoids, goserelin acetate, gramicidin D, granisetron HCl, hydroxyurea, idarubicin HCl, ifosfamide, interferon α-2b, irinotecan HCl, letrozole, leucovorin calcium, leuprolide acetate, levamisole HCl, lidocaine, lomustine, maytansinoid, mechlorethamine HCl, medroxyprogesterone acetate, megestrol acetate, melphalan HCl, mercaptipurine, mesna, methotrexate, methyltestosterone, mithramycin, mitomycin C, mitotane, mitoxantrone, nilutamide, octreotide acetate, ondansetron HCl, paclitaxel, pamidronate disodium, pentostatin, pilocarpine HCl, plimycin, polifeprosan 20 with carmustine implant, porfimer sodium, procaine, procarbazine HCl, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

Additional therapeutic agents that can be administered in combination with the TVM compositions disclosed herein can include bevacizumab, sutinib, sorafenib, 2-methoxyestradiol, finasunate, vatalanib, vandetanib, aflibercept, volociximab, etaracizumab, cilengitide, erlotinib, cetuximab, panitumumab, gefitinib, trastuzumab, atacicept, rituximab, alemtuzumab, aldesleukine, atlizumab, tocilizumab, temsirolimus, everolimus, lucatumumab, dacetuzumab, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, oblimersen, plitidepsin, talmapimod, enzastaurin, tipifarnib, perifosine, imatinib, dasatinib, lenalidomide, thalidomide, simvastatin, and celecoxib.

In one aspect of the present invention, the TVM or VM compositions disclosed herein are administered in combination with at least one immunosuppressive agent. The immunosuppressive agent may be selected from the group consisting of a calcineurin inhibitor, e.g. a cyclosporin or an ascomycin, e.g. Cyclosporin A (NEORAL®), tacrolimus, a mTOR inhibitor, e.g. rapamycin or a derivative thereof, e.g. Sirolimus (RAPAMUNE®), Everolimus (Certican®), temsirolimus, biolimus-7, biolimus-9, a rapalog, e.g. azathioprine, campath 1H, a S1P receptor modulator, e.g. fingolimod or an analogue thereof, an anti-IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAM®, THYMOGLOBULIN®, Brequinar Sodium, 15-deoxyspergualin, tresperimus, Leflunomide ARAVA®, anti-CD25, anti-IL2R, Basiliximab (SIMULECT®), Daclizumab (ZENAPAX®), mizorbine, methotrexate, dexamethasone, pimecrolimus (Elidel®), abatacept, belatacept, etanercept (Enbrel®), adalimumab (Humira®), infliximab (Remicade®), an anti-LFA-1 antibody, natalizumab (Antegren®), Enlimomab, ABX-CBL, antithymocyte immunoglobulin, siplizumab, and efalizumab.

In one aspect of the present invention, the TVM or VM composition described herein can be administered in combination with at least one anti-inflammatory agent. The anti-inflammatory agent can be a steroidal anti-inflammatory agent, a nonsteroidal anti-inflammatory agent, or a combination thereof. In some embodiments, anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof.

In one aspect of the present invention, the TVM or VM composition described herein can be administered in combination with at least one immunomodulatory agent.

In another embodiment, the TMV or VM composition can be used in combination with anti-viral therapy is used to treat viral complications of HSCT includes, but are not limited to, valgancyclovir, ganciclovir, acyclovir, cidofovir, foscarnet, and vidarabine. Immunosuppressive agents, such as corticosteroids, Rituximab, Leflunomide Azathioprene, and cyclosporine, are sometimes also used to temporarily reduce viral symptoms in patients and can be used in combination with the TMV or VM therapy. In addition, cytotoxic chemotherapy can be used to treat viral complications. A variety of agents have been used including cyclophosphamide, anthracyclines, vincristine, etoposide, and prednisone.

Methods of Manufacturing TAA and/or VAA T-Cell Subpopulations that Comprise the TVM and VM Compositions

T-cell subpopulations specific for multiple TAA or VAA to be combined into the TVM or VM composition for therapeutic administration described herein can be generated using any known method in the art or as described herein. Activated T-cell subpopulations that recognize at least one epitope of an antigen of a tumor or virus can be generated by any method known in the art or as described herein. Non-limiting exemplary methods of generating activated T-cell subpopulations that recognize at least one epitope of an antigen of a tumor or virus can be found in, for example Shafer et al., Leuk Lymphoma (2010) 51(5):870-880; Cruz et al., Clin Cancer Res., (2011) 17(22): 7058-7066; Quintarelli et al., Blood (2011) 117(12): 3353-3362; Hanley et al., Cytotherapy (2011) 13: 976-986; Gerdemann et al., Mol Ther (2012) 20(8) 1622-1632; and Chapuis et al., Sci Transl Med (2013) 5(174):174ra27, all incorporated herein by reference.

Generally, generating the T-cell subpopulations of the TVM or VM compositions of the present invention may involve (i) collecting a peripheral blood mononuclear cell product from a donor; (ii) determining the HLA subtype of the mononuclear cell product; (iii) separating the monocytes and the lymphocytes of the mononuclear cell product; (iv) generating and maturing dendritic cells (DCs) from the monocytes; (v) pulsing the DCs with one or multiple TAAs or VAAs, as desired; (vi) optionally carrying out a CD45RA+ selection to isolate naïve lymphocytes; (vii) stimulating the naïve lymphocytes with the peptide-pulsed DCs in the presence of a cytokine cocktail; (viii) repeating the T cell stimulation with fresh peptide-pulsed DCs or other peptide-pulsed antigen presenting cells in the presence of a cytokine cocktail; (ix) harvesting the T-cells and optionally cryopreserving for future use.

In some aspects, generating the T-cell subpopulations of the TVM or VM compositions of the present invention may involve (i) collecting a peripheral blood mononuclear cell product from a donor; (ii) determining the HLA subtype of the mononuclear cell product; (iii) separating the monocytes and the lymphocytes of the mononuclear cell product; (iv) generating and maturing dendritic cells (DCs) from the monocytes; (v) pulsing the DCs with one or multiple TAAs or VAAs, as desired; (vi) optionally carrying out a CD45RA+ selection to isolate naïve T-cells; (vii) stimulating the naïve T-cells with the peptide-pulsed DCs in the presence of a cytokine cocktail; (viii) repeating the T-cell stimulation with fresh peptide-pulsed DCs or other peptide-pulsed antigen presenting cells in the presence of a cytokine cocktail; (ix) harvesting the T-cells and optionally cryopreserving for future use.

Collecting a Peripheral Blood Mononuclear Cell Product from a Donor

The generation of T-cell subpopulations to be specific to one or multiple TAAs or VAAs generally requires a peripheral blood mononuclear cell (PBMC) product from a donor, either an allogeneic or autologous donor, as a starting material. Isolation of PBMCs is well known in the art. Non-limiting exemplary methods of isolating PBMCs are provided in Grievink, H. W., et al. (2016) “Comparison of three isolation techniques for human peripheral blood mononuclear cells: Cell recovery and viability, population composition, and cell functionality,” Biopreservation and BioBanking, which is incorporated herein by reference. The PBMC product can be isolated from whole blood, an apheresis sample, a leukapheresis sample, or a bone marrow sample provided by a donor. In some embodiments, the starting material is an apheresis sample, which provides a large number of initially starting mononuclear cells, potentially allowing a large number of different T-cell subpopulations to be generated. In some embodiments, the PBMC product is isolated from a sample containing peripheral blood mononuclear cells (PBMCs) provided by a donor. In some embodiments, the donor is a healthy donor. In some embodiments, the PBMC product is derived from cord blood. In some embodiments, the donor is the same donor providing stem cells for a hematopoietic stem cell transplant (HSCT).

### Determining HLA Subtype

When the T-cell subpopulations are generated from an allogeneic, healthy donor, the HLA subtype profile of the donor source is determined and characterized. Determining HLA subtype (i.e., typing the HLA loci) can be performed by any method known in the art. Non-limiting exemplary methods for determining HLA subtype can be found in Lange, V., et al., BMC Genomics (2014)15: 63; Erlich, H., Tissue Antigens (2012) 80:1-11; Bontadini, A., Methods (2012) 56:471-476; Dunn, P. P., Int J Immunogenet (2011) 38:463-473; and Hurley, C. K., “DNA-based typing of HLA for transplantation.” in Leffell, M. S., et al., eds., Handbook of Human Immunology, 1997. Boca Raton: CRC Press, each independently incorporated herein by reference. Preferably, the HLA-subtyping of each donor source is as complete as possible.

In some embodiments, the determined HLA subtypes include at least 4 HLA loci, preferably HLA-A, HLA-B, HLA-C, and HLA-DRB1. In some embodiments, the determined HLA subtypes include at least 6 HLA loci. In some embodiments, the determined HLA subtypes include at least 6 HLA loci. In some embodiments, the determined HLA subtypes include all of the known HLA loci. In general, typing more HLA loci is preferable for practicing the invention, since the more HLA loci that are typed, the more likely the allogeneic T-cell subpopulations selected will have highest activity relative to other allogeneic T-cell subpopulations that have HLA alleles or HLA allele combinations in common with the patient or the diseased cells in the patient.

### Separating the Monocytes and the Lymphocytes of the Peripheral Blood Mononuclear Cell Product

In general, the PBMC product may be separated into various cell-types, for example, into platelets, red blood cells, lymphocytes, and monocytes, and the lymphocytes and monocytes retained for initial generation of the T-cell subpopulations. The separation of PBMCs is known in the art. Non-limiting exemplary methods of separating monocytes and lymphocytes include Vissers et al., J Immunol Methods. 1988 Jun. 13; 110(2):203-7 and Wahl et al., Current Protocols in Immunology (2005) 7.6A.1-7.6A.10, which are incorporated herein by reference. For example, the separation of the monocytes can occur by plate adherence, by CD14+ selection, or other known methods. The monocyte fraction is generally retained in order to generate dendritic cells used as an antigen presenting cell in the T-cell subpopulation manufacture. The lymphocyte fraction of the PBMC product can be cryopreserved until needed, for example, aliquots of the lymphocyte fraction (˜5×10⁷ cells) can be cryopreserved separately for both Phytohemagglutinin (PHA) Blast expansion and T-cell subpopulation generation.

### Generating Dendritic Cells

The generation of mature dendritic cells used for antigen presentation to prime T-cells is well known in the art. Non-limiting exemplary methods are included in Nair et al., “Isolation and generation of human dendritic cells.” Current protocols in immunology (2012) 0 7: Unit7.32. doi:10.1002/0471142735.im0732s99 and Castiello et al., Cancer Immunol Immunother, 2011 April; 60(4):457-66, which are incorporated herein by reference. For example, the monocyte fraction can be plated into a closed system bioreactor such as the Quantum Cell Expansion System, and the cells allowed to adhere for 2-4 hours at which point 1,000 U/mL of IL-4 and 800 U/mL GM-CSF can be added. The concentration of GM-CSF and IL-4 can be maintained. The dendritic cells can be matured using a cytokine cocktail. In some embodiments the cytokine cocktail consists of LPS (30 ng/mL), IL-4 (1,000 U/mL), GM-CSF (800 U/mL), TNF-Alpha (10 ng/mL), IL-6 (100 ng/mL), and IL-1beta (10 ng/mL). The dendritic cell maturation generally occurs in 2 to 5 days. In some embodiments, the adherent DCs are harvested and counted using a hemocytometer. In some embodiments, a portion of the DCs are cryopreserved for additional further stimulations.

### Pulsing the Dendritic Cells

The non-mature and mature dendritic cells are pulsed with one or more tumor and or viral peptides, which can be individually selected, selected as an intentional optimized subset, or with a TAA or VAA overlapping peptide library. For example, the dendritic cells can be pulsed using one or more peptides, for example specific epitopes and/or a overlapping peptide library. Methods of pulsing a dendritic cell with a overlapping peptide library are known. For example, about 100 ng of one or more peptides of the TAA or VAA, for example a peptide library (PepMix), can be added per 10 million dendritic cells and incubated for about 30 to 120 minutes.

### Naïve T-Cell Selection of Lymphocytes

In order to increase the potential number of specific TAA or VAA activated T-cells and reduce T-cells that target other antigens, it is preferable to utilize naïve T-cells as a starting material. To isolate naïve T-cells, the lymphocytes can undergo a selection, for example CD45RA+ cells selection. CD45RA+ cell selection methods are generally known in the art. Non-limiting exemplary methods are found in Richards et al., Immune memory in CD4+ CD45RA+ T cells. Immunology. 1997; 91(3):331-339 and McBreen et al., J Virol. 2001 May; 75(9): 4091-4102, which are incorporated herein by reference. For example, to select for CD45RA⁺ cells, the cells can be labeled using 1 vial of CD45RA microbeads from Miltenyi Biotec per 1×10¹¹ cells after 5-30 minutes of incubation with 100 mL of CliniMACS buffer and approximately 3 mL of 10% human IVIG, 10 ug/mL DNAase I, and 200 mg/mL of magnesium chloride. After 30 minutes, cells will be washed sufficiently and resuspended in 20 mL of CliniMACS buffer. The bag will then be set up on the CLINIMACS Plus device and the selection program can be run according to manufacturer's recommendations. After the program is completed, cells can be counted, washed and resuspended in “CTL Media” consisting of 44.5% EHAA Click's, 44.5% Advanced RPMI, 10% Human Serum, and 1% GlutaMAX.

Stimulating Naïve T Cells with Peptide-Pulsed Dendritic Cells

Prior to stimulating naïve T-cells with the dendritic cells, it may be preferable to irradiate the DCs, for example, at 25 Gy. The DCs and naïve T-cells are then co-cultured. The naïve T-cells can be co-cultured in a ratio range of DCs to T cells of about 1:5-1:50, for example, 1:5; 1:10, 1:15, 1:20, 1:25, 1:30, 1:35, 1:40, 1:45, or about 1:50. The DCs and T-cells are generally co-cultured with cytokines. In some embodiments, the cytokines are selected from a group consisting of IL-6 (100 ng/mL), IL-7 (10 ng/mL), IL-15 (5 ng/mL), IL-12 (10 ng/mL), and IL-21 (10 ng/mL).

### Second T Cell Stimulation

In general, it may be preferable to further stimulate the T-cell subpopulations with one or additional stimulation procedures. The additional stimulation can be performed with, for example, fresh DCs pulsed with the same peptides as used in the first stimulation, similarly to as described above. In some embodiments, the cytokines used during the second stimulation are selected from a group consisting of IL-7 (10 ng/mL) and IL-2 (100 U/mL).

Alternatively, peptide-pulsed PHA blasts can be used as the antigen presenting cell. The use of peptide-pulsed PHA blasts to stimulate and expand T-cells are well known in the art. Non-limiting exemplary methods can be found in Weber et al., Clin Cancer Res. 2013 Sep. 15; 19(18): 5079-5091 and Ngo et al., J Immunother. 2014 May; 37(4): 193-203, which are incorporated herein by reference. The peptide-pulsed PHA blasts can be used to expand the T-cell subpopulation in a ratio range of PHA blasts to expanded T cells of 10:1-1:10. For example, the ratio of PHA blasts to T cells can be 10:1, between 10:1 and 9:1, between 9:1 and 8:1, between 8:1 and 7:1, between 7:1 and 6:1, between 6:1 and 5:1, between 5:1 and 4:1, between 4:1 and 3:1, between 3:1 and 2:1, between 2:1 and 1:1, between 1:1 and 1:2, between 1:2 and 1:3, between 1:3 and 1:4, between 1:4 and 1:5, between 1:5 and 1:6, between 1:6 and 1:7, between 1:7 and 1:8, between 1:8 and 1:9, between 1:9 and 1:10. In general, cytokines are included in the co-culture, and are selected from the group consisting of IL-7 (10 ng/mL) and IL-2 (100 U/mL).

Additional T-Cell Expansion and T-Cell Subpopulation Harvest Additional T cell stimulations may be necessary to generate the necessary number of T-cell subpopulations for use in the TVM or VM composition. Following any stimulation and expansion, the T-cell subpopulations are harvested, washed, and concentrated. In some embodiments, a solution containing a final concentration of 10% dimethyl sulfoxide (DMSO), 50% human serum albumin (HSA), and 40% Hank's Balanced Salt Solution (HBSS) will then be added to the cryopreservation bag. In some embodiments, the T-cell subpopulations will be cryopreserved in liquid nitrogen.

### Further Characterization of the T-Cell Subpopulations

The T-cell subpopulations for use in the TVM or VM composition of the present invention are HLA-typed and can be further characterized prior to use or inclusion in the TVM or VM composition. For example, each of the T-cell subpopulations may be further characterized by, for example, one or more of i) determining the TAA or VAA specificity of the T-cell subpopulation; ii) identifying the tumor associated antigen or virus associated antigen epitope(s) the T-cell subpopulation is specific to; iii) determining whether the T-cell subpopulation includes MHC Class I or Class II restricted subsets or a combination of both; iv) correlating antigenic activity through the T-cell's corresponding HLA-allele; and v) characterizing the T-cell subpopulation's immune effector subtype concentration, for example, the population of effector memory cells, central memory cells, γδ T-cells, CD8+, CD4+, NKT-cell.

### Determining the Tumor- or Virus-Associated Antigen Specificity of the T-Cell Subpopulation

The T-cell subpopulations of the TVM or VM composition can be further characterized by determining each T-cell subpopulation's specificity for its targeted antigen. Specificity can be determined using any known procedure, for example, an ELISA based immunospot assay (ELISpot). In some embodiments, tumor-associated antigen specificity of the T-cell subpopulation is determined by ELISpot assay. In some embodiments, virus-associated antigen specificity of the T-cell subpopulation is determined by ELISpot assay. ELISpot assays are widely used to monitor adaptive immune responses in both humans and animals. The method was originally developed from the standard ELISA assay to measure antibody secretion from B cells (Czerkinsky C. et al. (1983) A solid-phase enzyme-linked immunospot (ELISPOT) assay for enumeration of specific antibody-secreting cells. J. Immunol Methods 65: 109-21), which is incorporated herein by reference. The assay has since been adapted to detect secreted cytokines from T cells, for example IFN-γ, and is an essential tool for understanding the helper T cell response.

A T-cell ELISpot assay generally comprises the following steps:

i) a capture antibody specific for the chosen analyte, for example IFN-γ, is coated onto a PVDF plate;

ii) the plate is blocked, usually with a serum;

iii) the T-cell subpopulation is added along with the specific, targeted tumor- or virus-associated antigen;

iv) plates are incubated and secreted cytokines, for example IFN-γ, are captured by the immobilized antibody on the PVDF surface;

v) after washing, a biotinylated detection antibody is added to allow detection of the captured cytokine; and

vi) the secreted cytokine is visualized using an avidin-HRP or avidin-ALP conjugate and a colored precipitating substrate.

Each colored spot represents a cytokine secreting cell. The spots can be counted by eye or by using an automated plate-reader. Many different cytokines can be detected using this method including IL-2, IL-4, IL-17, IFN γ, TNFα, and granzyme B. The size of the spot is an indication of the per cell productivity and the avidity of the binding. The higher the avidity of the T cell recognition the higher the productivity resulting in large, well-defined spots.

### Identifying the TAA or VAA Epitope(s) the T-Cell Subpopulation is Specific to

The T-cell subpopulations of the TVM or VM composition can be further characterized by identifying the specific TAA or VAA epitope or epitopes to which the T-cell subpopulation is specific to. This may be especially useful when more than one TAA or VAA peptide is used to prime the T-cell subpopulation. Determining TAA or VAA epitope specificity is generally known in the art. Non-limiting exemplary methods include Ohminami et al., Blood. 2000 Jan. 1; 95(1):286-93; Oka et al., Immunogenetics. 2000 February; 51(2):99-107; and Bachinsky et al., Cancer Immun. 2005 Mar. 22; 5:6; Kuzushima et al., Blood (2003) 101:1460-1468; Kondo et al., Blood (2004) 103(2): 630-638; Hanley et al., Blood (2009) 114(9): 1958-1967; and Hanley et al., Cytotherapy (2011) 13: 976-986, which are each incorporated herein by reference. For example, to identify the epitopes with TAA or VAA specific activity antigen peptide libraries can be grouped into pools in which each peptide is represented in two or more pools as a quick screening tool in an Elispot assay, and the pools showing activity determined. Common peptides represented in both pools can then be further screened to identify the specific peptide epitopes which show activity.

### Determining the T-Cell Subpopulation's MHC-Class I or Class II Restricted Subsets

The T-cell subpopulations of the TVM or VM composition can be further characterized by determining the subpopulation's MHC Class I or Class II subset restriction response. This is done to determine whether epitope recognition is mediated by CD8+(class I) or CD4+(class II) T-cells. General methods for determining the MHC Class I or Class II response are generally known in the art. A non-limiting exemplary method is found in Weber et al., Clin Cancer Res. 2013 Sep. 15; 19(18): 5079-5091, which is incorporated herein by reference. For example, to determine HLA restriction response, T cells can be pre-incubated with class I or II blocking antibodies for 1 hour before the addition of antigen peptides in an ELISPOT assay using autologous peptide-pulsed PHA blasts as targets with unpulsed PHA blasts as a control. IFN [symbol] -secretion is measured in the presence of each blocking antibody. If, when pre-incubated with a class I blocking antibody, IFN [symbol] -secretion is reduced to background levels then this is indicative of a class I restriction and the epitope recognition is mediated by CD8+ T cells. If, when pre-incubated with a class II blocking antibody, IFN [symbol] -secretion is reduced to background levels then this is indicative of a class II restriction and the epitope recognition is mediated by CD4+ T cells.

The direct detection of antigen-specific T cells using tetramers of soluble peptide-major histocompatibilty complex (pMHC) molecules is widely used in both basic and clinical immunology. Tetrameric complexes of HLA molecules can be used to stain antigen-specific T cells in FACS analysis. In vitro synthesized soluble HLA-peptide complexes are used as tetrameric complexes to stain antigen specific T cells in FACS analysis (Altman et al., Science 274: 94-96, 1996). T-cell subpopulations specific for TAAs are stained with CD8 fluorescein isothiocyanate (FITC) and with phycoerythrin (PE)-labeled MHC pentamers at various timepoints during in vitro stimulation. Antigen specificity is measured by flow cytometry.

### Correlating Antigenic Activity Through the T-Cell's Corresponding HLA-Allele

The T-cell subpopulation can be further characterized by correlating antigenic activity through the T-cell subpopulation's corresponding HLA-allele. Correlating antigenic activity through the corresponding HLA-allele can be done using any known method. For example, in some embodiments, a HLA restriction assay is used to determine antigen activity through a corresponding allele. Methods to determine T cell restriction are known in the art and involve inhibition with locus specific antibodies, followed by antigen presentation assays (ELISPOT) with panels of cell lines matched or mismatched at the various loci of interest (see, e.g., (Oseroff et al., J Immunol (2010) 185(2): 943-955; Oseroff et al., J Immunol (2012) 189(2): 679-688; Wang Curr Protocols in immunol (2009) Chap. 20, page 10; Wilson et al., J. Virol. (2001) 75(9): 4195-4207), each independently incorporated herein by reference. Because epitope binding to HLA class II molecules is absolutely necessary (but not sufficient) for T cell activation, data from in vitro HLA binding assays has also been useful to narrow down the possible restrictions (Arlehamn et al., J Immunol (2012b) 188(10):5020-5031). This is usually accomplished by testing a given epitope for binding to the specific HLA molecules expressed in a specific donor and eliminating from further consideration HLA molecules to which the epitope does not bind. To determine the HLA restriction of the identified epitope, T cells can be plated in an IFN-γ ELISPOT assay with TAA peptide pulsed PHA blasts that match at a single allele, measuring the strongest antigen activity, and identifying the corresponding allele.

### Characterizing the T-cell Subpopulation's Immune Effector Subtype Concentration

The T-cell subpopulation is likely to be made up of different lymphocytic cell subsets, for example, a combination of CD4+ T-cells, CD8+ T-cells, CD3+/CD56+ Natural Killer T-cells (CD3+ NKT), and TCR γδ T-cells (γδ T-cells). In particular, the T-cell subpopulation likely include at least CD4+ T-cells and CD8+ T-cells that have been primed and are capable of targeting a single specific TAA for tumor killing and/or cross presentation. The T-cell subpopulation may further comprise activated γδ T-cells and/or activated CD3+/CD56+ NKT cells capable of mediating anti-tumor responses. Accordingly, the T-cell subpopulation may be further characterized by determining the population of various lymphocytic subtypes, and the further classification of such subtypes, for example, by determining the presence or absence of certain clusters of differentiation (CD) markers, or other cell surface markers, expressed by the cells and determinative of cell subtype.

In some embodiments, the T-cell subpopulation may be analyzed to determine CD8+ T-cell population, CD4+, T-cell population, γδ T-cell population, NKT-cell population, and other populations of lymphocytic subtypes. For example, the population of CD4+ T-cells within the T-cell subpopulation may be determined, and the CD4+ T-cell subtypes further determined. For example, the CD4+ T-cell population may be determined, and then further defined, for example, by identifying the population of T-helper 1 (Th1), T-helper 2 (Th2), T-helper 17 (Th17), regulatory T cell (Treg), follicular helper T-cell (Tfh), and T-helper 9 (Th9). Likewise, the other lymphocytic subtypes comprising the T-cell subpopulation can be determined and further characterized.

In addition, the T-cell subpopulation can be further characterized, for example, for the presence, or lack thereof, of one or more markers associated with, for example, maturation or exhaustion. T cell exhaustion (Tex) is a state of dysfunction that results from persistent antigen and inflammation, both of which commonly occur in tumor tissue. The reversal or prevention of exhaustion is a major area of research for tumor immunotherapy. Tex cell populations can be analyzed using multiple phenotypic parameters, either alone or in combination. Hallmarks commonly used to monitor T cell exhaustion are known in the art and include, but are not limited to, programmed cell death-1 (PD-1), CTLA-4/CD152 (Cytotoxic T-Lymphocyte Antigen 4), LAG-3 (Lymphocyte activation gene-3; CD223), TIM-3 (T cell immunoglobulin and mucin domain-3), 2B4/CD244/SLAMF4, CD160, and TIGIT (T cell Immunoreceptor with Ig and ITIM domains).

The T-cell subpopulations of the described compositions described herein can be subjected to further selection, if desired. For example, a particular T-cell subpopulation for inclusion in a TVM composition described herein can undergo further selection through depletion or enriching for a subpopulation. For example, following priming, expansion, and selection, the cells can be further selected for other cluster of differentiation (CD) markers, either positively or negatively. For example, following selection of for example CD4+ T-cells, the CD4+ T-cells can be further subjected to selection for, for example, a central memory T-cells (Tcm). For example, the enrichment for CD4+ Tcm cells comprises negative selection for cells expression a surface marker present on naïve T cells, such as CD45RA, or positive selection for cells expressing a surface marker present on Tcm cells and not present on naïve T-cells, for example CD45RO, CD62L, CCR7, CD27, CD127, and/or CD44. In addition, the T-cell subpopulations described herein can be further selected to eliminate cells expressing certain exhaustion markers, for example, programmed cell death-1 (PD-1), CTLA-4/CD152 (Cytotoxic T-Lymphocyte Antigen 4), LAG-3 (Lymphocyte activation gene-3; CD223), TIM-3 (T cell immunoglobulin and mucin domain-3), 2B4/CD244/SLAMF4, CD160, and TIGIT (T cell Immunoreceptor with Ig and ITIM domains)

Methods for characterizing lymphocytic cell subtypes are well known in the art, for example flow cytometry, which is described in Pockley et al., Curr Protoc Toxicol. 2015 Nov. 2; 66:18.8.1-34, which is incorporated herein by reference.

### Methods of Mesenchymal Stem Cell Expansion

Expansion of mesenchymal stem cells (MSC) can be performed by any method known in the art. Non-limiting exemplary methods for the expansion of mesenchymal stem cells can be found in Hanley et al., Cytotherapy (2013) 15(4): 416-422; Hanley et al., Cytotherapy (2014) 16(8): 1048-1058; Tom et al., U.S. Pat. No. 9,828,586; Tom et al., US2015/0247122; Antwiler US2007/0298497, each of which are incorporated herein by reference.

MSCs can be enriched and expanded from numerous sources, including bone marrow, cord blood, and adipose tissue, and have the potential to differentiate into chondrocytes, osteoblasts, and adipocytes. When grown under appropriate conditions the tri-lineage potential of these cells is maintained. However, during expansion, the telomeres shorten and unbiased differentiation into the three lineages could become polarized. Therefore, for therapeutic applications, obtaining clinically-relevant numbers of cells with a minimum number of cell passages and doublings is essential.

The manufacture of MSCs involves culturing whole adherent bone marrow (BM) cells or isolated bone marrow mononuclear cells (BMMNC). This heterogeneous cell population is initially plated in tissue culture flasks and the adherent cells, which contain the MSC progenitors, are passaged to produce a homogeneous population of MSCs that have a similar morphology to fibroblasts. Given the physical properties of MSCs (large size, adherence), expanding clinically-applicable amounts of MSCs can be difficult using conventional tissue culture methods. Alternatively, MSCs can be plated in cell factories and bioreactors for large-scale expansion. One example of a bioreactor includes, but is not limited to, the Quantum Cell Expansion System by Terumo BCT, Lakewood, Colo., USA.

Generally, isolating and expanding MSCs of the present invention in a bioreactor may involve i) collecting bone marrow from a donor; ii) priming and coating the cell expansion set in the bioreactor; iii) loading bone marrow into the bioreactor; iv) feeding the MSCs; v) harvesting MSCs; vi) performing additional passages; and vii) cryopreservation.

### Collecting Bone Marrow From a Donor

Bone marrow obtained by iliac crest aspiration is a common source for harvesting mesenchymal stem cells, other progenitor cells, and associated cytokine/growth factors. Because the use of bone marrow aspirate concentrate (BMAC) is currently approved by the United States Food and Drug Administration, it represents one of the few means for acquiring progenitor cells and growth factors for subsequent injection (Afizah et al., Tissue Eng. (2007) 13: 659-666). Bone marrow harvested by iliac crest aspiration can be performed by any method known in the art. Non-limiting exemplary methods for the bone marrow harvesting by iliac crest aspiration can be found in Chahla et al., Arthrosc Tech. (2017); 6 (2): e441-e445, which is incorporated herein by reference. Bone marrow aspiration kits are utilized in the clinical setting for this purpose, which comprise a bone marrow aspiration needle, trochar, a 30-mL syringe and an anticoagulant citrate dextrose solution.

### Priming and Coating the Cell Expansion Set in the Bioreactor

One day before loading bone marrow into the bioreactor, the disposable expansion set is loaded onto the bioreactor and the system is primed with phosphate buffered saline and the bioreactor is coated with fibronectin. After approximately 18 hours, the fibronectin is washed out with media.

Loading Bone Marrow into the Bioreactor

Once the bioreactor is primed and coated then 25-40 mL of bone marrow is transferred into a plasma transfer bag and filtered through a 165-225 μm filter. Cells are allowed to adhere to the hollow fibers in the bioreactor for approximately 48-96 hours.

### Feeding the MSCs

After the cells have adhered for 48-96 hours the lactate levels are measured by removing 2-3 mL of media from the sampling port. Cells are fed continuously with media, which is adjusted according to the glucose and lactate concentration of the media sample and the manufacturer's recommendations. Media is initially fed at a rate of 0.1 mL/min. Glucose and lactate measurements are typically measured twice daily (Aviva Accu-chek meter, Roche Diagnostics and LactatePlus Lactate Meter, Nova Biomedical). Once the lactate concentration reaches 4 mM, the inlet rate is doubled. The cells are ready to harvest 24-48 hours after the lactate levels reach 4 mM with an inlet rate of 0.4 mL/min.

### Harvesting the MSCs

The lactate level should be above 4 mM for the first passage. To harvest cells, the system is washed with phosphate buffered saline and then the cell inlet bag is filled with 180-200 mL of TrypLE Select. After 15 minutes of incubation, the TrypLE Select and the harvested cells are washed into the cell harvest bag using fresh medium.

### Performing Additional Passages

After the first passage, 2.0-3.5×10⁷ MSCs are loaded into a new expansion set either in the same bioreactor equipped with a new expansion set or a new bioreactor that is primed and coated as described above. Repeat the feeding step described above. Once the lactate level reaches 8 mM while the inlet rate is 1.5 mL/min for more than 12 hours then the cells are harvested as described above.

### Cryopreservation

Before cryopreservation, the MSCs are centrifuged at 500×g for 10 minutes and washed with a wash medium containing Plasmalyte (Baxter) and 5% human serum albumin (HSA). The cells are then centrifuged again and the cells are counted and frozen in 85% Plasmalyte, 10% dimethyl sulfoxide (DMSO), and 5% human serum albumin (HSA). Cells are frozen at 2.5×10⁷ cells/ml/vial.

### Further Characterization of the MSC Subpopulation

The MSC subpopulation for use in the TVM composition of the present invention can be further characterized prior to use or inclusion in the TVM composition. For example, each of the MSC subpopulations may be further characterized by, for example, one or more of i) measuring growth kinetics of MSC expansion; ii) enumerating colony forming units; iii) determining tri-lineage potential; iv) phenotyping; and v) measuring the suppression of T cell proliferation.

### Measuring Growth Kinetics of MSC Expansion

MSCs are plated in 96-well culture plates at 1×10³ cells/well. Population doubling time is measured during the cell growth log phase using CyQUANT, a fluorescence-based proliferation assay (Invitrogen). The cells are labeled at initiation with the CyQUANT reagent and tested daily for 7 days. Fluorescence is measured using a microplate reader. A standard curve is generated for each sample by plotting known numbers of MSC on 96-well tissue culture plates against fluorescence intensity values obtained after labeling with the CyQUANT reagent.

### Enumerating Colony Forming Units

An additional test of MSCs is their propensity to form colonies as measured by colony forming units (CFU). MSCs are harvested and immediately plated at 20 cells/cm² in 75 cm² flasks in Alpha-modified Minimum Essential Medium containing ribo- and deoxyribonucleotides supplemented with 10% Fetal Bovine Serum. Colony forming cells are allowed to grow for two weeks and then washed twice with PBS. Cultures are fixed in ethanol for 30 minutes at room temperature and stained with Giemsa stain. Colonies containing at least 40 cells are counted under a stereomicroscope.

### Determining Tri-Lineage Potential

Methods of measuring the potential of MSCs to differentiate into chondrocytes, osteoblasts, and adipocytes are known in the art. A non-limiting exemplary method is described in Pittinger et al., Science (1999) 284(5411): 143-147, which is incorporated herein by reference. MSCs are cultured under conditions that favor adipogenic, chondrogenic, or osteogenic differentiation. Adipogenic differentiation is induced by treatment with 1-methyl-3-isobutylxanthine, dexamethasone, insulin, and indomethacin. Induction is apparent after 1 to 3 weeks by the accumulation of lipid-rich vacuoles within cells and the expression of peroxisome proliferation-activated receptor [symbol] 2 (PPAR [symbol] 2), lipoprotein lipase (LPL) and the fatty acid binding protein aP2. To promote chondrogenic differentiation, MSCs are centrifuged to form a pelleted micromass and then cultured without serum and with transforming growth factor-β3. Type II collagen can be detected at 10 to 14 days with monoclonal antibody C4F6. The osteogenic differentiation is induced by dexamethasone, β-glycerol phosphate, and ascorbate in the presence of 10% v/v FBS. The MSCs form aggregates and increase expression of alkaline phosphatase and calcium accumulation.

### Phenotyping

MSCs are directly stained for the positive markers CD73, CD90, and CD105 as well as lineage markers CD45, CD34, CD14, CD19, and HLA-DRII and analyzed on a flow cytometer. Annexin-V and PI antibodies can be used as viability controls, and data analyzed with FlowJo Flow Cytometry software (Treestar, Ashland, Oreg., USA).

### Measuring the Suppression of T-Cell Proliferation

MSC lines are irradiated and plated in titrated numbers. Peripheral blood mononuclear cells (PBMCs) from healthy donors are labeled with carboxyfluorescein succinimidyl ester (CFSE, Sigma). CFSE-labeled PBMCs are then cultured alone (1:0 PBMC:MSC ratio) for use as a positive control or co-cultured with titrated numbers of MSCs ranging from 1:1 down to a 1:0.05 PBMC:MSC ratio. Soluble anti-human CD28 monoclonal antibodies (RnD Systems, Minneapolis, Minn., USA) are used to stimulate T cell populations. After four days in culture, cells are harvested and stained with anti-huCD4 APC (RnD Systems) to gauge the proliferation of CD4+ T cells by flow cytometry. Data acquisition can be performed with an Accuri C6 Flow Cytometer (BD Biosciences, San Jose, Calif.). CD4+ T cell proliferation (% CD4+/CFSE-low cells) is measured using a negative control gate set with non-stimulated PBMCs co-cultured at a 1:0.05 PBMC:MSC ratio (% CD4+/CFSE-high cells).

### Identifying the TVM or VM Composition Most Suitable for Administration

Characterization of each T-cell and MSC subpopulation composition allows for the selection of the most appropriate T-cell and MSC subpopulations for inclusion in the TVM or VM composition for any given patient. The MSC subpopulation choice is driven by the choice of T-cell subpopulation due to their lack of expression of Human Leukocyte Antigen (HLA)-class II and co-stimulatory molecules, which limits the immune response of the recipient to these cells. The goal is to match the product with the patient that has the both the highest HLA match and greatest TAA and VAA activity through the greatest number of shared alleles. In some embodiments, the T-cell subpopulations have at least one shared allele or allele combination with TAA activity through that allele or allele combination. In some embodiments, the T-cell subpopulations have at least one shared allele or allele combination with VAA activity through that allele or allele combination. In some embodiments, the T-cell subpopulation has greater than 1 shared allele or allele combination with TAA activity through that allele or allele combination. In some embodiments, the T-cell subpopulation has greater than 1 shared allele or allele combination with VAA activity through that allele or allele combination. In some embodiments, the T-cell subpopulation with the most shared alleles or allele combinations and highest specificity through those shared alleles and allele combinations is provided to a human in need thereof. For example, if T-cell subpopulation 1 is a 5/8 HLA match with the patient with TAA and VAA activity through 3 shared alleles or allele combinations while T-cell subpopulation 2 is a 6/8 HLA match with the patient with TAA and VAA activity through 1 shared allele the skilled practitioner would select T-cell subpopulation 1 as it has TAA and VAA activity through a greater number of shared alleles.

### Testing T-cell Subpopulation Reactivity Against Patient's Tumor

The cytolytic activity of an activated T-cell subpopulation comprising the TVM composition against a patient's tumor can be evaluated. A method of testing reactivity of T-cell subpopulations against tumor cells are well known. Non-limiting exemplary methods include Jedema et al., Blood (2004) 103:2677-2682; Noto et al., J Vis Exp. 2013; (82): 51105 and Baumgaertner et al., Bio-protocol “Chromium-51 (⁵¹Cr) Release Assay to Assess Human T Cells for Functional Avidity and Tumor Cell Recognition.” (2016) 6(16): e1906. For example, the T-cell subpopulation can be incubated with the patient's tumor and the percent lysis of the tumor cells determined. For example, a biopsy or blood sample will be collected from the patient. Target cells from the patient are fluorescence labeled with carboxyfluorescein succinimidyl ester (CFSE, Invitrogen), peptide-pulsed and incubated with activated T-cell subpopulations at a 40:1 effector-to-target ratios for 6-8 hrs. Ethidium homodimer (Invitrogen) is added after incubation to stain dead cells. Samples are acquired on a BD Fortessa Flow Cytometer. The number of live target cells is determined by gating on carboxyfluorescein succinimidyl ester-positive, ethidium homodimer-negative cells, and used to calculate cytolytic activity as follows: Lysis (%)=100−((live target cells/sample/live target cells control)×100).

T-cell subpopulations with the highest levels of reactivity against a patient's tumor can be selected for administration to the patient, providing a higher likelihood of successful therapeutic efficacy.

### Banked MSC and T-Cell Subpopulations Directed to Multiple Tumor- and Virus-Associated Antigens

The establishment of a T-cell and MSC subpopulation bank comprising discrete, characterized T-cell and MSC subpopulations for selection and inclusion in a TVM or VM composition bypasses the need for an immediately available donor and eliminates the wait required for autologous T cell production. Preparing MSC subpopulations by using donors, for example healthy volunteers or cord blood, allows the expansion and banking of MSC subpopulations readily available for administration. Preparing T-cell subpopulations directed to specific, known tumor and virus antigens by using donors, for example healthy volunteers or cord blood, allows the production and banking of T-cell subpopulations readily available for administration. Because the T-cell subpopulations are characterized, the selection of suitable T-cell subpopulations can be quickly determined based on minimal information from the patient, for example HLA-subtype and, optionally TAA expression profile for TAA T-cell subpopulations. From a single donor a T cell and MSC composition can be generated for use in multiple patients who share HLA alleles that have activity towards specific TAAs or VAAs. The T-cell subpopulation bank of the present invention includes a population of T-cell subpopulations which have been characterized as described herein. For example, the T-cell subpopulations of the bank are characterized as to HLA-subtype and one or more of i) TAA or VAA specificity of the T-cell subpopulation; ii) TAA or VAA epitope(s) the T-cell subpopulation is specific to; iii) T-cell subpopulation MHC Class I and Class II restricted subsets; iv) antigenic activity through the T-cell's corresponding HLA-allele; and v) immune effector subtype concentration, for example, the population of effector memory cells, central memory cells, γδ T-cells, CD8+, CD4+, NKT-cell. Because MSC subpopulations do not have co-stimulatory molecules and HLA Class II molecules, as well as low HLA Class I expression they can be used readily in TVM and VM compositions based on the donor source.

In some embodiments, the present invention is a method of generating a T-cell and MSC subpopulation bank comprising: (i) obtaining eligible donor samples; (ii) generating T-cell subpopulations specific to multiple TAAs and VAAs; (iii) isolating and expanding mesenchymal stem cells (iv) characterizing the T-cell subpopulations; (v) characterizing the MSC subpopulation (vi) cryopreserving the T-cell and MSC subpopulations; and (v) generating a database of T-cell and MSC subpopulation composition characterization data. In some embodiments, the T-cell subpopulations are stored according to their donor source. In some embodiments, the T-cell subpopulations are stored by TAA specificity. In some embodiments, the T-cell subpopulations are stored by VAA specificity. In some embodiments, the T-cell subpopulations are stored by human leukocyte antigen (HLA) subtype and restrictions. In some embodiments the MSC subpopulations are stored by donor source.

The banked MSC and T-cell subpopulations described herein are used to comprise a TVM composition for administration to a tumor patient following the determination of the patient's HLA subtype and, optionally, TAA expression profile of the patient's tumor. The banked MSC and T-cell subpopulations are used to comprise a VM composition for administration to a patient receiving a HSCT following the determination of the patient's HLA subtype.

### Example 1. Generation of T-Cell Subpopulations from Peripheral Blood Using Multiple-TAA Overlapping Peptide Libraries or Single TAA Overlapping Peptide Libraries

TAA-specific T-cell lines can be generated from total human blood peripheral mononuclear cells (Step 1) using a multiple-TAA overlapping peptide library approach. Alternatively, T-cell subpopulations can be generated using a TAA-overlapping peptide library to a single TAA, an overlapping peptide library further comprising HLA-restricted TAA epitopes, or specifically selected antigenic epitopes, wherein each T-cell subpopulation is primed and expanded to a single TAA, and subsequently recombined. Matured dendritic cells (DCs) are harvested and used as antigen presenting cells (APCs) and peptide-pulsed with a mix of three peptide libraries for WT1, Survivin, and PRAME (Step 2). T-cells are initially stimulated using a cytokine mix containing one or a combination of: IL-7, IL-12, IL-15, IL-6, and IL-27 (Step 3). Subsequent stimulations (Steps 4 and 5) are performed using irradiated DCs or irradiated phytohemagglutinin (PHA) blasts. Experimental procedures for each of these steps are provided below.

Step 1. Isolation of Mononuclear Cells

Heparinized peripheral blood is diluted in an equal volume of warm RPMI 1641 (Invitrogen) or PBS. In a 50 mL centrifuge tube, 10-15 mL of Lymphoprep (Axis-Shield) is overlayed with 20-30 mL of diluted blood. The mixture is centrifuged at 800×g for 20 minutes or 400× g for 40 minutes at ambient temperature, ensuring that acceleration and deceleration are set to “1” to prevent disrupting the interface. 1 mL of plasma aliquots are saved and stored at −80° C. The peripheral blood mononuclear cell (PBMC) interface is harvested into an equal volume of RPMI 1640, then centrifuge at 450× g for 10 minutes at ambient temperature, and the supernatant is aspirated. The pellet is loosened and the cells are resuspended in a volume of RPMI 1640 or PBS that yields an estimated 10×10⁶ cells/mL. An aliquot of cells is removed for counting using 50% red cell lysis buffer or Trypan blue and using a hemocytometer. The PBMCs are saved for DC generation using adherence (Step 2 below) and non-adherent cells are cryopreserved for use at initiation.

Step 2. Dendritic Cell (DC) Generation

PBMCs are centrifuged at 400×g for 5 minutes at ambient temperature, and the supernatant is aspirated. The cells are resuspended at approximately 5×10⁶ cells/mL in CellGenix DC medium containing 2 mM of Glutamax (Invitrogen), and the cells are plated in a 6-well plate (2 mL/well). The PBMC non-adherent fraction is removed after 1-2 hours, and the wells are rinsed with 2-5 mL of CellGenix DC medium or PBS and added to the harvested medium/non-adherent fraction. The non-adherent fraction is saved for later cryopreservation. 2 mL of DC medium containing 1,000 U/mL of IL-4 (R&D Systems) and 800 U/mL GM-CSF (CNMC Pharmacy) is added back to the adherent cells. All surrounding wells are filled with approximately 2 mL of sterile water or PBS to maintain the humidity within the plate, and the plate is placed in the incubator at 37° C. and 5% CO₂. On day 3 to 4, the cells are fed with 1,000 U/mL IL-4 and 800 U/mL GM-CSF. On day 5 to 6, the DCs are matured in 2 mL/well of DC medium containing lipopolysaccharide (LPS, Sigma) (30 ng/mL), IL-4 (1,000 U/mL), GM-CSF (800 U/mL), TNF-α (10 ng/mL, R&D Systems), IL-6 (100 ng/mL, CellGenix), and IL-1β (10 ng/mL, R&D Systems). The mature DCs are harvested on day 7 to 8 by gentle resuspension. The cells are counted using a hemocytometer. The DCs are transferred to a 15 mL centrifuge tube and centrifuged for 5 minutes at 400× g at ambient temperature. The supernatant is aspirated, and the pellet is resuspended by finger flicking, and 100 μL of appropriate overlapping peptide libraries mastermix (200 ng/peptide in 200 μL; PRAME, WT1, and Survivin overlapping peptide libraries; JPT Peptide Technologies) per 1-5×10⁶ cells is added to the DCs. The DCs and overlapping peptide libraries are mixed and transferred to the incubator. Alternatively, matured dendritic cells (DCs) are harvested and used as antigen presenting cells (APCs) and peptide-pulsed with single peptide libraries of WT1, Survivin, and PRAMS to generate 3 subpopulations of peptide-pulsed DCs. The mixture is incubated for 60-90 minutes at 37° C. and 5% CO₂.

Step 3. T-cell Population Initiation

After pulsing with overlapping peptide libraries, DCs are irradiated at 25 Gy. The DCs are washed with DC medium and centrifuged at 400×g for 5 minutes at ambient temperature. The supernatant is aspirated, and the wash step is repeated twice more. The cells are counted using a hemocytometer. The DCs are resuspended at 2-4×10⁵ cells/mL of CTL medium with 10% human serum (HS, Valley) for initiation. 1 mL of irradiated DCs/well is plated in a 24-well tissue culture treated plate.

Previously-frozen PBMCs from Step 1 are thawed at 37° C. and diluted in 10 mL of warm medium/1 mL of frozen cells. The PBMCs are centrifuged at 400×g for 5 minutes at ambient temperature and are resuspended in 5-10 mL of medium and a cell count is performed using a hemocytometer. The PBMCs are resuspended at 2×10⁶ cells/mL. DCs and PBMCs are recombined in the plate to stimulate CTL at a 1:10 to 1:5 ratio of DCs:CTL. Cytokines IL-7, IL-15, IL-6, and IL-12 are added to achieve a final concentration of IL-7 (10 ng/mL, R&D Systems)), IL-15 (5 ng/mL, CellGenix), IL-6 (100 ng/mL, CellGenix), and IL-12 (10 ng/mL, R&D Systems). All surrounding wells are filled with approximately 2 mL of PBS to maintain humidity within the plate. The cells are cultured in the incubator at 37° C. and 5% CO₂ for 7 to 8 days. A one half medium change is performed on day 4 to 5, with the wells being split 1:1 if nearly confluent.

Step 4. Second T-cell Stimulation in 24-Well Plate

The second stimulation of T-cells is performed using either Overlapping Peptide Library-Pulsed Autologous DCs (Procedure A) or Overlapping Peptide Library-Pulsed Autologous Phytohemagglutinin (PHA) Blasts (Procedure B) as antigen presenting cells.

Procedure A: Stimulation Using Overlapping Peptide Library-Pulsed Autologous DCs as Antigen Presenting Cells (APCs)

After pulsing with the appropriate overlapping peptide library (PRAME, WT1, and Survivin overlapping peptide library; JPT Peptide Technologies), DCs are irradiated at 25 Gy. DCs can be pulsed with mixtures of multiple overlapping peptide libraries (Multi-TAA) or with single overlapping peptide libraries and then combined after stimulation. The DCs are washed with DC medium and are centrifuged at 400×g for 5 minutes at ambient temperature. The supernatant is aspirated and the wash step is repeated twice more. The cells are counted using a hemocytometer. The DCs are resuspended at 0.5-2×10⁵ cells/mL of CTL medium with 10% HS (Valley) for initiation. Plate 1 mL of irradiated DCs/well (0.5-2×10⁵ cells) in a 24-well tissue culture treated plate. T-cells are counted using a hemocytometer. The cells are resuspended at 1×10⁶ cells/mL of T-cell medium supplemented with IL-7 (10 ng/mL final concentration, R&D Systems)) and IL-2 (100 U/mL final concentration, Proleukin) and 1 mL is aliquoted per well of the 24-well plate. The cells are cultured in the incubator at 37° C. and 5% CO₂ for 3 to 4 days. The medium is changed with IL-2 (˜100 U/mL final concentration, Proleukin) and cultured for another 3 to 4 days. Cells can be frozen after the second stimulation.

Procedure B: Stimulation Using Overlapping Peptide Library-Pulsed Autologous Phytohemagglutinin (PHA) Blasts as APCs

Autologous PHA blasts are harvested on day 7 by gentle resuspension, and cells are counted using a hemocytometer. The PHA blasts are transferred to a 15 mL centrifuge tube and are centrifuged for 5 minutes at 400× g at ambient temperature. The Supernatant is aspirated and the pellet is resuspended by finger flicking. 100 μL of appropriate overlapping peptide library mastermix (200 ng/peptide in 200 μL; PRAME, WT1, and Survivin overlapping peptide library; JPT Peptide Technologies) is added to PHA blasts per 1-10×10⁶ cells. Alternatively, PHA blasts are peptide-pulsed with single peptide libraries of WT1, Survivin, and PRAME to generate 3 subpopulations of peptide-pulsed PHA blasts. The PHA blasts are incubated for 30-60 minutes. The PHA blasts are resuspended in 5-10 mL of medium and irradiated at 50 Gy (or 100 Gy if used in G-rex). The PHA blasts are washed with CTL medium and centrifuged at 400×g for 5 minutes at ambient temperature. The supernatant is aspirated and the washing step is repeated twice more. A cell count is performed using a hemocytometer. The PHA blasts are resuspended at 0.5×10⁶ cells/mL of CTL medium to re-stimulate T-cells at an approximate ratio of 1:1 PHA blasts:T-cell. The T-cells are counted using a hemocytometer. The T-cells are resuspended at 0.5×10⁶ cells/mL of CTL medium supplemented with IL-7 (100 ng/mL final concentration; R&D Systems) and IL-2 (100 U/mL final concentration; Proleukin). One well of only PHA blasts is maintained as an irradiation control. The cells are cultured in the incubator at 37° C. and 5% CO₂ for 3 to 4 days. The medium is changed with IL-2 (100 U/mL final concentration; Proleukin) and the cells are cultured for another 3 to 4 days.

Step 5. Third T-cell Stimulation in G-Rex10 Using PHA Blasts as APCs

Autologous PHA blasts are harvested on day 7 by gentle resuspension, and cells are counted using a hemocytometer. The PHA blasts are transferred to a 15 mL centrifuge tube and are centrifuged for 5 minutes at 400× g at ambient temperature. The supernatant is aspirated, and the pellet is resuspended by finger flicking. 100 μL of appropriate overlapping peptid library mastermix (200 ng/peptide in 200 μL; PRAME, WT1, and Survivin overlapping peptide library; JPT Peptide Technologies) is added to PHA blasts per 1-10×10⁶ cells, and the PHA blasts are incubated for 30-60 minutes. Alternatively, PHA blasts are peptide-pulsed with single peptide libraries of WT1, Survivin, and PRAME to generate 3 subpopulations of peptide-pulsed PHA blasts. The PHA blasts are resuspended in 5-10 mL of medium and irradiated at 50 Gy (or 100 Gy if used in G-Rex). The PHA blasts are washed with CTL medium and are centrifuged at 400×g for 5 minutes at ambient temperature. The supernatant is aspirated, and the washing step is repeated twice more. Cells are counted using a hemocytometer. The PHA blasts are resuspended at 0.5×10⁶ cells/mL of CTL medium to re-stimulate T-cells at an approximate ratio of 1:1 PHA blasts'. 10 mL of cell suspension is added in the G-Rex10 and 1 mL/well (0.5×10⁶ PHA blasts) in the 24-well control plate. The T-cells were counted using a hemocytometer. The T-cells are resuspended at 05×10⁶ cells/mL of CTL medium, and 10 mL (5×10⁶ CTLs) was added in the G-Rex10 and 1 mL/well (0.5×10⁶ CTLs) in the 24-well control plate. The medium was supplemented with IL-7 (10 ng/mL final concentration; R&D Systems) and IL-2 (100 U/mL final concentration; Proleukin), and the cells are cultured in the incubator at 37° C. and 5% CO₂ for 3 to 4 days. One well of the 24 well plate is left with PHA blasts only as an irradiation control. The medium is changed with IL-2 (100 U/mL final concentration; Proleukin), and the cells are cultured for an additional 3 to 4 days.

### Example 2. Generation of MSC Subpopulations

### Step 1. Donor Screening

The starting material for the production of the present MSCs in this example is a bone marrow aspirate (“BMA”) obtained from a human donor. The BMA donor is screened for acceptance by testing a sample of blood against a panel of infectious diseases, and is accepted if the donor meets all criteria.

### Step 2. BMA Collection

Collection of the BMA takes place at an outpatient surgical center (e.g., 7 days after blood sample collection is performed). The donor is placed in the prone position and the bone marrow aspiration needle is inserted into the posterior iliac crest. The BMA collection procedure uses two syringes each containing 5 mL of 1,000 USP units/mL heparin sodium, which acts as an anticoagulant. As a result, the BMA material contains a small concentration (10,000 U/BMA) of heparin sodium. Up to 60 ml bone marrow is aspirated from the insertion site (from each side of the iliac crest), for example from 100 ml to 120 ml bone marrow in total.

Step 3. Isolation of Nucleated Bone Marrow Cells from BMA

The first step (day 1) in the isolation and expansion of human mesenchymal stem cells (hMSCs) involves the isolation of nucleated bone marrow cells from the BMA. A CytoMate® Cell Washer (Baxter Healthcare Corp., Deerfield, Ill.) connected with a fluid transfer set is used to transfer a Plasma-Lyte®A (Baxter, Deerfield, Ill.) and Hespan formulation to the BMA by using a Terumo sterile tube welder to fuse the BMA bag tubing lines together with the fluid transfer set.

In some embodiments of the present application, other cell washing or purification machines or techniques are used instead of a Cytomate Cell Washer; when the Cytomate is referred to herein, it is noted that any suitable replacement device for cell washing or purification machines or techniques may be employed instead. Hespan is utilized to agglutinate, sediment, and separate the majority of the red blood cells (RBCs) from the bone marrow nucleated cells. Using the “Fluid Transfer” program on the CytoMate® (Baxter, Deerfield, Ill.), the Plasma-Lyte®A (Baxter, Deerfield, Ill.) and Hespan formulation (6% Hetastarch) is then transferred into the BMA bag and the RBCs are allowed to settle for approximately 60 to 90 minutes until a distinct separation appears between the nucleated bone marrow cells and the RBCs. The nucleated bone marrow cells (top layer) are isolated from the BMA bag using a Fenwal plasma extractor to press the upper nucleated bone marrow cell layer into a transfer pack. The isolated nucleated bone marrow cells (INBMCs) are then transferred to the CytoMate® and processed by concentrating and washing the cells with culture medium (DMEM w/4 mM L-alanyl-L-glutamine+10% FBS). INBMCs are counted using a Hematology Analyzer (Beckman Coulter).

### Step 4. Isolation of MSCs

Following the cell count, the INBMCs are diluted to the target seeding concentration (e.g., 925×10⁶ INBMCs per 55 ml) and transferred to a 1.5 L culture medium bag using the “Transfer Volume” program on the CytoMate® to obtain the “Media Cell Suspension”. The “Media Cell Suspension” is used to seed the INBMCs into a CO² primed Nunc ten-stack cell factory (CF) at about 5,900 cells±about 20% per cm2 of growing surface. The CF is then placed in an incubator set at about 37±1° C. and about 5±2% CO², and ambient relative humidity. This is the primary culture (P0). After the initial seeding of the INBMCs, MSCs attach to the tissue culture plastic and grow to form a primary adherent population.

### Step 5. Feeds

Non-adherent cells are aspirated away during a feed change. The feed is an addition of 1.5 L of fresh culture medium to replace the existing culture medium (“spent medium”) that has been depleted of nutrients from cells growing in culture. A feed is performed every 3-4 days. During each feed, the CF is examined for integrity and appearance.

### Step 6. First Passage

After approximately about 21±3 days in culture, the primary culture (P0) is expanded (e.g., from one CF to approximately six CFs, or, optionally from one CF to approximately eight CFs) for the first passage (P1).

### Step 7. Trypsinizing

The spent medium in each CF is drained via gravity flow into an empty medium bag that is attached (e.g., sterile welded such as using a Terumo Sterile Tubing Welder) to a CF tubing set. After the CF has been completely drained, the “spent medium” bag is removed.

A “Stop Solution” bag and a “Trypsin-EDTA (0.05% Trypsin, 0.53 mM EDTA)” bag are attached (e.g., sterile welded) to the CF tubing set. Trypsin-EDTA approximately 400 mL) is added to the CF via gravity flow. Once the “Trypsin-EDTA” bag has been emptied, the CF is placed into a 37±1° C. and 5±2% CO² incubator for trypsinization.

Each CF is trypsinized for up to 30 minutes (e.g., up to about 15 to about 30 min or any range in-between). During the trypsinization period, the CFs are observed approximately every 8 minutes using an inverted microscope to determine the percentage of cells that have detached. When the percentage of detached cells is estimated to be >about 90%, the trypsinization is stopped by adding 100 mL of media “stop solution” (e.g., DMEM containing 10% FBS) into the CF via gravity flow. The duration of the trypsinization is recorded.

### Step 8. Washing

The trypsinized-stopped cell suspension is drained via gravity flow into a 600 mL transfer pack that is attached to a CF tubing set.

The trypsinized-stopped cell suspension is then washed using the CytoMate®.

### Step 9. Seeding

The hMSCs are counted on a Guava Personal Cytometer™ (Guava Technologies).

An amount of cells (e.g., about 37.5×10⁶ cells or about 37.5×10⁶±about 5%, about 10%, or about 20% cells) are added to 1.5 L culture medium bags on the CytoMate®. The culture medium bags now containing cells are then used to seed a corresponding number of CFs via sterile tubing connections (e.g. at about 5,900 cells/per cm²±about 5%, about 10%, about 15%, or about 20% per cm²). The seeded CFs are then placed in an incubator set at about 37±1° C. and about 5±2% CO², and ambient relative humidity for approximately 14±2 days, with feeds approximately every 3-4 days, as set forth in Example 7.

### Step 10. Second Passage

After approximately 14±2 days in culture, the CF (e.g., six-CF, or eight-CF) P1 cultures are expanded (e.g., into 36 CFs) for the second passage (P2).

The trypsinized-stopped cell suspensions of several CFs can be pooled before washing.

### Step 11. Harvest

After approximately 14±2 days in culture, the cultured hMSCs are harvested (e.g., after about 42 to about 56 days total over two passages). Each CF is processed as set forth above using Plasma-Lyte®. A containing about 1% Human Serum Albumin as a stop solution. The trypsinized-stopped cell suspensions of several CFs can be pooled before washing.

### Example 3. Generation of T-Cell Subpopulations from Peripheral Blood Using Multiple-VAA PepMixes

VAA-specific T-cell lines can be generated from total human blood peripheral mononuclear cells (Step 1). Matured dendritic cells (DCs) are harvested and used as antigen presenting cells (APCs) and peptide-pulsed with a mix of three peptide libraries for IE-1, pp65, EBNA1, LMP1, LMP2, Hexon, Penton, LT, VP-1, MP1, NP1, N, F, U14, and U90 (Step 2). T-cells are initially stimulated using a cytokine mix containing IL-7, IL-12, IL-15, IL-6, and IL-27 (Step 3). Subsequent stimulations (Steps 4 and 5) are performed using irradiated DCs or irradiated phytohemagglutinin (PHA) blasts. Experimental procedures for each of these steps are provided below.

### Step 1. Isolation of Mononuclear Cells

Heparinized peripheral blood is diluted in an equal volume of warm RPMI 1641 (Invitrogen) or PBS. In a 50 mL centrifuge tube, 10-15 mL of Lymphoprep (Axis-Shield) is overlaid with 20-30 mL of diluted blood. The mixture is centrifuged at 800×g for 20 minutes or 400× g for 40 minutes at ambient temperature, ensuring that acceleration and deceleration are set to “1” to prevent disrupting the interface. 1 mL of plasma aliquots are saved and stored at −80° C. The peripheral blood mononuclear cell (PBMC) interface is harvested into an equal volume of RPMI 1640, then centrifuge at 450× g for 10 minutes at ambient temperature, and the supernatant is aspirated. The pellet is loosened and the cells are resuspended in a volume of RPMI 1640 or PBS that yields an estimated 10×10⁶ cells/mL. An aliquot of cells is removed for counting using 50% red cell lysis buffer or Trypan blue and using a hemocytometer. The PBMCs are saved for DC generation using adherence (Step 2 below) and non-adherent cells are cryopreserved for use at initiation.

### Step 2. Dendritic Cell (DC) Generation

PBMCs are centrifuged at 400×g for 5 minutes at ambient temperature, and the supernatant is aspirated. The cells are resuspended at approximately 5×10⁶ cells/mL in CellGenix DC medium containing 2 mM of Glutamax (Invitrogen), and the cells are plated in a 6-well plate (2 mL/well). The PBMC non-adherent fraction is removed after 1-2 hours, and the wells are rinsed with 2-5 mL of CellGenix DC medium or PBS and added to the harvested medium/non-adherent fraction. The non-adherent fraction is saved for later cryopreservation. 2 mL of DC medium containing 1,000 U/mL of IL-4 (R&D Systems) and 800 U/mL GM-CSF (CNMC Pharmacy) is added back to the adherent cells. All surrounding wells are filled with approximately 2 mL of sterile water or PBS to maintain the humidity within the plate, and the plate is placed in the incubator at 37° C. and 5% CO². On day 3 to 4, the cells are fed with 1,000 U/mL IL-4 and 800 U/mL GM-CSF. On day 5 to 6, the DCs are matured in 2 mL/well of DC medium containing lipopolysaccharide (LPS, Sigma) (30 ng/mL), IL-4 (1,000 U/mL), GM-CSF (800 U/mL), TNF-α (10 ng/mL, R&D Systems), IL-6 (100 ng/mL, CellGenix), and IL-1β (10 ng/mL, R&D Systems). The mature DCs are harvested on day 7 to 8 by gentle resuspension. The cells are counted using a hemocytometer. The DCs are transferred to a 15 mL centrifuge tube and centrifuged for 5 minutes at 400× g at ambient temperature. The supernatant is aspirated, and the pellet is resuspended by finger flicking, and 100 μL of appropriate overlapping peptide library mastermix (200 ng/peptide in 200 μL; IE-1, pp65, EBNA1, LMP1, LMP2, Hexon, Penton, LT, VP-1, MP1, NP1, N, F, U14, and U90 overlapping peptide libraries; JPT Peptide Technologies) per 1-5×10⁶ cells is added to the DCs. The DCs and overlapping peptide libraries are mixed and transferred to the incubator. The mixture is incubated for 60-90 minutes at 37° C. and 5% CO2.

### Step 3. T-cell Population Initiation

After pulsing with overlapping peptide libraries, DCs are irradiated at 25 Gy. The DCs are washed with DC medium and centrifuged at 400×g for 5 minutes at ambient temperature. The supernatant is aspirated, and the wash step is repeated twice more. The cells are counted using a hemocytometer. The DCs are resuspended at 2-4×10⁵ cells/mL of CTL medium with 10% human serum (HS, Valley) for initiation. 1 mL of irradiated DCs/well is plated in a 24-well-tissue culture treated plate.

Previously-frozen PBMCs from Step 1 are thawed at 37° C. and diluted in 10 mL of warm medium/1 mL of frozen cells. The PBMCs are centrifuged at 400×g for 5 minutes at ambient temperature and are resuspended in 5-10 mL of medium and a cell count is performed using a hemocytometer. The PBMCs are resuspended at 2×106 cells/mL. DCs and PBMCs are recombined in the plate to stimulate CTL at a 1:10 to 1:5 ratio of DCs:CTL. Cytokines IL-7, IL-15, IL-6, and IL-12 are added to achieve a final concentration of IL-7 (10 ng/mL, R&D Systems)), IL-15 (5 ng/mL, CellGenix), IL-6 (100 ng/mL, CellGenix), and IL-12 (10 ng/mL, R&D Systems). All surrounding wells are filled with approximately 2 mL of PBS to maintain humidity within the plate. The cells are cultured in the incubator at 37° C. and 5% CO² for 7 to 8 days. A one-half medium change is performed on day 4 to 5, with the wells being split 1:1 if nearly confluent.

### Step 4. Second T-cell Stimulation in 24-Well Plate

The second stimulation of T-cells is performed using either Overlapping Peptide Library-Pulsed Autologous DCs (Procedure A) or Overlapping Peptide Library-Pulsed Autologous

Phytohemagglutinin (PHA) Blasts (Procedure B) as antigen presenting cells.

### Procedure A: Stimulation Using Overlapping Peptide Library-Pulsed Autologous DCs as Antigen Presenting Cells (APCs)

After pulsing with the appropriate overlapping peptide libraries (IE-1, pp65, EBNA1, LMP1, LMP2, Hexon, Penton, LT, VP-1, MP1, NP1, N, F, U14, and U90 overlapping peptide libraries; JPT Peptide Technologies), DCs are irradiated at 25 Gy. The DCs are washed with DC medium and are centrifuged at 400×g for 5 minutes at ambient temperature. The supernatant is aspirated and the wash step is repeated twice more. The cells are counted using a hemocytometer. The DCs are resuspended at 0.5-2×10⁵ cells/mL of CTL medium with 10% HS (Valley) for initiation. Plate 1 mL of irradiated DCs/well (0.5-2×10⁵ cells) in a 24-well tissue culture treated plate. T-cells are counted using a hemocytometer. The cells are resuspended at 1×10⁶ cells/mL of T-cell medium supplemented with IL-7 (10 ng/mL final concentration, R&D Systems)) and IL-2 (100 U/mL final concentration, Proleukin) and 1 mL is aliquoted per well of the 24-well plate. The cells are cultured in the incubator at 37° C. and 5% CO2 for 3 to 4 days. The medium is changed with IL-2 (˜100 U/mL final concentration, Proleukin) and cultured for another 3 to 4 days. Cells can be frozen after the second stimulation.

### Procedure B: Stimulation Using Overlapping Peptide Library-Pulsed Autologous Phytohemagglutinin (PHA) Blasts as APCs

Autologous PHA blasts are harvested on day 7 by gentle resuspension, and cells are counted using a hemocytometer. The PHA blasts are transferred to a 15 mL centrifuge tube and are centrifuged for 5 minutes at 400× g at ambient temperature. The Supernatant is aspirated and the pellet is resuspended by finger flicking. 100 μL of appropriate PepMix Mastermix (200 ng/peptide in 200 μL; IE-1, pp65, EBNA1, LMP1, LMP2, Hexon, Penton, LT, VP-1, MP1, NP1, N, F, U14, and U90 overlapping peptide libraries; JPT Peptide Technologies) is added to PHA blasts per 1-10×10⁶ cells. The PHA blasts are incubated for 30-60 minutes. The PHA blasts are resuspended in 5-10 mL of medium and irradiated at 50 Gy (or 100 Gy if used in G-rex). The PHA blasts are washed with CTL medium and centrifuged at 400×g for 5 minutes at ambient temperature. The supernatant is aspirated and the washing step is repeated twice more. A cell count is performed using a hemocytometer. The PHA blasts are resuspended at 0.5×10⁶ cells/mL of CTL medium to re-stimulate T-cells at an approximate ratio of 1:1 PHA blasts:T-cell. The T-cells are counted using a hemocytometer. The T-cells are resuspended at 0.5×10⁶ cells/mL of CTL medium supplemented with IL-7 (100 ng/mL final concentration; R&D Systems) and IL-2 (100 U/mL final concentration; Proleukin). One well of only PHA blasts is maintained as an irradiation control. The cells are cultured in the incubator at 37° C. and 5% CO² for 3 to 4 days. The medium is changed with IL-2 (100 U/mL final concentration; Proleukin) and the cells are cultured for another 3 to 4 days.

### Step 5. Third T-cell Stimulation in G-Rex10 Using PHA Blasts as APCs

Autologous PHA blasts are harvested on day 7 by gentle resuspension, and cells are counted using a hemocytometer. The PHA blasts are transferred to a 15 mL centrifuge tube and are centrifuged for 5 minutes at 400× g at ambient temperature. The supernatant is aspirated, and the pellet is resuspended by finger flicking. 100 μL of appropriate overlapping peptide library mastermix (200 ng/peptide in 200 μL; IE-1, pp65, EBNA1, EBNA2, LMP1, LMP2, Hexon, Penton, LT, VP-1, MP1, NP1, N, F, U14, and U90 overlapping peptide libraries; JPT Peptide Technologies) is added to PHA blasts per 1-10×10⁶ cells, and the PHA blasts are incubated for 30-60 minutes. The PHA blasts are resuspended in 5-10 mL of medium and irradiated at 50 Gy (or 100 Gy if used in G-Rex). Alternatively, PHA blasts are peptide-pulsed with single peptide libraries of selected viral antigens to generate multiple subpopulations of peptide-pulsed PHA blasts. The PHA blasts are washed with CTL medium and are centrifuged at 400×g for 5 minutes at ambient temperature. The supernatant is aspirated, and the washing step is repeated twice more. Cells are counted using a hemocytometer. The PHA blasts are resuspended at 0.5×106 cells/mL of CTL medium to re-stimulate T-cells at an approximate ratio of 1:1 PHA blasts. 10 mL of cell suspension is added in the G-Rex10 and 1 mL/well (0.5×10⁶ PHA blasts) in the 24-well control plate. The T-cells were counted using a hemocytometer. The T-cells are resuspended at 05×10⁶ cells/mL of CTL medium, and 10 mL (5×10⁶ CTLs) was added in the G-Rex10 and 1 mL/well (0.5×10⁶ CTLs) in the 24-well control plate. The medium was supplemented with IL-7 (10 ng/mL final concentration; R&D Systems) and IL-2 (100 U/mL final concentration; Proleukin), and the cells are cultured in the incubator at 37° C. and 5% CO² for 3 to 4 days. One well of the 24 well plate is left with PHA blasts only as an irradiation control. The medium is changed with IL-2 (100 U/mL final concentration; Proleukin), and the cells are cultured for an additional 3 to 4 days.

This specification has been described with reference to embodiments of the invention. The invention has been described with reference to assorted embodiments, which are illustrated by the accompanying Examples. The invention can, however, be embodied in different forms and should not be construed as limited to the embodiments set forth herein. Given the teaching herein, one of ordinary skill in the art will be able to modify the invention for a desired purpose and such variations are considered within the scope of the invention.

1. A cell composition comprising:
one or more primed and expanded T-cell subpopulations having specificity for one or more tumor associated antigens;
(ii) one or more primed and expanded T-cell subpopulations having specificity for one or more viral associated antigens; and
(iii) one or more mesenchymal stem cell (MSC) subpopulations.

2. (canceled)

3. The cell composition of claim 1, wherein the one or more tumor associated antigens are selected from the group consisting of WT1, PRAME, Survivin, NY-ESO-1, MAGE-A3, MAGE-A4, Pr3, Cyclin A1, SSX2, Neutrophil Elastase (NE), and combination thereof.

4. (canceled)

5. The cell composition of claim 1, wherein the one or more virus associated antigens are selected from the group consisting of immediate-early protein 1 (IE-1), immediate-early protein 2 (IE-2), 65 kDa phosphoprotein (pp65), EBNA-leader protein (EBNA-LP), EBNA1, EBNA2, EBNA3a, EBNA3b, EBNA3c, latent membrane protein 1 (LMP1), latent membrane protein 2 (LMP2), envelope glycoprotein GP350/GP340, BARF1 mRNA export factor EB2 (BMLF1), DNA polymerase processivity factor (BMRF1), trans-activator protein (BZLF1), hexon protein of Human adenovirus 3 (HAdV-3), penton protein of Human adenovirus 5 (HAdV-5), capsid protein VP-1, capsid protein VP-2, large T antigen, small T antigen, U14, U54, U90, fusion glycoprotein (F), major surface glycoprotein G, small hydrophobic protein (SH), nucleocapsid (N) protein, matrix protein (MP) 1, matrix protein (MP) 2, nucleocapsid protein (NP) 1, neuroaminidase, hemagglutinin (HA), protein E4, protein E5, protein E6, protein E7, late major capsid protein (L) 1, replication protein E1, replication protein E2, envelope glycoprotein gp160 (Env), Gag polyprotein, Nef protein, Pol polyprotein, and a combination thereof.

6. (canceled)

7. The cell composition of claim 1, wherein the one or more virus associated antigens comprise:
(a) a viral associated antigen selected from the group consisting of IE-1, pp65, and a combination thereof;
(b) a viral associated antigen selected from the group consisting of EBNA1, EBNA2 LMP1, LMP2, BARF1, BZLF1, and a combination thereof;
(c) a viral associated antigen selected from the group consisting of Hexon, Penton, and a combination thereof;
(d) a viral associated antigen selected from the group consisting of LT, VP-1, and a combination thereof;
(e) a viral associated antigen selected from the group consisting of MP1, NP1, and a combination thereof;
(f) a viral associated antigen selected from the group consisting of N, F, and a combination thereof; and
(g) a viral associated antigen selected from the group consisting of U14, U90, and a combination thereof.

8. The cell composition of claim 1, wherein the MSC subpopulation is from bone marrow or cord blood, and wherein the MSC subpopulation optionally comprises greater than 95% of cells having a positive antigen expression pattern CD29, CD105, CD73, and CD90, and less than 2% of cells having an antigen expression pattern CD45, CD34, CD3, CD14, CD19, and HLA-DR.

9. (canceled)

10. The cell composition of claim 1, wherein:
(a) the T-cell subpopulations of (i) are from an allogeneic donor; and/or
(b) the T-cell subpopulations of (ii) are from an allogeneic donor.

11-15. (canceled)

16. A method of treating a malignancy or tumor in a subject in need thereof, comprising administering an effective amount of the cell composition of claim 1 to the subject.

17. The method of claim 16, wherein:
the malignancy is a hematological malignancy or a solid tumor.

18-20. (canceled)

21. The method of claim 16, wherein the subject is receiving or has received an hematopoietic stem cell transplantation (HSCT).

22. A cell composition comprising:
(i) one or more primed and expanded T-cell subpopulations having specificity for one or more viral associated antigens; and
(ii) one or more mesenchymal stem cell (MSC) subpopulations.

23. The cell composition of claim 22, wherein the one or more virus associated antigens are selected from the group consisting of immediate-early protein 1 (IE-1), immediate early protein 2 (IE-2), 65 kDa phosphoprotein (pp65), EBNA-leader protein (EBNA-LP), EBNA1, EBNA2, EBNA3a, EBNA3b, EBNA3c, latent membrane protein 1 (LMP1), latent membrane protein 2 (LMP2); envelope glycoprotein GP350/GP340, BARF1 mRNA export factor EB2 (BMLF1), DNA polymerase processivity factor (BMRF1), trans-activator protein (BZLF1), hexon protein of Human adenovirus 3 (HAdV-3), penton protein of Human adenovirus 5 (HAdV-5), capsid protein VP-1, capsid protein VP-2, large T antigen, small T antigen, U14, U54, U90, fusion glycoprotein (F), major surface glycoprotein G, small hydrophobic protein (SH), nucleocapsid (N) protein, matrix protein (MP) 1, matrix protein (MP) 2, nucleocapsid protein (NP) 1, neuroaminidase, hemagglutinin (HA), protein E4, protein E5, protein E6, protein E7, late major capsid protein (L) 1, replication protein E1, replication protein E2, envelope glycoprotein gp160 (Env), Gag polyprotein, Nef protein, Pol polyprotein, and a combination thereof.

24. (canceled)

25. The cell composition of claim 22, wherein the one or more virus associated antigens comprise:
(a) a viral associated antigen selected from the group consisting of IE-1, pp65, and a combination thereof;
(b) a viral associated antigen selected from the group consisting of EBNA1, EBNA2, LMP1, LMP2, BARF1, BZLF1, and a combination thereof;
(c) a viral associated antigen selected from the group consisting of Hexon, Penton, and a combination thereof;
(d) a viral associated antigen selected from the group consisting of LT, VP-1, and a combination thereof;
(e) a viral associated antigen selected from the group consisting of MP1, NP1, and a combination thereof;
(f) a viral associated antigen selected from the group consisting of N, F, and a combination thereof; and
(g) a viral associated antigen selected from the group consisting of U14, U90, and a combination thereof.

26. The cell composition of claim 22, wherein the MSC subpopulation is from bone marrow or cord blood, and wherein the MSC subpopulation optionally comprises greater than 95% of cells having a positive antigen expression pattern CD29, CD105, CD73, and CD90, and less than 2% of cells having an antigen expression pattern CD45, CD34, CD3, CD14, CD19, and HLA-DR.

27. (canceled)

28. The cell composition of claim 22, wherein:
(a) the T-cell subpopulations are from an allogeneic donor and/or;
(b) the T-cell subpopulations are from cord blood.

29-30. (canceled)

31. A method of treating a non-malignant indication in a subject in need thereof, comprising administering an effective amount of the cell composition of claim 22 to the subject.

32. The method of claim 31, wherein:
the non-malignant indications is an autoimmune disease, a metabolic disorder, or a primary immune deficiency disorder.

33-35. (canceled)

36. The method of claim 31, wherein the subject is receiving or has received an hematopoietic stem cell transplantation (HSCT).

37. A method of treating a malignancy or tumor in a subject in need thereof, comprising:
(i) determining a human leukocyte antigen (HLA) subtype of the subject;
(ii) diagnosing a malignancy or tumor type of the subject;
(iii) identifying two or more tumor associated antigens associated with the tumor type for targeting with a tumor associated antigen (TAA)-specific T-cell subpopulation;
(iv) selecting at least one banked T-cell subpopulation for each targeted TAA, wherein the T-cell subpopulation selected has at least one shared allele or allele combination with the targeted TAA;
(v) identifying one or more viral associated antigens for targeting with viral associated antigen (VAA)-specific T-cell subpopulations;
(vi) selecting at least one banked T-cell subpopulation for each targeted VAA, wherein the T-cell subpopulation selected has at least one shared allele or allele combination with the targeted VAA;
(vii) selecting at least one banked mesenchymal stem cell (MSC) population;
(viii) combining each selected banked T-cell subpopulation and MSC population to create a cell composition; and
(ix) administering an effective amount of the cell composition to the subject.

38. A method of selecting a therapy for treating a malignancy or tumor in a subject in need thereof, comprising:
(i) determining a human leukocyte antigen (HLA) subtype of the subject;
(ii) determining a tumor associated antigen (TAA) expression profile of the malignancy or tumor;
(iii) identifying two or more tumor associated antigens expressed by the tumor for targeting with TAA-specific T-cell subpopulations;
(iv) selecting one banked T-cell subpopulation for each targeted TAA, wherein the T-cell subpopulation selected has at least one shared allele or allele combination with the targeted TAA;
(v) identifying one or more viral associated antigens for targeting with viral associated antigen (VAA)-specific T-cell subpopulations;
(vi) selecting at least one banked T-cell subpopulation for each targeted VAA, wherein the T-cell subpopulation selected has at least one shared allele or allele combination with the targeted VAA; and
(vii) selecting at least one banked mesenchymal stem cell (MSC) population.

39. A method of treating a non-malignant indication in a subject in need thereof, comprising:
(i) determining a human leukocyte antigen (HLA) subtype of the subject;
(ii) identifying one or more viral associated antigens for targeting with viral associated antigen (VAA)-specific T-cell subpopulations;
(iii) selecting at least one banked T-cell subpopulation for each targeted VAA, wherein the T-cell subpopulation selected has at least one shared allele or allele combination with the targeted VAA;
(iv) selecting at least one banked mesenchymal stem cell (MSC) population;
(v) combining each selected banked T-cell subpopulation and MSC population to create a T-cell/mesenchymal stem cell composition; and
(vi) administering an effective amount of the T-cell/mesenchymal stem cell composition to the subject.

40. (canceled)

41. A bank of T-cell subpopulations and mesenchymal stem cells (MSC) subpopulations comprising:
(i) one or more primed and expanded T-cell subpopulations having specificity for one or more tumor associated antigens;
(ii) one or more primed and expanded T-cell subpopulations having specificity for one or more viral associated antigens; and
(iii) one or more mesenchymal stem cell (MSC) subpopulations.

42. The bank of T-cell subpopulations and MSC subpopulations of claim 41, wherein:
the T-cell subpopulations of (i) or (ii) are from an allogeneic donor.

44-45. (canceled)

46. The T-cell composition of claim 10, wherein each of the T-cell subpopulations is primed and expanded using a group of peptides comprising peptides specific to each tumor associated antigen that are HLA-restricted to at least one of the donor's HLA-A alleles, one of the donor's HLA-B allele, and one of the donor's HLA-DR alleles.

47. (canceled)

48. The T-cell composition of claim 46, wherein:
(a) the HLA-A alleles are selected from a group comprising HLA-A*01, HLA-A*02:01, HLA-A*03, HLA-A*11:01, HLA-A*24:02, HLA-A*26, and HLA-A*68:01;
(b) the HLA-B alleles are selected from a group comprising HLA-B*07:02, HLA-B*08, HLA-B*15:01 (B62), HLA-B*18, HLA-B*27:05, HLA-B*35:01, and HLA-B*58:02; or
(c) the HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, and HLA-DRB1*1501 (DR2b).

49-66. (canceled)

67. The T-cell composition of claim 28, wherein each of the T-cell subpopulations is primed and expanded using a group of peptides comprising peptides specific to each viral associated antigen that are HLA-restricted to at least one of the donor's HLA-A alleles, one of the donor's HLA-B allele, and one of the donor's HLA-DR alleles.

68. (canceled)

69. The T-cell composition of claim 67, wherein:
(a) the HLA-A alleles are selected from a group comprising HLA-A*01, HLA-A*02:01, HLA-A*03, HLA-A*11:01, HLA-A*24:02, HLA-A*26, and HLA-A*68:01;
(b) the HLA-B alleles are selected from a group comprising HLA-B*07:02, HLA-B*08, HLA-B*15:01 (B62), HLA-B*18, HLA-B*27:05, HLA-B*35:01, and HLA-B*58:02, and/or
(c) the HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, and HLA-DRB1*1501 (DR2b).

70-83. (canceled)
